(19) United States
(12) Patent Application Publication
(10) Pub. No.: US 2025/0387484 A1
(43) Pub. Date: Dec. 25, 2025

(54) CD25-SPECIFIC ANTIBODIES AND USES THEREOF

(71) Applicant: The U.S.A., as represented by the Secretary, Department of Health and Human Services, Bethesda, MD (US)

(72) Inventors: Hisataka Kobayashi, Laurel, MD (US); Peter L. Choyke, Rockville, MD (US)

(73) Assignee: The U.S.A., as represented by the Secretary, Department of Health and Human Services, Bethesda, MD (US)

(21) Appl. No.: 18/878,317

(22) PCT Filed: Jun. 30, 2023
(86) PCT No.: PCT/US2023/069461

§ 371 Date: Dec. 23, 2024

Related U.S. Application Data
(60) Provisional application No. 63/357,215, filed on Jun. 30, 2022.

(51) Int. Cl. A61K 41/00; A61K 45/06; A61K 47/68; A61P 35/00; C07K 16/28

(52) U.S. Cl. CPC A61K 41/0071; A61K 45/06; A61K 47/6817; A61K 47/6849; A61P 35/00; C07K 16/2866; A61K 2121/00; C07K 2317/52; C07K 2317/565; C07K 2317/71; C07K 2317/94

(57) ABSTRACT

Monoclonal antibodies that specifically bind CD25, as well as conjugates of the anti-CD25 antibodies, are described. The CD25-specific monoclonal antibodies and conjugates thereof do not block binding of IL-2 to CD25 and induce little to no antibody-dependent cellular cytotoxicity (ADCC). The anti-CD25 antibodies and conjugates can be used, for example, to target photoimmunotherapy to T regulatory (Treg) cells in tumor beds to enhance the local host immune response to the tumor.

### CROSS REFERENCE TO RELATED APPLICATIONS

This application claims the benefit of U.S. Provisional Application No. 63/357,215, filed Jun. 30, 2022, which is herein incorporated by reference in its entirety.

### ACKNOWLEDGMENT OF GOVERNMENT SUPPORT

This invention was made with government support under ZIA BC 010656 awarded by the National Institutes of Health. The government has certain rights in the invention.

### FIELD

This disclosure concerns monoclonal antibodies that specifically bind CD25 (IL-2Rα), but do not significantly block binding of interleukin-2 (IL-2) to CD25 and induce little to no antibody-dependent cellular cytotoxicity (ADCC). This disclosure further concerns use of the CD25-specific antibodies, such as for targeting CD25-expressing T regulatory (Treg) cells in tumor beds.

### INCORPORATION OF ELECTRONIC SEQUENCE LISTING

The electronic sequence listing, submitted herewith as an XML file named 4239-107471-02.xml (245,918 bytes), created on Jun. 20, 2023, is herein incorporated by reference in its entirety.

### BACKGROUND

Near infrared photoimmunotherapy (NIR-PIT) is a cancer treatment that employs a targeted monoclonal antibody-photo-absorber conjugate. Following antibody localization of the antibody-photo-absorber conjugate to a tumor cell surface antigen, NIR light is used to induce highly selective cytolysis.

NIR-PIT induces rapid, necrotic cell death that yields innate immune ligands that activate dendritic cells (DCs), consistent with immunogenic cell death (ICD). A description of how NIR-PIT kills tumor cells is described in Sato et al. (ACS Cent. Sci. 4:1559-69, 2018). Briefly, following binding of the antibody-photo-absorber conjugate to its target, activation by NIR light causes physical changes in the shape of antibody-antigen complexes that induce physical stress within the cellular membrane, leading to increases in transmembrane water flow that eventually lead to cell bursting and necrotic cell death.

CD25 is a type I transmembrane protein present on activated T cells, activated B cells, some thymocytes, myeloid precursors, and oligodendrocytes. T regulatory (Treg) cells, which also express CD25, play an important role in immune suppression by permitting tumors to evade immune surveillance.

Although Treg cell depletion can result in tumor regression, systemic depletion of Treg cells can also induce autoimmune adverse events (Okada et al., Bioconjugate Chem 30(10):2624-2633, 2019). Thus, a need remains for methods of targeted depletion of CD25-positive Treg cells, such as in tumor beds, without negative systemic side effects.

### SUMMARY

Disclosed herein is a group of humanized anti-CD25 monoclonal antibodies that specifically bind CD25 (also known as the interleukin-2 receptor alpha chain—IL-2Rα). The disclosed antibodies do not significantly block binding of IL-2 to CD25 and induce little to no ADCC. In some examples, the disclosed CD25 antibodies have sub-nM affinity for CD25. The anti-CD25 antibodies can be used, for example, to target photoimmunotherapy to Treg cells in tumor beds, thereby enhancing the host immune response to tumors. Thus, in some examples, the disclosed CD25 antibodies are conjugated to IR700 (CD25 antibody-IR700 conjugate), and can be used to treat tumors, such as an immunogenic tumor. In some examples, the treatment is localized, and not systemic.

Provided herein are monoclonal antibodies that specifically bind CD25. The disclosed antibodies include at least a variable heavy (VH) domain and variable light (VL) domain. In some aspects, the VH domain complementarity determining region 1 (CDR), CDR2 and CDR3 sequences respectively include SEQ ID NO: 43, SEQ ID NO: 44 and SEQ ID NO: 45, and the VL domain CDR1, CDR2 and CDR3 sequences respectively include SEQ ID NO: 46, SEQ ID NO: 47 and SEQ ID NO: 48. In some examples, the VH domain and the VL domain CDR sequences are the CDR sequences of antibody 14564, 14569 or 14572. In some aspects, the monoclonal antibody further includes a constant region, such as a heavy chain constant region and/or a light chain constant region. In some aspects, the antibody does not significantly inhibit binding of IL-2 to CD25 and/or induces little to no ADCC.

Also provided are conjugates that include a CD25-specific monoclonal antibody disclosed herein and a photon absorber (referred to herein as “CD25 antibody-photon absorber conjugate” or “CD25 antibody-IR700 conjugate”), an effector molecule, or a detectable label. In some aspects, the photon absorber is IR700.

Compositions that include a pharmaceutically acceptable carrier and a disclosed antibody or conjugate are also provided.

Further provided are nucleic acid molecules that encode a CD25-specific monoclonal antibody disclosed herein or encode a VH domain or a VL domain thereof. Vectors that include a disclosed nucleic acid molecule, and isolated cells that include a vector or nucleic acid molecule disclosed herein are also provided.

Also provided are kits that include a monoclonal antibody, conjugate, composition, nucleic acid molecule, vector or isolated cell disclosed herein. In some aspects, the kit further includes buffer, cell culture media, transfection reagent(s), one or more check point inhibitors, one or more immunotherapies, one or more additional anti-cancer reagents (such as a chemotherapeutic agent or biologic) and/or instructional materials. In one example, the kit further includes a tumor-specific antibody-IR700 conjugate (e.g., cetuximab-IR700 or any other provided in Table 1).

Further provided are methods of treating cancer in a subject. In some aspects, the method includes administering to the subject a therapeutically effective amount of CD25 antibody-IR700 conjugate (wherein the CD25 antibody is one disclosed herein) and subsequently irradiating the subject and/or irradiating cancer cells in the subject at a wavelength of 660 to 740 nm and at a dose of at least 1 J/cm² or at least 4 J/cm². In some examples, the method further includes administering a second monoclonal antibody conjugated to IR700, wherein the second monoclonal antibody specifically binds a tumor antigen expressed by cells of the cancer (e.g., see Table 1).

The foregoing and other features of this disclosure will become more apparent from the following detailed description of several aspects which proceeds with reference to the accompanying figures.

### BRIEF DESCRIPTION OF THE DRAWINGS

FIG. 1 is a series of graphs showing binding of antibodies 14564, 14569 and 14572 to cynomolgus CD25 by surface plasmon resonance. Parental antibody VH2+VL4 was used as a control. Antibodies 14564, 14569 and 14572 bound CD25 with high affinity.

FIG. 2 is a graph showing binding of antibodies 14564, 14569 and 14572 to CD25-expressing KIT-225 cells (a human T cell line) by FACS analysis. Anti-CD25 antibody basiliximab was used as a positive control. Binding affinity of antibodies 14564, 14569 and 14572 to CD25 was similar to basiliximab.

FIG. 3 is a graph of showing binding affinity of antibodies 14564, 14569 and 14572 to CD25-expressing KIT-225 cells by FACS analysis. Anti-CD25 antibody basiliximab was used as a positive control. Also shown is the calculated effective concentration 50 (EC50) of each antibody.

FIGS. 4A-4B are graphs showing results of a cytotoxicity assay to determine whether antibodies 14564, 14569 and 14572 induce antibody-dependent cellular cytotoxicity (ADCC) of CD25-expressing cells. Basiliximab was used as a positive control. Only basiliximab induced ADCC of target cells.

FIG. 5A-5C show results of studies to evaluate targeting photoimmunotherapy (PIT) to CD25-expressing cells. (FIG. 5A) Binding of anti-hCD25-IR700 to KIT-225 cells. KIT-225 cells (0.5×10⁶) were incubated with 1 g of anti-hCD25 antibody 14564 conjugated to IR700 (anti-hCD25-IR700). For the blocking control (“Blocking+Anti-hCD25-IR700”), cells were incubated with 10 g non-conjugated antibody prior to anti-hCD25-IR700 incubation. Cells were stained with viability dye and analyzed by fluorescence-activated cells sorting (FACS). Unstained cells were used as a negative control. (FIG. 5B) Representative flow cytometry plots showing live/dead staining after PIT (0J=no NIR light; 5J=irradiated with 5 J/cm² NIR light; 20J=irradiated with 20 J/cm² NIR light). (FIG. 5C) Cell viability after hCD25-targeted PIT. KIT-225 cells (0.5×10⁶) were incubated with 1 μg of anti-hCD25-IR700 conjugate and 0, 5, 20 or 50 J/cm² NIR light was applied at 150 mW/cm². Cells were then stained with viability dye and analyzed by FACS. Results are shown as mean+SEM of five replicates.

FIG. 6 Anti-CD25 antibodies do not block binding of IL-2 to CD25. KIT-225 cells underwent TL-2 fasting for five days prior to performing a proliferation assay. Fifty thousand fasted cells in culture medium were pre-incubated with 0, 5 or 10 μg/ml of 14564, 14569, 14572 or the parental anti-CD25 antibody for 30 minutes at 37° C., and then cultured with or without 50 pM human IL-2 for 1.5 days. Cells cultured without antibody and with or without IL-2 were included as controls. ³H-thymidine (37 kBq) was pulsed, and 4 hours later, the cells were harvested, and the incorporated activity was measured using a β-counter. The results show that antibodies 14564, 14569, 14572 do not inhibit IL-2-induced proliferation of KIT-225.

FIGS. 7A-7C: hCD25-targeted PIT kills human Treg cells. Splenocytes obtained from MDA-MD 231 tumor-bearing hCD34-NSG humanized mice were incubated with 5 μg IR700-conjugated anti-CD25 antibody 14564 and then irradiated with 50 J/cm² NIR light at 150 mW/cm². Splenocytes cultured in the absence of antibody-IR700 conjugate and not irradiated were used as a control. The percentage of CD25+ Treg cells was evaluated by flow cytometry. (FIG. 7A) Representative dot plot showing CD25+ Treg cells for control (top) and CD25 PIT (bottom) splenocytes. (FIG. 7B) Graph showing the percentage of CD25+ Treg cells present in control and CD25 PIT splenocyte samples (n=5). (FIG. 7C) Gating strategy.

### SEQUENCE LISTING

The nucleic acid and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand. In the accompanying sequence listing: SEQ ID NOs: 1-42 are amino acid sequences of anti-CD25 monoclonal antibody VH and VL domains.
SEQ ID NOs: 43-48 are consensus amino acid sequences of anti-CD25 monoclonal antibody CDRs.
SEQ ID NOs: 49-56 are amino acid sequences of exemplary anti-CD25 monoclonal antibody CDRs.
SEQ ID NO: 57 is the amino acid sequence of an exemplary leader sequence.
SEQ ID NO: 58 is the amino acid sequence of a modified human IgG4 heavy chain constant region.
SEQ ID NO: 59 is the amino acid sequence of a human Ig kappa light chain constant region.
SEQ ID NOs: 60-101 are amino acid sequences of anti-CD25 antibody heavy and light chains.
SEQ ID NOs: 102-143 are nucleic acid sequences encoding anti-CD25 VH and VL domains.
SEQ ID NOs: 144-145 are nucleic acid sequences of exemplary leader sequences.
SEQ ID NO: 146 is a nucleic acid sequence encoding a modified human IgG4 heavy chain constant region.
SEQ ID NO: 147 is a nucleic acid sequence encoding a human Igκ light chain constant region.
SEQ ID NOs: 148-189 are nucleic acid sequences encoding anti-CD25 monoclonal antibody heavy and light chains.

### DETAILED DESCRIPTION

### I. Introduction

Therapeutic monoclonal antibodies (mAbs) are typically injected systemically to block specific biological functions (such as by binding to ligand binding sites of specific receptors) or to kill cells bound to the antibody by attracting immune cells to the target, leading to antibody dependent cell-mediated cytotoxicity (ADCC) or complement dependent cytotoxicity (CDC). While there has been some success with these strategies, pharmacologic doses of mAbs can also cause unexpected and undesirable side effects. The present disclosure describes the development of humanized anti-CD25 antibodies that are used as a localized delivery platform (in contrast to a systemic therapy), with no significant biologic or cytotoxic properties.

Two anti-CD25 antibodies developed for systemic therapy have previously been commercialized, daclizimab and basiliximab. Both antibodies block the IL-2a receptor, thus interfering with the maturation of activated effector CD8⁺ T cells. These two antibodies were developed for use as immunosuppressive agents against auto-immune diseases, such as multiple sclerosis (MS) in the case of daclizimab, and organ transplantation rejection in the case of basiliximab. However, because daclizimab and basiliximab also block proliferation of Treg cells, a potential side effect is autoimmune syndromes. In fact, daclizimab was withdrawn from the market in 2018 for complications related to inflammation of the brain. Basiliximab continues to be used as induction therapy to reduce immune responses to organ transplantation.

Photoimmunotherapy (PIT) is a cancer treatment that relies on a completely different principle than surgery, drug therapy (including immunotherapy) or radiation. A particular infrared dye (e.g., IR700) is conjugated to a target-specific antibody, thereby forming an antibody-photoabsorbing conjugate (APC). When the APC is injected systemically, it binds to a target receptor on the tumor cell membrane, a process that typically takes about 24 hours in humans. Near infrared (NIR) light is then applied to the tumor. This causes photochemical changes in the APC that result in damage to the portion of the cell membrane which is bound to the APC. By causing numerous such micro-perforations of the cell membrane, cells become irreversibly damaged and die. Within minutes of NIR light exposure, target cells swell, bleb and burst, which causes cell death. This type of treatment completely avoids the side effects of systemic mAb therapy.

The present disclosure describes humanized CD25-specific mAbs that are suitable for NIR-PIT because they do not possess any specific biological functions in the absence of conjugation to an effector molecule (such as IR700). CD25 is the IL-2a receptor, which is found on T regulatory (Treg) cells and activated effector T cells, among other immune cell types. Antibodies to CD25 can be blocking or non-blocking with respect to IL-2, a cytokine that causes cellular proliferation in immune cell subtypes. Non IL-2-blocking CD25-specific antibodies bind to CD25 without interfering with IL-2 binding and therefore have no direct pharmacokinetic effect. Thus, an APC directed toward the CD25 protein can selectively deplete Treg cells within the confines of the tumor, without having a systemic effect.

The present disclosure provides humanized anti-CD25 antibodies with at least the following features: (i) high affinity binding to CD25 that is not impaired by IR700 conjugation; (ii) little or no IL-2 blocking (such as non-IL-2 blocking); and (iii) little or no ADCC activity (such as a lack of ADCC activity). Based on these features, the disclosed anti-CD25 antibodies provide a superior delivery platform for effector molecules, including IR700, such as for the treatment of cancer.

### II. Abbreviations

ADCC antibody-dependent cellular cytotoxicity
CDR complementarity determining region
DC dendritic cell
FACS fluorescence-activated cell sorting
FLT fluorescence lifetime
J/cm² Joules per centimeter squared
IL-2 interleukin-2
IL-2R interleukin-2 receptor
LED light emitting diode
mAb monoclonal antibody
NIR near infrared light
PIT photoimmunotherapy
RU response unit
Treg T regulatory
VH variable heavy
VL variable light

### III. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. The sequences associated with all GenBank® Accession numbers referenced herein are incorporated by reference for the sequence available on Jun. 30, 2022. In order to facilitate review of the various aspects of the disclosure, the following explanations of specific terms are provided:

Abscopal effect: The treatment of a tumor, such as a metastasis, in a part of the body not the direct target of the local therapy (e.g., NIR-PIT). For example, irradiation of a particular tumor with NIR light in combination with appropriate antibody-IR700 conjugate(s) can reduce the size of a different tumor (e.g., a metastasis), not irradiated with NIR light. The non-irradiated/distant tumor in some examples (for example in a human) is at least 3 inches away from the tumor treated with NIR light, such as at least 4 inches, at least 5 inches, at least 10 inches, at least 12 inches, at least 18 inches, or at least 24 inches away from the tumor treated with NIR light.

Administration: To provide or give a subject an agent, such as an antibody, antibody-IR700 conjugate and/or an immunomodulator, by any effective route. Exemplary routes of administration include, but are not limited to, topical, systemic or local injection (such as subcutaneous, intramuscular, intradermal, intraperitoneal, intratumoral, intraosseous, intraprostatic, and intravenous), oral, ocular, sublingual, rectal, transdermal, intranasal, vaginal, and inhalation routes. In one example, administration is intravenous. In one example, administration is intraperitoneal. In one example, administration is not systemic. In one example, administration is local to a tumor or cancer.

Antibody: A polypeptide ligand comprising at least one variable region that recognizes and binds (such as specifically recognizes and specifically binds) an epitope of an antigen, such as a tumor-specific protein or a protein specifically expressed on immune cells, such as T cells (e.g., T regulatory cells). Mammalian immunoglobulin molecules are composed of a heavy (H) chain and a light (L) chain, each of which has a variable region, termed the variable heavy (V_{H}) region and the variable light (V_{L}) region, respectively. Together, the VH region and the V_{L} region are responsible for binding the antigen recognized by the antibody. Typically, a naturally occurring mammalian immunoglobulin has heavy chains and light chains interconnected by disulfide bonds. There are two types of light chain, lambda (λ) and kappa (κ).

There are five main heavy chain classes (or isotypes) of mammalian immunoglobulin, which determine the functional activity of an antibody molecule: IgM, IgD, IgG, IgA and IgE. Some mammals, such as camels, alpacas, and llamas, have heavy-chain antibodies that lack a light chain. Antibody isotypes not found in mammals include IgX, IgY, IgW and IgNAR. IgY is the primary antibody produced by birds and reptiles, and has some functionality similar to mammalian IgG and IgE. IgW and IgNAR antibodies are produced by cartilaginous fish such as sharks, while IgX antibodies are found in amphibians. IgNAR antibodies are heavy-chain antibodies.

Antibodies, such as those in an antibody-IR700 conjugate, include intact immunoglobulins and the variants and portions of antibodies, such as Fab fragments, Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), and heteroconjugate antibodies (such as, bispecific antibodies). See also, Pierce Catalog and Handbook, 1994-1995 (Pierce Chemical Co., Rockford, IL); Kuby, J., Immunology, 3^{rd} Ed., W. H. Freeman & Co., New York, 1997.

Antibody variable regions contain “framework” regions and hypervariable regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of well-known numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., JMB 273,927-948, 1997; the “Chothia” numbering scheme), Kunik et al. (see Kunik et al., PLoS Comput Biol 8:e1002388, 2012; and Kunik et al., Nucleic Acids Res 40:W521-524, 2012; “Paratome CDRs”) and the ImMunoGeneTics (IMGT) database (see, Lefranc, Nucleic Acids Res 29:207-9, 2001; the “IMGT” numbering scheme). The Kabat, Paratome and IMGT databases are maintained online. In addition, the AbRSA tool can be used to determine the CDR boundaries according to Kabat, IMGT or Chothia (online at alignedr.labshare.cn/aligncdr/abrsa.php).

A “single-domain antibody” refers to an antibody having a single domain (a variable domain) that is capable of specifically binding an antigen, or an epitope of an antigen, in the absence of an additional antibody domain. Single-domain antibodies include, for example, VH domain antibodies, V_{NAR} antibodies, camelid V_{H}H antibodies, and VL domain antibodies. V_{NAR} antibodies are produced by cartilaginous fish, such as nurse sharks, wobbegong sharks, spiny dogfish and bamboo sharks, which produce heavy-chain antibodies (IgNARs). Camelid V_{H}H antibodies are produced by several species including camel, llama, alpaca, and guanaco, which produce heavy chain antibodies that are naturally devoid of light chains.

A “monoclonal antibody” is an antibody produced by a single clone of lymphocytes or by a cell into which the coding sequence of a single antibody has been transfected. Monoclonal antibodies are produced by known methods. Monoclonal antibodies include humanized monoclonal antibodies.

A “chimeric antibody” has framework residues from one species, such as human, and CDRs (which generally confer antigen binding) from another species.

A “humanized” antibody is an immunoglobulin including a human framework region and one or more CDRs from a non-human (for example a mouse, rabbit, rat, shark, camel or synthetic) immunoglobulin. The non-human immunoglobulin providing the CDRs is termed a “donor,” and the human immunoglobulin providing the framework is termed an “acceptor.” In one aspect, all CDRs are from the donor immunoglobulin in a humanized immunoglobulin. Constant regions need not be present, but if they are, they are substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A humanized antibody binds to the same antigen as the donor antibody that provides the CDRs. Humanized or other monoclonal antibodies can have additional conservative amino acid substitutions which have substantially no effect on antigen binding or other immunoglobulin functions.

An antibody that “specifically binds” an antigen refers to the ability of individual antibodies to specifically immunoreact with an antigen, such as a tumor-specific antigen, relative to binding to unrelated proteins, such as non-tumor proteins, for example β-actin. For example, an CD25-specific binding agent binds substantially only the CD25 protein in vitro or in vivo. As used herein, the term “tumor-specific binding agent” includes tumor-specific antibodies (and fragments thereof) and other agents that bind substantially only to a tumor-specific protein in that preparation.

The binding is a non-random binding reaction between an antibody molecule and an antigenic determinant, such as CD25. The desired binding specificity is typically determined from the reference point of the ability of the antibody to differentially bind the target antigen and an unrelated antigen, and therefore distinguish between two different antigens, particularly where the two antigens have unique epitopes. An antibody that specifically binds to a particular epitope is referred to as a “specific antibody.”

Antibody-IR700 conjugate: A molecule that includes both an antibody, such as a CD25-specific antibody or a tumor-specific antibody, conjugated to IR700. In some examples, the antibody is a humanized antibody that specifically binds CD25, such as one disclosed herein. In other examples, the antibody is a monoclonal antibody that specifically binds to a surface protein on a cancer cell, such as a tumor-specific antigen.

Antigen (Ag): A compound, composition, or substance that can stimulate the production of antibodies or a T cell response in an animal, including compositions (such as one that includes a tumor-specific protein or CD25 protein) that are injected or absorbed into an animal. An antigen reacts with the products of specific humoral or cellular immunity, including those induced by heterologous antigens, such as the disclosed antigens. “Epitope” or “antigenic determinant” refers to the region of an antigen to which B and/or T cells respond. In one aspect, T cells respond to the epitope, when the epitope is presented in conjunction with an MHC molecule. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and nuclear magnetic resonance.

Examples of antigens include, but are not limited to, peptides, lipids, polysaccharides, and nucleic acids containing antigenic determinants, such as those recognized by an immune cell. In some examples, an antigen includes a tumor-specific protein or peptide (such as one found on the surface of a cell, such as a cancer cell) or immunogenic fragment thereof. In some examples, an antigen includes an immune cell-specific protein or peptide (such as one found on the surface of an immune cell, such as a T cell, such as a regulatory T cell) or immunogenic fragment thereof. In one example, the antigen is CD25, such as a mammalian CD25, such as mouse or human CD25.

Binding affinity: Affinity of an antibody or other antigen-binding molecule (such as an antibody-IR700 conjugate) for an antigen, such as CD25 or a tumor-specific antigen. In one aspect, affinity is calculated by a modification of the Scatchard method described by Frankel et al., Mol. Immunol., 16:101-106, 1979. In another aspect, binding affinity is measured by an antigen/antibody dissociation rate. In another aspect, binding affinity is measured by a competition radioimmunoassay. In another aspect, binding affinity is measured by ELISA. In some aspects, binding affinity is measured using the Octet system (ForteBio), which is based on bio-layer interferometry (BLI) technology. In other aspects, Kd is measured using surface plasmon resonance assays using a BIACORES-2000 or a BIACORES-3000 (BIAcore, Inc., Piscataway, N.J.). In other aspects, antibody affinity is measured by flow cytometry. An antibody or antibody conjugate that “specifically binds” an antigen (such as CD25) is an antibody or conjugate that binds the antigen with high affinity and does not significantly bind other unrelated antigens. In some examples, an antibody (such as an antibody in an antibody-IR700 conjugate) specifically binds to a target (such as a cell surface protein, such as CD25 or a tumor-specific protein) with a binding constant that is at least 10³ M⁻¹ greater, 10⁴ M⁻¹ greater or 10⁵ M⁻¹ greater than a binding constant for other molecules in a sample or subject. In some examples, an antibody (e.g., mAb) or fragments thereof, has an equilibrium constant (Kd) of 1 nM or less. For example, an antibody binds to a target, such as CD25 or a tumor-specific protein with a binding affinity of at least about 0.1×10⁻⁸ M, at least about 0.3×10⁻⁸ M, at least about 0.5×10⁻⁸ M, at least about 0.75×10⁻⁸ M, at least about 1.0×10⁻⁸ M, at least about 1.3×10⁻⁸ M at least about 1.5×10⁻⁸, or at least about 2.0×10⁻⁸ M.

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system. In one example, the cell killed by the disclosed compositions and methods is a cancer cell.

CD25 (IL-2 receptor alpha chain): (e.g., OMIM 147730) A type I transmembrane protein present on activated T cells, activated B cells, some thymocytes, myeloid precursors, and oligodendrocytes. CD25 has been used as a marker to identify CD4+FoxP3+ regulatory T cells in mice. CD25 is found on the surface of some cancer cells, including B-cell neoplasms, some acute nonlymphocytic leukemias, neuroblastomas, mastocytosis and tumor infiltrating lymphocytes. It functions as the receptor for HTLV-1 and is consequently expressed on neoplastic cells in adult T cell lymphoma/leukemia. Exemplary CD25 sequences can be found in the GenBank® database (e.g., Accession Nos. CAA44297.1, NP_000408.1, and NP_001295171.1).

Complementarity determining region (CDR): A region of hypervariable amino acid sequence that defines the binding affinity and specificity of an antibody. The light and heavy chains of a mammalian immunoglobulin each have three CDRs, designated L-CDR1, L-CDR2, L-CDR3 and H-CDR1, H-CDR2, H-CDR3, respectively.

Conjugate: In the context of the present disclosure, a “conjugate” is an antibody or antibody fragment (such as an antigen-binding fragment) covalently linked to an effector molecule or a second protein (such as a second antibody). The effector molecule can be, for example, a photon absorber (e.g., IR700), drug, toxin, therapeutic agent, detectable label, protein, nucleic acid, lipid, nanoparticle, carbohydrate or recombinant virus.

Conservative variant: In the context of the present disclosure, “conservative” amino acid substitutions are those substitutions that do not substantially affect or decrease the affinity of a protein, such as an antibody to CD25. As one example, a monoclonal antibody that specifically binds CD25 can include at most about 1, at most about 2, at most about 5, at most about 10, at most about 15, at most about 20, or at most about 25 conservative substitutions and specifically bind the CD25 polypeptide (for example with sub-nM affinity) but does not block IL-2 binding to CD25 or induce ADCC. The term “conservative variant” also includes the use of a substituted amino acid in place of an unsubstituted parent amino acid, provided that the variant retains activity. Non-conservative substitutions are those that reduce an activity (such as affinity) of a protein.

Conservative amino acid substitution tables providing functionally similar amino acids are well known. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

In some aspects herein, provided are amino acid sequences comprising no more than 10, no more than 9, no more than 8, no more than 7, no more than 6, no more than 5, no more than 4, no more than 3, no more than 2 or no more than 1 amino acid substitutions relative to any amino acid sequence disclosed herein.

Contacting: Placement in direct physical association, including both in solid and liquid form.

Contacting can occur in vitro, for example, with isolated cells, such as tumor cells, or in vivo by administering to a subject (such as a subject with a tumor, such as cancer).

Effector molecule: The portion of an antibody conjugate that is intended to have a desired effect on a cell to which the conjugate is targeted. The effector molecule can be, for example, a detectable label, a photon absorber (such as IR700), or a toxin. Effector molecules are also known as effector moieties, therapeutic agents, diagnostic agents, or similar terms. Therapeutic agents (or drugs) include such compounds as small molecules, nucleic acids, proteins, peptides, amino acids or derivatives, glycoproteins, radioisotopes, lipids, nanoparticles, carbohydrates, or recombinant viruses. Nucleic acid therapeutic and diagnostic moieties include antisense nucleic acids, derivatized oligonucleotides for covalent cross-linking with single or duplex DNA, and triplex forming oligonucleotides. Alternatively, the effector molecule can be contained within an encapsulation system, such as a nanoparticle, liposome or micelle, which is conjugated to the antibody. Encapsulation shields the effector molecule from direct exposure to the circulatory system. Means of preparing liposomes attached to antibodies are well known to those of skill in the art (see, for example, U.S. Pat. No. 4,957,735; and Connor et al., Pharm Ther 28:341-365, 1985).

Diagnostic agents or moieties include radioisotopes and other detectable labels (e.g., fluorophores, chemiluminescent agents, and enzymes). Radioactive isotopes include ³⁵S, ¹¹C, ¹³N, ¹⁵O, ¹⁸F, ¹⁹F, ^{99m}Tc, ¹³¹I, ³H, ¹⁴C, ¹⁵N, ⁹⁰Y, ⁹⁹Tc, ¹¹¹In and ¹²⁵I.

Framework region: Amino acid sequences interposed between CDRs. Framework regions include variable light and variable heavy framework regions. The framework regions serve to hold the CDRs in an appropriate orientation for antigen binding.

Heterologous: Originating from a separate genetic source or species.

Host cells: Cells in which a vector can be propagated and its DNA expressed. The cell may be prokaryotic or eukaryotic. In some examples, the prokaryotic cell is an E. coli cell. In some examples, the eukaryotic cell is a human cell. The term also includes any progeny of the subject host cell. It is understood that all progeny may not be identical to the parental cell since there may be mutations that occur during replication. However, such progeny are included when the term “host cell” is used.

Immune response: A response of a cell of the immune system, such as a B cell, T cell, or monocyte, to a stimulus. In one aspect, the response is specific for a particular antigen (an “antigen-specific response”). In one aspect, an immune response is a T cell response, such as a CD4+ response or a CD8+ response. In another aspect, the response is a B cell response, and results in the production of specific antibodies.

Immunomodulator: An immunomodulator is a substance that alters (for example, increases or decreases) one or more functions of the immune system. In some examples, an immunomodulator activates the immune system. In other examples, an immunomodulator inhibits activity of (or kills) immuno-suppressor cells. In some examples, an immunomodulator is a mAb, which can be attached to IR700, forming mAb-IR700, which can be used in the disclosed methods to target cells expressing a protein recognized by the mAb. Exemplary immunomodulators include the immunoactivators IL-15 and IFN-γ.

IR700 (IRDye® 700DX): A dye having the following formula:

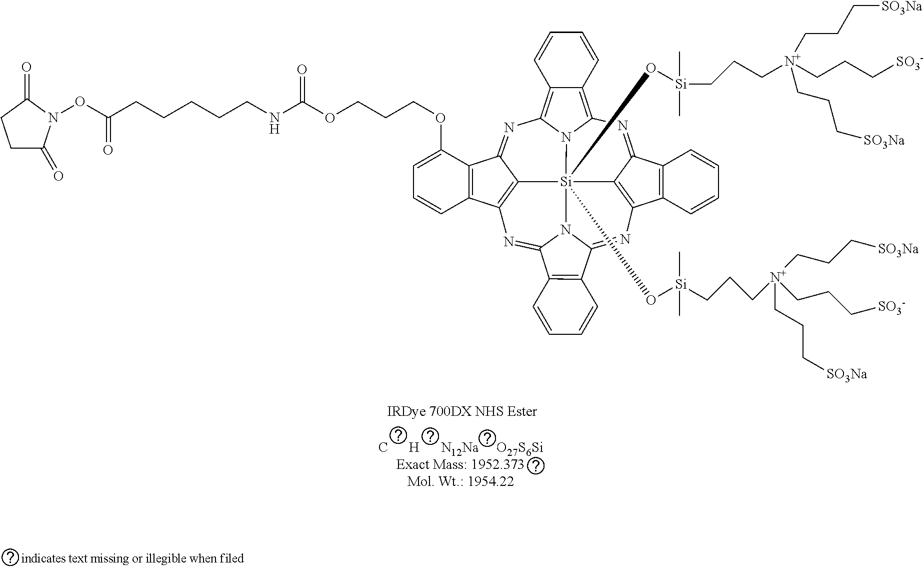

Amino-reactive IR700 is a relatively hydrophilic dye and can be covalently conjugated with an antibody using the NHS ester of IR700. IR700 also has more than 5-fold higher extinction coefficient (2.1×105 M⁻¹ cm⁻¹ at the absorption maximum of 689 nm) than conventional photosensitizers such as the hematoporphyrin derivative Photofrin® (1.2×10³M⁻¹ cm⁻¹ at 630 nm), meta-tetrahydroxyphenylchlorin; Foscan® (2.2×10⁴ M⁻¹ cm⁻¹ at 652 nm), and mono-L-aspartylchlorin e6; NPe6/Laserphyrin® (4.0×10⁴ M⁻¹ cm⁻¹ at 654 nm).

Label (or detectable label): A detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “labeled antibody” refers to incorporation of another molecule in the antibody. For example, the label is a detectable marker, such as the incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Various methods of labeling polypeptides and glycoproteins are known and may be used. Examples of labels for polypeptides include, but are not limited to, the following: radioisotopes or radionucleotides (such as ³⁵S, ¹¹C, ¹³N, ¹⁵O, ¹⁸F, ¹⁹F, ^{99m}Tc, ¹³¹I, ³H, ¹⁴C, ¹⁵N, ⁹⁰Y ⁹⁹Tc, ¹¹¹In and ¹²⁵I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as leucine zipper pair sequences, binding sites for secondary antibodies, metal binding domains, epitope tags), or magnetic agents, such as gadolinium chelates. In some aspects, labels are attached by spacer arms of various lengths to reduce potential steric hindrance.

Pharmaceutical composition: A chemical compound or composition capable of inducing a desired therapeutic or prophylactic effect when properly administered to a subject. A pharmaceutical composition can include a therapeutic agent, such as one or more antibodies, antibody-IR700 conjugates, and/or one or more immunoactivators. A therapeutic or pharmaceutical agent is one that alone or together with additional compound(s) induces the desired response (such as inducing a therapeutic or prophylactic effect when administered to a subject). In a particular example, a pharmaceutical composition includes a therapeutically effective amount of at least one antibody-IR700 conjugate.

Pharmaceutically acceptable vehicles: The pharmaceutically acceptable carriers (vehicles) useful in this disclosure are conventional. Remington: The Science and Practice of Pharmacy, The University of the Sciences in Philadelphia, Editor, Lippincott, Williams, & Wilkins, Philadelphia, PA, 21^{st} Edition (2005), describes compositions and formulations suitable for pharmaceutical delivery of one or more therapeutic compounds, such as one or more antibodies or antibody-IR700 conjugates disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Photoimmunotherapy (PIT): A molecularly targeted therapeutic that utilizes a target-specific photosensitizer based on a near infrared (NIR) phthalocyanine dye, IRDye700DX (IR700), conjugated to a monoclonal antibody (mAb) targeting a cellular protein (such as one on the cell surface, such as CD25 on the surface of Treg cells, or a tumor specific protein). In one example, the cell surface protein is one found specifically on cancer cells or immune cells, and thus PIT can be used to kill such cells. Cell death occurs when the antibody-IR700 conjugate binds to the cells and the cells are irradiated with NIR, while cells that do not express the cell surface protein recognized the antibody-IR700 conjugate are not killed in significant numbers.

Reducing Agent: An element or compound that loses (or “donates”) an electron to an electron recipient (oxidizing agent) in a redox chemical reaction. A reducing agent is thus oxidized when it loses electrons in the redox reaction. Reducing agents “reduce” (or, are “oxidized” by) oxidizing agents. Thus, one or more reducing agents can be used in combination with NIR-PIT in vitro and in vivo to reduce undesired non-specific tissue damage due to reactive oxygen species generated from IR700 following exposure to NIR light. For example, reducing agents can inactivate reactive oxygen species not bound or in the area of a tumor, and reduce undesired acute inflammation in other areas of the body. Exemplary reducing agents that can be used in the methods provided herein include L-cysteine, L-sodium ascorbate (L-NaAA), ascorbic acid (such as L- or R-ascorbic acid) and glutathione. In some examples, sodium azide is not used in vivo due to its toxicity. Thus, in some examples, the reducing agent used in the disclosed methods is not sodium azide. In one example, the reducing agent used in the disclosed methods is L-NaAA.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide or nucleic acid molecule will possess a relatively high degree of sequence identity when aligned using standard methods.

Methods of alignment of sequences for comparison are known. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of an antibody that specifically binds CD25 are typically characterized by possession of at least about 75%, for example at least about 80%, 90%, 95%, 96%, 97%, 98% or 99% sequence identity counted over the full-length alignment with the amino acid sequence of the antibody using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids and may possess sequence identities of at least 85% or at least 90% or 95% depending on their similarity to the reference sequence. Methods for determining sequence identity over such short windows are available at the NCBI website on the internet. These sequence identity ranges are provided for guidance only; it is entirely possible that strongly significant homologs could be obtained that fall outside of the ranges provided.

Subject or patient: A term that includes human and non-human mammals. In one example, the subject is a human or veterinary subject, such as a mouse, rat, dog, cat, or non-human primate. In some examples, the subject is a mammal (such as a human) who has cancer or is being treated for cancer.

Therapeutically effective amount: An amount of a composition (such as a CD25-specific antibody-IR700 conjugate) that alone, or together with an additional therapeutic agent(s) (such as an anti-tumor antigen-specific antibody-IR700 conjugate, an anti-CTLA4-IR700 molecule, an anti-PD-L1-IR700 molecule, an immunomodulator, a check point inhibitor, and/or a reducing agent) sufficient to achieve a desired effect in a subject, or in a cell, being treated with the agent. The effective amount of the agent (such as an antibody-IR700 conjugate, alone or in combination with other agents) can be dependent on several factors, including, but not limited to the subject or cells being treated, the particular therapeutic agent, and the manner of administration of the therapeutic composition. In one example, a therapeutically effective amount or concentration of an antibody-IR700 conjugate is one that is sufficient to prevent advancement (such as metastasis), delay progression, or to cause regression of a disease, or which is capable of reducing symptoms caused by the disease, such as cancer. In one example, a therapeutically effective amount or concentration of an antibody-IR700 conjugate is one that is sufficient to increase the survival time of a patient with a tumor. In one example, a therapeutically effective amount or concentration of a reducing agent is one that is sufficient to decrease edema, acute inflammatory reaction, or both, in a subject treated with NIR-PIT.

In one example, a desired response is to reduce or inhibit one or more symptoms associated with cancer. The one or more symptoms do not have to be completely eliminated for the composition to be effective. For example, treatment with a modality provided herein in some examples decreases the size of a tumor (such as the volume or weight of a tumor or metastasis of a tumor), for example by at least 20%, at least 50%, at least 80%, at least 90%, at least 95%, at least 98%, or even at least 100%, as compared to the tumor size in the absence of the treatment (or with only a subset of the therapy provided herein). In one particular example, treatment with a modality provided herein kills a population of cells (such as cancer cells), for example by killing at least 20%, at least 50%, at least 60%, at least 70%, at least 80%, at least 90%, at least 95%, at least 98%, or even at least 100% of the cells, as compared to the cell killing in the absence of the treatment (or with only a subset of the therapy provided herein). In one particular example, treatment with a modality provided herein increases the survival time of a patient with a tumor (or who has had a tumor recently removed), for example increases survival by at least 20%, at least 50%, at least 60%, at least 70%, at least 80%, at least 90%, at least 95%, at least 98%, at least 100%, at least 200%, or at least 500%, as compared to the survival time in the absence of the treatment (or with only a subset of the therapy provided herein). In some examples, treatment with a modality provided herein increases an amount of memory T cells in a subject, for example an increase of at least 20%, at least 50%, at least 60%, at least 70%, at least 80%, at least 90%, at least 95%, at least 98%, at least 100%, at least 200%, or at least 500%, as compared to an amount of memory T cells in the absence of the treatment (or with only a subset of the therapy provided herein. In some examples, treatment with a modality provided herein decreases an amount of Treg cells (such as FOXP3⁺CD25⁺CD4⁺ Treg cells) in a targeted tumor/tumor bed, for example a decrease of at least 20%, at least 50%, at least 60%, at least 70%, at least 80%, at least 90%, at least 95%, at least 98%, at least 100%, as compared to an amount of Treg cells in the targeted tumor/tumor bed in the absence of treatment (or with only a subset of the therapy provided herein). In some examples, treatment with a modality provided herein increases the CD8+/Treg ratio in a treated subject, for example increase by at least 20%, at least 50%, at least 60%, at least 70%, at least 80%, at least 90%, at least 95%, at least 98%, at least 100%, at least 200%, or at least 500%, as compared to the CD8+/Treg ratio in the absence of treatment (or with only a subset of the therapy provided herein). In some examples, combinations of these effects are archived by the disclosed methods.

The effective amount of a therapeutic agent administered to a human or veterinary subject can vary depending upon a number of factors associated with that subject, for example the overall health of the subject. An effective amount of an agent can be determined by varying the dosage of the composition(s) and measuring the resulting therapeutic response, such as the regression of a tumor. Effective amounts also can be determined through various in vitro, in vivo or in situ immunoassays. The disclosed agents can be administered in a single dose, or in several doses, as needed to obtain the desired response. However, the effective amount can be dependent on the treatment being applied, the subject being treated, the severity and type of the condition being treated, and the manner of administration.

In particular examples, a therapeutically effective dose of an antibody-IR700 conjugate is at least 0.5 milligram per 60 kilogram (mg/kg), at least 5 mg/60 kg, at least 10 mg/60 kg, at least 20 mg/60 kg, at least 30 mg/60 kg, at least 50 mg/60 kg, at least 75 mg/60 kg, at least 100 mg/60 kg, at least 125 mg/60 kg, at least 150 mg/60 kg, at least 175 mg/60 kg, at least 200 mg/60 kg, at least 225 mg/60 kg, at least 250 mg/60 kg, at least 300 mg/60 kg, at least 350 mg/60 kg, or at least 400 mg/60 kg, for example 0.5 to 250 mg/60 kg, such as a dose of 1 mg/60 kg, 2 mg/60 kg, 5 mg/60 kg, 20 mg/60 kg, 50 mg/60 kg, 75 mg/60 kg, 100 mg/60 kg, 125 mg/60 kg, 150 mg/60 kg, 175 mg/60 kg, 200 mg/60 kg, 300 mg/60 kg, or 400 mg/60 kg, for example when administered intravenously. In one non-limiting example, the therapeutically effective dose of the antibody-IR700 conjugate, such as an antibody-IR700 conjugate containing a disclosed CD25 antibody is 100 to 200 mg/60 kg. In another example, a therapeutically effective dose of an antibody-IR700 conjugate is at least 10 μg/kg, such as at least 100 μg/kg, at least 500 μg/kg, or at least 500 μg/kg, for example 10 μg/kg to 1000 μg/kg, such as a dose of 100 μg/kg, 250 μg/kg, about 500 μg/kg, 750 μg/kg, or 1000 μg/kg, for example when administered intratumorally or i.p. In one example, a therapeutically effective dose of an antibody-IR700 conjugate is at least 1 μg/ml, such as at least 500 μg/ml, such as between 20 μg/ml to 100 μg/ml, such as 10 μg/ml, 20 μg/ml, 30 μg/ml, 40 μg/ml, 50 μg/ml, 60 μg/ml, 70 μg/ml, 80 μg/ml, 90 μg/ml or 100 μg/ml administered in topical solution. However, one skilled in the art will recognize that higher or lower dosages also could be used, for example depending on the particular antibody-IR700 conjugate. In particular examples, such daily dosages are administered in one or more divided doses (such as 2, 3, or 4 doses) or in a single formulation. The disclosed antibody-IR700 conjugates can be administered alone, in the presence of a pharmaceutically acceptable carrier, in the presence of other therapeutic agents (such as other anti-neoplastic agents and/or reducing agents).

Generally a suitable dose of irradiation following administration of one or more antibody-IR700 conjugates (and in some examples also a reducing agent) is at least 1 J/cm² at a wavelength of 660-740 nm, for example, at least 4 J/cm² at a wavelength of 660-740 nm, at least 10 J/cm² at a wavelength of 660-740 nm, at least 20 J/cm² at a wavelength of 660-740 nm, at least 25 J/cm² at a wavelength of 660-740 nm, at least 50 J/cm² at a wavelength of 660-740 nm, or at least 100 J/cm² at a wavelength of 660-740 nm, for example 1 to 500 J/cm² at a wavelength of 660-740 nm, 10 to 100 J/cm² at a wavelength of 660-740 nm, or 20 to 60 J/cm² at a wavelength of 660-740 nm. In some examples, the wavelength is 660-710 nm, such as 680 nm or 690 nm. In specific examples, a suitable dose of irradiation following administration of the antibody-IR700 conjugate is at least 1 J/cm² at a wavelength of 680 or 690 nm for example, at least 4 J/cm² at a wavelength of 680 or 690 nm, at least 10 J/cm² at a wavelength of 680 or 690 nm, at least 20 J/cm² at a wavelength of 680 or 690 nm, at least 25 J/cm² at a wavelength of 680 or 690 nm, at least 50 J/cm² at a wavelength of 680 or 690 nm, or at least 100 J/cm² at a wavelength of 680 or 690 nm, for example 4 to 100 J/cm², 4 to 50 J/cm², or 10 to 25 J/cm² at a wavelength of 680 or 690 nm. In one non-limiting example, a suitable dose of irradiation following administration of the antibody-IR700 conjugate is 50 J/cm² at a wavelength of 690 nm. In particular examples, multiple irradiations are performed (such as at least 2, at least 3, or at least 4 irradiations, such as 2, 3, 4, 5, 6, 7, 8, 9 or 10 separate administrations), following administration of one or more antibody-IR700 conjugates (and in some examples also following administration of a second (tumor antigen-specific) monoclonal antibody-IR700 conjugate, an anti-CTLA4-IR700 conjugate, an anti-PD-L1-IR700 conjugate, an immunoactivator, a check point inhibitor, and/or a reducing agent).

Treating: A term when used to refer to the treatment of a cell or tissue with a therapeutic agent, includes contacting or incubating one or more agents (such as one or more CD25 antibody-IR700 conjugates and in some examples also a second (tumor antigen-specific) monoclonal antibody-IR700 conjugate, an anti-CTLA4-IR700 conjugate, an anti-PD-L1-IR700 conjugate, an immunoactivator, a check point inhibitor, and/or a reducing agent) with the cell or tissue and/or administering one or more agents to a subject, for example a subject with cancer. A treated cell is a cell that has been contacted with a desired composition in an amount and under conditions sufficient for the desired response. In one example, a treated cell is a cell that has been exposed to an antibody-IR700 conjugate under conditions sufficient for the antibody to bind to a surface protein on the cell, optionally contacted with a reducing agent, and irradiated with NIR light, until sufficient cell killing is achieved. In other examples, a treated subject is a subject that has been administered one or more antibody-IR700 conjugates under conditions sufficient for the antibody to bind to a surface protein on the cell, optionally administered one or more reducing agents, and irradiated with NIR light, until sufficient cell killing is achieved.

Tumor, neoplasia, malignancy or cancer: A neoplasm is an abnormal growth of tissue or cells which results from excessive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

Tumors include original (primary) tumors, recurrent tumors, and metastases (secondary) tumors. A tumor recurrence is the return of a tumor, at the same site as the original (primary) tumor, for example, after the tumor has been removed surgically, by drug or other treatment, or has otherwise disappeared. A metastasis is the spread of a tumor from one part of the body to another. Tumors formed from cells that have spread are called secondary tumors and contain cells that are like those in the original (primary) tumor.

There can be a recurrence of either a primary tumor or a metastasis Exemplary tumors, such as cancers, that can be treated with the disclosed methods include solid tumors, such as breast carcinomas (e.g. lobular and duct carcinomas), sarcomas, carcinomas of the lung (e.g., non-small cell carcinoma, large cell carcinoma, squamous carcinoma, and adenocarcinoma), mesothelioma of the lung, colorectal adenocarcinoma, head and neck cancers (e.g., adenocarcinoma, squamous cell carcinoma, metastatic squamous, such as cancers caused by HPV or Epstein-Barr virus, such as HPV16; can include cancers of the mouth, tongue, cheek, nasopharynx, throat, hypopharynx, oropharynx, larynx, and trachea), stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, and lymphatic tumors (including B-cell and T-cell malignant lymphoma). In one example, the tumor is an adenocarcinoma. In one example, the tumor is an oropharyngeal cancer. In one example the tumor is a squamous carcinoma, such as one of the head and neck or skin.

The methods can also be used to treat liquid tumors (e.g., hematological malignancies), such as a lymphatic, white blood cell, or other type of leukemia. In a specific example, the tumor treated is a tumor of the blood, such as a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), lymphomas (such as Hodgkin's lymphoma and non-Hodgkin's lymphoma), and myelomas.

Under conditions sufficient for: A phrase that is used to describe any environment that permits the desired activity. In one example, “under conditions sufficient for” includes administering an antibody-IR700 conjugate to a subject sufficient to allow the antibody-IR700 conjugate to bind to its targeted cell surface protein (such as a tumor-specific antigen or immune cell specific antigen, for example CD25). In particular aspects, the desired activity is killing the cells to which the antibody-IR700 conjugate is bound, following therapeutic irradiation of the cells.

Untreated cell: A cell that has not been contacted with a therapeutic agent, such as an antibody-IR700 conjugate and/or irradiation. In one example, an untreated cell is a cell that receives the vehicle in which the therapeutic agent(s) was delivered. Similarly, an untreated subject is a subject who has not been administered a therapeutic agent, such as an antibody-IR700 conjugate and/or irradiation. In one example, an untreated subject is a subject that receives the vehicle in which the therapeutic agent(s) was delivered. Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements. In some examples, the vector is a viral vector, such as a lentiviral vector, an adenovirus vector, or an adeno-associated virus (AAV) vector.

Disclosure of certain specific examples is not meant to exclude other aspects. In addition, any treatments described herein are not necessarily exclusive of other treatment but can be combined with other bioactive agents or treatment modalities.

### IV. Monoclonal Antibodies Specific for CD25

Described herein are monoclonal antibodies that bind human and cynomolgus CD25 (interleukin-2 receptor alpha; IL-2Rα) with high affinity. The disclosed antibodies specifically bind CD25, but do not inhibit binding of CD25 to IL-2, nor do the CD25-specific antibodies induce ADCC. In some examples, the CD25 antibodies provided herein, including variants thereof, have sub-nM affinity for CD25, such as human, cynomolgus or mouse CD25. The amino acid sequences of the VH domains and VL domains of 21 exemplary CD25-specific monoclonal antibodies are set forth herein as SEQ ID NOs: 1-42. The location of each CDR, as determined by IMGT, is indicated by underline in the sequences below. In each VH domain sequence, the IMGT CDR1, CDR2 and CDR3 sequences are at positions 31-35, 50-66 and 99-112, respectively; and in each VL domain sequence, the IMGT CDR1, CDR2 and CDR3 sequences are at positions 24-33, 49-55 and 88-96, respectively. Other numbering schemes, such as Chothia or Kabat, can also be used to determine the boundaries of each CDR. Consensus and exemplary CDR sequences are also provided below.

14554-VH
(SEQ ID NO: 1)
EVQLVESGGGLVQPGGSLRLSCAASGFWFSSYGMSWVRQAPGKGLELVSTINGYGDTWYYPDSV
KGRFTISRDNSKNTLYLQMNSLRAEDTAVYYCARDRDYTNSYYYAHDYWGQGTLVTVSS
14554-VL
(SEQ ID NO: 2)
EIVLTQSPATLSLSPGERATLSCRASSYVSFMHWLQQKPGQAPRPWIYATANLASGVPARFSGSGSG
TDYTLTISSLEPEDFAVYYCQQWSSNPPAFGGGTKVEIK
14555-VH
(SEQ ID NO: 3)
EVQLVESGGGLVQPGGSLRLSCAASGFTFSSWGMSWVRQAPGKGLELVSTINGYGDTTYYPDSVK
GRFTISRDNSKNTLYLQMNSLRAEDTAVYYCARDRDYTNSYYYAHDYWGQGTLVTVSS
14555-VL
(SEQ ID NO: 4)
EIVLTQSPATLSLSPGERATLSCRASSYVSFMHWLQQKPGQAPRPWIYATANLASGVPARFSGSGSG
TDYTLTISSLEPEDFAVYYCQQWSSNPPAFGGGTKVEIK
14556-VH
(SEQ ID NO: 5)
EVQLVESGGGLVQPGGSLRLSCAASGYTFSSYGMSWVRQAPGKGLELVSTINGYGDTTYYPDSVK
GRFTISRDNSKNTLYLQMNSLRAEDTAVYYCARDRDYSNSYYYALDYWGQGTLVTVSS
14556-VL
(SEQ ID NO: 6)
EIVLTQSPATLSLSPGERATLSCRASSYVSFMHWLQQKPGQAPRPWIYATANLASGVPARFSGSGSG
TDYTLTISSLEPEDFAVYYCQQWSSNPPAFGGGTKVEIK
14557-VH
(SEQ ID NO: 7)
EVQLVESGGGLVQPGGSLRLSCAASGYTFSSYGMSWVRQAPGKGLELVSTINGYGDTWYYPDSVK
GRFTISRDNSKNTLYLQMNSLRAEDTAVYYCARDRDYSNSYYYAHDYWGQGTLVTVSS
14557-VL
(SEQ ID NO: 8)
EIVLTQSPATLSLSPGERATLSCRASSWVSFMHWLQQKPGQAPRPWIYATQNLASGVPARFIGSGSG
TDYTLTISSLEPEDFAVYYCQQWSSNPPAFGGGTKVEIK
14558-VH
(SEQ ID NO: 9)
EVQLVESGGGLVQPGGSLRLSCAASGYWFSSYGMSWVRQAPGKGLELVSTINGYGDTTYYPDSVK
GRFTISRDNSKNTLYLQMNSLRAEDTAVYYCARDRDYTNSYYYAHDYWGQGTLVTVSS
14558-VL
(SEQ ID NO: 10)
EIVLTQSPATLSLSPGERATLSCRASSSVSFMHWLQQKPGQAPRPWIYATMNLASGVPARFSGSGSG
TDYTLTISSLEPEDFAVYYCQQWSSNPPAFGGGTKVEIK
14559-VH
(SEQ ID NO: 11)
EVQLVESGGGLVQPGGSLRLSCAASGFTFSSYGMSWVRQAPGKGLELVSTINGYGDTTYYPDSVK
GRFTISRDNSKNTLYLQMNSLRAEDTAVYYCARDRDYSNSYYYAHDYWGQGTLVTVSS
14559-VL
(SEQ ID NO: 12)
EIVLTQSPATLSLSPGERATLSCRASSWVSFMHWLQQKPGQAPRPWIYATQNLASGVPARFSGSGS
GTDYTLTISSLEPEDFAVYYCQQWSSNPPAFGGGTKVEIK
14560-VH
(SEQ ID NO: 13)
EVQLVESGGGLVQPGGSLRLSCAASGFTFSSYGMSWVRQAPGKGLELVSTINGYGDTTYYPDSVK
GRFTISRDNSKNTLYLQMNSLRAEDTAVYYCARDRDYTNSYYYAHDYWGQGTLVTVSS
14560-VL
(SEQ ID NO: 14)
EIVLTQSPATLSLSPGERATLSCRASSSVSFMHWLQQKPGQAPRPWIYATQNLASGVPARFSGSGSG
TDYTLTISSLEPEDFAVYYCQQWSSNPPAFGGGTKVEIK
14561-VH
(SEQ ID NO: 15)
EVQLVESGGGLVQPGGSLRLSCAASGYTFSSWGMSWVRQAPGKGLELVSTINGYGDTWYYPDSV
KGRFTISRDNSKNTLYLQMNSLRAEDTAVYYCARDRDYSNSYYYAHDYWGQGTLVTVSS
14561-VL
(SEQ ID NO: 16)
EIVLTQSPATLSLSPGERATLSCRASSYVSFMHWLQQKPGQAPRPWIYATMNLASGVPARFSGSGSG
TDYTLTISSLEPEDFAVYYCQQWSSNPPAFGGGTKVEIK
14562-VH
(SEQ ID NO: 17)
EVQLVESGGGLVQPGGSLRLSCAASGFTFSSYGMSWVRQAPGKGLELVSTINGYGDTWYYPDSVK
GRFTISRDNSKNTLYLQMNSLRAEDTAVYYCARDRDYSNSYYYAHDYWGQGTLVTVSS
14562-VL
(SEQ ID NO: 18)
EIVLTQSPATLSLSPGERATLSCRASSSVSFMHWLQQKPGQAPRPWIYATMNLASGVPARFSGSGSG
TDYTLTISSLEPEDFAVYYCQQWSSNPPAFGGGTKVEIK
14563-VH
(SEQ ID NO: 19)
EVQLVESGGGLVQPGGSLRLSCAASGYTFSSYGMSWVRQAPGKGLELVSTINGYGDTTYYPDSVK
GRFTISRDNSKNTLYLQMNSLRAEDTAVYYCARDRDYGNSYYYAHDYWGQGTLVTVSS
14563-VL
(SEQ ID NO: 20)
EIVLTQSPATLSLSPGERATLSCRASSYVSFMHWLQQKPGQAPRPWIYATMNLASGVPARFSGSGSG
TDYTLTISSLEPEDFAVYYCQQWSSNPPAFGGGTKVEIK
14564-VH
(SEQ ID NO: 21)
EVQLVESGGGLVQPGGSLRLSCAASGFTFSSWGMSWVRQAPGKGLELVSTINGYGDTTYYPDSVK
GRFTISRDNSKNTLYLQMNSLRAEDTAVYYCARDRDYSNSYYYAHDYWGQGTLVTVSS
14564-VL
(SEQ ID NO: 22)
EIVLTQSPATLSLSPGERATLSCRASSYVSFMHWLQQKPGQAPRPWIYATANLASGVPARFSGSGSG
TDYTLTISSLEPEDFAVYYCQQWSSNPPAFGGGTKVEIK
14565-VH
(SEQ ID NO: 23)
EVQLVESGGGLVQPGGSLRLSCAASGFTFSSYGMSWVRQAPGKGLELVSTINGYGDTWYYPDSVK
GRFTISRDNSKNTLYLQMNSLRAEDTAVYYCARDRDYGNSYYYAHDYWGQGTLVTVSS
14565-VL
(SEQ ID NO: 24)
EIVLTQSPATLSLSPGERATLSCRASSWVSFMHWLQQKPGQAPRPWIYATQNLASGVPARFSGSGS
GTDYTLTISSLEPEDFAVYYCQQWSSNPPAFGGGTKVEIK
14566-VH
(SEQ ID NO: 25)
EVQLVESGGGLVQPGGSLRLSCAASGFTFSSYGMSWVRQAPGKGLELVSTINGYGDTTYYPDSVK
GRFTISRDNSKNTLYLQMNSLRAEDTAVYYCARDRDYGNSYYYAHDYWGQGTLVTVSS
14566-VL
(SEQ ID NO: 26)
EIVLTQSPATLSLSPGERATLSCRASSWVSFMHWLQQKPGQAPRPWIYATMNLASGVPARFSGSGS
GTDYTLTISSLEPEDFAVYYCQQWSSNPPAFGGGTKVEIK
14567-VH
(SEQ ID NO: 27)
EVQLVESGGGLVQPGGSLRLSCAASGFWFSSWGMSWVRQAPGKGLELVSTINGYGDTTYYPDSVK
GRFTISRDNSKNTLYLQMNSLRAEDTAVYYCARDRDYTNSYYYAHDYWGQGTLVTVSS
14567-VL
(SEQ ID NO: 28)
EIVLTQSPATLSLSPGERATLSCRASSYVSFMHWLQQKPGQAPRPWIYATQNLASGVPARFSGSGSG
TDYTLTISSLEPEDFAVYYCQQWSSNPPAFGGGTKVEIK
14568-VH
(SEQ ID NO: 29)
EVQLVESGGGLVQPGGSLRLSCAASGFTFSSYGMSWVRQAPGKGLELVSTINGYGDTTYYPDSVK
GRFTISRDNSKNTLYLQMNSLRAEDTAVYYCARDRDYTNSYYYAHDYWGQGTLVTVSS
14568-VL
(SEQ ID NO: 30)
EIVLTQSPATLSLSPGERATLSCRASSYVSFMHWLQQKPGQAPRPWIYATMNLASGVPARFSGSGSG
TDYTLTISSLEPEDFAVYYCQQWSSNPPAFGGGTKVEIK
14569-VH
(SEQ ID NO: 31)
EVQLVESGGGLVQPGGSLRLSCAASGYWFSSWGMSWVRQAPGKGLELVSTINGYGDTTYYPDSV
KGRFTISRDNSKNTLYLQMNSLRAEDTAVYYCARDRDYSNSYYYAHDYWGQGTLVTVSS
14569-VL
(SEQ ID NO: 32)
EIVLTQSPATLSLSPGERATLSCRASSYVSFMHWLQQKPGQAPRPWIYATMNLASGVPARFSGSGSG
TDYTLTISSLEPEDFAVYYCQQWSSNPPAFGGGTKVEIK
14570-VH
(SEQ ID NO: 33)
EVQLVESGGGLVQPGGSLRLSCAASGFWFSSYGMSWVRQAPGKGLELVSTINGYGDTTYYPDSVK
GRFTISRDNSKNTLYLQMNSLRAEDTAVYYCARDRDYTNSYYYAHDYWGQGTLVTVSS
14570-VL
(SEQ ID NO: 34)
EIVLTQSPATLSLSPGERATLSCRASSSVSFMHWLQQKPGQAPRPWIYATANLASGVPARFSGSGSG
TDYTLTISSLEPEDFAVYYCQQWSSNPPAFGGGTKVEIK
14571-VH
(SEQ ID NO: 35)
EVQLVESGGGLVQPGGSLRLSCAASGYTFSSYGMSWVRQAPGKGLELVSTINGYGDTTYYPDSVK
GRFTISRDNSKNTLYLQMNSLRAEDTAVYYCARDRDYGNSYYYAHDYWGQGTLVTVSS
14571-VL
(SEQ ID NO: 36)
EIVLTQSPATLSLSPGERATLSCRASSWVSFMHWLQQKPGQAPRPWIYATMNLASGVPARFSGSGS
GTDYTLTISSLEPEDFAVYYCQQWSSNPPAFGGGTKVEIK
14572-VH
(SEQ ID NO: 37)
EVQLVESGGGLVQPGGSLRLSCAASGYWFSSYGMSWVRQAPGKGLELVSTINGYGDTTYYPDSVK
GRFTISRDNSKNTLYLQMNSLRAEDTAVYYCARDRDYTNSYYYAHDYWGQGTLVTVSS
14572-VL
(SEQ ID NO: 38)
EIVLTQSPATLSLSPGERATLSCRASSYVSFMHWLQQKPGQAPRPWIYATMNLASGVPARFSGSGSG
TDYTLTISSLEPEDFAVYYCQQWSSNPPAFGGGTKVEIK
14573-VH
(SEQ ID NO: 39)
EVQLVESGGGLVQPGGSLRLSCAASGFTFSSWGMSWVRQAPGKGLELVSTINGYGDTTYYPDSVK
GRFTISRDNSKNTLYLQMNSLRAEDTAVYYCARDRDYTNSYYYAHDYWGQGTLVTVSS
14573-VL
(SEQ ID NO: 40)
EIVLTQSPATLSLSPGERATLSCRASSYVSFMHWLQQKPGQAPRPWIYATSNLASGVPARFSGSGSG
TDYTLTISSLEPEDFAVYYCQQWSSNPPAFGGGTKVEIK
U579-WT-VH
(SEQ ID NO: 41)
EVQLVESGGGLVQPGGSLRLSCAASGFTFSSYGMSWVRQAPGKGLELVSTINGYGDTTYYPDSVK
GRFTISRDNSKNTLYLQMNSLRAEDTAVYYCARDRDYGNSYYYALDYWGQGTLVTVSS
U579-WT-VL
(SEQ ID NO: 42)
EIVLTQSPATLSLSPGERATLSCRASSSVSFMHWLQQKPGQAPRPWIYATSNLASGVPARFSGSGSG
TDYTLTISSLEPEDFAVYYCQQWSSNPPAFGGGTKVEIK

The following are consensus amino acid sequences for each VH domain CDR (HCDR) and each VL domain CDR (LCDR) based alignment of the 21 antibody sequences.

Consensus HCDR1:
(SEQ ID NO: 43)
SXGMS, where X is W or Y
Consensus HCDR2:
(SEQ ID NO: 44)
TINGYGDTXYYPDSVKG, where X is W or T
Consensus HCDR3:
(SEQ ID NO: 45)
DRDYX₁NSYYYAX₂DY, where X₁ is S or G; and X₂ is
L or H
Consensus LCDR1:
(SEQ ID NO: 46)
RASSXVSFMH, where X is S, W or Y
Consensus LCDR2:
(SEQ ID NO: 47)
ATXNLAS, where X is S, A, Q or M
Consensus LCDR3:
(SEQ ID NO: 48)
QQWSSNPPA

The following are specific amino acid sequences for exemplary VH domain CDRs (HCDR) and exemplary VL domain CDRs (LCDR).

Exemplary HCDR1:
(SEQ ID NO: 49)
SWGMS
or
(SEQ ID NO: 50)
SYGMS
Exemplary HCDR2:
(SEQ ID NO: 51)
TINGYGDTTYYPDSVKG
Exemplary HCDR3:
(SEQ ID NO: 52)
DRDYSNSYYYAHDY
or
(SEQ ID NO: 53)
DRDYTNSYYYAHDY
Exemplary LCDR1:
(SEQ ID NO: 54)
RASSYVSFMH
Exemplary LCDR2:
(SEQ ID NO: 55)
ATANLAS
or
(SEQ ID NO: 56)
ATMNLAS
Exemplary LCDR3:
(SEQ ID NO: 48)
QQWSSNPPA

Provided herein are monoclonal antibodies that specifically bind human CD25. The monoclonal antibodies include a variable heavy (VH) domain and a variable light (VL) domain. In some aspects, the VH domain includes a complementarity determining region 1 (CDR1), CDR2 and CDR3, and the amino acid sequences of the CDR1, CDR2 and CDR3 respectively include SEQ ID NO: 43, SEQ ID NO: 44 and SEQ ID NO: 45; and/or the VL domain includes a CDR1, CDR2 and CDR3, and the amino acid sequences of the CDR1, CDR2 and CDR3 respectively include SEQ ID NO: 46, SEQ ID NO: 47 and SEQ ID NO: 48.

In some examples, the amino acid sequences of the VH domain CDR1, CDR2 and CDR3 respectively include SEQ ID NO: 49, SEQ ID NO: 51 and SEQ ID NO: 52; and/or the amino acid sequences of the VL domain CDR1, CDR2 and CDR3 respectively include SEQ ID NO: 54, SEQ ID NO: 55 and SEQ ID NO: 48. In particular examples, the amino acid sequence of the VH domain is at least 90% identical to SEQ ID NO: 21 and the VH domain CDR1, CDR2 and CDR3 respectively include SEQ ID NO: 49, SEQ ID NO: 51 and SEQ ID NO: 52; and/or the amino acid sequence of the VL domain is at least 90% identical to SEQ ID NO: 22 and the VL domain CDR1, CDR2 and CDR3 respectively include SEQ ID NO: 54, SEQ ID NO: 55 and SEQ ID NO: 48.

In other examples, the amino acid sequences of the VH domain CDR1, CDR2 and CDR3 respectively include SEQ ID NO: 49, SEQ ID NO: 51 and SEQ ID NO: 52; and/or the amino acid sequences of the VL domain CDR1, CDR2 and CDR3 respectively include SEQ ID NO: 54, SEQ ID NO: 56 and SEQ ID NO: 48. In particular examples, the amino acid sequence of the VH domain is at least 90% identical to SEQ ID NO: 31 and the VH domain CDR1, CDR2 and/or CDR3 respectively include SEQ ID NO: 49, SEQ ID NO: 51 and SEQ ID NO: 52; and/or the amino acid sequence of the VL domain is at least 90% identical to SEQ ID NO: 32 and the VL domain CDR1, CDR2 and CDR3 respectively include SEQ ID NO: 54, SEQ ID NO: 56 and SEQ ID NO: 48.

In other examples, the amino acid sequences of the VH domain CDR1, CDR2 and CDR3 respectively include SEQ ID NO: 50, SEQ ID NO: 51, and SEQ ID NO: 53; and/or the amino acid sequences of the VL domain CDR1, CDR2 and CDR3 respectively include SEQ ID NO: 54, SEQ ID NO: 56, and SEQ ID NO: 48. In particular examples, the amino acid sequence of the VH domain is at least 90% identical to SEQ ID NO: 37 and the VH domain CDR1, CDR2 and CDR3 respectively include SEQ ID NO: 50, SEQ ID NO: 51 and SEQ ID NO: 53; and/or the amino acid sequence of the VL domain is at least 90% identical to SEQ ID NO: 38 and the VL domain CDR1, CDR2 and CDR3 respectively include SEQ ID NO: 54, SEQ ID NO: 56 and SEQ ID NO: 48.

In some aspects, the monoclonal antibodies include at least a portion of the amino acid sequence set forth as any one of SEQ ID NOs: 1-42, such as one or more (such as all three) CDR sequences from any one of SEQ ID NOs: 1-42. In some examples, the CDR locations are determined IMGT, Kabat. Paratome, Chothia, or a combination of one or more thereof.

In some examples, the VH domain of the antibody includes the CDR sequences of SEQ ID NO: 1 and the VL domain of the antibody includes the CDR sequences of SEQ ID NO: 2; the VH domain of the antibody includes the CDR sequences of SEQ ID NO: 3 and the VL domain of the antibody includes the CDR sequences of SEQ ID NO: 4; the VH domain of the antibody includes the CDR sequences of SEQ ID NO: 5 and the VL domain of the antibody includes the CDR sequences of SEQ ID NO: 6; the VH domain of the antibody includes the CDR sequences of SEQ ID NO: 7 and the VL domain of the antibody includes the CDR sequences of SEQ ID NO: 8; the VH domain of the antibody includes the CDR sequences of SEQ ID NO: 9 and the VL domain of the antibody includes the CDR sequences of SEQ ID NO: 10; the VH domain of the antibody includes the CDR sequences of SEQ ID NO: 11 and the VL domain of the antibody includes the CDR sequences of SEQ ID NO: 12; the VH domain of the antibody includes the CDR sequences of SEQ ID NO: 13 and the VL domain of the antibody includes the CDR sequences of SEQ ID NO: 14; the VH domain of the antibody includes the CDR sequences of SEQ ID NO: 15 and the VL domain of the antibody includes the CDR sequences of SEQ ID NO: 16; the VH domain of the antibody includes the CDR sequences of SEQ ID NO: 17 and the VL domain of the antibody includes the CDR sequences of SEQ ID NO: 18; the VH domain of the antibody includes the CDR sequences of SEQ ID NO: 19 and the VL domain of the antibody includes the CDR sequences of SEQ ID NO: 20; the VH domain of the antibody includes the CDR sequences of SEQ ID NO: 21 and the VL domain of the antibody includes the CDR sequences of SEQ ID NO: 22; the VH domain of the antibody includes the CDR sequences of SEQ ID NO: 23 and the VL domain of the antibody includes the CDR sequences of SEQ ID NO: 24; the VH domain of the antibody includes the CDR sequences of SEQ ID NO: 25 and the VL domain of the antibody includes the CDR sequences of SEQ ID NO: 26; the VH domain of the antibody includes the CDR sequences of SEQ ID NO: 27 and the VL domain of the antibody includes the CDR sequences of SEQ ID NO: 28; the VH domain of the antibody includes the CDR sequences of SEQ ID NO: 29 and the VL domain of the antibody includes the CDR sequences of SEQ ID NO: 30; the VH domain of the antibody includes the CDR sequences of SEQ ID NO: 31 and the VL domain of the antibody includes the CDR sequences of SEQ ID NO: 32; the VH domain of the antibody includes the CDR sequences of SEQ ID NO: 33 and the VL domain of the antibody includes the CDR sequences of SEQ ID NO: 34; the VH domain of the antibody includes the CDR sequences of SEQ ID NO: 35 and the VL domain of the antibody includes the CDR sequences of SEQ ID NO: 36; the VH domain of the antibody includes the CDR sequences of SEQ ID NO: 37 and the VL domain of the antibody includes the CDR sequences of SEQ ID NO: 38; the VH domain of the antibody includes the CDR sequences of SEQ ID NO: 39 and the VL domain of the antibody includes the CDR sequences of SEQ ID NO: 40; or the VH domain of the antibody includes the CDR sequences of SEQ ID NO: 41 and the VL domain of the antibody includes the CDR sequences of SEQ ID NO: 42. In particular examples, the VH domain CDR1, CDR2 and CDR3 are located at positions 31-35, 50-66 and 99-112, respectively, of any one of SEQ ID NOs: 1, 3, 5, 7, 9, 11, 13, 15, 17, 19, 21, 23, 25, 27, 29, 31, 33, 35, 37, 39 and 41; and/or the VL domain CDR1, CDR2 and CDR3 are located at positions 24-33, 49-55 and 88-96, respectively, of any one of SEQ ID NOs: 2, 4, 6, 8, 10, 12, 14, 16, 18, 20, 22, 24, 26, 28, 30, 32, 34, 36, 38, 40 and 42.

In specific examples, the amino acid sequence of the VH domain includes or consists of SEQ ID NO: 1 and the amino acid sequence of the VL domain includes or consists of SEQ ID NO: 2; the amino acid sequence of the VH domain includes or consists of SEQ ID NO: 3 and the amino acid sequence of the VL domain includes or consists of SEQ ID NO: 4; the amino acid sequence of the VH domain includes or consists of SEQ ID NO: 5 and the amino acid sequence of the VL domain includes or consists of SEQ ID NO: 6; the amino acid sequence of the VH domain includes or consists of SEQ ID NO: 7 and the amino acid sequence of the VL domain includes or consists of SEQ ID NO: 8; the amino acid sequence of the VH domain includes or consists of SEQ ID NO: 9 and the amino acid sequence of the VL domain includes or consists of SEQ ID NO: 10; the amino acid sequence of the VH domain includes or consists of SEQ ID NO: 11 and the amino acid sequence of the VL domain includes or consists of SEQ ID NO: 12; the amino acid sequence of the VH domain includes or consists of SEQ ID NO: 13 and the amino acid sequence of the VL domain includes or consists of SEQ ID NO: 14; the amino acid sequence of the VH domain includes or consists of SEQ ID NO: 15 and the amino acid sequence of the VL domain includes or consists of SEQ ID NO: 16; the amino acid sequence of the VH domain includes or consists of SEQ ID NO: 17 and the amino acid sequence of the VL domain includes or consists of SEQ ID NO: 18; the amino acid sequence of the VH domain includes or consists of SEQ ID NO: 19 and the amino acid sequence of the VL domain includes or consists of SEQ ID NO: 20; the amino acid sequence of the VH domain includes or consists of SEQ ID NO: 21 and the amino acid sequence of the VL domain includes or consists of SEQ ID NO: 22; the amino acid sequence of the VH domain includes or consists of SEQ ID NO: 23 and the amino acid sequence of the VL domain includes or consists of SEQ ID NO: 24; the amino acid sequence of the VH domain includes or consists of SEQ ID NO: 25 and the amino acid sequence of the VL domain includes or consists of SEQ ID NO: 26; the amino acid sequence of the VH domain includes or consists of SEQ ID NO: 27 and the amino acid sequence of the VL domain includes or consists of SEQ ID NO: 28; the amino acid sequence of the VH domain includes or consists of SEQ ID NO: 29 and the amino acid sequence of the VL domain includes or consists of SEQ ID NO: 30; the amino acid sequence of the VH domain includes or consists of SEQ ID NO: 31 and the amino acid sequence of the VL domain includes or consists of SEQ ID NO: 32; the amino acid sequence of the VH domain includes or consists of SEQ ID NO: 33 and the amino acid sequence of the VL domain includes or consists of SEQ ID NO: 34; the amino acid sequence of the VH domain includes or consists of SEQ ID NO: 35 and the amino acid sequence of the VL domain includes or consists of SEQ ID NO: 36; the amino acid sequence of the VH domain includes or consists of SEQ ID NO: 37 and the amino acid sequence of the VL domain includes or consists of SEQ ID NO: 38; the amino acid sequence of the VH domain includes or consists of SEQ ID NO: 39 and the amino acid sequence of the VL domain includes or consists of SEQ ID NO: 40; or the amino acid sequence of the VH domain includes or consists of SEQ ID NO: 41 and the amino acid sequence of the VL domain includes or consists of SEQ ID NO: 42.

In some aspects, the CD25-specific monoclonal antibody does not significantly block binding of IL-2 to CD25. In the context of the present disclosure, an antibody that does not significantly block binding of IL-2 to CD25 is an antibody that reduces binding of IL-2 to CD25 less than 10%, less than 9%, less than 8%, less than 7%, less than 6%, less than 5%, less than 4%, less than 3%, less than 2%, less than 1%, or 0% (no detectable reduction in binding) relative to binding of IL-2 to CD25 in the absence of the antibody. In some examples, the CD25-specific monoclonal antibody does not prevent maturation or proliferation of T cells, such as CD8+ T cells.

In some aspects, the CD25-specific monoclonal antibody induces little to no ADCC. In the context of the present disclosure, an antibody that induces little to no ADCC is an antibody that induces cell death of less than 10%, less than 9%, less than 8%, less than 7%, less than 6%, less than 5%, less than 4%, less than 3%, less than 2%, less than 1%, or 0% (no detectable cell death) of target cells in a cytotoxicity assay. In some examples, the CD25-specific monoclonal antibody induces cell death of less than 2% of target cells, such as less than 1.5%, 1%, 0.5%, or 0.25% of target cells. Cytotoxicity assays that can be used for evaluating antibodies for ADCC activity are well-known and an exemplary assay is described in Example 3. Suitable target cells are those that express human CD25, such as KIT-225 cells.

In some aspects of the disclosed monoclonal antibodies, the monoclonal antibody is an antigen-binding fragment selected from a Fab fragment, a Fab′ fragment, a F(ab)′₂ fragment, a single chain variable fragment (scFv) and a disulfide stabilized variable fragment (dsFv).

In some aspects, the monoclonal antibody is an IgG, such as IgG₄. In other aspects, the monoclonal antibody is an IgA, IgD, IgE or IgM.

In some aspects, the monoclonal antibody is a humanized, chimeric or synthetic monoclonal antibody.

Also provided herein are heavy chain and light chain sequences that include the VH domain and VL domain, respectively, of the CD25-specific monoclonal antibodies disclosed herein. In some aspects, the heavy chain and/or light chain amino acid sequence includes a leader sequence, such as MGWSCIILFLVATATGVHS (SEQ ID NO: 57). In some aspects, the heavy chain includes a modified human IgG4 constant region set forth as SEQ ID NO: 58. In some aspects, the light chain includes a human Ig kappa constant region as set forth as SEQ ID NO: 49. In other aspects, the heavy chain constant region is a constant region from a different isotype, such as IgG1, but is modified to prevent induction of ADCC.

Human (modified) IgG4 constant region
(SEQ ID NO: 58)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK
Human Ig kappa constant region
(SEQ ID NO: 59)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

14554-VH
(SEQ ID NO: 60)
MGWSCIILFLVATATGVHSEVQLVESGGGLVQPGGSLRLSCAASGFWFSSYGMSWVRQAPGKGLELVSTINGYGDTWYYP
DSVKGRFTISRDNSKNTLYLQMNSLRAEDTAVYYCARDRDYTNSYYYAHDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK
14554-VL
(SEQ ID NO: 61)
MGWSCIILFLVATATGVHSEIVLTQSPATLSLSPGERATLSCRASSYVSFMHWLQQKPGQAPRPWIYATANLASGVPARF
SGSGSGTDYTLTISSLEPEDFAVYYCQQWSSNPPAFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC
14555-VH
(SEQ ID NO: 62)
MGWSCIILFLVATATGVHSEVQLVESGGGLVQPGGSLRLSCAASGFTFSSWGMSWVRQAPGKGLELVSTINGYGDTTYYP
DSVKGRFTISRDNSKNTLYLQMNSLRAEDTAVYYCARDRDYTNSYYYAHDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK
14555-VL
(SEQ ID NO: 63)
MGWSCIILFLVATATGVHSEIVLTQSPATLSLSPGERATLSCRASSYVSFMHWLQQKPGQAPRPWIYATANLASGVPARF
SGSGSGTDYTLTISSLEPEDFAVYYCQQWSSNPPAFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC
14556-VH
(SEQ ID NO: 64)
MGWSCIILFLVATATGVHSEVQLVESGGGLVQPGGSLRLSCAASGYTFSSYGMSWVRQAPGKGLELVSTINGYGDTTYYP
DSVKGRFTISRDNSKNTLYLQMNSLRAEDTAVYYCARDRDYSNSYYYALDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK
14556-VL
(SEQ ID NO: 65)
MGWSCIILFLVATATGVHSEIVLTQSPATLSLSPGERATLSCRASSYVSFMHWLQQKPGQAPRPWIYATANLASGVPARF
SGSGSGTDYTLTISSLEPEDFAVYYCQQWSSNPPAFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC
14557-VH
(SEQ ID NO: 66)
MGWSCIILFLVATATGVHSEVQLVESGGGLVQPGGSLRLSCAASGYTFSSYGMSWVRQAPGKGLELVSTINGYGDTWYYP
DSVKGRFTISRDNSKNTLYLQMNSLRAEDTAVYYCARDRDYSNSYYYAHDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK
14557-VL
(SEQ ID NO: 67)
MGWSCIILFLVATATGVHSEIVLTQSPATLSLSPGERATLSCRASSWVSFMHWLQQKPGQAPRPWIYATQNLASGVPARF
IGSGSGTDYTLTISSLEPEDFAVYYCQQWSSNPPAFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC
14558-VH
(SEQ ID NO: 68)
MGWSCIILFLVATATGVHSEVQLVESGGGLVQPGGSLRLSCAASGYWFSSYGMSWVRQAPGKGLELVSTINGYGDTTYYP
DSVKGRFTISRDNSKNTLYLQMNSLRAEDTAVYYCARDRDYTNSYYYAHDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK
14558-VL
(SEQ ID NO: 69)
MGWSCIILFLVATATGVHSEIVLTQSPATLSLSPGERATLSCRASSSVSFMHWLQQKPGQAPRPWIYATMNLASGVPARF
SGSGSGTDYTLTISSLEPEDFAVYYCQQWSSNPPAFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC
14559-VH
(SEQ ID NO: 70)
MGWSCIILFLVATATGVHSEVQLVESGGGLVQPGGSLRLSCAASGFTFSSYGMSWVRQAPGKGLELVSTINGYGDTTYYP
DSVKGRFTISRDNSKNTLYLQMNSLRAEDTAVYYCARDRDYSNSYYYAHDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK
14559-VL
(SEQ ID NO: 71)
MGWSCIILFLVATATGVHSEIVLTQSPATLSLSPGERATLSCRASSWVSFMHWLQQKPGQAPRPWIYATQNLASGVPARF
SGSGSGTDYTLTISSLEPEDFAVYYCQQWSSNPPAFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC
14560-VH
(SEQ ID NO: 72)
MGWSCIILFLVATATGVHSEVQLVESGGGLVQPGGSLRLSCAASGFTFSSYGMSWVRQAPGKGLELVSTINGYGDTTYYP
DSVKGRFTISRDNSKNTLYLQMNSLRAEDTAVYYCARDRDYTNSYYYAHDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK
14560-VL
(SEQ ID NO: 73)
MGWSCIILFLVATATGVHSEIVLTQSPATLSLSPGERATLSCRASSSVSFMHWLQQKPGQAPRPWIYATQNLASGVPARF
SGSGSGTDYTLTISSLEPEDFAVYYCQQWSSNPPAFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC
14561-VH
(SEQ ID NO: 74)
MGWSCIILFLVATATGVHSEVQLVESGGGLVQPGGSLRLSCAASGYTFSSWGMSWVRQAPGKGLELVSTINGYGDTWYYP
DSVKGRFTISRDNSKNTLYLQMNSLRAEDTAVYYCARDRDYSNSYYYAHDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK
14561-VL
(SEQ ID NO: 75)
MGWSCIILFLVATATGVHSEIVLTQSPATLSLSPGERATLSCRASSYVSFMHWLQQKPGQAPRPWIYATMNLASGVPARF
SGSGSGTDYTLTISSLEPEDFAVYYCQQWSSNPPAFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC
14562-VH
(SEQ ID NO: 76)
MGWSCIILFLVATATGVHSEVQLVESGGGLVQPGGSLRLSCAASGFTFSSYGMSWVRQAPGKGLELVSTINGYGDTWYYP
DSVKGRFTISRDNSKNTLYLQMNSLRAEDTAVYYCARDRDYSNSYYYAHDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK
14562-VL
(SEQ ID NO: 77)
MGWSCIILFLVATATGVHSEIVLTQSPATLSLSPGERATLSCRASSSVSFMHWLQQKPGQAPRPWIYATMNLASGVPARF
SGSGSGTDYTLTISSLEPEDFAVYYCQQWSSNPPAFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC
14563-VH
(SEQ ID NO: 78)
MGWSCIILFLVATATGVHSEVQLVESGGGLVQPGGSLRLSCAASGYTFSSYGMSWVRQAPGKGLELVSTINGYGDTTYYP
DSVKGRFTISRDNSKNTLYLQMNSLRAEDTAVYYCARDRDYGNSYYYAHDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK
14563-VL
(SEQ ID NO: 79)
MGWSCIILFLVATATGVHSEIVLTQSPATLSLSPGERATLSCRASSYVSFMHWLQQKPGQAPRPWIYATMNLASGVPARF
SGSGSGTDYTLTISSLEPEDFAVYYCQQWSSNPPAFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC
14564-VH
(SEQ ID NO: 80)
MGWSCIILFLVATATGVHSEVQLVESGGGLVQPGGSLRLSCAASGFTFSSWGMSWVRQAPGKGLELVSTINGYGDTTYYP
DSVKGRFTISRDNSKNTLYLQMNSLRAEDTAVYYCARDRDYSNSYYYAHDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK
14564-VL
(SEQ ID NO: 81)
MGWSCIILFLVATATGVHSEIVLTQSPATLSLSPGERATLSCRASSYVSFMHWLQQKPGQAPRPWIYATANLASGVPARF
SGSGSGTDYTLTISSLEPEDFAVYYCQQWSSNPPAFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC
14565-VH
(SEQ ID NO: 82)
MGWSCIILFLVATATGVHSEVQLVESGGGLVQPGGSLRLSCAASGFTFSSYGMSWVRQAPGKGLELVSTINGYGDTWYYP
DSVKGRFTISRDNSKNTLYLQMNSLRAEDTAVYYCARDRDYGNSYYYAHDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK
14565-VL
(SEQ ID NO: 83)
MGWSCIILFLVATATGVHSEIVLTQSPATLSLSPGERATLSCRASSWVSFMHWLQQKPGQAPRPWIYATQNLASGVPARF
SGSGSGTDYTLTISSLEPEDFAVYYCQQWSSNPPAFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC
14566-VH
(SEQ ID NO: 84)
MGWSCIILFLVATATGVHSEVQLVESGGGLVQPGGSLRLSCAASGFTFSSYGMSWVRQAPGKGLELVSTINGYGDTTYYP
DSVKGRFTISRDNSKNTLYLQMNSLRAEDTAVYYCARDRDYGNSYYYAHDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK
14566-VL
(SEQ ID NO: 85)
MGWSCIILFLVATATGVHSEIVLTQSPATLSLSPGERATLSCRASSWVSFMHWLQQKPGQAPRPWIYATMNLASGVPARF
SGSGSGTDYTLTISSLEPEDFAVYYCQQWSSNPPAFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC
14567-VH
(SEQ ID NO: 86)
MGWSCIILFLVATATGVHSEVQLVESGGGLVQPGGSLRLSCAASGFWFSSWGMSWVRQAPGKGLELVSTINGYGDTTYYP
DSVKGRFTISRDNSKNTLYLQMNSLRAEDTAVYYCARDRDYTNSYYYAHDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK
14567-VL
(SEQ ID NO: 87)
MGWSCIILFLVATATGVHSEIVLTQSPATLSLSPGERATLSCRASSYVSFMHWLQQKPGQAPRPWIYATQNLASGVPARF
SGSGSGTDYTLTISSLEPEDFAVYYCQQWSSNPPAFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC
14568-VH
(SEQ ID NO: 88)
MGWSCIILFLVATATGVHSEVQLVESGGGLVQPGGSLRLSCAASGFTFSSYGMSWVRQAPGKGLELVSTINGYGDTTYYP
DSVKGRFTISRDNSKNTLYLQMNSLRAEDTAVYYCARDRDYTNSYYYAHDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK
14568-VL
(SEQ ID NO: 89)
MGWSCIILFLVATATGVHSEIVLTQSPATLSLSPGERATLSCRASSYVSFMHWLQQKPGQAPRPWIYATMNLASGVPARF
SGSGSGTDYTLTISSLEPEDFAVYYCQQWSSNPPAFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC
14569-VH
(SEQ ID NO: 90)
MGWSCIILFLVATATGVHSEVQLVESGGGLVQPGGSLRLSCAASGYWFSSWGMSWVRQAPGKGLELVSTINGYGDTTYYP
DSVKGRFTISRDNSKNTLYLQMNSLRAEDTAVYYCARDRDYSNSYYYAHDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK
14569-VL
(SEQ ID NO: 91)
MGWSCIILFLVATATGVHSEIVLTQSPATLSLSPGERATLSCRASSYVSFMHWLQQKPGQAPRPWIYATMNLASGVPARF
SGSGSGTDYTLTISSLEPEDFAVYYCQQWSSNPPAFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC
14570-VH
(SEQ ID NO: 92)
MGWSCIILFLVATATGVHSEVQLVESGGGLVQPGGSLRLSCAASGFWFSSYGMSWVRQAPGKGLELVSTINGYGDTTYYP
DSVKGRFTISRDNSKNTLYLQMNSLRAEDTAVYYCARDRDYTNSYYYAHDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK
14570-VL
(SEQ ID NO: 93)
MGWSCIILFLVATATGVHSEIVLTQSPATLSLSPGERATLSCRASSSVSFMHWLQQKPGQAPRPWIYATANLASGVPARF
SGSGSGTDYTLTISSLEPEDFAVYYCQQWSSNPPAFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC
14571-VH
(SEQ ID NO: 94)
MGWSCIILFLVATATGVHSEVQLVESGGGLVQPGGSLRLSCAASGYTFSSYGMSWVRQAPGKGLELVSTINGYGDTTYYP
DSVKGRFTISRDNSKNTLYLQMNSLRAEDTAVYYCARDRDYGNSYYYAHDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK
14571-VL
(SEQ ID NO: 95)
MGWSCIILFLVATATGVHSEIVLTQSPATLSLSPGERATLSCRASSWVSFMHWLQQKPGQAPRPWIYATMNLASGVPARF
SGSGSGTDYTLTISSLEPEDFAVYYCQQWSSNPPAFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC
14572-VH
(SEQ ID NO: 96)
MGWSCIILFLVATATGVHSEVQLVESGGGLVQPGGSLRLSCAASGYWFSSYGMSWVRQAPGKGLELVSTINGYGDTTYYP
DSVKGRFTISRDNSKNTLYLQMNSLRAEDTAVYYCARDRDYTNSYYYAHDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK
14572-VL
(SEQ ID NO: 97)
MGWSCIILFLVATATGVHSEIVLTQSPATLSLSPGERATLSCRASSYVSFMHWLQQKPGQAPRPWIYATMNLASGVPARF
SGSGSGTDYTLTISSLEPEDFAVYYCQQWSSNPPAFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC
14573-VH
(SEQ ID NO: 98)
MGWSCIILFLVATATGVHSEVQLVESGGGLVQPGGSLRLSCAASGFTFSSWGMSWVRQAPGKGLELVSTINGYGDTTYYP
DSVKGRFTISRDNSKNTLYLQMNSLRAEDTAVYYCARDRDYTNSYYYAHDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK
14573-VL
(SEQ ID NO: 99)
MGWSCIILFLVATATGVHSEIVLTQSPATLSLSPGERATLSCRASSYVSFMHWLQQKPGQAPRPWIYATSNLASGVPARF
SGSGSGTDYTLTISSLEPEDFAVYYCQQWSSNPPAFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC
U579-WT-VH
(SEQ ID NO: 100)
MGWSCIILFLVATATGVHSEVQLVESGGGLVQPGGSLRLSCAASGFTFSSYGMSWVRQAPGKGLELVSTINGYGDTTYYP
DSVKGRFTISRDNSKNTLYLQMNSLRAEDTAVYYCARDRDYGNSYYYALDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK
U579-WT-VL
(SEQ ID NO: 101)
MGWSCIILFLVATATGVHSEIVLTQSPATLSLSPGERATLSCRASSSVSFMHWLQQKPGQAPRPWIYATSNLASGVPARF
SGSGSGTDYTLTISSLEPEDFAVYYCQQWSSNPPAFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC

In some aspects disclosed herein, the monoclonal antibody further includes a constant region, such as a constant region that includes at least one modification to increase half-life, stability and/or function of the monoclonal antibody. In some examples, the constant region includes a heavy chain constant region and/or a light chain constant region. In particular examples, the heavy chain constant region is a human IgG4 constant region, such as the human IgG4 constant region having the amino acid sequence of SEQ ID 50 NO: 58, and/or the light chain constant region is a human Igκ light chain, such as the human Igκ light chain having the amino acid sequence of SEQ ID NO: 59.

In specific non-limiting examples, the monoclonal antibody includes a heavy chain and a light chain, wherein the heavy chain and light chain include or consist of the amino acid sequences respectively set forth as: SEQ ID NO: 60 and SEQ ID NO: 61; SEQ ID NO: 62 and SEQ ID NO: 63; SEQ ID NO: 64 and SEQ ID NO: 65; SEQ ID NO: 66 and SEQ ID NO: 67; SEQ ID NO: 68 and SEQ ID NO: 69; SEQ ID NO: 70 and SEQ ID NO: 71; SEQ ID NO: 72 and SEQ ID NO: 73; SEQ ID NO: 74 and SEQ ID NO: 75; SEQ ID NO: 76 and SEQ ID NO: 77; SEQ ID NO: 78 and SEQ ID NO: 79; SEQ ID NO: 80 and SEQ ID NO: 81; SEQ ID NO: 82 and SEQ ID NO: 83; SEQ ID NO: 84 and SEQ ID NO: 85; SEQ ID NO: 86 and SEQ ID NO: 87; SEQ ID NO: 88 and SEQ ID NO: 89; SEQ ID NO: 90 and SEQ ID NO: 91; SEQ ID NO: 92 and SEQ ID NO: 93; SEQ ID NO: 94 and SEQ ID NO: 95; SEQ ID NO: 96 and SEQ ID NO: 97; SEQ ID NO: 98 and SEQ ID NO: 99; or SEQ ID NO: 100 and SEQ ID NO: 101.

Also provided are compositions that include a CD25-specific monoclonal antibody disclosed herein and a pharmaceutically acceptable carrier. The disclosed CD25-specific monoclonal antibodies can also be part of a kit.

### IV. Antibody Conjugates

The CD25-specific monoclonal antibodies disclosed herein can be conjugated to an agent, such as a photon absorber (e.g., IR700), an effector molecule, or a detectable label, using any number of suitable means. Both covalent and noncovalent attachment means may be used. Various photon absorber, effector molecules and detectable markers can be used, including (but not limited to) IR700, toxins and radioactive agents such as ¹²⁵I, ³²P, ¹⁴C, ³H and ³⁵S and other labels, targeting moieties and ligands.

In some aspects, provided herein are conjugates that include a CD25-specific monoclonal antibody disclosed herein and a photon absorber, an effector molecule or a detectable label. In some examples, the photon absorber is IR700; the effector molecule is a toxin; or the detectable label is a fluorescent, enzymatic, or radioactive label.

Also provided herein are compositions that include a conjugate disclosed herein and a pharmaceutically acceptable carrier.

The choice of a particular photon absorber, effector molecule or detectable marker depends on the particular target molecule or cell, and the desired biological effect. Thus, for example, the effector molecule can be a cytotoxin that is used to bring about the death of a particular target cell (such as a CD25-positive cell).

In some aspects, the photon absorber is IR700 (IRDye® 700DX), which is commercially available from Rakuten Medical (San Diego, CA). Amino-reactive IR700 is a relatively hydrophilic dye and can be covalently conjugated with an antibody using the NHS ester of IR700.

The procedure for attaching a photon absorber, effector molecule or detectable marker to an antibody varies according to the chemical structure of the conjugate. Polypeptides typically contain a variety of functional groups; such as carboxylic acid (COOH), free amine (—NH₂) or sulfhydryl (—SH) groups, which are available for reaction with a suitable functional group on a polypeptide to result in the binding of the photon absorber, effector molecule or detectable marker. Alternatively, the antibody is derivatized to expose or attach additional reactive functional groups. The derivatization may involve attachment of any of a number of known linker molecules such as those available from Pierce Chemical Company, Rockford, IL. The linker can be any molecule used to join the antibody to the photon absorber, effector molecule or detectable marker. The linker is capable of forming covalent bonds to both the antibody and to the photon absorber/effector molecule/detectable marker. Suitable linkers include, but are not limited to, straight or branched-chain carbon linkers, heterocyclic carbon linkers, or peptide linkers. Where the antibody and the effector molecule or detectable marker are polypeptides, the linkers may be joined to the constituent amino acids through their side groups (such as through a disulfide linkage to cysteine) or to the alpha carbon amino and carboxyl groups of the terminal amino acids.

The antibody can be conjugated to a detectable marker; for example, a detectable marker capable of detection by ELISA, spectrophotometry, flow cytometry, microscopy or diagnostic imaging techniques (such as computed tomography (CT), computed axial tomography (CAT) scans, magnetic resonance imaging (MRI), nuclear magnetic resonance imaging NMRI), magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, green fluorescent protein (GFP), and yellow fluorescent protein (YFP). An antibody or antigen binding fragment can also be conjugated with enzymes that are useful for detection, such as horseradish peroxidase, β-galactosidase, luciferase, alkaline phosphatase, glucose oxidase and the like. When an antibody or antigen binding fragment is conjugated with a detectable enzyme, it can be detected by adding additional reagents that the enzyme uses to produce a reaction product that can be discerned. For example, when the agent horseradish peroxidase is present the addition of hydrogen peroxide and diaminobenzidine leads to a colored reaction product, which is visually detectable. An antibody or antigen binding fragment may also be conjugated with biotin, and detected through indirect measurement of avidin or streptavidin binding. It should be noted that the avidin itself can be conjugated with an enzyme or a fluorescent label.

The antibody can be conjugated with a paramagnetic agent, such as gadolinium. Paramagnetic agents such as superparamagnetic iron oxide are also of use as labels. Antibodies can also be conjugated with lanthanides (such as europium and dysprosium), and manganese. An antibody may also be labeled with a predetermined polypeptide epitopes recognized by a secondary reporter (such as leucine zipper pair sequences, binding sites for secondary antibodies, metal binding domains, epitope tags).

The antibody can also be conjugated with a radiolabeled amino acid. The radiolabel may be used for both diagnostic and therapeutic purposes. For instance, the radiolabel may be used to detect CD25-expressing cells by x-ray, emission spectra, or other diagnostic techniques. Examples of labels for polypeptides include, but are not limited to, the following radioisotopes or radionucleotides: ³H, ¹⁴C, ¹⁵N, ³⁵S, ⁹⁰Y, ⁹⁹Tc, ¹¹³In, ¹²⁵I, ¹³¹I.

Any known means of detecting detectable markers can be used. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The average number of photon absorber, effector molecule or detectable marker moieties per antibody in a conjugate can range, for example, from 1 to 20 moieties per antibody. In certain aspects, the average number of photon absorber, effector molecule or detectable marker moieties per antibody in a conjugate range from about 1 to about 2, from about 1 to about 3, about 1 to about 8; from about 2 to about 6; from about 3 to about 5; about 2 to about 3; or from about 3 to about 4. In specific non-limiting examples, the conjugate includes one CD25-specific monoclonal antibody conjugated to two or three IR700 molecules. The loading (for example, effector molecule/antibody ratio) of an conjugate may be controlled in different ways, for example, by: (i) limiting the molar excess of effector molecule-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number or position of linker-effector molecule attachments.

### V. Nucleic Acid Molecules

Nucleic acid molecules (for example, DNA, RNA, or cDNA molecules) encoding the amino acid sequences of the disclosed CD25-specific antibodies and conjugates are provided. Nucleic acids encoding these molecules can readily be produced using the amino acid sequences provided herein (such as the CDR sequences and V_{H} and V_{L} sequences), sequences available in the art (such as framework or constant region sequences), and the genetic code. In several aspects, a nucleic acid molecule can encode the V_{H}, the V_{L}, or both the V_{H} and V_{L} (for example in a bicistronic expression vector) of a disclosed antibody. In several aspects, the nucleic acid molecules can be expressed in a host cell (such as a mammalian cell) to produce a disclosed antibody.

The genetic code can be used to construct a variety of functionally equivalent nucleic acids, such as nucleic acids that differ in sequence, but which encode the same antibody sequence, or encode a conjugate or fusion protein including the V_{L} and/or V_{H} nucleic acid sequence.

Provided below are nucleic acid sequences of the VH and VL domains of the disclosed CD25-specific monoclonal antibodies (SEQ ID NOs: 102-143), exemplary leader sequences (SEQ ID NOs: 144 and 145), an exemplary human IgG4 constant region coding sequence (SEQ ID NO: 146) and an exemplary human Igκ light chain coding sequence (SEQ ID NO: 147). Further provided are nucleic acid sequences encoding complete heavy chain and light chains for the disclosed monoclonal antibodies (SEQ ID NOs: 148-189).

14554-VH (SEQ ID NO: 102)
GAGGTTCAACTAGTAGAATCAGGTGGAGGGTTGGTGCAGCCGGGTGGTAGCCTCCGCCTGAGCTGTGCTGCGAGCGGTTT
CTGGTTCAGCTCCTACGGCATGAGCTGGGTTCGTCAGGCACCGGGCAAAGGCCTGGAACTGGTGTCCACCATCAACGGTT
ATGGCGATACGTGGTATTACCCGGATAGCGTTAAAGGCCGTTTTACCATTAGCCGTGACAACTCTAAGAACACCTTATAT
CTGCAAATGAATTCACTGCGCGCAGAGGACACTGCCGTTTATTACTGCGCGCGTGACAGAGACTACACTAATTCCTATTA
CTACGCGCACGATTACTGGGGTCAAGGTACGTTGGTCACCGTGTCTTCG
14554-VL (SEQ ID NO: 103)
GAAATAGTACTAACACAATCACCCGCTACTTTGTCACTCTCGCCGGGTGAGCGCGCGACCCTGTCTTGTCGTGCCAGCTC
CTACGTGAGCTTTATGCACTGGCTGCAGCAAAAACCGGGTCAGGCACCGCGTCCGTGGATTTATGCGACGGCCAATCTGG
CTAGCGGTGTTCCGGCGCGTTTCAGCGGCAGCGGCTCTGGCACTGACTACACCTTGACCATCAGCTCTCTGGAACCGGAA
GATTTCGCTGTGTATTACTGCCAGCAATGGAGCTCCAACCCACCGGCGTTTGGTGGTGGCACCAAAGTTGAGATCAAG
14555-VH (SEQ ID NO: 104)
GAGGTTCAACTAGTAGAATCAGGTGGAGGGTTGGTGCAGCCGGGTGGTAGCCTCCGCCTGAGCTGTGCTGCGAGCGGTTT
CACCTTCAGCTCCTGGGGCATGAGCTGGGTTCGTCAGGCACCGGGCAAAGGCCTGGAACTGGTGTCCACCATCAACGGTT
ATGGCGATACGACCTATTACCCGGATAGCGTTAAAGGCCGTTTTACCATTAGCCGTGACAACTCTAAGAACACCTTATAT
CTGCAAATGAATTCACTGCGCGCAGAGGACACTGCCGTTTATTACTGCGCGCGTGACAGAGACTACACTAATTCCTATTA
CTACGCGCACGATTACTGGGGTCAAGGTACGTTGGTCACCGTGTCTTCG
14555-VL (SEQ ID NO: 105)
GAAATAGTACTAACACAATCACCCGCTACTTTGTCACTCTCGCCGGGTGAGCGCGCGACCCTGTCTTGTCGTGCCAGCTC
CTACGTGAGCTTTATGCACTGGCTGCAGCAAAAACCGGGTCAGGCACCGCGTCCGTGGATTTATGCGACGGCCAATCTGG
CTAGCGGTGTTCCGGCGCGTTTCAGCGGCAGCGGCTCTGGCACTGACTACACCTTGACCATCAGCTCTCTGGAACCGGAA
GATTTCGCTGTGTATTACTGCCAGCAATGGAGCTCCAACCCACCGGCGTTTGGTGGTGGCACCAAAGTTGAGATCAAG
14556-VH (SEQ ID NO: 106)
GAGGTTCAACTAGTAGAATCAGGTGGAGGGTTGGTGCAGCCGGGTGGTAGCCTCCGCCTGAGCTGTGCTGCGAGCGGTTA
CACCTTCAGCTCCTACGGCATGAGCTGGGTTCGTCAGGCACCGGGCAAAGGCCTGGAACTGGTGTCCACCATCAACGGTT
ATGGCGATACGACCTATTACCCGGATAGCGTTAAAGGCCGTTTTACCATTAGCCGTGACAACTCTAAGAACACCTTATAT
CTGCAAATGAATTCACTGCGCGCAGAGGACACTGCCGTTTATTACTGCGCGCGTGACAGAGACTACTCTAATTCCTATTA
CTACGCGCTGGATTACTGGGGTCAAGGTACGTTGGTCACCGTGTCTTCG
14556-VL (SEQ ID NO: 107)
GAAATAGTACTAACACAATCACCCGCTACTTTGTCACTCTCGCCGGGTGAGCGCGCGACCCTGTCTTGTCGTGCCAGCTC
CTACGTGAGCTTTATGCACTGGCTGCAGCAAAAACCGGGTCAGGCACCGCGTCCGTGGATTTATGCGACGGCCAATCTGG
CTAGCGGTGTTCCGGCGCGTTTCAGCGGCAGCGGCTCTGGCACTGACTACACCTTGACCATCAGCTCTCTGGAACCGGAA
GATTTCGCTGTGTATTACTGCCAGCAATGGAGCTCCAACCCACCGGCGTTTGGTGGTGGCACCAAAGTTGAGATCAAG
14557-VH (SEQ ID NO: 108)
GAGGTTCAACTAGTAGAATCAGGTGGAGGGTTGGTGCAGCCGGGTGGTAGCCTCCGCCTGAGCTGTGCTGCGAGCGGTTA
CACCTTCAGCTCCTACGGCATGAGCTGGGTTCGTCAGGCACCGGGCAAAGGCCTGGAACTGGTGTCCACCATCAACGGTT
ATGGCGATACGTGGTATTACCCGGATAGCGTTAAAGGCCGTTTTACCATTAGCCGTGACAACTCTAAGAACACCTTATAT
CTGCAAATGAATTCACTGCGCGCAGAGGACACTGCCGTTTATTACTGCGCGCGTGACAGAGACTACTCTAATTCCTATTA
CTACGCGCACGATTACTGGGGTCAAGGTACGTTGGTCACCGTGTCTTCG
14557-VL (SEQ ID NO: 109)
GAAATAGTACTAACACAATCACCCGCTACTTTGTCACTCTCGCCGGGTGAGCGCGCGACCCTGTCTTGTCGTGCCAGCTC
CTGGGTGAGCTTTATGCACTGGCTGCAGCAAAAACCGGGTCAGGCACCGCGTCCGTGGATTTATGCGACGCAGAATCTGG
CTAGCGGTGTTCCGGCGCGTTTCATCGGCAGCGGCTCTGGCACTGACTACACCTTGACCATCAGCTCTCTGGAACCGGAA
GATTTCGCTGTGTATTACTGCCAGCAATGGAGCTCCAACCCACCGGCGTTTGGTGGTGGCACCAAAGTTGAGATCAAG
14558-VH (SEQ ID NO: 110)
GAGGTTCAACTAGTAGAATCAGGTGGAGGGTTGGTGCAGCCGGGTGGTAGCCTCCGCCTGAGCTGTGCTGCGAGCGGTTA
CTGGTTCAGCTCCTACGGCATGAGCTGGGTTCGTCAGGCACCGGGCAAAGGCCTGGAACTGGTGTCCACCATCAACGGTT
ATGGCGATACGACCTATTACCCGGATAGCGTTAAAGGCCGTTTTACCATTAGCCGTGACAACTCTAAGAACACCTTATAT
CTGCAAATGAATTCACTGCGCGCAGAGGACACTGCCGTTTATTACTGCGCGCGTGACAGAGACTACACTAATTCCTATTA
CTACGCGCACGATTACTGGGGTCAAGGTACGTTGGTCACCGTGTCTTCG
14558-VL (SEQ ID NO: 111)
GAAATAGTACTAACACAATCACCCGCTACTTTGTCACTCTCGCCGGGTGAGCGCGCGACCCTGTCTTGTCGTGCCAGCTC
CTCCGTGAGCTTTATGCACTGGCTGCAGCAAAAACCGGGTCAGGCACCGCGTCCGTGGATTTATGCGACGATGAATCTGG
CTAGCGGTGTTCCGGCGCGTTTCAGCGGCAGCGGCTCTGGCACTGACTACACCTTGACCATCAGCTCTCTGGAACCGGAA
GATTTCGCTGTGTATTACTGCCAGCAATGGAGCTCCAACCCACCGGCGTTTGGTGGTGGCACCAAAGTTGAGATCAAG
14559-VH (SEQ ID NO: 112)
GAGGTTCAACTAGTAGAATCAGGTGGAGGGTTGGTGCAGCCGGGTGGTAGCCTCCGCCTGAGCTGTGCTGCGAGCGGTTT
CACCTTCAGCTCCTACGGCATGAGCTGGGTTCGTCAGGCACCGGGCAAAGGCCTGGAACTGGTGTCCACCATCAACGGTT
ATGGCGATACGACCTATTACCCGGATAGCGTTAAAGGCCGTTTTACCATTAGCCGTGACAACTCTAAGAACACCTTATAT
CTGCAAATGAATTCACTGCGCGCAGAGGACACTGCCGTTTATTACTGCGCGCGTGACAGAGACTACTCTAATTCCTATTA
CTACGCGCACGATTACTGGGGTCAAGGTACGTTGGTCACCGTGTCTTCG
14559-VL (SEQ ID NO: 113)
GAAATAGTACTAACACAATCACCCGCTACTTTGTCACTCTCGCCGGGTGAGCGCGCGACCCTGTCTTGTCGTGCCAGCTC
CTGGGTGAGCTTTATGCACTGGCTGCAGCAAAAACCGGGTCAGGCACCGCGTCCGTGGATTTATGCGACGCAGAATCTGG
CTAGCGGTGTTCCGGCGCGTTTCAGCGGCAGCGGCTCTGGCACTGACTACACCTTGACCATCAGCTCTCTGGAACCGGAA
GATTTCGCTGTGTATTACTGCCAGCAATGGAGCTCCAACCCACCGGCGTTTGGTGGTGGCACCAAAGTTGAGATCAAG
14560-VH (SEQ ID NO: 114)
GAGGTTCAACTAGTAGAATCAGGTGGAGGGTTGGTGCAGCCGGGTGGTAGCCTCCGCCTGAGCTGTGCTGCGAGCGGTTT
CACCTTCAGCTCCTACGGCATGAGCTGGGTTCGTCAGGCACCGGGCAAAGGCCTGGAACTGGTGTCCACCATCAACGGTT
ATGGCGATACGACCTATTACCCGGATAGCGTTAAAGGCCGTTTTACCATTAGCCGTGACAACTCTAAGAACACCTTATAT
CTGCAAATGAATTCACTGCGCGCAGAGGACACTGCCGTTTATTACTGCGCGCGTGACAGAGACTACACTAATTCCTATTA
CTACGCGCACGATTACTGGGGTCAAGGTACGTTGGTCACCGTGTCTTCG
14560-VL (SEQ ID NO: 115)
GAAATAGTACTAACACAATCACCCGCTACTTTGTCACTCTCGCCGGGTGAGCGCGCGACCCTGTCTTGTCGTGCCAGCTC
CTCCGTGAGCTTTATGCACTGGCTGCAGCAAAAACCGGGTCAGGCACCGCGTCCGTGGATTTATGCGACGCAGAATCTGG
CTAGCGGTGTTCCGGCGCGTTTCAGCGGCAGCGGCTCTGGCACTGACTACACCTTGACCATCAGCTCTCTGGAACCGGAA
GATTTCGCTGTGTATTACTGCCAGCAATGGAGCTCCAACCCACCGGCGTTTGGTGGTGGCACCAAAGTTGAGATCAAG
14561-VH (SEQ ID NO: 116)
GAGGTTCAACTAGTAGAATCAGGTGGAGGGTTGGTGCAGCCGGGTGGTAGCCTCCGCCTGAGCTGTGCTGCGAGCGGTTA
CACCTTCAGCTCCTGGGGCATGAGCTGGGTTCGTCAGGCACCGGGCAAAGGCCTGGAACTGGTGTCCACCATCAACGGTT
ATGGCGATACGTGGTATTACCCGGATAGCGTTAAAGGCCGTTTTACCATTAGCCGTGACAACTCTAAGAACACCTTATAT
CTGCAAATGAATTCACTGCGCGCAGAGGACACTGCCGTTTATTACTGCGCGCGTGACAGAGACTACTCTAATTCCTATTA
CTACGCGCACGATTACTGGGGTCAAGGTACGTTGGTCACCGTGTCTTCG
14561-VL (SEQ ID NO: 117)
GAAATAGTACTAACACAATCACCCGCTACTTTGTCACTCTCGCCGGGTGAGCGCGCGACCCTGTCTTGTCGTGCCAGCTC
CTACGTGAGCTTTATGCACTGGCTGCAGCAAAAACCGGGTCAGGCACCGCGTCCGTGGATTTATGCGACGATGAATCTGG
CTAGCGGTGTTCCGGCGCGTTTCAGCGGCAGCGGCTCTGGCACTGACTACACCTTGACCATCAGCTCTCTGGAACCGGAA
GATTTCGCTGTGTATTACTGCCAGCAATGGAGCTCCAACCCACCGGCGTTTGGTGGTGGCACCAAAGTTGAGATCAAG
14562-VH (SEQ ID NO: 118)
GAGGTTCAACTAGTAGAATCAGGTGGAGGGTTGGTGCAGCCGGGTGGTAGCCTCCGCCTGAGCTGTGCTGCGAGCGGTTT
CACCTTCAGCTCCTACGGCATGAGCTGGGTTCGTCAGGCACCGGGCAAAGGCCTGGAACTGGTGTCCACCATCAACGGTT
ATGGCGATACGTGGTATTACCCGGATAGCGTTAAAGGCCGTTTTACCATTAGCCGTGACAACTCTAAGAACACCTTATAT
CTGCAAATGAATTCACTGCGCGCAGAGGACACTGCCGTTTATTACTGCGCGCGTGACAGAGACTACTCTAATTCCTATTA
CTACGCGCACGATTACTGGGGTCAAGGTACGTTGGTCACCGTGTCTTCG
14562-VL (SEQ ID NO: 119)
GAAATAGTACTAACACAATCACCCGCTACTTTGTCACTCTCGCCGGGTGAGCGCGCGACCCTGTCTTGTCGTGCCAGCTC
CTCCGTGAGCTTTATGCACTGGCTGCAGCAAAAACCGGGTCAGGCACCGCGTCCGTGGATTTATGCGACGATGAATCTGG
CTAGCGGTGTTCCGGCGCGTTTCAGCGGCAGCGGCTCTGGCACTGACTACACCTTGACCATCAGCTCTCTGGAACCGGAA
GATTTCGCTGTGTATTACTGCCAGCAATGGAGCTCCAACCCACCGGCGTTTGGTGGTGGCACCAAAGTTGAGATCAAG
14563-VII(SEQ ID NO: 120)
GAGGTTCAACTAGTAGAATCAGGTGGAGGGTTGGTGCAGCCGGGTGGTAGCCTCCGCCTGAGCTGTGCTGCGAGCGGTTA
CACCTTCAGCTCCTACGGCATGAGCTGGGTTCGTCAGGCACCGGGCAAAGGCCTGGAACTGGTGTCCACCATCAACGGTT
ATGGCGATACGACCTATTACCCGGATAGCGTTAAAGGCCGTTTTACCATTAGCCGTGACAACTCTAAGAACACCTTATAT
CTGCAAATGAATTCACTGCGCGCAGAGGACACTGCCGTTTATTACTGCGCGCGTGACAGAGACTACGGTAATTCCTATTA
CTACGCGCACGATTACTGGGGTCAAGGTACGTTGGTCACCGTGTCTTCG
14563-VL (SEQ ID NO: 121)
GAAATAGTACTAACACAATCACCCGCTACTTTGTCACTCTCGCCGGGTGAGCGCGCGACCCTGTCTTGTCGTGCCAGCTC
CTACGTGAGCTTTATGCACTGGCTGCAGCAAAAACCGGGTCAGGCACCGCGTCCGTGGATTTATGCGACGATGAATCTGG
CTAGCGGTGTTCCGGCGCGTTTCAGCGGCAGCGGCTCTGGCACTGACTACACCTTGACCATCAGCTCTCTGGAACCGGAA
GATTTCGCTGTGTATTACTGCCAGCAATGGAGCTCCAACCCACCGGCGTTTGGTGGTGGCACCAAAGTTGAGATCAAG
14564-VH (SEQ ID NO: 122)
GAGGTTCAACTAGTAGAATCAGGTGGAGGGTTGGTGCAGCCGGGTGGTAGCCTCCGCCTGAGCTGTGCTGCGAGCGGTTT
CACCTTCAGCTCCTGGGGCATGAGCTGGGTTCGTCAGGCACCGGGCAAAGGCCTGGAACTGGTGTCCACCATCAACGGTT
ATGGCGATACGACCTATTACCCGGATAGCGTTAAAGGCCGTTTTACCATTAGCCGTGACAACTCTAAGAACACCTTATAT
CTGCAAATGAATTCACTGCGCGCAGAGGACACTGCCGTTTATTACTGCGCGCGTGACAGAGACTACTCTAATTCCTATTA
CTACGCGCACGATTACTGGGGTCAAGGTACGTTGGTCACCGTGTCTTCG
14564-VL (SEQ ID NO: 123)
GAAATAGTACTAACACAATCACCCGCTACTTTGTCACTCTCGCCGGGTGAGCGCGCGACCCTGTCTTGTCGTGCCAGCTC
CTACGTGAGCTTTATGCACTGGCTGCAGCAAAAACCGGGTCAGGCACCGCGTCCGTGGATTTATGCGACGGCCAATCTGG
CTAGCGGTGTTCCGGCGCGTTTCAGCGGCAGCGGCTCTGGCACTGACTACACCTTGACCATCAGCTCTCTGGAACCGGAA
GATTTCGCTGTGTATTACTGCCAGCAATGGAGCTCCAACCCACCGGCGTTTGGTGGTGGCACCAAAGTTGAGATCAAG
14565-VH (SEQ ID NO: 124)
GAGGTTCAACTAGTAGAATCAGGTGGAGGGTTGGTGCAGCCGGGTGGTAGCCTCCGCCTGAGCTGTGCTGCGAGCGGTTT
CACCTTCAGCTCCTACGGCATGAGCTGGGTTCGTCAGGCACCGGGCAAAGGCCTGGAACTGGTGTCCACCATCAACGGTT
ATGGCGATACGTGGTATTACCCGGATAGCGTTAAAGGCCGTTTTACCATTAGCCGTGACAACTCTAAGAACACCTTATAT
CTGCAAATGAATTCACTGCGCGCAGAGGACACTGCCGTTTATTACTGCGCGCGTGACAGAGACTACGGTAATTCCTATTA
CTACGCGCACGATTACTGGGGTCAAGGTACGTTGGTCACCGTGTCTTCG
14565-VL (SEQ ID NO: 125)
GAAATAGTACTAACACAATCACCCGCTACTTTGTCACTCTCGCCGGGTGAGCGCGCGACCCTGTCTTGTCGTGCCAGCTC
CTGGGTGAGCTTTATGCACTGGCTGCAGCAAAAACCGGGTCAGGCACCGCGTCCGTGGATTTATGCGACGCAGAATCTGG
CTAGCGGTGTTCCGGCGCGTTTCAGCGGCAGCGGCTCTGGCACTGACTACACCTTGACCATCAGCTCTCTGGAACCGGAA
GATTTCGCTGTGTATTACTGCCAGCAATGGAGCTCCAACCCACCGGCGTTTGGTGGTGGCACCAAAGTTGAGATCAAG
14566-VH (SEQ ID NO: 126)
GAGGTTCAACTAGTAGAATCAGGTGGAGGGTTGGTGCAGCCGGGGGTAGCCTCCGCCTGAGCTGTGCTGCGAGCGGTTT
CACCTTCAGCTCCTACGGCATGAGCTGGGTTCGTCAGGCACCGGGCAAAGGCCTGGAACTGGTGTCCACCATCAACGGTT
ATGGCGATACGACCTATTACCCGGATAGCGTTAAAGGCCGTTTTACCATTAGCCGTGACAACTCTAAGAACACCTTATAT
CTGCAAATGAATTCACTGCGCGCAGAGGACACTGCCGTTTATTACTGCGCGCGTGACAGAGACTACGGTAATTCCTATTA
CTACGCGCACGATTACTGGGGTCAAGGTACGTTGGTCACCGTGTCTTCG
14566-VL (SEQ ID NO: 127)
GAAATAGTACTAACACAATCACCCGCTACTTTGTCACTCTCGCCGGGTGAGCGCGCGACCCTGTCTTGTCGTGCCAGCTC
CTGGGTGAGCTTTATGCACTGGCTGCAGCAAAAACCGGGTCAGGCACCGCGTCCGTGGATTTATGCGACGATGAATCTGG
CTAGCGGTGTTCCGGCGCGTTTCAGCGGCAGCGGCTCTGGCACTGACTACACCTTGACCATCAGCTCTCTGGAACCGGAA
GATTTCGCTGTGTATTACTGCCAGCAATGGAGCTCCAACCCACCGGCGTTTGGTGGTGGCACCAAAGTTGAGATCAAG
14567-VH (SEQ ID NO: 128)
GAGGTTCAACTAGTAGAATCAGGTGGAGGGTTGGTGCAGCCGGGTGGTAGCCTCCGCCTGAGCTGTGCTGCGAGCGGTTT
CTGGTTCAGCTCCTGGGGCATGAGCTGGGTTCGTCAGGCACCGGGCAAAGGCCTGGAACTGGTGTCCACCATCAACGGTT
ATGGCGATACGACCTATTACCCGGATAGCGTTAAAGGCCGTTTTACCATTAGCCGTGACAACTCTAAGAACACCTTATAT
CTGCAAATGAATTCACTGCGCGCAGAGGACACTGCCGTTTATTACTGCGCGCGTGACAGAGACTACACTAATTCCTATTA
CTACGCGCACGATTACTGGGGTCAAGGTACGTTGGTCACCGTGTCTTCG
14567-VL (SEQ ID NO: 129)
GAAATAGTACTAACACAATCACCCGCTACTTTGTCACTCTCGCCGGGTGAGCGCGCGACCCTGTCTTGTCGTGCCAGCTC
CTACGTGAGCTTTATGCACTGGCTGCAGCAAAAACCGGGTCAGGCACCGCGTCCGTGGATTTATGCGACGCAGAATCTGG
CTAGCGGTGTTCCGGCGCGTTTCAGCGGCAGCGGCTCTGGCACTGACTACACCTTGACCATCAGCTCTCTGGAACCGGAA
GATTTCGCTGTGTATTACTGCCAGCAATGGAGCTCCAACCCACCGGCGTTTGGTGGTGGCACCAAAGTTGAGATCAAG
14568-VH (SEQ ID NO: 130)
GAGGTTCAACTAGTAGAATCAGGTGGAGGGTTGGTGCAGCCGGGTGGTAGCCTCCGCCTGAGCTGTGCTGCGAGCGGTTT
CACCTTCAGCTCCTACGGCATGAGCTGGGTTCGTCAGGCACCGGGCAAAGGCCTGGAACTGGTGTCCACCATCAACGGTT
ATGGCGATACGACCTATTACCCGGATAGCGTTAAAGGCCGTTTTACCATTAGCCGTGACAACTCTAAGAACACCTTATAT
CTGCAAATGAATTCACTGCGCGCAGAGGACACTGCCGTTTATTACTGCGCGCGTGACAGAGACTACACTAATTCCTATTA
CTACGCGCACGATTACTGGGGTCAAGGTACGTTGGTCACCGTGTCTTCG
14568-VL (SEQ ID NO: 131)
GAAATAGTACTAACACAATCACCCGCTACTTTGTCACTCTCGCCGGGTGAGCGCGCGACCCTGTCTTGTCGTGCCAGCTC
CTACGTGAGCTTTATGCACTGGCTGCAGCAAAAACCGGGTCAGGCACCGCGTCCGTGGATTTATGCGACGATGAATCTGG
CTAGCGGTGTTCCGGCGCGTTTCAGCGGCAGCGGCTCTGGCACTGACTACACCTTGACCATCAGCTCTCTGGAACCGGAA
GATTTCGCTGTGTATTACTGCCAGCAATGGAGCTCCAACCCACCGGCGTTTGGTGGTGGCACCAAAGTTGAGATCAAG
14569-VH (SEQ ID NO: 132)
GAGGTTCAACTAGTAGAATCAGGTGGAGGGTTGGTGCAGCCGGGTGGTAGCCTCCGCCTGAGCTGTGCTGCGAGCGGTTA
CTGGTTCAGCTCCTGGGGCATGAGCTGGGTTCGTCAGGCACCGGGCAAAGGCCTGGAACTGGTGTCCACCATCAACGGTT
ATGGCGATACGACCTATTACCCGGATAGCGTTAAAGGCCGTTTTACCATTAGCCGTGACAACTCTAAGAACACCTTATAT
CTGCAAATGAATTCACTGCGCGCAGAGGACACTGCCGTTTATTACTGCGCGCGTGACAGAGACTACTCTAATTCCTATTA
CTACGCGCACGATTACTGGGGTCAAGGTACGTTGGTCACCGTGTCTTCG
14569-VL (SEQ ID NO: 133)
GAAATAGTACTAACACAATCACCCGCTACTTTGTCACTCTCGCCGGGTGAGCGCGCGACCCTGTCTTGTCGTGCCAGCTC
CTACGTGAGCTTTATGCACTGGCTGCAGCAAAAACCGGGTCAGGCACCGCGTCCGTGGATTTATGCGACGATGAATCTGG
CTAGCGGTGTTCCGGCGCGTTTCAGCGGCAGCGGCTCTGGCACTGACTACACCTTGACCATCAGCTCTCTGGAACCGGAA
GATTTCGCTGTGTATTACTGCCAGCAATGGAGCTCCAACCCACCGGCGTTTGGTGGTGGCACCAAAGTTGAGATCAAG
14570-VH (SEQ ID NO: 134)
GAGGTTCAACTAGTAGAATCAGGTGGAGGGTTGGTGCAGCCGGGTGGTAGCCTCCGCCTGAGCTGTGCTGCGAGCGGTTT
CTGGTTCAGCTCCTACGGCATGAGCTGGGTTCGTCAGGCACCGGGCAAAGGCCTGGAACTGGTGTCCACCATCAACGGTT
ATGGCGATACGACCTATTACCCGGATAGCGTTAAAGGCCGTTTTACCATTAGCCGTGACAACTCTAAGAACACCTTATAT
CTGCAAATGAATTCACTGCGCGCAGAGGACACTGCCGTTTATTACTGCGCGCGTGACAGAGACTACACTAATTCCTATTA
CTACGCGCACGATTACTGGGGTCAAGGTACGTTGGTCACCGTGTCTTCG
14570-VL (SEQ ID NO: 135)
GAAATAGTACTAACACAATCACCCGCTACTTTGTCACTCTCGCCGGGTGAGCGCGCGACCCTGTCTTGTCGTGCCAGCTC
CTCCGTGAGCTTTATGCACTGGCTGCAGCAAAAACCGGGTCAGGCACCGCGTCCGTGGATTTATGCGACGGCCAATCTGG
CTAGCGGTGTTCCGGCGCGTTTCAGCGGCAGCGGCTCTGGCACTGACTACACCTTGACCATCAGCTCTCTGGAACCGGAA
GATTTCGCTGTGTATTACTGCCAGCAATGGAGCTCCAACCCACCGGCGTTTGGTGGTGGCACCAAAGTTGAGATCAAG
14571-VH (SEQ ID NO: 136)
GAGGTTCAACTAGTAGAATCAGGTGGAGGGTTGGTGCAGCCGGGTGGTAGCCTCCGCCTGAGCTGTGCTGCGAGCGGTTA
CACCTTCAGCTCCTACGGCATGAGCTGGGTTCGTCAGGCACCGGGCAAAGGCCTGGAACTGGTGTCCACCATCAACGGTT
ATGGCGATACGACCTATTACCCGGATAGCGTTAAAGGCCGTTTTACCATTAGCCGTGACAACTCTAAGAACACCTTATAT
CTGCAAATGAATTCACTGCGCGCAGAGGACACTGCCGTTTATTACTGCGCGCGTGACAGAGACTACGGTAATTCCTATTA
CTACGCGCACGATTACTGGGGTCAAGGTACGTTGGTCACCGTGTCTTCG
14571-VL (SEQ ID NO: 137)
GAAATAGTACTAACACAATCACCCGCTACTTTGTCACTCTCGCCGGGTGAGCGCGCGACCCTGTCTTGTCGTGCCAGCTC
CTGGGTGAGCTTTATGCACTGGCTGCAGCAAAAACCGGGTCAGGCACCGCGTCCGTGGATTTATGCGACGATGAATCTGG
CTAGCGGTGTTCCGGCGCGTTTCAGCGGCAGCGGCTCTGGCACTGACTACACCTTGACCATCAGCTCTCTGGAACCGGAA
GATTTCGCTGTGTATTACTGCCAGCAATGGAGCTCCAACCCACCGGCGTTTGGTGGTGGCACCAAAGTTGAGATCAAG
14572-VH (SEQ ID NO: 138)
GAGGTTCAACTAGTAGAATCAGGTGGAGGGTTGGTGCAGCCGGGTGGTAGCCTCCGCCTGAGCTGTGCTGCGAGCGGTTA
CTGGTTCAGCTCCTACGGCATGAGCTGGGTTCGTCAGGCACCGGGCAAAGGCCTGGAACTGGTGTCCACCATCAACGGTT
ATGGCGATACGACCTATTACCCGGATAGCGTTAAAGGCCGTTTTACCATTAGCCGTGACAACTCTAAGAACACCTTATAT
CTGCAAATGAATTCACTGCGCGCAGAGGACACTGCCGTTTATTACTGCGCGCGTGACAGAGACTACACTAATTCCTATTA
CTACGCGCACGATTACTGGGGTCAAGGTACGTTGGTCACCGTGTCTTCG
14572-VL (SEQ ID NO: 139)
GAAATAGTACTAACACAATCACCCGCTACTTTGTCACTCTCGCCGGGTGAGCGCGCGACCCTGTCTTGTCGTGCCAGCTC
CTACGTGAGCTTTATGCACTGGCTGCAGCAAAAACCGGGTCAGGCACCGCGTCCGTGGATTTATGCGACGATGAATCTGG
CTAGCGGTGTTCCGGCGCGTTTCAGCGGCAGCGGCTCTGGCACTGACTACACCTTGACCATCAGCTCTCTGGAACCGGAA
GATTTCGCTGTGTATTACTGCCAGCAATGGAGCTCCAACCCACCGGCGTTTGGTGGTGGCACCAAAGTTGAGATCAAG
14573-VH (SEQ ID NO: 140)
GAGGTTCAACTAGTAGAATCAGGTGGAGGGTTGGTGCAGCCGGGTGGTAGCCTCCGCCTGAGCTGTGCTGCGAGCGGTTT
CACCTTCAGCTCCTGGGGCATGAGCTGGGTTCGTCAGGCACCGGGCAAAGGCCTGGAACTGGTGTCCACCATCAACGGTT
ATGGCGATACGACCTATTACCCGGATAGCGTTAAAGGCCGTTTTACCATTAGCCGTGACAACTCTAAGAACACCTTATAT
CTGCAAATGAATTCACTGCGCGCAGAGGACACTGCCGTTTATTACTGCGCGCGTGACAGAGACTACACTAATTCCTATTA
CTACGCGCACGATTACTGGGGTCAAGGTACGTTGGTCACCGTGTCTTCG
14573-VL (SEQ ID NO: 141)
GAAATAGTACTAACACAATCACCCGCTACTTTGTCACTCTCGCCGGGTGAGCGCGCGACCCTGTCTTGTCGTGCCAGCTC
CTACGTGAGCTTTATGCACTGGCTGCAGCAAAAACCGGGTCAGGCACCGCGTCCGTGGATTTATGCGACGAGCAATCTGG
CTAGCGGTGTTCCGGCGCGTTTCAGCGGCAGCGGCTCTGGCACTGACTACACCTTGACCATCAGCTCTCTGGAACCGGAA
GATTTCGCTGTGTATTACTGCCAGCAATGGAGCTCCAACCCACCGGCGTTTGGTGGTGGCACCAAAGTTGAGATCAAG
U579-WT-VH (SEQ ID NO: 142)
GAGGTTCAACTAGTAGAATCAGGTGGAGGGTTGGTGCAGCCGGGTGGTAGCCTCCGCCTGAGCTGTGCTGCGAGCGGTTT
CACCTTCAGCTCCTACGGCATGAGCTGGGTTCGTCAGGCACCGGGCAAAGGCCTGGAACTGGTGTCCACCATCAACGGTT
ATGGCGATACGACCTATTACCCGGATAGCGTTAAAGGCCGTTTTACCATTAGCCGTGACAACTCTAAGAACACCTTATAT
CTGCAAATGAATTCACTGCGCGCAGAGGACACTGCCGTTTATTACTGCGCGCGTGACAGAGACTACGGTAATTCCTATTA
CTACGCGCTGGATTACTGGGGTCAAGGTACGTTGGTCACCGTGTCTTCG
U579-WT-VL (SEQ ID NO: 143)
GAAATAGTACTAACACAATCACCCGCTACTTTGTCACTCTCGCCGGGTGAGCGCGCGACCCTGTCTTGTCGTGCCAGCTC
CTCCGTGAGCTTTATGCACTGGCTGCAGCAAAAACCGGGTCAGGCACCGCGTCCGTGGATTTATGCGACGAGCAATCTGG
CTAGCGGTGTTCCGGCGCGTTTCAGCGGCAGCGGCTCTGGCACTGACTACACCTTGACCATCAGCTCTCTGGAACCGGAA
GATTTCGCTGTGTATTACTGCCAGCAATGGAGCTCCAACCCACCGGCGTTTGGTGGTGGCACCAAAGTTGAGATCAAG
Leader for VH (SEQ ID NO: 144):
ATGGGCTGGTCTTGTATTATTCTGTTTCTGGTCGCAACTGCTACAGGCGTGCATTCT
Leader for VL (SEQ ID NO: 145):
ATGGGCTGGTCATGTATTATTCTGTTTCTGGTCGCAACTGCTACAGGGGTCCATAGT
Human IgG4 constant region (SEQ ID NO: 146):
GCCAGCACCAAGGGCCCTTCCGTGTTTCCCCTGGCCCCTTGCTCCCGGTCCACATCTGAGAGCACCGCCGCCCTGGGCTG
TCTGGTGAAGGACTACTTCCCAGAGCCCGTGACCGTGAGCTGGAACAGCGGCGCCCTGACAAGCGGCGTGCACACATTTC
CCGCCGTGCTGCAGAGCTCCGGCCTGTACTCCCTGTCTAGCGTGGTGACAGTGCCTTCCTCTAGCCTGGGCACCAAGACA
TATACCTGTAACGTGGACCACAAGCCAAGCAATACCAAGGTGGATAAGCGGGTGGAGTCTAAGTACGGCCCTCCTTGCCC
TCCATGTCCTGCTCCAGAGTTTCTGGGCGGCCCTTCCGTGTTCCTGTTTCCACCCAAACCAAAGGACACACTGATGATCT
CTAGAACACCAGAGGTGACCTGCGTGGTGGTGGACGTGAGCCAGGAGGATCCCGAGGTGCAGTTCAACTGGTACGTGGAT
GGCGTGGAGGTGCACAATGCCAAGACCAAGCCAAGAGAGGAGCAGTTTAACTCTACATACAGGGTGGTGAGCGTGCTGAC
CGTGCTGCACCAGGATTGGCTCAACGGCAAGGAGTATAAGTGCAAGGTGTCCAATAAGGGCCTGCCCTCCTCTATCGAGA
AGACAATCTCTAAGGCTAAGGGCCAGCCAAGAGAGCCTCAGGTGTACACCCTGCCTCCAAGCCAGGAGGAGATGACAAAG
AACCAGGTGTCCCTGACATGTCTGGTGAAGGGCTTCTATCCCTCCGACATCGCCGTGGAGTGGGAGTCTAATGGCCAGCC
TGAGAACAATTACAAGACCACACCCCCTGTGCTGGACTCTGATGGCAGCTTCTTTCTGTATTCCAGGCTGACCGTGGATA
AGTCTCGGTGGCAGGAGGGCAACGTGTTCAGCTGCTCTGTGATGCACGAAGCCCTGCATAATCACTATACTCAGAAAAGT
CTGTCACTGTCACTGGGAAAG
Human immunoglobulin kappa light chain (SEQ ID NO: 147):
AGGACAGTGGCCGCCCCAAGCGTGTTCATCTTTCCCCCTTCCGACGAGCAGCTGAAGTCTGGCACCGCCAGCGTGGTGTG
CCTGCTGAACAACTTCTACCCTCGGGAGGCCAAGGTCCAGTGGAAGGTGGATAACGCCCTGCAGTCTGGCAATAGCCAGG
AGTCCGTGACCGAGCAGGACTCTAAGGATAGCACATATTCCCTGTCTAGCACCCTGACACTGAGCAAGGCCGATTACGAG
AAGCACAAGGTGTATGCCTGTGAAGTCACCCATCAGGGGCTGTCATCACCCGTCACTAAGTCATTCAATCGCGGAGAATG
C
14554-VH (SEQ ID NO: 148)
ATGGGCTGGTCTTGTATTATTCTGTTTCTGGTCGCAACTGCTACAGGCGTGCATTCTGAGGTTCAACTAGTAGAATCAGG
TGGAGGGTTGGTGCAGCCGGGTGGTAGCCTCCGCCTGAGCTGTGCTGCGAGCGGTTTCTGGTTCAGCTCCTACGGCATGA
GCTGGGTTCGTCAGGCACCGGGCAAAGGCCTGGAACTGGTGTCCACCATCAACGGTTATGGCGATACGTGGTATTACCCG
GATAGCGTTAAAGGCCGTTTTACCATTAGCCGTGACAACTCTAAGAACACCTTATATCTGCAAATGAATTCACTGCGCGC
AGAGGACACTGCCGTTTATTACTGCGCGCGTGACAGAGACTACACTAATTCCTATTACTACGCGCACGATTACTGGGGTC
AAGGTACGTTGGTCACCGTGTCTTCGGCCAGCACCAAGGGCCCTTCCGTGTTTCCCCTGGCCCCTTGCTCCCGGTCCACA
TCTGAGAGCACCGCCGCCCTGGGCTGTCTGGTGAAGGACTACTTCCCAGAGCCCGTGACCGTGAGCTGGAACAGCGGCGC
CCTGACAAGCGGCGTGCACACATTTCCCGCCGTGCTGCAGAGCTCCGGCCTGTACTCCCTGTCTAGCGTGGTGACAGTGC
CTTCCTCTAGCCTGGGCACCAAGACATATACCTGTAACGTGGACCACAAGCCAAGCAATACCAAGGTGGATAAGCGGGTG
GAGTCTAAGTACGGCCCTCCTTGCCCTCCATGTCCTGCTCCAGAGTTTCTGGGCGGCCCTTCCGTGTTCCTGTTTCCACC
CAAACCAAAGGACACACTGATGATCTCTAGAACACCAGAGGTGACCTGCGTGGTGGTGGACGTGAGCCAGGAGGATCCCG
AGGTGCAGTTCAACTGGTACGTGGATGGCGTGGAGGTGCACAATGCCAAGACCAAGCCAAGAGAGGAGCAGTTTAACTCT
ACATACAGGGTGGTGAGCGTGCTGACCGTGCTGCACCAGGATTGGCTCAACGGCAAGGAGTATAAGTGCAAGGTGTCCAA
TAAGGGCCTGCCCTCCTCTATCGAGAAGACAATCTCTAAGGCTAAGGGCCAGCCAAGAGAGCCTCAGGTGTACACCCTGC
CTCCAAGCCAGGAGGAGATGACAAAGAACCAGGTGTCCCTGACATGTCTGGTGAAGGGCTTCTATCCCTCCGACATCGCC
GTGGAGTGGGAGTCTAATGGCCAGCCTGAGAACAATTACAAGACCACACCCCCTGTGCTGGACTCTGATGGCAGCTTCTT
TCTGTATTCCAGGCTGACCGTGGATAAGTCTCGGTGGCAGGAGGGCAACGTGTTCAGCTGCTCTGTGATGCACGAAGCCC
TGCATAATCACTATACTCAGAAAAGTCTGTCACTGTCACTGGGAAAG
14554-VL (SEQ ID NO: 149)
ATGGGCTGGTCATGTATTATTCTGTTTCTGGTCGCAACTGCTACAGGGGTCCATAGTGAAATAGTACTAACACAATCACC
CGCTACTTTGTCACTCTCGCCGGGTGAGCGCGCGACCCTGTCTTGTCGTGCCAGCTCCTACGTGAGCTTTATGCACTGGC
TGCAGCAAAAACCGGGTCAGGCACCGCGTCCGTGGATTTATGCGACGGCCAATCTGGCTAGCGGTGTTCCGGCGCGTTTC
AGCGGCAGCGGCTCTGGCACTGACTACACCTTGACCATCAGCTCTCTGGAACCGGAAGATTTCGCTGTGTATTACTGCCA
GCAATGGAGCTCCAACCCACCGGCGTTTGGTGGTGGCACCAAAGTTGAGATCAAGAGGACAGTGGCCGCCCCAAGCGTGT
TCATCTTTCCCCCTTCCGACGAGCAGCTGAAGTCTGGCACCGCCAGCGTGGTGTGCCTGCTGAACAACTTCTACCCTCGG
GAGGCCAAGGTCCAGTGGAAGGTGGATAACGCCCTGCAGTCTGGCAATAGCCAGGAGTCCGTGACCGAGCAGGACTCTAA
GGATAGCACATATTCCCTGTCTAGCACCCTGACACTGAGCAAGGCCGATTACGAGAAGCACAAGGTGTATGCCTGTGAAG
TCACCCATCAGGGGCTGTCATCACCCGTCACTAAGTCATTCAATCGCGGAGAATGC
14555-VH (SEQ ID NO: 150)
ATGGGCTGGTCTTGTATTATTCTGTTTCTGGTCGCAACTGCTACAGGCGTGCATTCTGAGGTTCAACTAGTAGAATCAGG
TGGAGGGTTGGTGCAGCCGGGTGGTAGCCTCCGCCTGAGCTGTGCTGCGAGCGGTTTCACCTTCAGCTCCTGGGGCATGA
GCTGGGTTCGTCAGGCACCGGGCAAAGGCCTGGAACTGGTGTCCACCATCAACGGTTATGGCGATACGACCTATTACCCG
GATAGCGTTAAAGGCCGTTTTACCATTAGCCGTGACAACTCTAAGAACACCTTATATCTGCAAATGAATTCACTGCGCGC
AGAGGACACTGCCGTTTATTACTGCGCGCGTGACAGAGACTACACTAATTCCTATTACTACGCGCACGATTACTGGGGTC
AAGGTACGTTGGTCACCGTGTCTTCGGCCAGCACCAAGGGCCCTTCCGTGTTTCCCCTGGCCCCTTGCTCCCGGTCCACA
TCTGAGAGCACCGCCGCCCTGGGCTGTCTGGTGAAGGACTACTTCCCAGAGCCCGTGACCGTGAGCTGGAACAGCGGCGC
CCTGACAAGCGGCGTGCACACATTTCCCGCCGTGCTGCAGAGCTCCGGCCTGTACTCCCTGTCTAGCGTGGTGACAGTGC
CTTCCTCTAGCCTGGGCACCAAGACATATACCTGTAACGTGGACCACAAGCCAAGCAATACCAAGGTGGATAAGCGGGTG
GAGTCTAAGTACGGCCCTCCTTGCCCTCCATGTCCTGCTCCAGAGTTTCTGGGCGGCCCTTCCGTGTTCCTGTTTCCACC
CAAACCAAAGGACACACTGATGATCTCTAGAACACCAGAGGTGACCTGCGTGGTGGTGGACGTGAGCCAGGAGGATCCCG
AGGTGCAGTTCAACTGGTACGTGGATGGCGTGGAGGTGCACAATGCCAAGACCAAGCCAAGAGAGGAGCAGTTTAACTCT
ACATACAGGGTGGTGAGCGTGCTGACCGTGCTGCACCAGGATTGGCTCAACGGCAAGGAGTATAAGTGCAAGGTGTCCAA
TAAGGGCCTGCCCTCCTCTATCGAGAAGACAATCTCTAAGGCTAAGGGCCAGCCAAGAGAGCCTCAGGTGTACACCCTGC
CTCCAAGCCAGGAGGAGATGACAAAGAACCAGGTGTCCCTGACATGTCTGGTGAAGGGCTTCTATCCCTCCGACATCGCC
GTGGAGTGGGAGTCTAATGGCCAGCCTGAGAACAATTACAAGACCACACCCCCTGTGCTGGACTCTGATGGCAGCTTCTT
TCTGTATTCCAGGCTGACCGTGGATAAGTCTCGGTGGCAGGAGGGCAACGTGTTCAGCTGCTCTGTGATGCACGAAGCCC
TGCATAATCACTATACTCAGAAAAGTCTGTCACTGTCACTGGGAAAG
14555-VL (SEQ ID NO: 151)
ATGGGCTGGTCATGTATTATTCTGTTTCTGGTCGCAACTGCTACAGGGGTCCATAGTGAAATAGTACTAACACAATCACC
CGCTACTTTGTCACTCTCGCCGGGTGAGCGCGCGACCCTGTCTTGTCGTGCCAGCTCCTACGTGAGCTTTATGCACTGGC
TGCAGCAAAAACCGGGTCAGGCACCGCGTCCGTGGATTTATGCGACGGCCAATCTGGCTAGCGGTGTTCCGGCGCGTTTC
AGCGGCAGCGGCTCTGGCACTGACTACACCTTGACCATCAGCTCTCTGGAACCGGAAGATTTCGCTGTGTATTACTGCCA
GCAATGGAGCTCCAACCCACCGGCGTTTGGTGGTGGCACCAAAGTTGAGATCAAGAGGACAGTGGCCGCCCCAAGCGTGT
TCATCTTTCCCCCTTCCGACGAGCAGCTGAAGTCTGGCACCGCCAGCGTGGTGTGCCTGCTGAACAACTTCTACCCTCGG
GAGGCCAAGGTCCAGTGGAAGGTGGATAACGCCCTGCAGTCTGGCAATAGCCAGGAGTCCGTGACCGAGCAGGACTCTAA
GGATAGCACATATTCCCTGTCTAGCACCCTGACACTGAGCAAGGCCGATTACGAGAAGCACAAGGTGTATGCCTGTGAAG
TCACCCATCAGGGGCTGTCATCACCCGTCACTAAGTCATTCAATCGCGGAGAATGC
14556-VH (SEQ ID NO: 152)
ATGGGCTGGTCTTGTATTATTCTGTTTCTGGTCGCAACTGCTACAGGCGTGCATTCTGAGGTTCAACTAGTAGAATCAGG
TGGAGGGTTGGTGCAGCCGGGTGGTAGCCTCCGCCTGAGCTGTGCTGCGAGCGGTTACACCTTCAGCTCCTACGGCATGA
GCTGGGTTCGTCAGGCACCGGGCAAAGGCCTGGAACTGGTGTCCACCATCAACGGTTATGGCGATACGACCTATTACCCG
GATAGCGTTAAAGGCCGTTTTACCATTAGCCGTGACAACTCTAAGAACACCTTATATCTGCAAATGAATTCACTGCGCGC
AGAGGACACTGCCGTTTATTACTGCGCGCGTGACAGAGACTACTCTAATTCCTATTACTACGCGCTGGATTACTGGGGTC
AAGGTACGTTGGTCACCGTGTCTTCGGCCAGCACCAAGGGCCCTTCCGTGTTTCCCCTGGCCCCTTGCTCCCGGTCCACA
TCTGAGAGCACCGCCGCCCTGGGCTGTCTGGTGAAGGACTACTTCCCAGAGCCCGTGACCGTGAGCTGGAACAGCGGCGC
CCTGACAAGCGGCGTGCACACATTTCCCGCCGTGCTGCAGAGCTCCGGCCTGTACTCCCTGTCTAGCGTGGTGACAGTGC
CTTCCTCTAGCCTGGGCACCAAGACATATACCTGTAACGTGGACCACAAGCCAAGCAATACCAAGGTGGATAAGCGGGTG
GAGTCTAAGTACGGCCCTCCTTGCCCTCCATGTCCTGCTCCAGAGTTTCTGGGCGGCCCTTCCGTGTTCCTGTTTCCACC
CAAACCAAAGGACACACTGATGATCTCTAGAACACCAGAGGTGACCTGCGTGGTGGTGGACGTGAGCCAGGAGGATCCCG
AGGTGCAGTTCAACTGGTACGTGGATGGCGTGGAGGTGCACAATGCCAAGACCAAGCCAAGAGAGGAGCAGTTTAACTCT
ACATACAGGGTGGTGAGCGTGCTGACCGTGCTGCACCAGGATTGGCTCAACGGCAAGGAGTATAAGTGCAAGGTGTCCAA
TAAGGGCCTGCCCTCCTCTATCGAGAAGACAATCTCTAAGGCTAAGGGCCAGCCAAGAGAGCCTCAGGTGTACACCCTGC
CTCCAAGCCAGGAGGAGATGACAAAGAACCAGGTGTCCCTGACATGTCTGGTGAAGGGCTTCTATCCCTCCGACATCGCC
GTGGAGTGGGAGTCTAATGGCCAGCCTGAGAACAATTACAAGACCACACCCCCTGTGCTGGACTCTGATGGCAGCTTCTT
TCTGTATTCCAGGCTGACCGTGGATAAGTCTCGGTGGCAGGAGGGCAACGTGTTCAGCTGCTCTGTGATGCACGAAGCCC
TGCATAATCACTATACTCAGAAAAGTCTGTCACTGTCACTGGGAAAG
14556-VL (SEQ ID NO: 153)
ATGGGCTGGTCATGTATTATTCTGTTTCTGGTCGCAACTGCTACAGGGGTCCATAGTGAAATAGTACTAACACAATCACC
CGCTACTTTGTCACTCTCGCCGGGTGAGCGCGCGACCCTGTCTTGTCGTGCCAGCTCCTACGTGAGCTTTATGCACTGGC
TGCAGCAAAAACCGGGTCAGGCACCGCGTCCGTGGATTTATGCGACGGCCAATCTGGCTAGCGGTGTTCCGGCGCGTTTC
AGCGGCAGCGGCTCTGGCACTGACTACACCTTGACCATCAGCTCTCTGGAACCGGAAGATTTCGCTGTGTATTACTGCCA
GCAATGGAGCTCCAACCCACCGGCGTTTGGTGGTGGCACCAAAGTTGAGATCAAGAGGACAGTGGCCGCCCCAAGCGTGT
TCATCTTTCCCCCTTCCGACGAGCAGCTGAAGTCTGGCACCGCCAGCGTGGTGTGCCTGCTGAACAACTTCTACCCTCGG
GAGGCCAAGGTCCAGTGGAAGGTGGATAACGCCCTGCAGTCTGGCAATAGCCAGGAGTCCGTGACCGAGCAGGACTCTAA
GGATAGCACATATTCCCTGTCTAGCACCCTGACACTGAGCAAGGCCGATTACGAGAAGCACAAGGTGTATGCCTGTGAAG
TCACCCATCAGGGGCTGTCATCACCCGTCACTAAGTCATTCAATCGCGGAGAATGC
14557-VH (SEQ ID NO: 154)
ATGGGCTGGTCTTGTATTATTCTGTTTCTGGTCGCAACTGCTACAGGCGTGCATTCTGAGGTTCAACTAGTAGAATCAGG
TGGAGGGTTGGTGCAGCCGGGTGGTAGCCTCCGCCTGAGCTGTGCTGCGAGCGGTTACACCTTCAGCTCCTACGGCATGA
GCTGGGTTCGTCAGGCACCGGGCAAAGGCCTGGAACTGGTGTCCACCATCAACGGTTATGGCGATACGTGGTATTACCCG
GATAGCGTTAAAGGCCGTTTTACCATTAGCCGTGACAACTCTAAGAACACCTTATATCTGCAAATGAATTCACTGCGCGC
AGAGGACACTGCCGTTTATTACTGCGCGCGTGACAGAGACTACTCTAATTCCTATTACTACGCGCACGATTACTGGGGTC
AAGGTACGTTGGTCACCGTGTCTTCGGCCAGCACCAAGGGCCCTTCCGTGTTTCCCCTGGCCCCTTGCTCCCGGTCCACA
TCTGAGAGCACCGCCGCCCTGGGCTGTCTGGTGAAGGACTACTTCCCAGAGCCCGTGACCGTGAGCTGGAACAGCGGCGC
CCTGACAAGCGGCGTGCACACATTTCCCGCCGTGCTGCAGAGCTCCGGCCTGTACTCCCTGTCTAGCGTGGTGACAGTGC
CTTCCTCTAGCCTGGGCACCAAGACATATACCTGTAACGTGGACCACAAGCCAAGCAATACCAAGGTGGATAAGCGGGTG
GAGTCTAAGTACGGCCCTCCTTGCCCTCCATGTCCTGCTCCAGAGTTTCTGGGCGGCCCTTCCGTGTTCCTGTTTCCACC
CAAACCAAAGGACACACTGATGATCTCTAGAACACCAGAGGTGACCTGCGTGGTGGTGGACGTGAGCCAGGAGGATCCCG
AGGTGCAGTTCAACTGGTACGTGGATGGCGTGGAGGTGCACAATGCCAAGACCAAGCCAAGAGAGGAGCAGTTTAACTCT
ACATACAGGGTGGTGAGCGTGCTGACCGTGCTGCACCAGGATTGGCTCAACGGCAAGGAGTATAAGTGCAAGGTGTCCAA
TAAGGGCCTGCCCTCCTCTATCGAGAAGACAATCTCTAAGGCTAAGGGCCAGCCAAGAGAGCCTCAGGTGTACACCCTGC
CTCCAAGCCAGGAGGAGATGACAAAGAACCAGGTGTCCCTGACATGTCTGGTGAAGGGCTTCTATCCCTCCGACATCGCC
GTGGAGTGGGAGTCTAATGGCCAGCCTGAGAACAATTACAAGACCACACCCCCTGTGCTGGACTCTGATGGCAGCTTCTT
TCTGTATTCCAGGCTGACCGTGGATAAGTCTCGGTGGCAGGAGGGCAACGTGTTCAGCTGCTCTGTGATGCACGAAGCCC
TGCATAATCACTATACTCAGAAAAGTCTGTCACTGTCACTGGGAAAG
14557-VL (SEQ ID NO: 155)
ATGGGCTGGTCATGTATTATTCTGTTTCTGGTCGCAACTGCTACAGGGGTCCATAGTGAAATAGTACTAACACAATCACC
CGCTACTTTGTCACTCTCGCCGGGTGAGCGCGCGACCCTGTCTTGTCGTGCCAGCTCCTGGGTGAGCTTTATGCACTGGC
TGCAGCAAAAACCGGGTCAGGCACCGCGTCCGTGGATTTATGCGACGCAGAATCTGGCTAGCGGTGTTCCGGCGCGTTTC
ATCGGCAGCGGCTCTGGCACTGACTACACCTTGACCATCAGCTCTCTGGAACCGGAAGATTTCGCTGTGTATTACTGCCA
GCAATGGAGCTCCAACCCACCGGCGTTTGGTGGTGGCACCAAAGTTGAGATCAAGAGGACAGTGGCCGCCCCAAGCGTGT
TCATCTTTCCCCCTTCCGACGAGCAGCTGAAGTCTGGCACCGCCAGCGTGGTGTGCCTGCTGAACAACTTCTACCCTCGG
GAGGCCAAGGTCCAGTGGAAGGTGGATAACGCCCTGCAGTCTGGCAATAGCCAGGAGTCCGTGACCGAGCAGGACTCTAA
GGATAGCACATATTCCCTGTCTAGCACCCTGACACTGAGCAAGGCCGATTACGAGAAGCACAAGGTGTATGCCTGTGAAG
TCACCCATCAGGGGCTGTCATCACCCGTCACTAAGTCATTCAATCGCGGAGAATGC
14558-VH (SEQ ID NO: 156)
ATGGGCTGGTCTTGTATTATTCTGTTTCTGGTCGCAACTGCTACAGGCGTGCATTCTGAGGTTCAACTAGTAGAATCAGG
TGGAGGGTTGGTGCAGCCGGGTGGTAGCCTCCGCCTGAGCTGTGCTGCGAGCGGTTACTGGTTCAGCTCCTACGGCATGA
GCTGGGTTCGTCAGGCACCGGGCAAAGGCCTGGAACTGGTGTCCACCATCAACGGTTATGGCGATACGACCTATTACCCG
GATAGCGTTAAAGGCCGTTTTACCATTAGCCGTGACAACTCTAAGAACACCTTATATCTGCAAATGAATTCACTGCGCGC
AGAGGACACTGCCGTTTATTACTGCGCGCGTGACAGAGACTACACTAATTCCTATTACTACGCGCACGATTACTGGGGTC
AAGGTACGTTGGTCACCGTGTCTTCGGCCAGCACCAAGGGCCCTTCCGTGTTTCCCCTGGCCCCTTGCTCCCGGTCCACA
TCTGAGAGCACCGCCGCCCTGGGCTGTCTGGTGAAGGACTACTTCCCAGAGCCCGTGACCGTGAGCTGGAACAGCGGCGC
CCTGACAAGCGGCGTGCACACATTTCCCGCCGTGCTGCAGAGCTCCGGCCTGTACTCCCTGTCTAGCGTGGTGACAGTGC
CTTCCTCTAGCCTGGGCACCAAGACATATACCTGTAACGTGGACCACAAGCCAAGCAATACCAAGGTGGATAAGCGGGTG
GAGTCTAAGTACGGCCCTCCTTGCCCTCCATGTCCTGCTCCAGAGTTTCTGGGCGGCCCTTCCGTGTTCCTGTTTCCACC
CAAACCAAAGGACACACTGATGATCTCTAGAACACCAGAGGTGACCTGCGTGGTGGTGGACGTGAGCCAGGAGGATCCCG
AGGTGCAGTTCAACTGGTACGTGGATGGCGTGGAGGTGCACAATGCCAAGACCAAGCCAAGAGAGGAGCAGTTTAACTCT
ACATACAGGGTGGTGAGCGTGCTGACCGTGCTGCACCAGGATTGGCTCAACGGCAAGGAGTATAAGTGCAAGGTGTCCAA
TAAGGGCCTGCCCTCCTCTATCGAGAAGACAATCTCTAAGGCTAAGGGCCAGCCAAGAGAGCCTCAGGTGTACACCCTGC
CTCCAAGCCAGGAGGAGATGACAAAGAACCAGGTGTCCCTGACATGTCTGGTGAAGGGCTTCTATCCCTCCGACATCGCC
GTGGAGTGGGAGTCTAATGGCCAGCCTGAGAACAATTACAAGACCACACCCCCTGTGCTGGACTCTGATGGCAGCTTCTT
TCTGTATTCCAGGCTGACCGTGGATAAGTCTCGGTGGCAGGAGGGCAACGTGTTCAGCTGCTCTGTGATGCACGAAGCCC
TGCATAATCACTATACTCAGAAAAGTCTGTCACTGTCACTGGGAAAG
14558-VL (SEQ ID NO: 157)
ATGGGCTGGTCATGTATTATTCTGTTTCTGGTCGCAACTGCTACAGGGGTCCATAGTGAAATAGTACTAACACAATCACC
CGCTACTTTGTCACTCTCGCCGGGTGAGCGCGCGACCCTGTCTTGTCGTGCCAGCTCCTCCGTGAGCTTTATGCACTGGC
TGCAGCAAAAACCGGGTCAGGCACCGCGTCCGTGGATTTATGCGACGATGAATCTGGCTAGCGGTGTTCCGGCGCGTTTC
AGCGGCAGCGGCTCTGGCACTGACTACACCTTGACCATCAGCTCTCTGGAACCGGAAGATTTCGCTGTGTATTACTGCCA
GCAATGGAGCTCCAACCCACCGGCGTTTGGTGGTGGCACCAAAGTTGAGATCAAGAGGACAGTGGCCGCCCCAAGCGTGT
TCATCTTTCCCCCTTCCGACGAGCAGCTGAAGTCTGGCACCGCCAGCGTGGTGTGCCTGCTGAACAACTTCTACCCTCGG
GAGGCCAAGGTCCAGTGGAAGGTGGATAACGCCCTGCAGTCTGGCAATAGCCAGGAGTCCGTGACCGAGCAGGACTCTAA
GGATAGCACATATTCCCTGTCTAGCACCCTGACACTGAGCAAGGCCGATTACGAGAAGCACAAGGTGTATGCCTGTGAAG
TCACCCATCAGGGGCTGTCATCACCCGTCACTAAGTCATTCAATCGCGGAGAATGC
14559-VH (SEQ ID NO: 158)
ATGGGCTGGTCTTGTATTATTCTGTTTCTGGTCGCAACTGCTACAGGCGTGCATTCTGAGGTTCAACTAGTAGAATCAGG
TGGAGGGTTGGTGCAGCCGGGTGGTAGCCTCCGCCTGAGCTGTGCTGCGAGCGGTTTCACCTTCAGCTCCTACGGCATGA
GCTGGGTTCGTCAGGCACCGGGCAAAGGCCTGGAACTGGTGTCCACCATCAACGGTTATGGCGATACGACCTATTACCCG
GATAGCGTTAAAGGCCGTTTTACCATTAGCCGTGACAACTCTAAGAACACCTTATATCTGCAAATGAATTCACTGCGCGC
AGAGGACACTGCCGTTTATTACTGCGCGCGTGACAGAGACTACTCTAATTCCTATTACTACGCGCACGATTACTGGGGTC
AAGGTACGTTGGTCACCGTGTCTTCGGCCAGCACCAAGGGCCCTTCCGTGTTTCCCCTGGCCCCTTGCTCCCGGTCCACA
TCTGAGAGCACCGCCGCCCTGGGCTGTCTGGTGAAGGACTACTTCCCAGAGCCCGTGACCGTGAGCTGGAACAGCGGCGC
CCTGACAAGCGGCGTGCACACATTTCCCGCCGTGCTGCAGAGCTCCGGCCTGTACTCCCTGTCTAGCGTGGTGACAGTGC
CTTCCTCTAGCCTGGGCACCAAGACATATACCTGTAACGTGGACCACAAGCCAAGCAATACCAAGGTGGATAAGCGGGTG
GAGTCTAAGTACGGCCCTCCTTGCCCTCCATGTCCTGCTCCAGAGTTTCTGGGCGGCCCTTCCGTGTTCCTGTTTCCACC
CAAACCAAAGGACACACTGATGATCTCTAGAACACCAGAGGTGACCTGCGTGGTGGTGGACGTGAGCCAGGAGGATCCCG
AGGTGCAGTTCAACTGGTACGTGGATGGCGTGGAGGTGCACAATGCCAAGACCAAGCCAAGAGAGGAGCAGTTTAACTCT
ACATACAGGGTGGTGAGCGTGCTGACCGTGCTGCACCAGGATTGGCTCAACGGCAAGGAGTATAAGTGCAAGGTGTCCAA
TAAGGGCCTGCCCTCCTCTATCGAGAAGACAATCTCTAAGGCTAAGGGCCAGCCAAGAGAGCCTCAGGTGTACACCCTGC
CTCCAAGCCAGGAGGAGATGACAAAGAACCAGGTGTCCCTGACATGTCTGGTGAAGGGCTTCTATCCCTCCGACATCGCC
GTGGAGTGGGAGTCTAATGGCCAGCCTGAGAACAATTACAAGACCACACCCCCTGTGCTGGACTCTGATGGCAGCTTCTT
TCTGTATTCCAGGCTGACCGTGGATAAGTCTCGGTGGCAGGAGGGCAACGTGTTCAGCTGCTCTGTGATGCACGAAGCCC
TGCATAATCACTATACTCAGAAAAGTCTGTCACTGTCACTGGGAAAG
14559-VL (SEQ ID NO: 159)
ATGGGCTGGTCATGTATTATTCTGTTTCTGGTCGCAACTGCTACAGGGGTCCATAGTGAAATAGTACTAACACAATCACC
CGCTACTTTGTCACTCTCGCCGGGTGAGCGCGCGACCCTGTCTTGTCGTGCCAGCTCCTGGGTGAGCTTTATGCACTGGC
TGCAGCAAAAACCGGGTCAGGCACCGCGTCCGTGGATTTATGCGACGCAGAATCTGGCTAGCGGTGTTCCGGCGCGTTTC
AGCGGCAGCGGCTCTGGCACTGACTACACCTTGACCATCAGCTCTCTGGAACCGGAAGATTTCGCTGTGTATTACTGCCA
GCAATGGAGCTCCAACCCACCGGCGTTTGGTGGTGGCACCAAAGTTGAGATCAAGAGGACAGTGGCCGCCCCAAGCGTGT
TCATCTTTCCCCCTTCCGACGAGCAGCTGAAGTCTGGCACCGCCAGCGTGGTGTGCCTGCTGAACAACTTCTACCCTCGG
GAGGCCAAGGTCCAGTGGAAGGTGGATAACGCCCTGCAGTCTGGCAATAGCCAGGAGTCCGTGACCGAGCAGGACTCTAA
GGATAGCACATATTCCCTGTCTAGCACCCTGACACTGAGCAAGGCCGATTACGAGAAGCACAAGGTGTATGCCTGTGAAG
TCACCCATCAGGGGCTGTCATCACCCGTCACTAAGTCATTCAATCGCGGAGAATGC
14560-VH (SEQ ID NO: 160)
ATGGGCTGGTCTTGTATTATTCTGTTTCTGGTCGCAACTGCTACAGGCGTGCATTCTGAGGTTCAACTAGTAGAATCAGG
TGGAGGGTTGGTGCAGCCGGGTGGTAGCCTCCGCCTGAGCTGTGCTGCGAGCGGTTTCACCTTCAGCTCCTACGGCATGA
GCTGGGTTCGTCAGGCACCGGGCAAAGGCCTGGAACTGGTGTCCACCATCAACGGTTATGGCGATACGACCTATTACCCG
GATAGCGTTAAAGGCCGTTTTACCATTAGCCGTGACAACTCTAAGAACACCTTATATCTGCAAATGAATTCACTGCGCGC
AGAGGACACTGCCGTTTATTACTGCGCGCGTGACAGAGACTACACTAATTCCTATTACTACGCGCACGATTACTGGGGTC
AAGGTACGTTGGTCACCGTGTCTTCGGCCAGCACCAAGGGCCCTTCCGTGTTTCCCCTGGCCCCTTGCTCCCGGTCCACA
TCTGAGAGCACCGCCGCCCTGGGCTGTCTGGTGAAGGACTACTTCCCAGAGCCCGTGACCGTGAGCTGGAACAGCGGCGC
CCTGACAAGCGGCGTGCACACATTTCCCGCCGTGCTGCAGAGCTCCGGCCTGTACTCCCTGTCTAGCGTGGTGACAGTGC
CTTCCTCTAGCCTGGGCACCAAGACATATACCTGTAACGTGGACCACAAGCCAAGCAATACCAAGGTGGATAAGCGGGTG
GAGTCTAAGTACGGCCCTCCTTGCCCTCCATGTCCTGCTCCAGAGTTTCTGGGCGGCCCTTCCGTGTTCCTGTTTCCACC
CAAACCAAAGGACACACTGATGATCTCTAGAACACCAGAGGTGACCTGCGTGGTGGTGGACGTGAGCCAGGAGGATCCCG
AGGTGCAGTTCAACTGGTACGTGGATGGCGTGGAGGTGCACAATGCCAAGACCAAGCCAAGAGAGGAGCAGTTTAACTCT
ACATACAGGGTGGTGAGCGTGCTGACCGTGCTGCACCAGGATTGGCTCAACGGCAAGGAGTATAAGTGCAAGGTGTCCAA
TAAGGGCCTGCCCTCCTCTATCGAGAAGACAATCTCTAAGGCTAAGGGCCAGCCAAGAGAGCCTCAGGTGTACACCCTGC
CTCCAAGCCAGGAGGAGATGACAAAGAACCAGGTGTCCCTGACATGTCTGGTGAAGGGCTTCTATCCCTCCGACATCGCC
GTGGAGTGGGAGTCTAATGGCCAGCCTGAGAACAATTACAAGACCACACCCCCTGTGCTGGACTCTGATGGCAGCTTCTT
TCTGTATTCCAGGCTGACCGTGGATAAGTCTCGGTGGCAGGAGGGCAACGTGTTCAGCTGCTCTGTGATGCACGAAGCCC
TGCATAATCACTATACTCAGAAAAGTCTGTCACTGTCACTGGGAAAG
14560-VL (SEQ ID NO: 161)
ATGGGCTGGTCATGTATTATTCTGTTTCTGGTCGCAACTGCTACAGGGGTCCATAGTGAAATAGTACTAACACAATCACC
CGCTACTTTGTCACTCTCGCCGGGTGAGCGCGCGACCCTGTCTTGTCGTGCCAGCTCCTCCGTGAGCTTTATGCACTGGC
TGCAGCAAAAACCGGGTCAGGCACCGCGTCCGTGGATTTATGCGACGCAGAATCTGGCTAGCGGTGTTCCGGCGCGTTTC
AGCGGCAGCGGCTCTGGCACTGACTACACCTTGACCATCAGCTCTCTGGAACCGGAAGATTTCGCTGTGTATTACTGCCA
GCAATGGAGCTCCAACCCACCGGCGTTTGGTGGTGGCACCAAAGTTGAGATCAAGAGGACAGTGGCCGCCCCAAGCGTGT
TCATCTTTCCCCCTTCCGACGAGCAGCTGAAGTCTGGCACCGCCAGCGTGGTGTGCCTGCTGAACAACTTCTACCCTCGG
GAGGCCAAGGTCCAGTGGAAGGTGGATAACGCCCTGCAGTCTGGCAATAGCCAGGAGTCCGTGACCGAGCAGGACTCTAA
GGATAGCACATATTCCCTGTCTAGCACCCTGACACTGAGCAAGGCCGATTACGAGAAGCACAAGGTGTATGCCTGTGAAG
TCACCCATCAGGGGCTGTCATCACCCGTCACTAAGTCATTCAATCGCGGAGAATGC
14561-VH (SEQ ID NO: 162)
ATGGGCTGGTCTTGTATTATTCTGTTTCTGGTCGCAACTGCTACAGGCGTGCATTCTGAGGTTCAACTAGTAGAATCAGG
TGGAGGGTTGGTGCAGCCGGGTGGTAGCCTCCGCCTGAGCTGTGCTGCGAGCGGTTACACCTTCAGCTCCTGGGGCATGA
GCTGGGTTCGTCAGGCACCGGGCAAAGGCCTGGAACTGGTGTCCACCATCAACGGTTATGGCGATACGTGGTATTACCCG
GATAGCGTTAAAGGCCGTTTTACCATTAGCCGTGACAACTCTAAGAACACCTTATATCTGCAAATGAATTCACTGCGCGC
AGAGGACACTGCCGTTTATTACTGCGCGCGTGACAGAGACTACTCTAATTCCTATTACTACGCGCACGATTACTGGGGTC
AAGGTACGTTGGTCACCGTGTCTTCGGCCAGCACCAAGGGCCCTTCCGTGTTTCCCCTGGCCCCTTGCTCCCGGTCCACA
TCTGAGAGCACCGCCGCCCTGGGCTGTCTGGTGAAGGACTACTTCCCAGAGCCCGTGACCGTGAGCTGGAACAGCGGCGC
CCTGACAAGCGGCGTGCACACATTTCCCGCCGTGCTGCAGAGCTCCGGCCTGTACTCCCTGTCTAGCGTGGTGACAGTGC
CTTCCTCTAGCCTGGGCACCAAGACATATACCTGTAACGTGGACCACAAGCCAAGCAATACCAAGGTGGATAAGCGGGTG
GAGTCTAAGTACGGCCCTCCTTGCCCTCCATGTCCTGCTCCAGAGTTTCTGGGCGGCCCTTCCGTGTTCCTGTTTCCACC
CAAACCAAAGGACACACTGATGATCTCTAGAACACCAGAGGTGACCTGCGTGGTGGTGGACGTGAGCCAGGAGGATCCCG
AGGTGCAGTTCAACTGGTACGTGGATGGCGTGGAGGTGCACAATGCCAAGACCAAGCCAAGAGAGGAGCAGTTTAACTCT
ACATACAGGGTGGTGAGCGTGCTGACCGTGCTGCACCAGGATTGGCTCAACGGCAAGGAGTATAAGTGCAAGGTGTCCAA
TAAGGGCCTGCCCTCCTCTATCGAGAAGACAATCTCTAAGGCTAAGGGCCAGCCAAGAGAGCCTCAGGTGTACACCCTGC
CTCCAAGCCAGGAGGAGATGACAAAGAACCAGGTGTCCCTGACATGTCTGGTGAAGGGCTTCTATCCCTCCGACATCGCC
GTGGAGTGGGAGTCTAATGGCCAGCCTGAGAACAATTACAAGACCACACCCCCTGTGCTGGACTCTGATGGCAGCTTCTT
TCTGTATTCCAGGCTGACCGTGGATAAGTCTCGGTGGCAGGAGGGCAACGTGTTCAGCTGCTCTGTGATGCACGAAGCCC
TGCATAATCACTATACTCAGAAAAGTCTGTCACTGTCACTGGGAAAG
14561-VL (SEQ ID NO: 163)
ATGGGCTGGTCATGTATTATTCTGTTTCTGGTCGCAACTGCTACAGGGGTCCATAGTGAAATAGTACTAACACAATCACC
CGCTACTTTGTCACTCTCGCCGGGTGAGCGCGCGACCCTGTCTTGTCGTGCCAGCTCCTACGTGAGCTTTATGCACTGGC
TGCAGCAAAAACCGGGTCAGGCACCGCGTCCGTGGATTTATGCGACGATGAATCTGGCTAGCGGTGTTCCGGCGCGTTTC
AGCGGCAGCGGCTCTGGCACTGACTACACCTTGACCATCAGCTCTCTGGAACCGGAAGATTTCGCTGTGTATTACTGCCA
GCAATGGAGCTCCAACCCACCGGCGTTTGGTGGTGGCACCAAAGTTGAGATCAAGAGGACAGTGGCCGCCCCAAGCGTGT
TCATCTTTCCCCCTTCCGACGAGCAGCTGAAGTCTGGCACCGCCAGCGTGGTGTGCCTGCTGAACAACTTCTACCCTCGG
GAGGCCAAGGTCCAGTGGAAGGTGGATAACGCCCTGCAGTCTGGCAATAGCCAGGAGTCCGTGACCGAGCAGGACTCTAA
GGATAGCACATATTCCCTGTCTAGCACCCTGACACTGAGCAAGGCCGATTACGAGAAGCACAAGGTGTATGCCTGTGAAG
TCACCCATCAGGGGCTGTCATCACCCGTCACTAAGTCATTCAATCGCGGAGAATGC
14562-VH (SEQ ID NO: 164)
ATGGGCTGGTCTTGTATTATTCTGTTTCTGGTCGCAACTGCTACAGGCGTGCATTCTGAGGTTCAACTAGTAGAATCAGG
TGGAGGGTTGGTGCAGCCGGGTGGTAGCCTCCGCCTGAGCTGTGCTGCGAGCGGTTTCACCTTCAGCTCCTACGGCATGA
GCTGGGTTCGTCAGGCACCGGGCAAAGGCCTGGAACTGGTGTCCACCATCAACGGTTATGGCGATACGTGGTATTACCCG
GATAGCGTTAAAGGCCGTTTTACCATTAGCCGTGACAACTCTAAGAACACCTTATATCTGCAAATGAATTCACTGCGCGC
AGAGGACACTGCCGTTTATTACTGCGCGCGTGACAGAGACTACTCTAATTCCTATTACTACGCGCACGATTACTGGGGTC
AAGGTACGTTGGTCACCGTGTCTTCGGCCAGCACCAAGGGCCCTTCCGTGTTTCCCCTGGCCCCTTGCTCCCGGTCCACA
TCTGAGAGCACCGCCGCCCTGGGCTGTCTGGTGAAGGACTACTTCCCAGAGCCCGTGACCGTGAGCTGGAACAGCGGCGC
CCTGACAAGCGGCGTGCACACATTTCCCGCCGTGCTGCAGAGCTCCGGCCTGTACTCCCTGTCTAGCGTGGTGACAGTGC
CTTCCTCTAGCCTGGGCACCAAGACATATACCTGTAACGTGGACCACAAGCCAAGCAATACCAAGGTGGATAAGCGGGTG
GAGTCTAAGTACGGCCCTCCTTGCCCTCCATGTCCTGCTCCAGAGTTTCTGGGCGGCCCTTCCGTGTTCCTGTTTCCACC
CAAACCAAAGGACACACTGATGATCTCTAGAACACCAGAGGTGACCTGCGTGGTGGTGGACGTGAGCCAGGAGGATCCCG
AGGTGCAGTTCAACTGGTACGTGGATGGCGTGGAGGTGCACAATGCCAAGACCAAGCCAAGAGAGGAGCAGTTTAACTCT
ACATACAGGGTGGTGAGCGTGCTGACCGTGCTGCACCAGGATTGGCTCAACGGCAAGGAGTATAAGTGCAAGGTGTCCAA
TAAGGGCCTGCCCTCCTCTATCGAGAAGACAATCTCTAAGGCTAAGGGCCAGCCAAGAGAGCCTCAGGTGTACACCCTGC
CTCCAAGCCAGGAGGAGATGACAAAGAACCAGGTGTCCCTGACATGTCTGGTGAAGGGCTTCTATCCCTCCGACATCGCC
GTGGAGTGGGAGTCTAATGGCCAGCCTGAGAACAATTACAAGACCACACCCCCTGTGCTGGACTCTGATGGCAGCTTCTT
TCTGTATTCCAGGCTGACCGTGGATAAGTCTCGGTGGCAGGAGGGCAACGTGTTCAGCTGCTCTGTGATGCACGAAGCCC
TGCATAATCACTATACTCAGAAAAGTCTGTCACTGTCACTGGGAAAG
14562-VL (SEQ ID NO: 165)
ATGGGCTGGTCATGTATTATTCTGTTTCTGGTCGCAACTGCTACAGGGGTCCATAGTGAAATAGTACTAACACAATCACC
CGCTACTTTGTCACTCTCGCCGGGTGAGCGCGCGACCCTGTCTTGTCGTGCCAGCTCCTCCGTGAGCTTTATGCACTGGC
TGCAGCAAAAACCGGGTCAGGCACCGCGTCCGTGGATTTATGCGACGATGAATCTGGCTAGCGGTGTTCCGGCGCGTTTC
AGCGGCAGCGGCTCTGGCACTGACTACACCTTGACCATCAGCTCTCTGGAACCGGAAGATTTCGCTGTGTATTACTGCCA
GCAATGGAGCTCCAACCCACCGGCGTTTGGTGGTGGCACCAAAGTTGAGATCAAGAGGACAGTGGCCGCCCCAAGCGTGT
TCATCTTTCCCCCTTCCGACGAGCAGCTGAAGTCTGGCACCGCCAGCGTGGTGTGCCTGCTGAACAACTTCTACCCTCGG
GAGGCCAAGGTCCAGTGGAAGGTGGATAACGCCCTGCAGTCTGGCAATAGCCAGGAGTCCGTGACCGAGCAGGACTCTAA
GGATAGCACATATTCCCTGTCTAGCACCCTGACACTGAGCAAGGCCGATTACGAGAAGCACAAGGTGTATGCCTGTGAAG
TCACCCATCAGGGGCTGTCATCACCCGTCACTAAGTCATTCAATCGCGGAGAATGC
14563-VH (SEQ ID NO: 166)
ATGGGCTGGTCTTGTATTATTCTGTTTCTGGTCGCAACTGCTACAGGCGTGCATTCTGAGGTTCAACTAGTAGAATCAGG
TGGAGGGTTGGTGCAGCCGGGTGGTAGCCTCCGCCTGAGCTGTGCTGCGAGCGGTTACACCTTCAGCTCCTACGGCATGA
GCTGGGTTCGTCAGGCACCGGGCAAAGGCCTGGAACTGGTGTCCACCATCAACGGTTATGGCGATACGACCTATTACCCG
GATAGCGTTAAAGGCCGTTTTACCATTAGCCGTGACAACTCTAAGAACACCTTATATCTGCAAATGAATTCACTGCGCGC
AGAGGACACTGCCGTTTATTACTGCGCGCGTGACAGAGACTACGGTAATTCCTATTACTACGCGCACGATTACTGGGGTC
AAGGTACGTTGGTCACCGTGTCTTCGGCCAGCACCAAGGGCCCTTCCGTGTTTCCCCTGGCCCCTTGCTCCCGGTCCACA
TCTGAGAGCACCGCCGCCCTGGGCTGTCTGGTGAAGGACTACTTCCCAGAGCCCGTGACCGTGAGCTGGAACAGCGGCGC
CCTGACAAGCGGCGTGCACACATTTCCCGCCGTGCTGCAGAGCTCCGGCCTGTACTCCCTGTCTAGCGTGGTGACAGTGC
CTTCCTCTAGCCTGGGCACCAAGACATATACCTGTAACGTGGACCACAAGCCAAGCAATACCAAGGTGGATAAGCGGGTG
GAGTCTAAGTACGGCCCTCCTTGCCCTCCATGTCCTGCTCCAGAGTTTCTGGGCGGCCCTTCCGTGTTCCTGTTTCCACC
CAAACCAAAGGACACACTGATGATCTCTAGAACACCAGAGGTGACCTGCGTGGTGGTGGACGTGAGCCAGGAGGATCCCG
AGGTGCAGTTCAACTGGTACGTGGATGGCGTGGAGGTGCACAATGCCAAGACCAAGCCAAGAGAGGAGCAGTTTAACTCT
ACATACAGGGTGGTGAGCGTGCTGACCGTGCTGCACCAGGATTGGCTCAACGGCAAGGAGTATAAGTGCAAGGTGTCCAA
TAAGGGCCTGCCCTCCTCTATCGAGAAGACAATCTCTAAGGCTAAGGGCCAGCCAAGAGAGCCTCAGGTGTACACCCTGC
CTCCAAGCCAGGAGGAGATGACAAAGAACCAGGTGTCCCTGACATGTCTGGTGAAGGGCTTCTATCCCTCCGACATCGCC
GTGGAGTGGGAGTCTAATGGCCAGCCTGAGAACAATTACAAGACCACACCCCCTGTGCTGGACTCTGATGGCAGCTTCTT
TCTGTATTCCAGGCTGACCGTGGATAAGTCTCGGTGGCAGGAGGGCAACGTGTTCAGCTGCTCTGTGATGCACGAAGCCC
TGCATAATCACTATACTCAGAAAAGTCTGTCACTGTCACTGGGAAAG
14563-VL (SEQ ID NO: 167)
ATGGGCTGGTCATGTATTATTCTGTTTCTGGTCGCAACTGCTACAGGGGTCCATAGTGAAATAGTACTAACACAATCACC
CGCTACTTTGTCACTCTCGCCGGGTGAGCGCGCGACCCTGTCTTGTCGTGCCAGCTCCTACGTGAGCTTTATGCACTGGC
TGCAGCAAAAACCGGGTCAGGCACCGCGTCCGTGGATTTATGCGACGATGAATCTGGCTAGCGGTGTTCCGGCGCGTTTC
AGCGGCAGCGGCTCTGGCACTGACTACACCTTGACCATCAGCTCTCTGGAACCGGAAGATTTCGCTGTGTATTACTGCCA
GCAATGGAGCTCCAACCCACCGGCGTTTGGTGGTGGCACCAAAGTTGAGATCAAGAGGACAGTGGCCGCCCCAAGCGTGT
TCATCTTTCCCCCTTCCGACGAGCAGCTGAAGTCTGGCACCGCCAGCGTGGTGTGCCTGCTGAACAACTTCTACCCTCGG
GAGGCCAAGGTCCAGTGGAAGGTGGATAACGCCCTGCAGTCTGGCAATAGCCAGGAGTCCGTGACCGAGCAGGACTCTAA
GGATAGCACATATTCCCTGTCTAGCACCCTGACACTGAGCAAGGCCGATTACGAGAAGCACAAGGTGTATGCCTGTGAAG
TCACCCATCAGGGGCTGTCATCACCCGTCACTAAGTCATTCAATCGCGGAGAATGC
14564-VH (SEQ ID NO: 168)
ATGGGCTGGTCTTGTATTATTCTGTTTCTGGTCGCAACTGCTACAGGCGTGCATTCTGAGGTTCAACTAGTAGAATCAGG
TGGAGGGTTGGTGCAGCCGGGTGGTAGCCTCCGCCTGAGCTGTGCTGCGAGCGGTTTCACCTTCAGCTCCTGGGGCATGA
GCTGGGTTCGTCAGGCACCGGGCAAAGGCCTGGAACTGGTGTCCACCATCAACGGTTATGGCGATACGACCTATTACCCG
GATAGCGTTAAAGGCCGTTTTACCATTAGCCGTGACAACTCTAAGAACACCTTATATCTGCAAATGAATTCACTGCGCGC
AGAGGACACTGCCGTTTATTACTGCGCGCGTGACAGAGACTACTCTAATTCCTATTACTACGCGCACGATTACTGGGGTC
AAGGTACGTTGGTCACCGTGTCTTCGGCCAGCACCAAGGGCCCTTCCGTGTTTCCCCTGGCCCCTTGCTCCCGGTCCACA
TCTGAGAGCACCGCCGCCCTGGGCTGTCTGGTGAAGGACTACTTCCCAGAGCCCGTGACCGTGAGCTGGAACAGCGGCGC
CCTGACAAGCGGCGTGCACACATTTCCCGCCGTGCTGCAGAGCTCCGGCCTGTACTCCCTGTCTAGCGTGGTGACAGTGC
CTTCCTCTAGCCTGGGCACCAAGACATATACCTGTAACGTGGACCACAAGCCAAGCAATACCAAGGTGGATAAGCGGGTG
GAGTCTAAGTACGGCCCTCCTTGCCCTCCATGTCCTGCTCCAGAGTTTCTGGGCGGCCCTTCCGTGTTCCTGTTTCCACC
CAAACCAAAGGACACACTGATGATCTCTAGAACACCAGAGGTGACCTGCGTGGTGGTGGACGTGAGCCAGGAGGATCCCG
AGGTGCAGTTCAACTGGTACGTGGATGGCGTGGAGGTGCACAATGCCAAGACCAAGCCAAGAGAGGAGCAGTTTAACTCT
ACATACAGGGTGGTGAGCGTGCTGACCGTGCTGCACCAGGATTGGCTCAACGGCAAGGAGTATAAGTGCAAGGTGTCCAA
TAAGGGCCTGCCCTCCTCTATCGAGAAGACAATCTCTAAGGCTAAGGGCCAGCCAAGAGAGCCTCAGGTGTACACCCTGC
CTCCAAGCCAGGAGGAGATGACAAAGAACCAGGTGTCCCTGACATGTCTGGTGAAGGGCTTCTATCCCTCCGACATCGCC
GTGGAGTGGGAGTCTAATGGCCAGCCTGAGAACAATTACAAGACCACACCCCCTGTGCTGGACTCTGATGGCAGCTTCTT
TCTGTATTCCAGGCTGACCGTGGATAAGTCTCGGTGGCAGGAGGGCAACGTGTTCAGCTGCTCTGTGATGCACGAAGCCC
TGCATAATCACTATACTCAGAAAAGTCTGTCACTGTCACTGGGAAAG
14564-VL (SEQ ID NO: 169)
ATGGGCTGGTCATGTATTATTCTGTTTCTGGTCGCAACTGCTACAGGGGTCCATAGTGAAATAGTACTAACACAATCACC
CGCTACTTTGTCACTCTCGCCGGGTGAGCGCGCGACCCTGTCTTGTCGTGCCAGCTCCTACGTGAGCTTTATGCACTGGC
TGCAGCAAAAACCGGGTCAGGCACCGCGTCCGTGGATTTATGCGACGGCCAATCTGGCTAGCGGTGTTCCGGCGCGTTTC
AGCGGCAGCGGCTCTGGCACTGACTACACCTTGACCATCAGCTCTCTGGAACCGGAAGATTTCGCTGTGTATTACTGCCA
GCAATGGAGCTCCAACCCACCGGCGTTTGGTGGTGGCACCAAAGTTGAGATCAAGAGGACAGTGGCCGCCCCAAGCGTGT
TCATCTTTCCCCCTTCCGACGAGCAGCTGAAGTCTGGCACCGCCAGCGTGGTGTGCCTGCTGAACAACTTCTACCCTCGG
GAGGCCAAGGTCCAGTGGAAGGTGGATAACGCCCTGCAGTCTGGCAATAGCCAGGAGTCCGTGACCGAGCAGGACTCTAA
GGATAGCACATATTCCCTGTCTAGCACCCTGACACTGAGCAAGGCCGATTACGAGAAGCACAAGGTGTATGCCTGTGAAG
TCACCCATCAGGGGCTGTCATCACCCGTCACTAAGTCATTCAATCGCGGAGAATGC
14565-VH (SEQ ID NO: 170)
ATGGGCTGGTCTTGTATTATTCTGTTTCTGGTCGCAACTGCTACAGGCGTGCATTCTGAGGTTCAACTAGTAGAATCAGG
TGGAGGGTTGGTGCAGCCGGGTGGTAGCCTCCGCCTGAGCTGTGCTGCGAGCGGTTTCACCTTCAGCTCCTACGGCATGA
GCTGGGTTCGTCAGGCACCGGGCAAAGGCCTGGAACTGGTGTCCACCATCAACGGTTATGGCGATACGTGGTATTACCCG
GATAGCGTTAAAGGCCGTTTTACCATTAGCCGTGACAACTCTAAGAACACCTTATATCTGCAAATGAATTCACTGCGCGC
AGAGGACACTGCCGTTTATTACTGCGCGCGTGACAGAGACTACGGTAATTCCTATTACTACGCGCACGATTACTGGGGTC
AAGGTACGTTGGTCACCGTGTCTTCGGCCAGCACCAAGGGCCCTTCCGTGTTTCCCCTGGCCCCTTGCTCCCGGTCCACA
TCTGAGAGCACCGCCGCCCTGGGCTGTCTGGTGAAGGACTACTTCCCAGAGCCCGTGACCGTGAGCTGGAACAGCGGCGC
CCTGACAAGCGGCGTGCACACATTTCCCGCCGTGCTGCAGAGCTCCGGCCTGTACTCCCTGTCTAGCGTGGTGACAGTGC
CTTCCTCTAGCCTGGGCACCAAGACATATACCTGTAACGTGGACCACAAGCCAAGCAATACCAAGGTGGATAAGCGGGTG
GAGTCTAAGTACGGCCCTCCTTGCCCTCCATGTCCTGCTCCAGAGTTTCTGGGCGGCCCTTCCGTGTTCCTGTTTCCACC
CAAACCAAAGGACACACTGATGATCTCTAGAACACCAGAGGTGACCTGCGTGGTGGTGGACGTGAGCCAGGAGGATCCCG
AGGTGCAGTTCAACTGGTACGTGGATGGCGTGGAGGTGCACAATGCCAAGACCAAGCCAAGAGAGGAGCAGTTTAACTCT
ACATACAGGGTGGTGAGCGTGCTGACCGTGCTGCACCAGGATTGGCTCAACGGCAAGGAGTATAAGTGCAAGGTGTCCAA
TAAGGGCCTGCCCTCCTCTATCGAGAAGACAATCTCTAAGGCTAAGGGCCAGCCAAGAGAGCCTCAGGTGTACACCCTGC
CTCCAAGCCAGGAGGAGATGACAAAGAACCAGGTGTCCCTGACATGTCTGGTGAAGGGCTTCTATCCCTCCGACATCGCC
GTGGAGTGGGAGTCTAATGGCCAGCCTGAGAACAATTACAAGACCACACCCCCTGTGCTGGACTCTGATGGCAGCTTCTT
TCTGTATTCCAGGCTGACCGTGGATAAGTCTCGGTGGCAGGAGGGCAACGTGTTCAGCTGCTCTGTGATGCACGAAGCCC
TGCATAATCACTATACTCAGAAAAGTCTGTCACTGTCACTGGGAAAG
14565-VL (SEQ ID NO: 171)
ATGGGCTGGTCATGTATTATTCTGTTTCTGGTCGCAACTGCTACAGGGGTCCATAGTGAAATAGTACTAACACAATCACC
CGCTACTTTGTCACTCTCGCCGGGTGAGCGCGCGACCCTGTCTTGTCGTGCCAGCTCCTGGGTGAGCTTTATGCACTGGC
TGCAGCAAAAACCGGGTCAGGCACCGCGTCCGTGGATTTATGCGACGCAGAATCTGGCTAGCGGTGTTCCGGCGCGTTTC
AGCGGCAGCGGCTCTGGCACTGACTACACCTTGACCATCAGCTCTCTGGAACCGGAAGATTTCGCTGTGTATTACTGCCA
GCAATGGAGCTCCAACCCACCGGCGTTTGGTGGTGGCACCAAAGTTGAGATCAAGAGGACAGTGGCCGCCCCAAGCGTGT
TCATCTTTCCCCCTTCCGACGAGCAGCTGAAGTCTGGCACCGCCAGCGTGGTGTGCCTGCTGAACAACTTCTACCCTCGG
GAGGCCAAGGTCCAGTGGAAGGTGGATAACGCCCTGCAGTCTGGCAATAGCCAGGAGTCCGTGACCGAGCAGGACTCTAA
GGATAGCACATATTCCCTGTCTAGCACCCTGACACTGAGCAAGGCCGATTACGAGAAGCACAAGGTGTATGCCTGTGAAG
TCACCCATCAGGGGCTGTCATCACCCGTCACTAAGTCATTCAATCGCGGAGAATGC
14566-VH (SEQ ID NO: 172)
ATGGGCTGGTCTTGTATTATTCTGTTTCTGGTCGCAACTGCTACAGGCGTGCATTCTGAGGTTCAACTAGTAGAATCAGG
TGGAGGGTTGGTGCAGCCGGGTGGTAGCCTCCGCCTGAGCTGTGCTGCGAGCGGTTTCACCTTCAGCTCCTACGGCATGA
GCTGGGTTCGTCAGGCACCGGGCAAAGGCCTGGAACTGGTGTCCACCATCAACGGTTATGGCGATACGACCTATTACCCG
GATAGCGTTAAAGGCCGTTTTACCATTAGCCGTGACAACTCTAAGAACACCTTATATCTGCAAATGAATTCACTGCGCGC
AGAGGACACTGCCGTTTATTACTGCGCGCGTGACAGAGACTACGGTAATTCCTATTACTACGCGCACGATTACTGGGGTC
AAGGTACGTTGGTCACCGTGTCTTCGGCCAGCACCAAGGGCCCTTCCGTGTTTCCCCTGGCCCCTTGCTCCCGGTCCACA
TCTGAGAGCACCGCCGCCCTGGGCTGTCTGGTGAAGGACTACTTCCCAGAGCCCGTGACCGTGAGCTGGAACAGCGGCGC
CCTGACAAGCGGCGTGCACACATTTCCCGCCGTGCTGCAGAGCTCCGGCCTGTACTCCCTGTCTAGCGTGGTGACAGTGC
CTTCCTCTAGCCTGGGCACCAAGACATATACCTGTAACGTGGACCACAAGCCAAGCAATACCAAGGTGGATAAGCGGGTG
GAGTCTAAGTACGGCCCTCCTTGCCCTCCATGTCCTGCTCCAGAGTTTCTGGGCGGCCCTTCCGTGTTCCTGTTTCCACC
CAAACCAAAGGACACACTGATGATCTCTAGAACACCAGAGGTGACCTGCGTGGTGGTGGACGTGAGCCAGGAGGATCCCG
AGGTGCAGTTCAACTGGTACGTGGATGGCGTGGAGGTGCACAATGCCAAGACCAAGCCAAGAGAGGAGCAGTTTAACTCT
ACATACAGGGTGGTGAGCGTGCTGACCGTGCTGCACCAGGATTGGCTCAACGGCAAGGAGTATAAGTGCAAGGTGTCCAA
TAAGGGCCTGCCCTCCTCTATCGAGAAGACAATCTCTAAGGCTAAGGGCCAGCCAAGAGAGCCTCAGGTGTACACCCTGC
CTCCAAGCCAGGAGGAGATGACAAAGAACCAGGTGTCCCTGACATGTCTGGTGAAGGGCTTCTATCCCTCCGACATCGCC
GTGGAGTGGGAGTCTAATGGCCAGCCTGAGAACAATTACAAGACCACACCCCCTGTGCTGGACTCTGATGGCAGCTTCTT
TCTGTATTCCAGGCTGACCGTGGATAAGTCTCGGTGGCAGGAGGGCAACGTGTTCAGCTGCTCTGTGATGCACGAAGCCC
TGCATAATCACTATACTCAGAAAAGTCTGTCACTGTCACTGGGAAAG
14566-VL (SEQ ID NO: 173)
ATGGGCTGGTCATGTATTATTCTGTTTCTGGTCGCAACTGCTACAGGGGTCCATAGTGAAATAGTACTAACACAATCACC
CGCTACTTTGTCACTCTCGCCGGGTGAGCGCGCGACCCTGTCTTGTCGTGCCAGCTCCTGGGTGAGCTTTATGCACTGGC
TGCAGCAAAAACCGGGTCAGGCACCGCGTCCGTGGATTTATGCGACGATGAATCTGGCTAGCGGTGTTCCGGCGCGTTTC
AGCGGCAGCGGCTCTGGCACTGACTACACCTTGACCATCAGCTCTCTGGAACCGGAAGATTTCGCTGTGTATTACTGCCA
GCAATGGAGCTCCAACCCACCGGCGTTTGGTGGTGGCACCAAAGTTGAGATCAAGAGGACAGTGGCCGCCCCAAGCGTGT
TCATCTTTCCCCCTTCCGACGAGCAGCTGAAGTCTGGCACCGCCAGCGTGGTGTGCCTGCTGAACAACTTCTACCCTCGG
GAGGCCAAGGTCCAGTGGAAGGTGGATAACGCCCTGCAGTCTGGCAATAGCCAGGAGTCCGTGACCGAGCAGGACTCTAA
GGATAGCACATATTCCCTGTCTAGCACCCTGACACTGAGCAAGGCCGATTACGAGAAGCACAAGGTGTATGCCTGTGAAG
TCACCCATCAGGGGCTGTCATCACCCGTCACTAAGTCATTCAATCGCGGAGAATGC
14567-VH (SEQ ID NO: 174)
ATGGGCTGGTCTTGTATTATTCTGTTTCTGGTCGCAACTGCTACAGGCGTGCATTCTGAGGTTCAACTAGTAGAATCAGG
TGGAGGGTTGGTGCAGCCGGGTGGTAGCCTCCGCCTGAGCTGTGCTGCGAGCGGTTTCTGGTTCAGCTCCTGGGGCATGA
GCTGGGTTCGTCAGGCACCGGGCAAAGGCCTGGAACTGGTGTCCACCATCAACGGTTATGGCGATACGACCTATTACCCG
GATAGCGTTAAAGGCCGTTTTACCATTAGCCGTGACAACTCTAAGAACACCTTATATCTGCAAATGAATTCACTGCGCGC
AGAGGACACTGCCGTTTATTACTGCGCGCGTGACAGAGACTACACTAATTCCTATTACTACGCGCACGATTACTGGGGTC
AAGGTACGTTGGTCACCGTGTCTTCGGCCAGCACCAAGGGCCCTTCCGTGTTTCCCCTGGCCCCTTGCTCCCGGTCCACA
TCTGAGAGCACCGCCGCCCTGGGCTGTCTGGTGAAGGACTACTTCCCAGAGCCCGTGACCGTGAGCTGGAACAGCGGCGC
CCTGACAAGCGGCGTGCACACATTTCCCGCCGTGCTGCAGAGCTCCGGCCTGTACTCCCTGTCTAGCGTGGTGACAGTGC
CTTCCTCTAGCCTGGGCACCAAGACATATACCTGTAACGTGGACCACAAGCCAAGCAATACCAAGGTGGATAAGCGGGTG
GAGTCTAAGTACGGCCCTCCTTGCCCTCCATGTCCTGCTCCAGAGTTTCTGGGCGGCCCTTCCGTGTTCCTGTTTCCACC
CAAACCAAAGGACACACTGATGATCTCTAGAACACCAGAGGTGACCTGCGTGGTGGTGGACGTGAGCCAGGAGGATCCCG
AGGTGCAGTTCAACTGGTACGTGGATGGCGTGGAGGTGCACAATGCCAAGACCAAGCCAAGAGAGGAGCAGTTTAACTCT
ACATACAGGGTGGTGAGCGTGCTGACCGTGCTGCACCAGGATTGGCTCAACGGCAAGGAGTATAAGTGCAAGGTGTCCAA
TAAGGGCCTGCCCTCCTCTATCGAGAAGACAATCTCTAAGGCTAAGGGCCAGCCAAGAGAGCCTCAGGTGTACACCCTGC
CTCCAAGCCAGGAGGAGATGACAAAGAACCAGGTGTCCCTGACATGTCTGGTGAAGGGCTTCTATCCCTCCGACATCGCC
GTGGAGTGGGAGTCTAATGGCCAGCCTGAGAACAATTACAAGACCACACCCCCTGTGCTGGACTCTGATGGCAGCTTCTT
TCTGTATTCCAGGCTGACCGTGGATAAGTCTCGGTGGCAGGAGGGCAACGTGTTCAGCTGCTCTGTGATGCACGAAGCCC
TGCATAATCACTATACTCAGAAAAGTCTGTCACTGTCACTGGGAAAG
14567-VL (SEQ ID NO: 175)
ATGGGCTGGTCATGTATTATTCTGTTTCTGGTCGCAACTGCTACAGGGGTCCATAGTGAAATAGTACTAACACAATCACC
CGCTACTTTGTCACTCTCGCCGGGTGAGCGCGCGACCCTGTCTTGTCGTGCCAGCTCCTACGTGAGCTTTATGCACTGGC
TGCAGCAAAAACCGGGTCAGGCACCGCGTCCGTGGATTTATGCGACGCAGAATCTGGCTAGCGGTGTTCCGGCGCGTTTC
AGCGGCAGCGGCTCTGGCACTGACTACACCTTGACCATCAGCTCTCTGGAACCGGAAGATTTCGCTGTGTATTACTGCCA
GCAATGGAGCTCCAACCCACCGGCGTTTGGTGGTGGCACCAAAGTTGAGATCAAGAGGACAGTGGCCGCCCCAAGCGTGT
TCATCTTTCCCCCTTCCGACGAGCAGCTGAAGTCTGGCACCGCCAGCGTGGTGTGCCTGCTGAACAACTTCTACCCTCGG
GAGGCCAAGGTCCAGTGGAAGGTGGATAACGCCCTGCAGTCTGGCAATAGCCAGGAGTCCGTGACCGAGCAGGACTCTAA
GGATAGCACATATTCCCTGTCTAGCACCCTGACACTGAGCAAGGCCGATTACGAGAAGCACAAGGTGTATGCCTGTGAAG
TCACCCATCAGGGGCTGTCATCACCCGTCACTAAGTCATTCAATCGCGGAGAATGC
14568-VH (SEQ ID NO: 176)
ATGGGCTGGTCTTGTATTATTCTGTTTCTGGTCGCAACTGCTACAGGCGTGCATTCTGAGGTTCAACTAGTAGAATCAGG
TGGAGGGTTGGTGCAGCCGGGTGGTAGCCTCCGCCTGAGCTGTGCTGCGAGCGGTTTCACCTTCAGCTCCTACGGCATGA
GCTGGGTTCGTCAGGCACCGGGCAAAGGCCTGGAACTGGTGTCCACCATCAACGGTTATGGCGATACGACCTATTACCCG
GATAGCGTTAAAGGCCGTTTTACCATTAGCCGTGACAACTCTAAGAACACCTTATATCTGCAAATGAATTCACTGCGCGC
AGAGGACACTGCCGTTTATTACTGCGCGCGTGACAGAGACTACACTAATTCCTATTACTACGCGCACGATTACTGGGGTC
AAGGTACGTTGGTCACCGTGTCTTCGGCCAGCACCAAGGGCCCTTCCGTGTTTCCCCTGGCCCCTTGCTCCCGGTCCACA
TCTGAGAGCACCGCCGCCCTGGGCTGTCTGGTGAAGGACTACTTCCCAGAGCCCGTGACCGTGAGCTGGAACAGCGGCGC
CCTGACAAGCGGCGTGCACACATTTCCCGCCGTGCTGCAGAGCTCCGGCCTGTACTCCCTGTCTAGCGTGGTGACAGTGC
CTTCCTCTAGCCTGGGCACCAAGACATATACCTGTAACGTGGACCACAAGCCAAGCAATACCAAGGTGGATAAGCGGGTG
GAGTCTAAGTACGGCCCTCCTTGCCCTCCATGTCCTGCTCCAGAGTTTCTGGGCGGCCCTTCCGTGTTCCTGTTTCCACC
CAAACCAAAGGACACACTGATGATCTCTAGAACACCAGAGGTGACCTGCGTGGTGGTGGACGTGAGCCAGGAGGATCCCG
AGGTGCAGTTCAACTGGTACGTGGATGGCGTGGAGGTGCACAATGCCAAGACCAAGCCAAGAGAGGAGCAGTTTAACTCT
ACATACAGGGTGGTGAGCGTGCTGACCGTGCTGCACCAGGATTGGCTCAACGGCAAGGAGTATAAGTGCAAGGTGTCCAA
TAAGGGCCTGCCCTCCTCTATCGAGAAGACAATCTCTAAGGCTAAGGGCCAGCCAAGAGAGCCTCAGGTGTACACCCTGC
CTCCAAGCCAGGAGGAGATGACAAAGAACCAGGTGTCCCTGACATGTCTGGTGAAGGGCTTCTATCCCTCCGACATCGCC
GTGGAGTGGGAGTCTAATGGCCAGCCTGAGAACAATTACAAGACCACACCCCCTGTGCTGGACTCTGATGGCAGCTTCTT
TCTGTATTCCAGGCTGACCGTGGATAAGTCTCGGTGGCAGGAGGGCAACGTGTTCAGCTGCTCTGTGATGCACGAAGCCC
TGCATAATCACTATACTCAGAAAAGTCTGTCACTGTCACTGGGAAAG
14568-VL (SEQ ID NO: 177)
ATGGGCTGGTCATGTATTATTCTGTTTCTGGTCGCAACTGCTACAGGGGTCCATAGTGAAATAGTACTAACACAATCACC
CGCTACTTTGTCACTCTCGCCGGGTGAGCGCGCGACCCTGTCTTGTCGTGCCAGCTCCTACGTGAGCTTTATGCACTGGC
TGCAGCAAAAACCGGGTCAGGCACCGCGTCCGTGGATTTATGCGACGATGAATCTGGCTAGCGGTGTTCCGGCGCGTTTC
AGCGGCAGCGGCTCTGGCACTGACTACACCTTGACCATCAGCTCTCTGGAACCGGAAGATTTCGCTGTGTATTACTGCCA
GCAATGGAGCTCCAACCCACCGGCGTTTGGTGGTGGCACCAAAGTTGAGATCAAGAGGACAGTGGCCGCCCCAAGCGTGT
TCATCTTTCCCCCTTCCGACGAGCAGCTGAAGTCTGGCACCGCCAGCGTGGTGTGCCTGCTGAACAACTTCTACCCTCGG
GAGGCCAAGGTCCAGTGGAAGGTGGATAACGCCCTGCAGTCTGGCAATAGCCAGGAGTCCGTGACCGAGCAGGACTCTAA
GGATAGCACATATTCCCTGTCTAGCACCCTGACACTGAGCAAGGCCGATTACGAGAAGCACAAGGTGTATGCCTGTGAAG
TCACCCATCAGGGGCTGTCATCACCCGTCACTAAGTCATTCAATCGCGGAGAATGC
14569-VH (SEQ ID NO: 178)
ATGGGCTGGTCTTGTATTATTCTGTTTCTGGTCGCAACTGCTACAGGCGTGCATTCTGAGGTTCAACTAGTAGAATCAGG
TGGAGGGTTGGTGCAGCCGGGTGGTAGCCTCCGCCTGAGCTGTGCTGCGAGCGGTTACTGGTTCAGCTCCTGGGGCATGA
GCTGGGTTCGTCAGGCACCGGGCAAAGGCCTGGAACTGGTGTCCACCATCAACGGTTATGGCGATACGACCTATTACCCG
GATAGCGTTAAAGGCCGTTTTACCATTAGCCGTGACAACTCTAAGAACACCTTATATCTGCAAATGAATTCACTGCGCGC
AGAGGACACTGCCGTTTATTACTGCGCGCGTGACAGAGACTACTCTAATTCCTATTACTACGCGCACGATTACTGGGGTC
AAGGTACGTTGGTCACCGTGTCTTCGGCCAGCACCAAGGGCCCTTCCGTGTTTCCCCTGGCCCCTTGCTCCCGGTCCACA
TCTGAGAGCACCGCCGCCCTGGGCTGTCTGGTGAAGGACTACTTCCCAGAGCCCGTGACCGTGAGCTGGAACAGCGGCGC
CCTGACAAGCGGCGTGCACACATTTCCCGCCGTGCTGCAGAGCTCCGGCCTGTACTCCCTGTCTAGCGTGGTGACAGTGC
CTTCCTCTAGCCTGGGCACCAAGACATATACCTGTAACGTGGACCACAAGCCAAGCAATACCAAGGTGGATAAGCGGGTG
GAGTCTAAGTACGGCCCTCCTTGCCCTCCATGTCCTGCTCCAGAGTTTCTGGGCGGCCCTTCCGTGTTCCTGTTTCCACC
CAAACCAAAGGACACACTGATGATCTCTAGAACACCAGAGGTGACCTGCGTGGTGGTGGACGTGAGCCAGGAGGATCCCG
AGGTGCAGTTCAACTGGTACGTGGATGGCGTGGAGGTGCACAATGCCAAGACCAAGCCAAGAGAGGAGCAGTTTAACTCT
ACATACAGGGTGGTGAGCGTGCTGACCGTGCTGCACCAGGATTGGCTCAACGGCAAGGAGTATAAGTGCAAGGTGTCCAA
TAAGGGCCTGCCCTCCTCTATCGAGAAGACAATCTCTAAGGCTAAGGGCCAGCCAAGAGAGCCTCAGGTGTACACCCTGC
CTCCAAGCCAGGAGGAGATGACAAAGAACCAGGTGTCCCTGACATGTCTGGTGAAGGGCTTCTATCCCTCCGACATCGCC
GTGGAGTGGGAGTCTAATGGCCAGCCTGAGAACAATTACAAGACCACACCCCCTGTGCTGGACTCTGATGGCAGCTTCTT
TCTGTATTCCAGGCTGACCGTGGATAAGTCTCGGTGGCAGGAGGGCAACGTGTTCAGCTGCTCTGTGATGCACGAAGCCC
TGCATAATCACTATACTCAGAAAAGTCTGTCACTGTCACTGGGAAAG
14569-VL (SEQ ID NO: 179)
ATGGGCTGGTCATGTATTATTCTGTTTCTGGTCGCAACTGCTACAGGGGTCCATAGTGAAATAGTACTAACACAATCACC
CGCTACTTTGTCACTCTCGCCGGGTGAGCGCGCGACCCTGTCTTGTCGTGCCAGCTCCTACGTGAGCTTTATGCACTGGC
TGCAGCAAAAACCGGGTCAGGCACCGCGTCCGTGGATTTATGCGACGATGAATCTGGCTAGCGGTGTTCCGGCGCGTTTC
AGCGGCAGCGGCTCTGGCACTGACTACACCTTGACCATCAGCTCTCTGGAACCGGAAGATTTCGCTGTGTATTACTGCCA
GCAATGGAGCTCCAACCCACCGGCGTTTGGTGGTGGCACCAAAGTTGAGATCAAGAGGACAGTGGCCGCCCCAAGCGTGT
TCATCTTTCCCCCTTCCGACGAGCAGCTGAAGTCTGGCACCGCCAGCGTGGTGTGCCTGCTGAACAACTTCTACCCTCGG
GAGGCCAAGGTCCAGTGGAAGGTGGATAACGCCCTGCAGTCTGGCAATAGCCAGGAGTCCGTGACCGAGCAGGACTCTAA
GGATAGCACATATTCCCTGTCTAGCACCCTGACACTGAGCAAGGCCGATTACGAGAAGCACAAGGTGTATGCCTGTGAAG
TCACCCATCAGGGGCTGTCATCACCCGTCACTAAGTCATTCAATCGCGGAGAATGC
14570-VH (SEQ ID NO: 180)
ATGGGCTGGTCTTGTATTATTCTGTTTCTGGTCGCAACTGCTACAGGCGTGCATTCTGAGGTTCAACTAGTAGAATCAGG
TGGAGGGTTGGTGCAGCCGGGTGGTAGCCTCCGCCTGAGCTGTGCTGCGAGCGGTTTCTGGTTCAGCTCCTACGGCATGA
GCTGGGTTCGTCAGGCACCGGGCAAAGGCCTGGAACTGGTGTCCACCATCAACGGTTATGGCGATACGACCTATTACCCG
GATAGCGTTAAAGGCCGTTTTACCATTAGCCGTGACAACTCTAAGAACACCTTATATCTGCAAATGAATTCACTGCGCGC
AGAGGACACTGCCGTTTATTACTGCGCGCGTGACAGAGACTACACTAATTCCTATTACTACGCGCACGATTACTGGGGTC
AAGGTACGTTGGTCACCGTGTCTTCGGCCAGCACCAAGGGCCCTTCCGTGTTTCCCCTGGCCCCTTGCTCCCGGTCCACA
TCTGAGAGCACCGCCGCCCTGGGCTGTCTGGTGAAGGACTACTTCCCAGAGCCCGTGACCGTGAGCTGGAACAGCGGCGC
CCTGACAAGCGGCGTGCACACATTTCCCGCCGTGCTGCAGAGCTCCGGCCTGTACTCCCTGTCTAGCGTGGTGACAGTGC
CTTCCTCTAGCCTGGGCACCAAGACATATACCTGTAACGTGGACCACAAGCCAAGCAATACCAAGGTGGATAAGCGGGTG
GAGTCTAAGTACGGCCCTCCTTGCCCTCCATGTCCTGCTCCAGAGTTTCTGGGCGGCCCTTCCGTGTTCCTGTTTCCACC
CAAACCAAAGGACACACTGATGATCTCTAGAACACCAGAGGTGACCTGCGTGGTGGTGGACGTGAGCCAGGAGGATCCCG
AGGTGCAGTTCAACTGGTACGTGGATGGCGTGGAGGTGCACAATGCCAAGACCAAGCCAAGAGAGGAGCAGTTTAACTCT
ACATACAGGGTGGTGAGCGTGCTGACCGTGCTGCACCAGGATTGGCTCAACGGCAAGGAGTATAAGTGCAAGGTGTCCAA
TAAGGGCCTGCCCTCCTCTATCGAGAAGACAATCTCTAAGGCTAAGGGCCAGCCAAGAGAGCCTCAGGTGTACACCCTGC
CTCCAAGCCAGGAGGAGATGACAAAGAACCAGGTGTCCCTGACATGTCTGGTGAAGGGCTTCTATCCCTCCGACATCGCC
GTGGAGTGGGAGTCTAATGGCCAGCCTGAGAACAATTACAAGACCACACCCCCTGTGCTGGACTCTGATGGCAGCTTCTT
TCTGTATTCCAGGCTGACCGTGGATAAGTCTCGGTGGCAGGAGGGCAACGTGTTCAGCTGCTCTGTGATGCACGAAGCCC
TGCATAATCACTATACTCAGAAAAGTCTGTCACTGTCACTGGGAAAG
14570-VL (SEQ ID NO: 181)
ATGGGCTGGTCATGTATTATTCTGTTTCTGGTCGCAACTGCTACAGGGGTCCATAGTGAAATAGTACTAACACAATCACC
CGCTACTTTGTCACTCTCGCCGGGTGAGCGCGCGACCCTGTCTTGTCGTGCCAGCTCCTCCGTGAGCTTTATGCACTGGC
TGCAGCAAAAACCGGGTCAGGCACCGCGTCCGTGGATTTATGCGACGGCCAATCTGGCTAGCGGTGTTCCGGCGCGTTTC
AGCGGCAGCGGCTCTGGCACTGACTACACCTTGACCATCAGCTCTCTGGAACCGGAAGATTTCGCTGTGTATTACTGCCA
GCAATGGAGCTCCAACCCACCGGCGTTTGGTGGTGGCACCAAAGTTGAGATCAAGAGGACAGTGGCCGCCCCAAGCGTGT
TCATCTTTCCCCCTTCCGACGAGCAGCTGAAGTCTGGCACCGCCAGCGTGGTGTGCCTGCTGAACAACTTCTACCCTCGG
GAGGCCAAGGTCCAGTGGAAGGTGGATAACGCCCTGCAGTCTGGCAATAGCCAGGAGTCCGTGACCGAGCAGGACTCTAA
GGATAGCACATATTCCCTGTCTAGCACCCTGACACTGAGCAAGGCCGATTACGAGAAGCACAAGGTGTATGCCTGTGAAG
TCACCCATCAGGGGCTGTCATCACCCGTCACTAAGTCATTCAATCGCGGAGAATGC
14571-VH (SEQ ID NO: 182)
ATGGGCTGGTCTTGTATTATTCTGTTTCTGGTCGCAACTGCTACAGGCGTGCATTCTGAGGTTCAACTAGTAGAATCAGG
TGGAGGGTTGGTGCAGCCGGGTGGTAGCCTCCGCCTGAGCTGTGCTGCGAGCGGTTACACCTTCAGCTCCTACGGCATGA
GCTGGGTTCGTCAGGCACCGGGCAAAGGCCTGGAACTGGTGTCCACCATCAACGGTTATGGCGATACGACCTATTACCCG
GATAGCGTTAAAGGCCGTTTTACCATTAGCCGTGACAACTCTAAGAACACCTTATATCTGCAAATGAATTCACTGCGCGC
AGAGGACACTGCCGTTTATTACTGCGCGCGTGACAGAGACTACGGTAATTCCTATTACTACGCGCACGATTACTGGGGTC
AAGGTACGTTGGTCACCGTGTCTTCGGCCAGCACCAAGGGCCCTTCCGTGTTTCCCCTGGCCCCTTGCTCCCGGTCCACA
TCTGAGAGCACCGCCGCCCTGGGCTGTCTGGTGAAGGACTACTTCCCAGAGCCCGTGACCGTGAGCTGGAACAGCGGCGC
CCTGACAAGCGGCGTGCACACATTTCCCGCCGTGCTGCAGAGCTCCGGCCTGTACTCCCTGTCTAGCGTGGTGACAGTGC
CTTCCTCTAGCCTGGGCACCAAGACATATACCTGTAACGTGGACCACAAGCCAAGCAATACCAAGGTGGATAAGCGGGTG
GAGTCTAAGTACGGCCCTCCTTGCCCTCCATGTCCTGCTCCAGAGTTTCTGGGCGGCCCTTCCGTGTTCCTGTTTCCACC
CAAACCAAAGGACACACTGATGATCTCTAGAACACCAGAGGTGACCTGCGTGGTGGTGGACGTGAGCCAGGAGGATCCCG
AGGTGCAGTTCAACTGGTACGTGGATGGCGTGGAGGTGCACAATGCCAAGACCAAGCCAAGAGAGGAGCAGTTTAACTCT
ACATACAGGGTGGTGAGCGTGCTGACCGTGCTGCACCAGGATTGGCTCAACGGCAAGGAGTATAAGTGCAAGGTGTCCAA
TAAGGGCCTGCCCTCCTCTATCGAGAAGACAATCTCTAAGGCTAAGGGCCAGCCAAGAGAGCCTCAGGTGTACACCCTGC
CTCCAAGCCAGGAGGAGATGACAAAGAACCAGGTGTCCCTGACATGTCTGGTGAAGGGCTTCTATCCCTCCGACATCGCC
GTGGAGTGGGAGTCTAATGGCCAGCCTGAGAACAATTACAAGACCACACCCCCTGTGCTGGACTCTGATGGCAGCTTCTT
TCTGTATTCCAGGCTGACCGTGGATAAGTCTCGGTGGCAGGAGGGCAACGTGTTCAGCTGCTCTGTGATGCACGAAGCCC
TGCATAATCACTATACTCAGAAAAGTCTGTCACTGTCACTGGGAAAG
14571-VL (SEQ ID NO: 183)
ATGGGCTGGTCATGTATTATTCTGTTTCTGGTCGCAACTGCTACAGGGGTCCATAGTGAAATAGTACTAACACAATCACC
CGCTACTTTGTCACTCTCGCCGGGTGAGCGCGCGACCCTGTCTTGTCGTGCCAGCTCCTGGGTGAGCTTTATGCACTGGC
TGCAGCAAAAACCGGGTCAGGCACCGCGTCCGTGGATTTATGCGACGATGAATCTGGCTAGCGGTGTTCCGGCGCGTTTC
AGCGGCAGCGGCTCTGGCACTGACTACACCTTGACCATCAGCTCTCTGGAACCGGAAGATTTCGCTGTGTATTACTGCCA
GCAATGGAGCTCCAACCCACCGGCGTTTGGTGGTGGCACCAAAGTTGAGATCAAGAGGACAGTGGCCGCCCCAAGCGTGT
TCATCTTTCCCCCTTCCGACGAGCAGCTGAAGTCTGGCACCGCCAGCGTGGTGTGCCTGCTGAACAACTTCTACCCTCGG
GAGGCCAAGGTCCAGTGGAAGGTGGATAACGCCCTGCAGTCTGGCAATAGCCAGGAGTCCGTGACCGAGCAGGACTCTAA
GGATAGCACATATTCCCTGTCTAGCACCCTGACACTGAGCAAGGCCGATTACGAGAAGCACAAGGTGTATGCCTGTGAAG
TCACCCATCAGGGGCTGTCATCACCCGTCACTAAGTCATTCAATCGCGGAGAATGC
14572-VH (SEQ ID NO: 184)
ATGGGCTGGTCTTGTATTATTCTGTTTCTGGTCGCAACTGCTACAGGCGTGCATTCTGAGGTTCAACTAGTAGAATCAGG
TGGAGGGTTGGTGCAGCCGGGTGGTAGCCTCCGCCTGAGCTGTGCTGCGAGCGGTTACTGGTTCAGCTCCTACGGCATGA
GCTGGGTTCGTCAGGCACCGGGCAAAGGCCTGGAACTGGTGTCCACCATCAACGGTTATGGCGATACGACCTATTACCCG
GATAGCGTTAAAGGCCGTTTTACCATTAGCCGTGACAACTCTAAGAACACCTTATATCTGCAAATGAATTCACTGCGCGC
AGAGGACACTGCCGTTTATTACTGCGCGCGTGACAGAGACTACACTAATTCCTATTACTACGCGCACGATTACTGGGGTC
AAGGTACGTTGGTCACCGTGTCTTCGGCCAGCACCAAGGGCCCTTCCGTGTTTCCCCTGGCCCCTTGCTCCCGGTCCACA
TCTGAGAGCACCGCCGCCCTGGGCTGTCTGGTGAAGGACTACTTCCCAGAGCCCGTGACCGTGAGCTGGAACAGCGGCGC
CCTGACAAGCGGCGTGCACACATTTCCCGCCGTGCTGCAGAGCTCCGGCCTGTACTCCCTGTCTAGCGTGGTGACAGTGC
CTTCCTCTAGCCTGGGCACCAAGACATATACCTGTAACGTGGACCACAAGCCAAGCAATACCAAGGTGGATAAGCGGGTG
GAGTCTAAGTACGGCCCTCCTTGCCCTCCATGTCCTGCTCCAGAGTTTCTGGGCGGCCCTTCCGTGTTCCTGTTTCCACC
CAAACCAAAGGACACACTGATGATCTCTAGAACACCAGAGGTGACCTGCGTGGTGGTGGACGTGAGCCAGGAGGATCCCG
AGGTGCAGTTCAACTGGTACGTGGATGGCGTGGAGGTGCACAATGCCAAGACCAAGCCAAGAGAGGAGCAGTTTAACTCT
ACATACAGGGTGGTGAGCGTGCTGACCGTGCTGCACCAGGATTGGCTCAACGGCAAGGAGTATAAGTGCAAGGTGTCCAA
TAAGGGCCTGCCCTCCTCTATCGAGAAGACAATCTCTAAGGCTAAGGGCCAGCCAAGAGAGCCTCAGGTGTACACCCTGC
CTCCAAGCCAGGAGGAGATGACAAAGAACCAGGTGTCCCTGACATGTCTGGTGAAGGGCTTCTATCCCTCCGACATCGCC
GTGGAGTGGGAGTCTAATGGCCAGCCTGAGAACAATTACAAGACCACACCCCCTGTGCTGGACTCTGATGGCAGCTTCTT
TCTGTATTCCAGGCTGACCGTGGATAAGTCTCGGTGGCAGGAGGGCAACGTGTTCAGCTGCTCTGTGATGCACGAAGCCC
TGCATAATCACTATACTCAGAAAAGTCTGTCACTGTCACTGGGAAAG
14572-VL (SEQ ID NO: 185)
ATGGGCTGGTCATGTATTATTCTGTTTCTGGTCGCAACTGCTACAGGGGTCCATAGTGAAATAGTACTAACACAATCACC
CGCTACTTTGTCACTCTCGCCGGGTGAGCGCGCGACCCTGTCTTGTCGTGCCAGCTCCTACGTGAGCTTTATGCACTGGC
TGCAGCAAAAACCGGGTCAGGCACCGCGTCCGTGGATTTATGCGACGATGAATCTGGCTAGCGGTGTTCCGGCGCGTTTC
AGCGGCAGCGGCTCTGGCACTGACTACACCTTGACCATCAGCTCTCTGGAACCGGAAGATTTCGCTGTGTATTACTGCCA
GCAATGGAGCTCCAACCCACCGGCGTTTGGTGGTGGCACCAAAGTTGAGATCAAGAGGACAGTGGCCGCCCCAAGCGTGT
TCATCTTTCCCCCTTCCGACGAGCAGCTGAAGTCTGGCACCGCCAGCGTGGTGTGCCTGCTGAACAACTTCTACCCTCGG
GAGGCCAAGGTCCAGTGGAAGGTGGATAACGCCCTGCAGTCTGGCAATAGCCAGGAGTCCGTGACCGAGCAGGACTCTAA
GGATAGCACATATTCCCTGTCTAGCACCCTGACACTGAGCAAGGCCGATTACGAGAAGCACAAGGTGTATGCCTGTGAAG
TCACCCATCAGGGGCTGTCATCACCCGTCACTAAGTCATTCAATCGCGGAGAATGC
14573-VH (SEQ ID NO: 186)
ATGGGCTGGTCTTGTATTATTCTGTTTCTGGTCGCAACTGCTACAGGCGTGCATTCTGAGGTTCAACTAGTAGAATCAGG
TGGAGGGTTGGTGCAGCCGGGTGGTAGCCTCCGCCTGAGCTGTGCTGCGAGCGGTTTCACCTTCAGCTCCTGGGGCATGA
GCTGGGTTCGTCAGGCACCGGGCAAAGGCCTGGAACTGGTGTCCACCATCAACGGTTATGGCGATACGACCTATTACCCG
GATAGCGTTAAAGGCCGTTTTACCATTAGCCGTGACAACTCTAAGAACACCTTATATCTGCAAATGAATTCACTGCGCGC
AGAGGACACTGCCGTTTATTACTGCGCGCGTGACAGAGACTACACTAATTCCTATTACTACGCGCACGATTACTGGGGTC
AAGGTACGTTGGTCACCGTGTCTTCGGCCAGCACCAAGGGCCCTTCCGTGTTTCCCCTGGCCCCTTGCTCCCGGTCCACA
TCTGAGAGCACCGCCGCCCTGGGCTGTCTGGTGAAGGACTACTTCCCAGAGCCCGTGACCGTGAGCTGGAACAGCGGCGC
CCTGACAAGCGGCGTGCACACATTTCCCGCCGTGCTGCAGAGCTCCGGCCTGTACTCCCTGTCTAGCGTGGTGACAGTGC
CTTCCTCTAGCCTGGGCACCAAGACATATACCTGTAACGTGGACCACAAGCCAAGCAATACCAAGGTGGATAAGCGGGTG
GAGTCTAAGTACGGCCCTCCTTGCCCTCCATGTCCTGCTCCAGAGTTTCTGGGCGGCCCTTCCGTGTTCCTGTTTCCACC
CAAACCAAAGGACACACTGATGATCTCTAGAACACCAGAGGTGACCTGCGTGGTGGTGGACGTGAGCCAGGAGGATCCCG
AGGTGCAGTTCAACTGGTACGTGGATGGCGTGGAGGTGCACAATGCCAAGACCAAGCCAAGAGAGGAGCAGTTTAACTCT
ACATACAGGGTGGTGAGCGTGCTGACCGTGCTGCACCAGGATTGGCTCAACGGCAAGGAGTATAAGTGCAAGGTGTCCAA
TAAGGGCCTGCCCTCCTCTATCGAGAAGACAATCTCTAAGGCTAAGGGCCAGCCAAGAGAGCCTCAGGTGTACACCCTGC
CTCCAAGCCAGGAGGAGATGACAAAGAACCAGGTGTCCCTGACATGTCTGGTGAAGGGCTTCTATCCCTCCGACATCGCC
GTGGAGTGGGAGTCTAATGGCCAGCCTGAGAACAATTACAAGACCACACCCCCTGTGCTGGACTCTGATGGCAGCTTCTT
TCTGTATTCCAGGCTGACCGTGGATAAGTCTCGGTGGCAGGAGGGCAACGTGTTCAGCTGCTCTGTGATGCACGAAGCCC
TGCATAATCACTATACTCAGAAAAGTCTGTCACTGTCACTGGGAAAG
14573-VL (SEQ ID NO: 187)
ATGGGCTGGTCATGTATTATTCTGTTTCTGGTCGCAACTGCTACAGGGGTCCATAGTGAAATAGTACTAACACAATCACC
CGCTACTTTGTCACTCTCGCCGGGTGAGCGCGCGACCCTGTCTTGTCGTGCCAGCTCCTACGTGAGCTTTATGCACTGGC
TGCAGCAAAAACCGGGTCAGGCACCGCGTCCGTGGATTTATGCGACGAGCAATCTGGCTAGCGGTGTTCCGGCGCGTTTC
AGCGGCAGCGGCTCTGGCACTGACTACACCTTGACCATCAGCTCTCTGGAACCGGAAGATTTCGCTGTGTATTACTGCCA
GCAATGGAGCTCCAACCCACCGGCGTTTGGTGGTGGCACCAAAGTTGAGATCAAGAGGACAGTGGCCGCCCCAAGCGTGT
TCATCTTTCCCCCTTCCGACGAGCAGCTGAAGTCTGGCACCGCCAGCGTGGTGTGCCTGCTGAACAACTTCTACCCTCGG
GAGGCCAAGGTCCAGTGGAAGGTGGATAACGCCCTGCAGTCTGGCAATAGCCAGGAGTCCGTGACCGAGCAGGACTCTAA
GGATAGCACATATTCCCTGTCTAGCACCCTGACACTGAGCAAGGCCGATTACGAGAAGCACAAGGTGTATGCCTGTGAAG
TCACCCATCAGGGGCTGTCATCACCCGTCACTAAGTCATTCAATCGCGGAGAATGC
U579-WT-VH (SEQ ID NO: 188)
ATGGGCTGGTCTTGTATTATTCTGTTTCTGGTCGCAACTGCTACAGGCGTGCATTCTGAGGTTCAACTAGTAGAATCAGG
TGGAGGGTTGGTGCAGCCGGGTGGTAGCCTCCGCCTGAGCTGTGCTGCGAGCGGTTTCACCTTCAGCTCCTACGGCATGA
GCTGGGTTCGTCAGGCACCGGGCAAAGGCCTGGAACTGGTGTCCACCATCAACGGTTATGGCGATACGACCTATTACCCG
GATAGCGTTAAAGGCCGTTTTACCATTAGCCGTGACAACTCTAAGAACACCTTATATCTGCAAATGAATTCACTGCGCGC
AGAGGACACTGCCGTTTATTACTGCGCGCGTGACAGAGACTACGGTAATTCCTATTACTACGCGCTGGATTACTGGGGTC
AAGGTACGTTGGTCACCGTGTCTTCGGCCAGCACCAAGGGCCCTTCCGTGTTTCCCCTGGCCCCTTGCTCCCGGTCCACA
TCTGAGAGCACCGCCGCCCTGGGCTGTCTGGTGAAGGACTACTTCCCAGAGCCCGTGACCGTGAGCTGGAACAGCGGCGC
CCTGACAAGCGGCGTGCACACATTTCCCGCCGTGCTGCAGAGCTCCGGCCTGTACTCCCTGTCTAGCGTGGTGACAGTGC
CTTCCTCTAGCCTGGGCACCAAGACATATACCTGTAACGTGGACCACAAGCCAAGCAATACCAAGGTGGATAAGCGGGTG
GAGTCTAAGTACGGCCCTCCTTGCCCTCCATGTCCTGCTCCAGAGTTTCTGGGCGGCCCTTCCGTGTTCCTGTTTCCACC
CAAACCAAAGGACACACTGATGATCTCTAGAACACCAGAGGTGACCTGCGTGGTGGTGGACGTGAGCCAGGAGGATCCCG
AGGTGCAGTTCAACTGGTACGTGGATGGCGTGGAGGTGCACAATGCCAAGACCAAGCCAAGAGAGGAGCAGTTTAACTCT
ACATACAGGGTGGTGAGCGTGCTGACCGTGCTGCACCAGGATTGGCTCAACGGCAAGGAGTATAAGTGCAAGGTGTCCAA
TAAGGGCCTGCCCTCCTCTATCGAGAAGACAATCTCTAAGGCTAAGGGCCAGCCAAGAGAGCCTCAGGTGTACACCCTGC
CTCCAAGCCAGGAGGAGATGACAAAGAACCAGGTGTCCCTGACATGTCTGGTGAAGGGCTTCTATCCCTCCGACATCGCC
GTGGAGTGGGAGTCTAATGGCCAGCCTGAGAACAATTACAAGACCACACCCCCTGTGCTGGACTCTGATGGCAGCTTCTT
TCTGTATTCCAGGCTGACCGTGGATAAGTCTCGGTGGCAGGAGGGCAACGTGTTCAGCTGCTCTGTGATGCACGAAGCCC
TGCATAATCACTATACTCAGAAAAGTCTGTCACTGTCACTGGGAAAG
U579-WT-VL (SEQ ID NO: 189)
ATGGGCTGGTCATGTATTATTCTGTTTCTGGTCGCAACTGCTACAGGGGTCCATAGTGAAATAGTACTAACACAATCACC
CGCTACTTTGTCACTCTCGCCGGGTGAGCGCGCGACCCTGTCTTGTCGTGCCAGCTCCTCCGTGAGCTTTATGCACTGGC
TGCAGCAAAAACCGGGTCAGGCACCGCGTCCGTGGATTTATGCGACGAGCAATCTGGCTAGCGGTGTTCCGGCGCGTTTC
AGCGGCAGCGGCTCTGGCACTGACTACACCTTGACCATCAGCTCTCTGGAACCGGAAGATTTCGCTGTGTATTACTGCCA
GCAATGGAGCTCCAACCCACCGGCGTTTGGTGGTGGCACCAAAGTTGAGATCAAGAGGACAGTGGCCGCCCCAAGCGTGT
TCATCTTTCCCCCTTCCGACGAGCAGCTGAAGTCTGGCACCGCCAGCGTGGTGTGCCTGCTGAACAACTTCTACCCTCGG
GAGGCCAAGGTCCAGTGGAAGGTGGATAACGCCCTGCAGTCTGGCAATAGCCAGGAGTCCGTGACCGAGCAGGACTCTAA
GGATAGCACATATTCCCTGTCTAGCACCCTGACACTGAGCAAGGCCGATTACGAGAAGCACAAGGTGTATGCCTGTGAAG
TCACCCATCAGGGGCTGTCATCACCCGTCACTAAGTCATTCAATCGCGGAGAATGC

Thus, in some aspects, the nucleic acid molecule encodes a VH domain, a VL domain, or both a VH domain and VL domain of a CD25-specific monoclonal antibody. In some examples, the nucleic acid molecule encodes a complete heavy chain, a complete light chain, or both a complete heavy chain and a complete light chain of a CD25-specific monoclonal antibody.

In specific aspects, the nucleic acid molecule includes the nucleotide sequence of any one of SEQ ID NOs: 102-143, or a degenerate variant thereof. In some examples, the nucleic acid molecule includes the nucleotide sequence of any one of SEQ ID NOs: 102, 104, 106, 108, 110, 112, 114, 116, 118, 120, 122, 124, 126, 128, 130, 132, 134, 136, 138, 140 and 142, or a degenerate variant thereof; and the nucleotide sequence of any one of SEQ ID NOs: 103, 105, 107, 109, 111, 113, 115, 117, 119, 121, 123, 125, 127, 129, 131, 133, 135, 137, 139, 141 and 143, or a degenerate variant thereof.

In other specific aspects, the nucleic acid molecule includes the nucleotide sequence of any one of SEQ ID NOs: 148-189, or a degenerate variant thereof. In some examples, the nucleic acid molecule includes the nucleotide sequence of any one of SEQ ID NOs: 148, 150, 152, 154, 156, 158, 160, 162, 164, 166, 168, 170, 172, 174, 176, 178, 180, 182, 184, 186 and 188, or a degenerate variant thereof; and the nucleotide sequence of any one of SEQ ID NOs: 149, 151, 153, 155, 157, 159, 161, 163, 165, 167, 169, 171, 173, 175, 177, 179, 181, 183, 185, 187 and 189, or a degenerate variant thereof.

In some aspects, a disclosed nucleic acid molecule further includes one or more stop codons at the 3′ terminus, such as TGA, TAA or TGATAA.

In particular examples, the nucleic acid molecule includes: the nucleotide sequence of SEQ ID NO: 122, or a degenerate variant thereof; the nucleotide sequence of SEQ ID NO: 123, or a degenerate variant thereof; the nucleotide sequences of SEQ ID NO: 122 and SEQ ID NO: 123, or degenerate variants thereof; the nucleotide sequence of SEQ ID NO: 132, or a degenerate variant thereof; the nucleotide sequence of SEQ ID NO: 133, or a degenerate variant thereof; the nucleotide sequences of SEQ ID NO: 132 and SEQ ID NO: 133, or degenerate variants thereof; the nucleotide sequence of SEQ ID NO: 138, or a degenerate variant thereof; the nucleotide sequence of SEQ ID NO: 139, or a degenerate variant thereof; the nucleotide sequences of SEQ ID NO: 138 and SEQ ID NO: 139, or degenerate variants thereof; the nucleotide sequence of SEQ ID NO: 168, or a degenerate variant thereof; the nucleotide sequence of SEQ ID NO: 169, or a degenerate variant thereof; the nucleotide sequences of SEQ ID NO: 168 and SEQ ID NO: 169, or degenerate variants thereof; the nucleotide sequence of SEQ ID NO: 178, or a degenerate variant thereof; the nucleotide sequence of SEQ ID NO: 179, or a degenerate variant thereof; the nucleotide sequences of SEQ ID NO: 178 and SEQ ID NO: 179, or degenerate variants thereof; the nucleotide sequence of SEQ ID NO: 184, or a degenerate variant thereof; the nucleotide sequence of SEQ ID NO: 185, or a degenerate variant thereof; or the nucleotide sequences of SEQ ID NO: 184 and SEQ ID NO: 185, or degenerate variants thereof.

Further provided are vectors that include one or more of the nucleic acid molecules disclosed herein. In some aspects, the vector is a viral vector, such as a lentiviral vector, an adenovirus vector or an adeno-associated virus vector.

Also provided are isolated host cells that include a disclosed nucleic acid molecule or vector. In some aspects, the isolated host cells are eukaryotic cells, such as mammalian cells. In other aspects, the isolated host cells are prokaryotic cells.

### V. Kits

Also provided are kits that include a CD25-specific monoclonal antibody, a conjugate, a composition, a nucleic acid molecule, a vector and/or isolated host cells disclosed herein. In some aspects, the kit further includes buffer, cell culture media, one or more check point inhibitors, one or more immunotherapies, one or more additional anti-cancer reagents (such as a chemotherapeutic agent or biologic), one or more reducing agents, one or more transfection reagents and/or instructional materials.

For example, kits for treating cancer in a subject are provided. The kits can include a disclosed CD25-specific monoclonal antibody, a CD25 antibody-IR700 conjugate, or a composition that includes the CD25-specific monoclonal antibody or CD25 antibody-IR700 conjugate. More than one of the disclosed CD25-specific antibodies, CD25 antibody-IR700 conjugates, or compositions including such molecules can be included in the kit.

In some examples, the kit further includes one or more buffers, such as PBS.

In some examples, the kit further includes one or more cell culture media, such as those used to propagate prokaryotic or eukaryotic cells in vitro. Such culture media can be solid, liquid, or powder. In one example, the cell culture media is DMEM or RPMI. In one example, the cell culture media is a bacterial culture media. In one example, the cell culture media is one optimized for transfection.

In some examples, the kit further includes one or more transfection reagents, such as liposomal, high-lipid based or non-liposomal based reagents (such as calcium phosphate-based).

In some examples, the kit further includes one or more check point inhibitors, such as those that reduce or inhibit the function of PD-1, PD-L1, and/or CTLA-4. In one example, the kit further includes a PD-L1 antibody, such as atezolizumab, avelumab, durvalumab, or cosibelimab (in some examples such mAbs are conjugated to IR700, such as atezolizumab-IR700, avelumab-IR700, durvalumab-IR700, or cosibelimab-IR700). In one example, the kit further includes a PD-1 antibody, such as nivolumab, pembrolizumab, cemiplimab, or dostarlimab (in some examples such mAbs are conjugated to IR700). In one example, the kit further includes a CTLA-4 antibody, such as ipilimumab or tremelimumab (in some examples such mAbs are conjugated to IR700).

In some examples, the kit further includes one or more one or more immunotherapies, such as one or more immunomodulators and/or immunoactivators listed below, such as IL-2, IL-15, IL-7, IL-12, and/or IL-21.

In some examples, the kit further includes one or more one or more chemotherapeutic agents, such as one or more listed below, such as one or more microtubule binding agents, DNA intercalators or cross-linkers, DNA synthesis inhibitors, DNA and/or RNA transcription inhibitors, antibodies, enzymes, enzyme inhibitors, and gene regulators. In one example, the kit further includes one or more of carboplatin, cisplatin, paclitaxel, docetaxel, doxorubicin, epirubicin, topotecan, irinotecan, gemcitabine, iazofurine, gemcitabine, etoposide, vinorelbine, tamoxifen, valspodar, cyclophosphamide, methotrexate, fluorouracil, mitoxantrone, Doxil (liposome encapsulated doxiorubicine) and vinorelbine.

In some examples, the kit further includes one or more one or more biologics, such as one or more of 3F8, Abagovomab, Adecatumumab, Afutuzumab, Alacizumab, Alemtuzumab, Altumomab pentetate, Anatumomab mafenatox, Apolizumab, Arcitumomab, Bavituximab, Bectumomab, Belimumab, Besilesomab, Bevacizumab, Bivatuzumab mertansine, Blinatumomab, Brentuximab vedotin, Cantuzumab mertansine, Capromab pendetide, Catumaxomab, CC49, Cetuximab, Citatuzumab bogatox, Cixutumumab, Clivatuzumab tetraxetan, Conatumumab, Dacetuzumab, Detumomab, Ecromeximab, Eculizumab, Edrecolomab, Epratuzumab, Ertumaxomab, Etaracizumab, Farletuzumab, Figitumumab, Galiximab, Gemtuzumab ozogamicin, Girentuximab, Glembatumumab vedotin, Ibritumomab tiuxetan, Igovomab, Imciromab, Intetumumab, Inotuzumab ozogamicin, Ipilimumab, Iratumumab, Labetuzumab, Lexatumumab, Lintuzumab, Lorvotuzumab mertansine, Lucatumumab, Lumiliximab, Mapatumumab, Matuzumab, Mepolizumab, Metelimumab, Milatuzumab, Mitumomab, Morolimumab, Nacolomab tafenatox, Naptuinomab estafenatox, Necitumumab, Nimotuzumab, Nofetuinomab merpentan, Ofatumumab, Olaratumab, Oportuzumab monatox, Oregovomab, Panitumumab, Pentumonab, Pertuzumab, Pintumomab, Pritumumab, Ramucirumab, Rilotumumab, Rituximab, Robatumumab, Satumomab pendetide, Sibrotuzumab, Sonepcizumab, Tacatuzumab tetraxetan, Taplitumomab paptox, Tenatumomab, TGN1412, Ticilimumab (tremelimumab), Tigatuzumab, TNX-650, Trastuzumab, Tremelimumab, Tucotuzumab celmoleukin, Veltuzumab, Volociximab, Votumumab, or Zalutumumab.

In one example, the kit further includes a tumor-specific antibody-IR700 conjugate, such as cetuximab-IR700, trastuzumab-IR700, tivozanib-IR700, or any other antibody-IR700 conjugate provided in Table 1.

In some examples, the kit further includes one or more reducing agents, such as one or more of L-cysteine, L-sodium ascorbate (L-NaAA), ascorbic acid (such as L- or R-ascorbic acid) and glutathione. In a specific example, the kit further includes L-NaAA.

The kit can include a container and a label or package insert on or associated with the container. Suitable containers include, for example, bottles, vials, syringes, etc. The containers may be formed from a variety of materials such as glass or plastic. The container typically holds a composition including one or more of the disclosed CD25-specific monoclonal antibodies, CD25 antibody-IR700 conjugates, or compositions. In several aspects, the container may have a sterile access port (for example the container may be an intravenous solution bag or a vial having a stopper pierceable by a hypodermic injection needle).

A label or package insert indicates that the composition is used for treating the particular condition. The label or package insert typically will further include instructions for use of the antibodies, conjugates, or compositions included in the kit. The package insert typically includes instructions customarily included in commercial packages of therapeutic products that contain information about the indications, usage, dosage, administration, contraindications and/or warnings concerning the use of such therapeutic products. The instructional materials may be written, in an electronic form or may be visual. The kits may also include additional components to facilitate the particular application for which the kit is designed.

### VI. Methods of Treating Cancer

Also provided herein are methods for treating cancer in a subject. The cancer can be a solid tumor or a hematological malignancy, such as one that is immunogenic (e.g., rich in T cells). The methods include administering to the subject a therapeutically effective amount of a conjugate that includes a CD25-specific monoclonal antibody and a photon absorber, or a composition thereof, as disclosed herein, and subsequently irradiating the subject and/or irradiating cancer cells in the subject at a wavelength of 660 to 740 nm and at a dose of at least 1 J/cm² (such as at least 4 J/cm²). In some aspects, the cancer is a cancer of the breast, liver, lung, esophagus, stomach, colon, ovary, prostate, pancreas, brain, cervix, kidney, bone, skin, head and neck, oropharynx or blood. In some aspects, the conjugate is administered locally at or near the site of a tumor. In other aspects, the conjugate is administered systemically, and the effect of the therapy is local to the cancer (e.g., no significant adverse systemic effects).

In some aspects, the method further includes administering a therapeutically effective amount of a second monoclonal antibody conjugated to IR700, wherein the second monoclonal antibody specifically binds a tumor antigen expressed by cells of the cancer. In some examples, the tumor antigen is selected from epidermal growth factor receptor (EGFR/HER1), mesothelin, prostate specific membrane antigen (PSMA), HER2/ERBB2, CD3, CD18, CD20, CD25 (IL-2Rα receptor), CD30, CD33, CD44, CD52, CD133, CD206, carcinoembryonic antigen (CEA), alpha-fetoprotein (AFP), Lewis Y, tumor-associated glycoprotein 72 (TAG72), vascular endothelial growth factor (VEGF), VEGF receptor (VEGFR), epithelial cell adhesion molecule (EpCAM), ephrin type-A receptor 2 (EphA2), glypican-1 (GPC1), glypican-2 (GPC2), glypican-3 (GPC3), gpA33, a mucin, CAIX, a folate-binding protein, a ganglioside, integrin aVp3, integrin α5β3,1, Erb-B2 receptor tyrosine kinase 3 (ERBB3), MET proto-oncogene, receptor tyrosine kinase (MET), insulin like growth factor 1 receptor (IGF1R), ephrin type-A receptor 3 (EPHA3), tumor necrosis factor-related apoptosis-inducing ligand receptor 1 (TRAILR1), TRAILR2, receptor activator of nuclear factor kappa-B ligand (RANKL), fibroblast activation protein (FAP), tenascin, BCR complex, gp72, HLA-DR 10j3, HLA-DR antigen, IgE, CA 242, polymorphic epithelial mucin (PEM) antigen, SK-1 antigen. In some aspects, the method further includes administering a therapeutically effective amount of a second monoclonal antibody conjugated to IR700, wherein the second monoclonal antibody specifically binds a checkpoint inhibitor, such as programmed death 1 (PD-1), CTLA4, and programmed death ligand (PD-L1). Specific exemplary tumor antigen and checkpoint inhibitor antibodies that can be used are provided herein. In some examples, both the CD25-specific conjugate and the second monoclonal antibody conjugated to IR700 are administered prior to irradiation. In some examples, the CD25-specific conjugate and the second monoclonal antibody conjugated to IR700 are administered intravenously.

In some aspects, the methods provided herein further include administering to the subject one or more additional cancer therapies. In some examples, the one or more additional cancer therapies includes an immunoactivator, chemotherapy, radiotherapy and/or surgery.

Immunoactivators include immune system activators and/or one or more inhibitors of immuno-suppressor cells (for example in the presence of a pharmaceutically acceptable carrier, such as a pharmaceutically and physiologically acceptable fluid). In a specific example, the immunoactivator is IL-15. In another specific example, the immunoactivator is interferon gamma. In some examples, the one or more immunoactivators are administered to the subject concurrently (for example, simultaneously or substantially simultaneously) with the one or more antibody-IR700 conjugates, for example in the same composition. In other examples, the one or more CD25-specific conjugates and the one or more immunoactivators are administered to the subject sequentially (in either order), for example, separated by at least about 1 hour of one another (for example, within about 5 minutes, about 10 minutes, about 15 minutes, about 20 minutes, about 30 minutes, about 40 minutes, about 50 minutes, about 60 minutes, about 2 hours, about 12 hours, about 24 hours, about 48 hours, about 3 days, about 4 days, about 5 days, about 6 days, or about 7 days).

In some examples, the subject is further administered a therapeutically effective amount of one or more reducing agents, for example to reduce edema that can result from NIR-PIT. In some examples, antibody-IR700 conjugate(s) and the reducing agent(s) are present in a single composition and administered simultaneously. In other examples, the antibody-IR700 conjugate and reducing agent(s) are present in separate compositions, and administered simultaneously or contemporaneously, or sequentially (e.g., the antibody-IR700 conjugate(s) followed by the reducing agent(s), for example with at least 1 minute, at least 5 minutes, at least 30 minutes, at least 60 minutes, at least 12 hours, or at least 24 hours in between).

After administering the CD25 antibody-IR700 conjugate, and optionally one or more tumor antigen-targeted antibody-IR700 conjugates and/or one or more checkpoint inhibitor antibody-IR700 conjugates, the conjugate(s) are allowed to accumulate in the targeted tumor or immune cells of the tumor. The tumor cells and other cells in the tumor bed (or the subject having the cancer) are then irradiated under conditions that permit killing of the cells to which the antibody-IR700 conjugate(s) are bound, for example irradiation at a wavelength of 660 to 740 nm (such as 600 to 710 nm) at a dose of at least 1 J/cm² (for example 680 nm or 690 nm at a dose of 4 to 60 J/cm², such as a dose of 4 to 25 J/cm², 4 to 10 J/cm², 20 to 50 J/cm² or 20 to 30 J/cm²).

In one example, there is at least about 10 minutes, at least about 30 minutes, at least about 1 hour, at least about 4 hours, at least about 8 hours, at least about 12 hours, at least about 24 hours, or at least about 48 hours (such as about 1 to 4 hours, 30 minutes to 1 hour, 10 minutes to 60 minutes, 30 minutes to 8 hours, 2 to 10 hours, 12 to 24 hours, 18 to 36 hours, or 24 to 48 hours) in between administering the antibody-IR700 conjugate(s) and the irradiation. In one example, the one or more antibody-IR700 conjugate(s) are administered (e.g., i.v., i.p, or intratumorally) and at least about 10 minutes, at least about 30 minutes, at least about 1 hour, at least about 4 hours, at least about 8 hours, at least about 12 hours, at least about 24 hours, or at least about 48 hours (such as about 1 to 4 hours, 30 minutes to 1 hour, 10 minutes to 60 minutes, 30 minutes to 8 hours, 2 to 10 hours, 12 to 24 hours, 18 to 36 hours, or 24 to 48 hours, such as about 12 hours or about 24 hours) later, the tumor (or the subject) is irradiated. The one or more immunoactivators can be administered (for example systemically, such as i.v. or i.p.) before or after the one or more antibody-IR700 conjugate(s), and before or after the irradiation. In some examples, the one or more immunoactivators are administered before and after irradiation, for example, at least one dose of immunoactivators prior to irradiation and at least one dose of immunoactivators after irradiation (such as 24 hours before and one or more of 24, 48, 72, 96, or more hours after irradiation). In additional examples, a dose of immunoactivators may also be administered on the same day as at least one irradiation treatment. The one or more reducing agents can be administered (for example systemically, such as i.v. or i.p.) before or after the one or more antibody-IR700 conjugate(s) but before the irradiation (such as at least 5 minutes before, at least 10 minutes before, at least 15 minutes before, or at least 30 minutes before, for example 5 to 60 minutes prior to irradiation).

In some examples, multiple doses of the one or more of the antibody-IR700 conjugate(s) are administered to the subject, such as at least 2, at least 3, at least 4, at least 5, at least 6, at least 7, at least 8, at least 9, or at least 10 separate doses (or administrations). In some examples, multiple doses of the one or more CTLA4 antibody-IR700 conjugates, the one or more PD-L1 antibody-IR700 conjugates, the one or more PD-1 antibody-IR700 conjugates, or combinations thereof, are administered to the subject, such as at least 2, at least 3, at least 4, at least 5, at least 6, at least 7, at least 8, at least 9, or at least 10 separate doses (or administrations). In some examples, multiple doses of the one or more immunoactivators are administered to the subject, such as at least 2, at least 3, at least 4, at least 5, at least 6, at least 7, at least 8, at least 9, or at least 10 separate doses (or administrations). In some examples, multiple doses of the one or more reducing agents are administered to the subject, such as at least 2, at least 3, at least 4, at least 5, at least 6, at least 7, at least 8, at least 9, or at least 10 separate doses (or administrations). In some examples, multiple doses of the irradiation with NIR are administered to the subject, such as at least 2, at least 3, at least 4, at least 5, at least 6, at least 7, at least 8, at least 9, or at least 10 separate doses (or administrations). In a specific example, the subject is administered at least one dose of the one or more of the antibody-IR700 conjugate(s), at least two doses of one or more immunoactivators and at least two separate NIR irradiation administrations.

The NIR excitation light wavelength allows penetration of at least several centimeters into tissues. For example, by using fiber-coupled laser diodes with diffuser tips, NIR light can be delivered within several centimeters of otherwise inaccessible tumors located deep with respect to the body surface. In addition to treating solid cancers, circulating tumor cells (including, but not limited to hematological malignancies) can be targeted since they can be excited when they traverse superficial vessels (for example using the NIR LED wearable devices described herein).

In some examples, the disclosed methods kill at least 10%, for example at least 20%, at least 40%, at least 50%, at least 80%, at least 90%, at least 95%, at least 98%, or more of the treated target cells (such as cancer cells expressing a tumor-specific antigen, or CD25+ immune cells in the tumor bed, such as Tregs in the tumor bed), for example relative to the absence of treatment with the disclosed methods.

In some examples, the disclosed methods decrease the weight, volume or size of a metastasis, such as by at least 10%, at least 20%, at least 40%, at least 50%, at least 80%, at least 90%, at least 95%, at least 98%, or even 100%, for example relative to the absence of treatment with the disclosed methods. In some examples the disclosed methods have abscopal effects, and decrease the weight, volume or size of a metastasis that itself has not been irradiated at a wavelength of 660 to 740 nm at a dose of at least 1 J cm⁻² (such as at a dose of at least 4 J cm⁻², at least 10 J cm⁻², such as at a dose of 4 to 50 J cm⁻², 1 to 10 J cm⁻², or 1 to 5 J cm⁻²) and is located distant from the irradiated area of the tumor or lesion. In some examples the weight, volume or size of a non-irradiated metastasis decreases by at least 10%, at least 20%, at least 40%, at least 50%, at least 80%, at least 90%, at least 95%, at least 98%, or even 100% by treatment with the disclosed methods.

By selective killing of Treg cells (such as CD4⁺Foxp3⁺ Tregs) relative to other immune cells, the methods are capable of killing Treg cells that are irradiated more effectively than other immune cells such as, for example, Tregs not in the tumor bed. In one example, the disclosed methods decrease Tregs (such as FOXP3+CD4+ Treg cells in the tumor bed, but not in other parts of the body, such as the spleen or regional lymph nodes). For example, the disclosed methods can decrease the number of Treg cells in the tumor bed by at least 10%, for example by at least 20%, at least 40%, at least 50%, at least 80%, at least 90%, at least 95%, at least 98%, or more, relative to the absence of treatment with the disclosed methods (e.g., without administration of one or more antibody-IR700 conjugates and/or without NIR irradiation). In some examples, the disclosed methods do not substantially decrease the number of Treg cells in the spleen or regional lymph nodes of the tumor, for example decrease no more than 10%, no more than 5%, no more than 1%, or no more than 0.5%, relative to the absence of treatment with the disclosed methods (e.g., without administration of one or more antibody-IR700 conjugates and/or without NIR irradiation).

In some examples, the disclosed methods decrease intra-tumoral blood perfusion by at least 10%, for example by at least 20%, at least 40%, at least 50%, at least 80%, at least 90%, at least 95%, at least 98%, or more, relative to the absence of treatment the disclosed methods (e.g., without administration of one or more antibody-IR700 conjugates and/or without NIR irradiation).

In some examples, the disclosed methods decrease one or more symptoms associated with a tumor, a recurrence, and/or a metastatic tumor. In one example, the disclosed methods slow the growth of a tumor, such as by at least 10%, for example by at least 20%, at least 40%, at least 50%, at least 80%, at least 90%, or more, relative to the absence of treatment with the disclosed methods (e.g., without administration of one or more antibody-IR700 conjugates and/or without NIR irradiation). In one example, the disclosed methods reduce or eliminates tumor recurrence, such as by at least 10%, for example by at least 20%, at least 40%, at least 50%, at least 80%, at least 90%, at least 95%, at least 98%, at least 99% or even 100%, relative to the absence of treatment with the disclosed methods (e.g., without administration of one or more antibody-IR700 conjugates and/or without NIR irradiation).

In some examples, the disclosed methods increase a subject's (such as a subject with a tumor or who has had a tumor previously removed) survival time, for example relative to the absence of treatment with the disclosed methods (e.g., without administration of one or more antibody-IR700 conjugates and/or without NIR irradiation). In some examples, the survival time of a subject increases at least 20%, at least 25%, at least 40%, at least 50%, at least 80%, at least 90%, or more, for example relative to the absence of treatment with the disclosed methods (e.g., without administration of one or more antibody-IR700 conjugates and/or without NIR irradiation). In some examples, the survival time of a subject increases by at least 2 months, at least 3 months, at least 4 months, at least 5 months, at least 6 months, at least 9 months, at least 1 year, at least 1.5 years, at least 2 years, at least 3 years, at least 4 years, at least 5 years or more, for example relative to the absence of treatment with the disclosed methods (e.g., without administration of one or more antibody-IR700 conjugates and/or without NIR irradiation). For example, the disclosed methods in some examples increase a subject's progression-free survival time or disease-free survival time (for example, lack of recurrence of the primary tumor or lack of metastasis) by at least 1 months, at least 2 months, at least 3 months, at least 6 months, at least 12 months, at least 18 months, at least 24 months, at least 36 months, at least 48 months, at least 60 months, or more, relative to average survival time in the absence of treatment with the disclosed methods (e.g., without administration of one or more antibody-IR700 conjugates and/or without NIR irradiation).

In some examples, use of one or more reducing agents in combination with NIR-PIT reduces edema in the treated subject by at least 10%, at least 20%, at least 30%, at least 40%, at least 50%, at least 60%, at least 75%, at least 80%, at least 90%, or even at least 95%, as compared to the amount of edema without use of one or more reducing agents with NIR-PIT. In some examples, use of one or more reducing agents in combination with NIR-PIT reduces acute inflammatory reaction in the treated subject by at least 10%, at least 20%, at least 30%, at least 40%, at least 50%, at least 60%, at least 75%, at least 80%, at least 90%, or even at least 95%, as compared to the amount of acute inflammatory reaction without use of one or more reducing agents with NIR-PIT.

In one example, combinations of the effects listed above are achieved with the disclosed methods.

The disclosed methods can be used to treat fixed tumors in the body as well as hematological malignancies and/or tumors in the circulation (e.g., leukemia cells, metastases, and/or circulating tumor cells). However, circulating cells, by their nature, cannot be exposed to light for very long. Thus, if the cell to be killed is one that is circulating throughout the body, the methods can be accomplished by using a device that can be worn, or that covers parts of the body. For example, such a device can be worn for extended time periods. Everyday wearable items (e.g., wristwatches, jewelry (such as a necklace or bracelet), blankets, clothing (e.g., underwear, socks, and shoe inserts) and other everyday wearable items) which incorporate NIR emitting light emitting diodes (LEDs) and a battery pack, can be used. Such devices produce light on the skin underlying the device over long periods leading to continual exposure of light to superficial vessels over prolonged periods. Circulating tumor cells are exposed to the light as they transit thru the area underlying the device. As an example, a wristwatch or bracelet version of this device can include a series of NIR LEDs with battery power pack to be worn for most of the day. After administration of the one or more CD25 antibody-IR700 conjugates (e.g., in combination with one or more tumor-specific Ab-IR700 conjugates or check point inhibitor Ah-IR700 conjugates (e.g., CTLA4 antibody-IR700 conjugates, PD-L1 antibody-IR700 conjugates, PD-L1 antibody-IR700 conjugates) one or more reducing agents, one or more immunoactivators, or combinations thereof, systemically, such as i.v. and/or i.p.), circulating cells bind antibody-IR700 conjugates and become susceptible to killing by PIT. As these cells flow within the vessels adjacent to the LED present in the everyday wearable item (e.g., bracelet or wristwatch), they would be exposed to NIR light rendering them susceptible to cell killing. The dose of light may be adjustable according to diagnosis and cell type.

In some examples, the method further includes monitoring the therapy, such as killing of tumor cells, killing of Tregs, or both. In such examples, the subject is administered one or more antibody-IR700 conjugates (such as CD25-specific antibody-IR700 conjugates and optionally tumor-specific antibody-IR700 conjugates and/or check point inhibitor Ab-IR700 conjugates), for example in combination with one or more reducing agents, one or more immunoactivators, or combinations thereof, and irradiated as described herein. However, a lower dose of an antibody-IR700 conjugate and NIR light can be used for monitoring (as cell killing may not be required, just monitoring of the therapy). In one example, the amount of antibody-IR700 conjugate administered for monitoring is at least 2-fold less (such as at least 3-, 4-, 5-, 6-, 7-, 8-, 9-, or 10-fold less than the therapeutic dose). In one example, the amount of antibody-IR700 conjugate administered for monitoring is at least 20% or at least 25% less than the therapeutic dose. In one example, the amount of NIR light used for monitoring is at least 1/1000 or at least 1/10,000 of the therapeutic dose. This permits detection of the cells being treated. For example, by using such methods, the size of the tumor and metastases can be monitored.

In some examples, the method is useful during surgery, such as endoscopic procedures. For example, after the antibody-IR700 conjugates (such as CD25-specific antibody-IR700 conjugates and optionally tumor-specific antibody-IR700 conjugates and/or check point inhibitor Ab-IR700 conjugates) are administered, for example in combination with one or more reducing agents, one or more immunoactivators, or combinations thereof, and the cells/subject irradiated as described above, this not only results in cell killing, but permits a surgeon or other medical care provider to visualize the margins of a tumor, and help ensure that resection of the tumor (such as a tumor of the skin, breast, lung, colon, head and neck, or prostate) is complete and that the margins are clear. In some examples, a lower dose of the antibody-IR700 conjugate can be used for visualization, such as at least 2-fold less (such as at least 3-, 4-, 5-, 6-, 7-, 8-, 9-, or 10-fold less than the therapeutic dose).

The one or more antibody-IR700 conjugates (e.g., CD25-specific antibody-IR700 conjugates and optionally tumor-specific antibody-IR700 conjugates and/or check point inhibitor Ab-IR700 conjugates), one or more reducing agents, and the one or more immunoactivators, can be administered locally or systemically, for example to subjects having a tumor, such as a cancer, or who has had a tumor previously removed (for example via surgery). Although specific examples are provided, one skilled in the art will appreciate that alternative methods of administration of the disclosed therapeutic agents can be used. Such methods may include for example, the use of catheters or implantable pumps to provide continuous infusion over a period of several hours to several days into the subject in need of treatment.

In one example, the one or more antibody-IR700 conjugates (e.g., CD25-specific antibody-IR700 conjugates and optionally tumor-specific antibody-IR700 conjugates and/or check point inhibitor Ab-IR700 conjugates), one or more reducing agents, and the one or more immunoactivators are administered by parenteral means, including direct injection or infusion into a tumor (intratumorally) or organ (e.g., prostate). In some examples, the one or more antibody-IR700 conjugates (e.g., CD25-specific antibody-IR700 conjugates and optionally tumor-specific antibody-IR700 conjugates and/or check point inhibitor Ab-IR700 conjugates), one or more reducing agents, and the one or more immunoactivators are administered to the tumor by applying the agents to the tumor, for example by local injection of therapeutic agents, bathing the tumor in a solution containing the therapeutic agents, or by pouring the therapeutic agents onto the tumor.

In addition, or alternatively, the disclosed compositions can be administered systemically, for example intravenously, intramuscularly, subcutaneously, intradermally, intraperitoneally, subcutaneously, or orally, to a subject having a tumor (such as cancer). The one or more antibody-IR700 conjugates (e.g., CD25-specific antibody-IR700 conjugates and optionally tumor-specific antibody-IR700 conjugates and/or check point inhibitor Ab-IR700 conjugates), one or more reducing agents, and the one or more immunoactivators may be administered by the same or different routes. In one example, the one or more antibody-IR700 conjugates are administered intravenously and the one or more reducing agents delivered intraperitoneally. In one example, the one or more antibody-IR700 conjugates and the one or more immunoactivators are administered intravenously and/or intratumorally, and the one or more reducing agents delivered intraperitoneally. In another example, the one or more antibody-IR700 conjugates (e.g., CD25-specific antibody-IR700 conjugates and optionally tumor-specific antibody-IR700 conjugates and/or check point inhibitor Ab-IR700 conjugates), one or more reducing agents, and the one or more immunoactivators are administered systemically (for example, intravenously or intraperitoneally). In one example, the one or more CD25-specific antibody-IR700 conjugates and optionally tumor-specific antibody-IR700 conjugates and/or check point inhibitor Ab-IR700 conjugates, one or more reducing agents, and the one or more immunoactivators are administered intraperitoneally. In one example, the one or more CD25-specific antibody-IR700 conjugates, one or more tumor-specific antibody-IR700 conjugates, the one or more immunoactivators, and the one or more reducing agents are administered intravenously.

The dosages of the therapeutic agents provided herein to be administered to a subject are not subject to absolute limits, but may depend on the nature of the composition, its active ingredients and its potential unwanted side effects (e.g., immune response against the antibody), the subject being treated and the type of condition being treated, and the manner of administration. Generally, the dose will be a therapeutically effective amount, such as an amount sufficient to achieve a desired biological effect, for example an amount that is effective to decrease the size (e.g., volume and/or weight) of the tumor, or attenuate further growth of the tumor, or decrease undesired symptoms of the tumor.

For intravenous administration of antibody-IR700 conjugates, exemplary dosages for administration to a subject for a single treatment can range from 0.5 to 200 mg/60 kg of body weight, 1 to 100 mg/60 kg of body weight, 1 to 50 mg/60 kg of body weight, 1 to 20 mg/60 kg of body weight, for example about 1 or 2 mg/kg of body weight. In yet another example, a therapeutically effective amount of intraperitoneally or intratumorally administered antibody-IR700 conjugates is 10 μg to 5000 μg of antibody-IR700 conjugate per 1 kg of body weight, such as 10 μg/kg to 1000 μg/kg, 10 μg/kg to 500 μg/kg, or 100 μg/kg to 1000 μg/kg. In one example, the dose of antibody-IR700 conjugates administered to a human patient is at least 50 mg, such as at least 100 mg, at least 300 mg, at least 500 mg, at least 750 mg, or even 1 g.

For intravenous administration of reducing agents, exemplary dosages for administration to a subject for a single treatment can range from 0.5 to 300 g/60 kg of body weight, 1 to 300 g/60 kg of body weight, 1 to 50 g/60 kg of body weight, 1 to 20 g/60 kg of body weight, 1 to 10 g/60 kg of body weight, 10 to 300 g/60 kg of body weight, such as 1, 2, 5, 10, 20, 50, 100, 200 or 300 g/60 kg of body weight. In yet another example, a therapeutically effective amount of intraperitoneally or intratumorally administered reducing agents is 10 mg to 5000 mg of reducing agents per 1 kg of body weight, such as 10 mg/kg to 1000 mg/kg, 10 mg/kg to 500 mg/kg, or 100 mg/kg to 1000 mg/kg. In one example, the dose of reducing agents administered to a human patient is at least 1 g, at least 10 g, at least 20 g, at least 50 g, at least 100 g, at least 200 g, at least 300 g, such as 1, 2, 5, 10, 20, 50, 100, 200 or 300 g.

Treatments with the disclosed antibody-IR700 conjugates (including CD25-specific antibody-IR700 conjugates, check point inhibitor-specific antibody-IR700 conjugates, and/or tumor-specific antibody-IR700 conjugates), one or more reducing agents, and/or one or more immunoactivators can be completed in a single day or may be done repeatedly on multiple days with the same or a different dosage. Repeated treatments may be done on the same day, on successive days, or every 1-3 days, every 3-7 days, every 1-2 weeks, every 2-4 weeks, every 1-2 months, or at even longer intervals. In some examples, the antibody-IR700 conjugates, one or more reducing agents, and/or one or more immunoactivators are administered on the same day. In other examples, the antibody-IR700 conjugates, one or more reducing agents, and/or one or more immunoactivators are administered on different days, such as the one or more immunoactivators administered the day before the antibody-IR700 conjugates, or the antibody-IR700 conjugates administered the day before the one or more reducing agents). In one non-limiting example, the one or more antibody-IR700 conjugates and one or more immunoactivators are administered to the subject on the same day and repeated doses of the one or more immunoactivators (at the same or different dosing level) are administered to the subject (for example, 1, 2, 3, 4, 5, or more additional doses of the one or more immunoactivators) on successive days, or every 1-3 days, every 3-7 days, every 1-2 weeks, every 2-4 weeks, every 1-2 months, or at even longer intervals. In some examples, the amount of the repeated doses of the one or more immunoactivators is reduced compared to the initial dose (for example, reduced by 50%).

In additional aspects, the methods also include administering to the subject one or more additional therapeutic agents. As described in International Patent Application Publication No. WO 2013/009475 (incorporated by reference herein in its entirety), there is about an 8 hour window following irradiation (for example irradiation at a wavelength of 660 to 710 nm at a dose of at least 10 J/cm², at least 20 J/cm², at least 30 J/cm², at least 40 J/cm², at least 50 J/cm², at least 70 J/cm², at least 80 J/cm² or at least 100 J/cm², such as at least 10 to 100 J/cm²), during which uptake of additional agents (e.g., nano-sized agents, such as those about at least 1 nm in diameter, at least 10 nm in diameter, at least 100 nm in diameter, or at least 200 nm in diameter, such as 1 to 500 nm in diameter) by the PIT-treated cells is enhanced. Thus, one or more additional therapeutic agents can further be administered to the subject contemporaneously or sequentially with the PIT. In one example, the additional therapeutic agents are administered after the irradiation, for example, about 0 to 8 hours after irradiating the cell (such as at least 10 minutes, at least 30 minutes, at least 60 minutes, at least 2 hours, at least 3 hours, at least 4, hours, at least 5 hours, at least 6 hours, or at least 7 hours after the irradiation, for example no more than 10 hours, no more than 9 hours, or no more than 8 hours, such as 1 hour to 10 hours, 1 hour to 9 hours 1 hour to 8 hours, 2 hours to 8 hours, or 4 hours to 8 hours after irradiation). In another example, the additional therapeutic agents are administered just before the irradiation (such as about 10 minutes to 120 minutes before irradiation, such as 10 minutes to 60 minutes or 10 minutes to 30 minutes before irradiation). Additional therapeutic agents that can be used are discussed below.

In additional aspects, methods are provided that permit detection or monitoring of cell killing in real-time. Such methods are useful for example, to ensure sufficient amounts of antibody-IR700 conjugates (including CD25-specific antibody-IR700 conjugates, tumor-specific antibody-IR700 conjugates and/or CTLA4−, PD-1, and PD-L1-antibody-IR700 conjugates), one or more reducing agents, and/or one or more immunoactivators, or sufficient amounts of irradiation, were delivered to the cell or tumor to promote cell killing. These methods permit detection of cell killing before morphological changes become evident. In one example, the methods include contacting a cell having a cell surface protein with a therapeutically effective amount of one or more antibody-IR700 conjugates (including CD25-specific antibody-IR700 conjugates, tumor-specific antibody-IR700 conjugates and/or check point inhibitor-antibody-IR700 conjugates), one or more reducing agents, and/or one or more immunoactivators; irradiating the cell at a wavelength of 660 to 740 nm and at a dose of at least 4 J/cm² or at least 10 J/cm²; and detecting the cell with fluorescence lifetime (FLT) imaging about 0 to 48 hours after irradiating the cell (such as at least 1 hour, at least 2 hours, at least 4 hours, at least 6 hours, at least 12 hours, at least 18 hours, at least 24 hours, at least 36 hours, at least 48 hours, or at least 72 hours after irradiating the cell, for example 1 minute to 30 minutes, 10 minutes to 30 minutes, 10 minutes to 1 hour, 1 hour to 8 hours, 6 hours to 24 hours, or 6 hours to 48 hours after irradiating the cell), thereby detecting the cell killing in real-time. Shortening FLT serves as an indicator of acute membrane damage induced by PIT. Thus, the cell is irradiated under conditions sufficient to shorten IR700 FLT by at least 25%, such as at least 40%, at least 50%, at least 60% or at least 75%. In one example, the cell is irradiated at a wavelength of 660 nm to 740 nm (such as 680 nm to 700 nm, such as 680 or 690 nm) and at a dose of at least 1 J/cm², at least 4 J/cm², at least 10 J/cm², at least 20 J/cm², at least 30 J/cm², at least 40 J/cm², at least 50 J/cm² or at least 60 J/cm², such as 10 to 60 J/cm², 20 to 50 J/cm², 20 to 25 J/cm², 30 to 50 J/cm², 4 to 50 J/cm² or 1 to 10 J/cm².

In some examples, methods of detecting cell killing in real time includes contacting the cell with one or more additional therapeutic agents, for example about 0 to 8 hours after irradiating the cell. The real-time imaging can occur before or after contacting the cell with one or more additional therapeutic agents. For example, if insufficient cell killing occurs after administration of the one or more antibody-IR700 conjugates (including CD25-specific antibody-IR700 conjugates, tumor-specific antibody-IR700 conjugates and/or check point inhibitor-antibody-IR700 conjugates), one or more reducing agents, and/or one or more immunoactivators, as determined by the real-timing imaging, then the cell can be contacted with one or more additional therapeutic agents. However, in some examples, the cell is contacted with the antibody-IR700 conjugates (including CD25-specific antibody-IR700 conjugates, tumor-specific antibody-IR700 conjugates and/or check point inhibitor-antibody-IR700 conjugates), one or more reducing agents, and/or one or more immunoactivators, and additional therapeutic agents, prior to detecting the cell killing in real-time.

### Exemplary Cells

The target cell can be a cell that is not desired or whose growth is not desired, such as a cancer cell (e.g., a tumor cell) or an immune cell (e.g., T cell, such as a Treg). The cells can be present in a mammal to be treated, such as a subject (for example, a human or veterinary subject) with cancer. Any target cell can be treated with the claimed methods. In some aspects, the target cell is a CD25+ Treg cell. In some examples, the target cell expresses a cell surface protein that is not substantially found on the surface of other normal (desired) cells, an antibody can be selected that specifically binds to such protein, and an antibody-IR700 conjugate generated for that protein. In one example, the cell surface protein is a tumor-specific protein (e.g., antigen), such as EGFR. In one non-limiting example, the cell surface protein is CTLA4 (for example to target CD4⁺Foxp3⁺ Tregs in the tumor bed). In one non-limiting example, the cell surface protein is PD-L1, PD-1, or CTLA4.

In some examples in which the subject is administered a second antibody-IR700 conjugate (such as a tumor antigen specific-antibody IR700 conjugate), the tumor cell is a cancer cell, such as a cell in a patient with cancer. Exemplary cells that can be killed with the disclosed methods include cells of the following tumors: a hematological malignancy such as a leukemia, including acute leukemia (such as acute lymphocytic leukemia, acute myelocytic leukemia, and myeloblastic, promyelocytic, myelomonocytic, monocytic and erythroleukemia), chronic leukemias (such as chronic myelocytic (granulocytic) leukemia and chronic lymphocytic leukemia), polycythemia vera, lymphoma, Hodgkin's disease, non-Hodgkin's lymphoma, multiple myeloma, Waldenstrdm's macroglobulinemia, heavy chain disease). In another example the cell is a solid tumor cell, such as cells from sarcomas and carcinomas, fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, pancreatic cancer, breast cancer, ovarian cancer, prostate cancer, hepatocellular carcinoma, lung cancer, colorectal cancer, squamous cell carcinoma, a head and neck cancer (such as head and neck squamous cell carcinoma), basal cell carcinoma, adenocarcinoma (for example adenocarcinoma of the pancreas, colon, ovary, lung, breast, stomach, prostate, cervix, or esophagus), sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS cancers (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma). In one example the tumor is an oropharyngeal cancer.

In a specific example, the cell is a lung cancer cell.

In a specific example, the cell is a breast cancer cell.

In a specific example, the cell is a colon cancer cell.

In a specific example, the cell is a stomach cancer cell.

In a specific example, the cell is a kidney cancer cell.

In a specific example, the cell is a head and neck cancer cell.

In a specific example, the cell is a prostate cancer cell.

In a specific example, the cell is an oropharyngeal cancer cell.

In a specific example, the cell is an esophageal cancer cell,

In a specific example, the cell is a squamous cell carcinoma cancer cell, such as one of a head and neck or skin cancer).

In some examples, the cancer or cancer cell treated is one at a site distant from the tumor irradiated, such as a distant metastasis that does not receive irradiation at a wavelength of 660 to 740 nm and at a dose of at least 1 J/cm², such as one 1 mm away, 1 inch away, 2 inches away, 3 inches away, 4 inches away, 6 inches away, 12 inches away, or more, as the methods can have abscopal effects.

In some examples, the cancer or cancer cell treated is moderately or highly immunogenic, that is able to provoke an immune response. In some examples, a NIR-PIT method that includes administration of CTLA4 antibody-IR700, PD-1 antibody-IR700 and/or PD-L1 antibody-IR700 is used to treat a moderately or highly immunogenic tumor. Examples of moderately or highly immunogenic cancers include melanoma, lung cancer (e.g., NSCLC), colon cancer, and renal cell cancer. In one example, a highly immunogenic cancer is a prostate cancer. In some examples, the cancer or cancer cell treated is not highly immunogenic, that is unable to provoke an immune response. In some examples, NIR-PIT with CTLA4 antibody-IR700, PD-1 antibody-IR700, and/or PD-L1 antibody-IR700 is used in combination with anti-tumor-IL700 (such as anti-EGFR-IR700 or anti-PSA antigen-IR700) to treat a low immunogenic tumor. Examples of low immunogenic cancers include oral squamous cell cancer, breast cancer, pancreatic cancer, and multiple myeloma. In some examples, a tumor immunogenicity score is used to determine whether a cancer is low, moderately, or highly immunogenic (e.g., see Wang et al., eLift, 8:e49020, 2019).

### Exemplary Subjects

In some examples, the disclosed methods are used to treat a subject who has cancer or a subject with a tumor, such as a tumor described herein. In some examples, the tumor has been previously treated, such as surgically or chemically removed, and the disclosed methods are used subsequently to kill any remaining undesired tumor cells that may remain in the patient and/or reduce recurrence or metastasis of the tumor. Tn some examples, a subject treated with a disclosed CD25 antibody-IR700 conjugate has a highly or moderately immunogenic cancer.

The disclosed methods can be used to treat any mammalian subject (such as a human or veterinary subject, such as a dog or cat), such as a human, who has a tumor, such as a cancer, or has had such previously removed or treated. Subjects in need of the disclosed therapies can include human subjects having cancer, such as a cancer that express a tumor-specific protein on the cell surface that can specifically bind to a tumor-specific antibody-IR700 conjugate, or a cancer infiltrated with immune cells that can bind to an immune cell specific antibody-IR700 molecule (e.g., CTLA4 antibody-IR700). For example, the disclosed methods can be used as initial treatment for cancer either alone, or in combination with radiation or other chemotherapy or surgery. The disclosed methods can also be used in patients who have failed previous radiation or chemotherapy. Thus, in some examples, the subject is one who has received other therapies, but those other therapies have not provided a desired therapeutic response. The disclosed methods can also be used in patients with localized and/or metastatic cancer and/or a recurrence of a primary tumor.

In some examples, the method includes selecting a subject who will benefit from the disclosed therapies, such as selecting a subject having a tumor that expresses a cell surface protein (such as a tumor-specific protein) that can specifically bind to an antibody-IR700 conjugate. For example, if the subject is determined to have a breast cancer that expresses HER2, the subject can be selected to be treated with an anti-HER2-IR700 conjugate, such as Trastuzumab-IR700, in combination with a CD25-specific antibody-IR700 conjugate, for example in combination with CTLA4 antibody-IR700 conjugates, PD-1 antibody-IR700 conjugates, PD-L1 antibody-IR700 conjugates, one or more reducing agents, one or more immunoactivators, or combinations thereof, and the subject is subsequently irradiated as described herein.

### Exemplary Cell Surface Proteins

In some aspects of the disclosed methods, a CD25-specific antibody-IR700 conjugate is administered in combination with an antibody-IR700 conjugate targeted to a protein on the cell surface of a tumor or cancer cell. In one example, the protein on the cell surface of the target cell to be killed is not present in significant amounts on other cells. For example, the cell surface protein can be a receptor that is only found on the target cell type.

In a specific example, the cell surface protein is a cancer- or tumor-specific protein (also referred to as a tumor-specific antigen or tumor-associated antigen), such as members of the EGF receptor family (e.g., HER1, 2, 3, and 4) and cytokine receptors (e.g., CD20, CD25, IL-13R, CD5, CD52, etc.). Thus, in some examples, the cell surface protein is an antigen expressed on the cell membrane of tumor cells. Tumor-specific proteins are proteins that are unique to cancer cells or are much more abundant on them, as compared to other cells, such as normal cells. For example, HER2 is primarily found in breast cancers, while HER1 is primarily found in adenocarcinomas, which can be found in many organs, such as the pancreas, breast, prostate and colon.

Exemplary tumor-specific proteins that can be found on a target cell (and to which an antibody specific for that protein can be used to formulate an antibody-IR700 molecule), include but are not limited to: any of the various MAGEs (Melanoma-Associated Antigen E), including MAGE 1 (e.g., GenBank Accession Nos. M77481 and AAA03229), MAGE 2 (e.g., GenBank Accession Nos. L18920 and AAA17729), MAGE 3 (e.g., GenBank Accession Nos. U03735 and AAA17446), MAGE 4 (e.g., GenBank Accession Nos. D32075 and A06841.1), etc.; any of the various tyrosinases (e.g., GenBank Accession Nos. U01873 and AAB60319); mutant ras; mutant p53 (e.g., GenBank Accession Nos. X54156, CAA38095 and AA494311); p97 melanoma antigen (e.g., GenBank Accession Nos. M12154 and AAA59992); human milk fat globule (HMFG) associated with breast tumors (e.g., GenBank Accession Nos. S56151 and AAB19771); any of the various BAGEs (Human B melanoma-Associated Antigen E), including BAGE1 (e.g., GenBank Accession No. Q13072) and BAGE2 (e.g., GenBank Accession Nos. NM_182482 and NP_872288), any of the various GAGEs (G antigen), including GAGE1 (e.g., GenBank Accession No. Q13065) or any of GAGE2-6; various gangliosides, CD25 (e.g., GenBank Accession Nos. NP_000408.1 and NM_000417.2).

Other tumor-specific antigens include the HPV 16/18 and E6/E7 antigens associated with cervical cancers (e.g., GenBank Accession Nos. NC_001526, FJ952142.1, ADB94605, ADB94606, and U89349), mucin (MUC 1)-KLH antigen associated with breast carcinoma (e.g., GenBank Accession Nos. J03651 and AAA35756), CEA (carcinoembryonic antigen) associated with colorectal cancer (e.g., GenBank Accession Nos. X98311 and CAA66955), gp100 (e.g., GenBank Accession Nos. S73003 and AAC60634) associated with for example melanoma, MARTI antigens associated with melanoma (e.g., GenBank Accession No. NP_005502), cancer antigen 125 (CA125, also known as mucin 16 or MUC16) associated with ovarian and other cancers (e.g., GenBank Accession Nos. NM_024690 and NP_078966); alpha-fetoprotein (AFP) associated with liver cancer (e.g., GenBank Accession Nos. NM_001134 and NP_001125); Lewis Y antigen associated with colorectal, biliary, breast, small-cell lung, and other cancers; tumor-associated glycoprotein 72 (TAG72) associated with adenocarcinomas; glypican 1 (GPC1 associated with pancreatic cancer, glioma, and breast cancer), glypican 2 (associated with neuroblastoma) and glypican 3 (associated with hepatocellular carcinoma), and the PSA antigen associated with prostate cancer (e.g., GenBank Accession Nos. X14810 and CAA32915).

Other exemplary tumor-specific proteins include, but are not limited to, PMSA (prostate membrane specific antigen; e.g., GenBank Accession Nos. AAA60209 and AAB81971.1) associated with solid tumor neovasculature, as well prostate cancer; HER-2 (human epidermal growth factor receptor 2, e.g., GenBank Accession Nos. M16789.1, M16790.1, M16791.1, M16792.1 and AAA58637) associated with breast cancer, ovarian cancer, stomach cancer and uterine cancer, HER-1 (e.g., GenBank Accession Nos. NM_005228 and NP_005219) associated with lung cancer, anal cancer, and glioblastoma as well as adenocarcinomas; NY-ESO-1 (e.g. GenBank Accession Nos. U87459 and AAB49693) associated with melanoma, sarcomas, testicular carcinomas, and other cancers, hTERT (aka telomerase) (e.g., GenBank Accession. Nos. NM_198253 and NP_937983 (variant 1), NM_198255 and NP_937986 (variant 2)); proteinase 3 (e.g., GenBank Accession Nos. M29142, M75154, M96839, X55668, NM 00277, M96628, X56606, CAA39943 and AAA36342), and Wilms tumor 1 (WT-1, e.g. GenBank Accession Nos. NM_000378 and NP_000369 (variant A), NM_024424 and NP_077742 (variant B), NM_024425 and NP_077743 (variant C), and NM_024426 and NP_077744 (variant D)).

In one example, the tumor-specific protein is EGFR, and in some examples the EGFR antibody-IR700 is or includes panitumumab or cetuximab.

In one example, the tumor-specific protein is PD-L1, and in some examples the PD-L1 antibody-IR700 is or includes atezolizumab, avelumab, durvalumab, or cosibelimab.

In one example the tumor-specific protein is CD52 (e.g., GenBank Accession. Nos. AAH27495.1 and CAI15846.1) associated with chronic lymphocytic leukemia; CD33 (e.g., GenBank Accession. Nos. NM_023068 and CAD36509.1) associated with acute myelogenous leukemia; or CD20 (e.g., GenBank Accession. Nos. NP_068769 NP_031667) associated with non-Hodgkin lymphoma.

In a specific example, the tumor-specific protein is CD44 (e.g., OMIM 107269, GenBank Accession. Nos. ACI46596.1 and NP_000601.3). CD44 is a marker of cancer stem-like cells and various types of cancers and is implicated in intercellular adhesion, cell migration, cell spatial orientation, and promotion of matrix-derived survival signal. High expression of CD44 on the plasma membrane of tumors can be associated with tumor aggressiveness and poor outcome.

Thus, the disclosed methods can be used to treat any cancer that expresses a tumor-specific protein.

### Exemplary Antibody-IR700 Conjugates

Because cell surface protein sequences are publicly available (for example as described above), making or purchasing antibodies (or other small molecules that can be conjugated to IR700) specific for such proteins can be accomplished. For example, if the tumor-specific protein HER2 is selected as a target, antibodies specific for HER2 (such as Trastuzuab) can be purchased or generated and attached to the IR700 dye. Other specific examples are provided in Table 1 and elsewhere herein. In one example, the antibody conjugated to IR700 is a humanized monoclonal antibody.

In one example the antibody-IR700 conjugate is an EGFR-antibody-IR700 molecule, such as, panitumumab-IR700 or cetuximab-IR700.

Antibody-IR700 molecules can be generated using methods such as those described in WO 2013/009475 (incorporated by reference herein in its entirety).

TABLE 1
Exemplary tumor-specific antigens and antibodies

| Tumor-Specific | | Exemplary Antibody/ |
|---|---|---|
| Antigen | Exemplary Tumors | Small Molecules |
| HER1 | Adenocarcinoma (e.g., | Cetuximab, panitumumab, zalutumumab, |
| | colorectal cancer, esophageal | nimotuzumab, matuzumab, necitumumab, |
| | cancer, gastric cancer, head | imgatuzumab, 806. Small molecule inhibitors |
| | and neck cancer) | gefitinib, erlotinib, and lapatinib can also be |
| | | conjugated to IR700. |
| HER2 | breast cancer, ovarian | Trastuzumab (Herceptin ®), pertuzumab |
| | cancer, stomach cancer, | (Perjeta ®, Omnitarg ®) |
| | uterine cancer | |
| HER3 | Breast, colon, lung, ovarian, | Patritumab, Duligotumab, MM-121 |
| | prostate, and head and neck | |
| | squamous cell cancer | |
| CA 242 | Colorectal, gastric, | Mouse clone M62862 and M62861 |
| | pancreatic, hepatocellular | |
| | cancer | |
| CD19 | B cell lymphoma, CLL, ALL | GBR 401, MEDI-551, Blinatumomab |
| | | (Blincyto ®) |
| CD20 | Non-Hodgkin lymphoma | Tositumomab (Bexxar ®); Rituximab (Rituxan, |
| | | Mabthera); Ibritumomab tiuxetan (Zevalin, for |
| | | example in combination with yttrium-90 or |
| | | indium-111 therapy); Ofatumumab (Arzerra ®), |
| | | veltuzumab, obinutuzumab, ublituximab, |
| | | ocaratuzumab |
| CD22 | Non-Hodgkin's lymphoma, | Narnatumab, inotuzumab ozogamicin, |
| | CLL, hairy cell leukemia, | moxetumomab pasudotox |
| | ALL, solid tumors | |
| CD25 | T-cell lymphoma | Daclizumab (Zenapax), Basiliximab |
| CD30 | Hodgkin's lymphoma | Brentuximab vedotin (ADCETRIS ®), |
| | | iratumumab |
| CD33 | Acute myelogenous | Gemtuzumab (Mylotarg, for example in |
| | leukemia | combination with calicheamicin therapy); |
| | | lintuzumab |
| CD37 | CLL, non-Hodgkin | Otlertuzumab |
| | lymphoma, mantle cell | |
| | lymphoma | |
| CD38 | Multiple myeloma | Daratumumab |
| CD40 | Multiple myeloma, non- | Lucatumumab, dacetuzumab |
| | Hodgkin's or Hodgkin's | |
| | lymphoma | |
| CD44 | Cancer stem cells, breast, | bivatuzumab |
| | prostate, renal, head and | RG3756 |
| | neck cancer, lymphoma, | |
| | leukemia | |
| CD52 | chronic lymphocytic | Alemtuzumab (Campath) |
| | leukemia | |
| CD56 | Small cell lung cancer, | Lorvotuzumab mertansine |
| | ovarian cancer | |
| CD70 | Renal cell carcinoma | Vorsetuzumab mafodotin |
| CD74 | Multiple myeloma | Milatuzumab |
| CD140 | Glioblastoma, non-small cell | Tovetumab |
| | lung cancer | |
| CAIX | Renal cell carcinoma | Girentuximab, tivozanib, cG250 |
| CEA | colorectal cancer, some | Arcitumomab (CEA-scan (Fab fragment, |
| | gastric cancers, biliary | approved by FDA), colo101; Labetuzumab |
| | cancer | (CEA-Cide ®) |
| Cancer antigen 125 | ovarian cancer, | OC125 monoclonal antibody |
| (CA125) | mesothelioma, breast cancer | |
| Alpha-fetoprotein | hepatocellular carcinoma | ⁹⁰Y-tacatuzumab tetraxetan; ab75705 (available |
| (AFP) | | from Abcam) and other commercially available |
| | | AFP antibodies |
| Cytokeratin | Colorectal cancer | ^{99m}Tc- Votumumab (HUMASPECT ®) |
| EGFL7 | Non-small cell lung cancer, | Parsatuzumab |
| | colorectal cancer | |
| EpCAM | Epithelial tumors (breast, | IGN101, oportuzumab monatox, tucotuzumab |
| | colon and lung) | celmoleukin, adecatumumab |
| EPHA3 | Lung, kidney and colon | KB004, IIIA4 |
| | tumors, melanoma, glioma | |
| | and hematological | |
| | malignancies | |
| FAP | Colon, breast, lung, | Sibrotuzumab, F19 |
| | pancreas, and head and neck | |
| | tumors | |
| Fibronectin | Hodgkin's lymphoma | Radretumab |
| Folate-binding | Ovarian cancer | MOv18 and MORAb-003 (farletuzumab) |
| protein | | |
| Folate receptor alpha | Ovarian cancer | Farletuzumab |
| Frizzled receptor | Breast, pancreatic, non-small | Vantictumab |
| | cell lung cancer | |
| Gangliosides | Neuroectodermal tumors and | 3F8, ch14.18, KW-2871 |
| (e.g., GD2, GD3 and | some epithelial tumors | |
| GM2) | | |
| gpA33 | Colorectal cancer | huA33 |
| glypican-1 | pancreatic cancer, glioma, | A-10, clone 01a033 |
| | and breast cancer) | |
| glypican-2 | neuroblastoma | CT3, hCT3 |
| glypican-3 | HCC | YP7, hYP7, GC33 |
| HGF | Solid tumors | Rilotumumab, ficlatuzumab |
| IGF1R | Glioma, lung, breast, head | Cixutumumab, dalotuzumab, figitumumab, |
| | and neck, prostate and | ganitumab, robatumumab, teprotumumab, |
| | thyroid cancer | AVE1642, IMC-A12, MK-0646, R1507, and CP |
| | | 751871 |
| IGLF2 | Breast cancer; | Dusigitumab |
| | Hepatocellular carcinoma; | |
| | Solid tumors | |
| IL-6 | renal cell cancer, prostate | Siltuximab |
| | cancer, Castleman's disease | |
| Integrin αVβ3 | Tumor vasculature | Etaracizumab (ABEGRIN ®), intetumumab |
| Integrin α5β1 | Tumor vasculature | Volociximab |
| Lewis Y | colorectal cancer, biliary | B3 (Humanized), hu3S193, IgN311 |
| | cancer | |
| Mesothelin | Mesothelioma, pancreatic | Amatuximab |
| | cancer | |
| MET | Breast, ovarian, and lung | AMG 102, METMAB, SCH 900105 |
| | cancer | |
| Mucins | Breast, colon, lung and | Pemtumomab (THERAGYN ®), cantuzumab |
| | ovarian cancer | mertansine, ⁹⁰Y clivatuzumab tetraxetan, |
| | | oregovomab (OVAREX ®) |
| PDGFR-alpha | Soft tissue sarcoma | Olaratumab |
| Phosphatidylserine | Breast, pancreatic, prostate, | Bavituximab |
| | non-small cell lung cancer, | |
| | hepatocellular carcinoma | |
| PSMA | Prostate cancer | J591 |
| RANKL | Prostate cancer, bone | Denosumab (XGEVA ®) |
| | metastases | |
| Scatter factor | Non-small cell lung, | Onartuzumab |
| receptor kinase | stomach, glioblastoma | |
| SLAMF7 (CD319) | Multiple myeloma | Elotuzumab |
| Syndecan 1 | Multiple myeloma, breast, | Indatuximab ravtansine |
| | bladder cancer | |
| TAG72 | adenocarcinomas including | B72.3 (FDA-approved monoclonal antibody), |
| | colorectal, pancreatic, | CC49 (minretumomab) |
| | gastric, ovarian, endometrial, | |
| | mammary, and non-small | |
| | cell lung cancer | |
| Tenascin | Glioma, breast and prostate | 81C6 |
| | tumors | |
| TRAILR1 | Colon, lung and pancreas | Mapatumumab (HGS-ETR1) |
| | tumors and hematological | |
| | malignancies | |
| TRAILR2 | Non-small cell lung cancer, | Conatumumab, lexatumumab, mapatumumab, |
| | non-Hodgkin's lymphoma, | tigatuzumab, HGS-ETR2, CS-1008 |
| | multiple myeloma | |
| Vascular endothelial | Colorectal cancer | Bevacizumab (Avastin ®) |
| growth factor | | |
| VEGFR | Epithelium-derived solid | IM-2C6, CDP791 |
| | lumors | |
| VEGFR2 | Gastric, non-small cell lung, | Ramucirumab (Cyramza ™) |
| | colorectal cancer | |
| Vimentin | Brain cancer | Pritumumab |

Exemplary antibodies that can be conjugated to IR700, and used in the disclosed methods, include 3F8, Abagovomab, Adecatumumab, Afutuzumab, Alacizumab, Alemtuzumab, Altumomab pentetate, Anatumomab mafenatox, Apolizumab, Arcitumomab, Bavituximab, Bectumomab, Belimumab, Besilesomab, Bevacizumab, Bivatuzumab mertansine, Blinatumomab, Brentuximab vedotin, Cantuzumab mertansine, Capromab pendetide, Catumaxomab, CC49, Cetuxinab, Citatuzumab bogatox, Cixutumumab, Clivatuzumab tetraxetan, Conatumumab, Dacetuzumab, Detumomab, Ecromeximab, Eculizumab, Edrecolomab, Epratuzumab, Ertumaxomab, Etaracizumab, Farletuzumab, Figitumumab, Galiximab, Gemtuzumab ozogamicin, Girentuximab, Glembatumumab vedotin, Ibritumomab tiuxetan, Igovomab, Imciromab, Intetumumab, Inotuzumab ozogamicin. Ipilimumab, Iratumumab, Labetuzumab, Lexatumumab, Lintuzumab, Lorvotuzumab mertansine, Lucatumumab, Lumiliximab, Mapatumumab, Matuzumab, Mepolizumab, Metelimumab, Milatuzumab, Mitumomab. Morolimumab, Nacolomab tafenatox, Naptumomab estafenatox, Necitumumab, Nimotuzumab, Nofetumomab merpentan, Ofatumumab, Olaratumab, Oportuzumab monatox, Oregovomab, Panitumumab, Pemtumomab, Pertuzumab, Pintumomab, Pritumumab, Ramucirumab. Rilotumumab, Rituximab, Robatumumab, Satumomab pendetide, Sibrotuzumab, Sonepcizumab, Tacatuzumab tetraxetan, Taplitumomab paptox, Tenatumomab, TGN1412, Ticilimumab (tremelimumab), Tigatuzumab, TNX-650, Trastuzumab, Tremelimumab, Tucotuzumab celmoleukin, Veltuzumab, Volociximab, Votumumab, and Zalutumumab.

In some examples, the cell surface protein recognized by the antibody conjugated to IR700, is one found on an immune cell, such as a Treg. In one example, the protein is CTLA4. In one example, the antibody-IR700 molecule is a CTLA4-antibody-IR700 molecule, such as ipilimumab-IR700 or tremelimumab-IR700.

In one example, a patient is treated with at least two different antibody-IR700 conjugates specific for cancer cell surface antigens. In one example, the two different antibody-IR700 conjugates are specific for the same protein (such as HER-2), but are specific for different epitopes of the protein (such as epitope 1 and epitope 2 of HER-2). In another example, the two different antibody-IR700 conjugates are specific for two different proteins or antigens. For example, anti-HER1-IR700 and anti-HER2-IR700 could be injected together as a cocktail to facilitate killing of cells bearing either HER1 or HER2.

### Immunomodulators/Immunoactivators

Immunoactivators of use in the disclosed methods include agents or compositions that activate the immune system and/or inhibit immuno-suppressor cells (also referred to herein as suppressor cells). Inhibition of immuno-suppressor cells and/or activation of immune responses may increase tumor cell killing and lead to production of memory T cells, which can provide a “vaccine” effect against recurrences and/or distant tumors or metastases. In some examples, one or more immunoactivators are used in combination with a CD25-specific antibody-IR700 conjugate disclosed herein, one or more tumor-specific antibody-IR700 molecules, and/or one or more reducing agents.

In some aspects, the immunoactivator is an inhibitor of immuno-suppressor cells, for example, an agent that inhibits or reduces activity of immuno-suppressor cells. In some cases, the immunomodulator kills immuno-suppressor cells. In some examples, the immuno-suppressor cells are regulatory T (Treg) cells. In some examples, not all of the suppressor cells are killed in vivo, as such could lead to development of autoimmunity. Thus, in some examples, the method reduces the activity or number of immuno-suppressor cells in an area of subject, such as in the area of a tumor or an area that used to have a tumor, by at least 50%, at least 60%, at least 75%, at least 80%, at least 90%, or at least 95%. In some examples, the method reduces the total number of suppressor cells in a subject by at least 50%, at least 60%, at least 75%, at least 80%, at least 90%, or at least 95%.

Inhibitors of immuno-suppressor cells include tyrosine kinase inhibitors (such as sorafenib, sunitinib, and imatinib), or chemotherapeutic agents (such as cyclophosphamide or interleukin-toxin fusions, for example denileukin difitox (IL2-diphtheria toxin fusion), or other antibodies that bind to suppressor cell surface proteins (such as those described below). In other examples, inhibitors of immuno-suppressor cells include immune checkpoint inhibitors, for example, anti-PD-L1 antagonizing antibodies, thereby preventing PD-L1 from binding to PD-1 (referred to herein as PD-1/PD-L1 mAb-mediated immune checkpoint blockade (ICB)). Thus, in some examples, the immunomodulator is a PD-L1 antagonizing antibody, such as atezolizumab, avelumab, durvalumab, cosibelimab, KN035 (envafolimab), BMS-936559. BMS935559, MEDI-4736, MPDL-3280A, or MEDI-4737 (or other examples provided herein). In some examples, the immunomodulator is a PD-1 antagonizing antibody, such as nivolumab, pembrolizumab, cemiplimab, or dostarlimab. Checkpoint inhibitors also include CTLA-4 antibodies, including ipilimumab and tremelimumab. The inhibitor of immuno-suppressor cells can also be a LAG-3 or B7-H3 antagonist, such as BMS-986016 and MGA271. In some examples, two or more of the inhibitors of immuno-suppressor cells can be administered to a subject. In one non-limiting example, a subject is administered an anti-PD1 and an anti-LAG-3 antibody.

In some examples, the agent that inhibits or reduces activity of suppressor cells includes one or more antibody-IR700 conjugates, wherein the antibody specifically binds to a suppressor cell surface protein, such as CD4, C—X—C chemokine receptor type 4 (CXCR4), C—C chemokine receptor type 4 (CCR4), cytotoxic T-lymphocyte-associated protein 4 (CTLA4), glucocorticoid induced TNF receptor (GITR), OX40, folate receptor 4 (FR4), CD16, CD56, CD8, CD122, CD23, CD163, CD206, CD11b, Gr-1, CD14, interleukin 4 receptor alpha chain (IL-4Ra), interleukin-1 receptor alpha (IL-1Ra), interleukin-1 decoy receptor, CD103, fibroblast activation protein (FAP), CXCR2, CD33, CD66b, or V-domain immunoglobulin suppressor of T cell activation (VISTA).

In other aspects, the immunomodulator is an immune system activator (immunoactivator). In some examples, an immune system activator stimulates (activates) one or more T cells and/or natural killer (NK) cells. In one example, the immune system activator includes one or more interleukin (IL), such as IL-2, IL-15, IL-7, IL-12, and/or IL-21. In a non-limiting example, the immunomodulator includes or consists of IL-15. In a non-limiting example, the immunomodulator includes IL-2 and IL-15. In a non-limiting example, the immunomodulator includes or consists of interferon gamma. In another example, the immune system activator includes one or more agonists to co-stimulatory receptors, such as 4-1BB, OX40, or GITR. In a non-limiting example, the immunomodulator includes stimulatory anti-4-1BB, anti-OX40, and/or anti-GITR antibodies.

In some examples, one or more (such as 1, 2, 3, 4, 5, or more) doses of the immunomodulator is administered to the subject. Thus, administering the immunomodulator can be completed in a single day, or may be done repeatedly on multiple days with the same or a different dosage (such as administering at least 2 different times, 3 different times, 4 different times 5 different times or 10 different times). In some examples, the repeated administrations are the same dose. In other examples, the repeated administrations are different does (such as a subsequent dose that is higher than the preceding dose or a subsequent dose that is lower than the preceding dose). Repeated administration of the immunomodulator may be done on the same day, on successive days, every other day, every 1-3 days, every 3-7 days, every 1-2 weeks, every 2-4 weeks, every 1-2 months, or at even longer intervals. In some examples, at least one dose of the immunomodulator is administered prior to irradiation and at least one dose of the immunomodulator is administered after irradiation.

### Irradiation

After the subject is administered one or more antibody-IR700 conjugates, the subject (or a tumor in the subject) is irradiated. As only cells expressing the target protein (such as CD25) will be recognized by the antibody, only those cells will have sufficient amounts of the antibody-IR700 conjugates associated with it to it to kill the cells. This decreases the likelihood of undesired side effects, such as killing of non-tumor cells, as the irradiation will only kill the cells to which the antibody-IR700 conjugates are bound, not the other cells.

The subject (for example, a tumor in the subject) is irradiated with a therapeutic dose of radiation at a wavelength of 660-740 nm, such as 660-710 nm, 660-700 nm, 680-700 nm, 670-690 nm, for example, 680 nm or 690 nm. In particular examples, the cells, the tumor, or the subject is irradiated at a dose of at least 1 J/cm², such as at least 4 J/cm², at least 10 J/cm², at least 20 J/cm², at least 25 J/cm², at least 30 J/cm², at least 50 J/cm², at least 100 J/cm², or at least 500 J/cm², for example, 1-1000 J/cm², 1-500 J/cm², 1-100 J/cm², 4-50 J/cm², 4-25 J/cm², 30-50 J/cm², 10-100 J/cm², 20-30 J/cm², 1 to 10 J/cm², 4 to 10 J/cm², 20-50 J/cm² or 10-50 J/cm². In particular examples, the subject (or tumor in the subject) is irradiated at a wavelength of 690 nm and a dose of 50 J/cm².

The subject can be irradiated one or more times. Thus, irradiation can be completed in a single day, or may be done repeatedly on multiple days with the same or a different dosage (such as irradiation at least 2 different times, 3 different times, 4 different times 5 different times or 10 different times). In some examples, the repeated irradiations are the same dose. In other examples, the repeated irradiations are different does (such as a subsequent dose that is higher than the preceding dose or a subsequent dose that is lower than the preceding dose). Repeated irradiations may be done on the same day, on successive days, every other day, every 1-3 days, every 3-7 days, every 1-2 weeks, every 2-4 weeks, every 1-2 months, or at even longer intervals. In one example, a first irradiation is 50 J/cm² and a second irradiation is at 100 J/cm², where the irradiations are on consecutive days (for example, about 24 hours apart). In one example, a first irradiation is 10-50 J/cm² and a second irradiation is at 10-50 J/cm², where the irradiations are on consecutive days (for example, about 24 hours apart).

In some examples, the irradiation is provided with a wearable device incorporating an NIR LED. In other examples, another type of device that can be used with the disclosed methods is a flashlight-like device with NIR LEDs. Such a device can be used for focal therapy of lesions during surgery, or incorporated into endoscopes to apply NIR light to body surfaces after the administration of one or more PIT agents. Such devices can be used by physicians or qualified health personnel to direct treatment to particular targets on the body.

### Treatment Using Wearable NIR LEDs

As described herein, the disclosed methods are highly specific for cancer cells and/or Tregs in the tumor bed. However, to kill the cells circulating in the body or present on the skin, the patient can wear a device that incorporates an NIR LED. In some examples, the patient uses at least two devices, for example an article of clothing or jewelry during the day, and a blanket at night. In some examples, the patient uses at least two devices at the same time, for example two articles of clothing. These devices make it possible to expose the patient to NIR light using portable everyday articles of clothing and jewelry so that treatment remains private and does not interfere with everyday activities. In some examples, the device can be worn discreetly during the day for PIT therapy. Exemplary devices incorporating an NIR LED are disclosed in International Patent Application Publication No. WO 2013/009475 (incorporated by reference herein).

In one example, the patient is administered one or more antibody-IR700 molecules, for example in combination with one or more reducing agents and/or one or more immunoactivators using the methods described herein. The patient then wears a device that incorporates an NIR LED, permitting long-term therapy and treatment of tumor cells that are present in the blood or lymph or on the skin. In some examples, the dose is at least at least 1 J/cm², at least at least 4 J/cm², at least 10 J/cm², at least 20 J/cm², at least 30 J/cm², at least 40 J/cm², or at least 50 J/cm², such as 10 to 100 J/cm², 10 to 50 J/cm², such as 20 J/cm² or 30 J/cm². In some examples, administration of the one or more antibody—IR700 conjugates, for example in combination with one or more reducing agents and/or one or more immunoactivators, is repeated over a period of time (such as bi-weekly or monthly), to ensure therapeutic levels are present in the body.

In some examples, the patient wears or uses the device, or a combination of devices, for at least 1 week, such as at least 2 weeks, at least 4 weeks, at least 8 weeks, at least 12 weeks, at least 4 months, at least 6 months, or even at least 1 year. In some examples, the patient wears or uses the device, or combination of devices, for at least 4 hours a day, such as at least 12 hours a day, at least 16 hours a day, at least 18 hours a day, or 24 hours a day. It is quite possible that multiple devices of a similar “everyday” nature (blankets, bracelets, necklaces, underwear, socks, shoe inserts) could be worn by the same patient during the treatment period. At night the patient can use the NIR LED blanket or other covering.

### Administration of Additional Therapies

As discussed above, prior to, during, or following administration of one or more antibody-IR700 conjugates, for example in combination with one or more reducing agents and/or one or more immunoactivators, and irradiation, the subject can receive one or more other therapies. In one example, the subject receives one or more treatments to remove or reduce the tumor prior to administration of the one or more antibody-IR700 conjugates, for example in combination with one or more reducing agents and/or one or more immunoactivators, and irradiation. In other examples, additional treatments or therapeutic agents (such as anti-neoplastic agents) can be administered to the subject to be treated, for example, after the irradiation, for example, about 0 to 8 hours after irradiating the cell (such as at least 10 minutes, at least 30 minutes, at least 60 minutes, at least 2 hours, at least 3 hours, at least 4, hours, at least 5 hours, at least 6 hours, or at least 7 hours after the irradiation, for example no more than 10 hours, no more than 9 hours, or no more than 8 hours, such as 1 hour to 10 hours, 1 hour to 9 hours 1 hour to 8 hours, 2 hours to 8 hours, or 4 hours to 8 hours after irradiation). In another example, the additional therapeutic agents are administered just before the irradiation (such as about 10 minutes to 120 minutes before irradiation, such as 10 minutes to 60 minutes or 10 minutes to 30 minutes before irradiation).

Examples of such therapies that can be used in combination with the disclosed methods, which in some examples, enhance accessibility of the tumor to additional therapeutic agents for about 8 hours after the PIT, include but are not limited to, surgical treatment for removal or reduction of the tumor (such as surgical resection, cryotherapy, or chemoembolization), as well as anti-tumor pharmaceutical treatments which can include radiotherapeutic agents, anti-neoplastic chemotherapeutic agents, antibiotics, alkylating agents and antioxidants, kinase inhibitors, and other agents. In some examples, the additional therapeutic agent is conjugated to a nanoparticle. Particular examples of additional therapeutic agents that can be used include microtubule binding agents, DNA intercalators or cross-linkers, DNA synthesis inhibitors, DNA and/or RNA transcription inhibitors, antibodies, enzymes, enzyme inhibitors, and gene regulators. These agents (which are administered at a therapeutically effective amount) and treatments can be used alone or in combination.

“Microtubule binding agent” refers to an agent that interacts with tubulin to stabilize or destabilize microtubule formation thereby inhibiting cell division. Examples of microtubule binding agents that can be used in conjunction with the disclosed methods include, without limitation, paclitaxel, docetaxel, vinblastine, vindesine, vinorelbine (navelbine), the epothilones, colchicine, dolastatin 15, nocodazole, podophyllotoxin and rhizoxin. Analogs and derivatives of such compounds also can be used. For example, suitable epothilones and epothilone analogs are described in International Publication No. WO 2004/018478. Taxoids, such as paclitaxel and docetaxel, as well as the analogs of paclitaxel taught by U.S. Pat. Nos. 6,610,860; 5,530,020; and 5,912,264 can be used.

The following classes of compounds can be used with the methods disclosed herein: suitable DNA and/or RNA transcription regulators, including, without limitation, actinomycin D, daunorubicin, doxorubicin and derivatives and analogs thereof also are suitable for use in combination with the disclosed therapies. DNA intercalators and cross-linking agents that can be administered to a subject include, without limitation, cisplatin, carboplatin, oxaliplatin, mitomycins, such as mitomycin C, bleomycin, chlorambucil, cyclophosphamide and derivatives and analogs thereof. DNA synthesis inhibitors suitable for use as therapeutic agents include, without limitation, methotrexate, 5-fluoro-5′-deoxyuridine, 5-fluorouracil and analogs thereof. Examples of suitable enzyme inhibitors include, without limitation, camptothecin, etoposide, formestane, trichostatin and derivatives and analogs thereof. Suitable compounds that affect gene regulation include agents that result in increased or decreased expression of one or more genes, such as raloxifene, 5-azacytidine, 5-aza-2′-deoxycytidine, tamoxifen, 4-hydroxytamoxifen, mifepristone and derivatives and analogs thereof. Kinase inhibitors include Gleevec® (imatinib), Iressa® (gefitinib), and Tarceva® (erlotinib) that prevent phosphorylation and activation of growth factors.

Non-limiting examples of anti-angiogenic agents include molecules, such as proteins, enzymes, polysaccharides, oligonucleotides, DNA, RNA, and recombinant vectors, and small molecules that function to reduce or even inhibit blood vessel growth. Examples of suitable angiogenesis inhibitors include, without limitation, angiostatin K1-3, staurosporine, genistein, fumagillin, medroxyprogesterone, suramin, interferon-alpha, metalloproteinase inhibitors, platelet factor 4, somatostatin, thromohospondin, endostatin, thalidomide, and derivatives and analogs thereof. For example, in some aspects the anti-angiogenesis agent is an antibody that specifically binds to VEGF (e.g., Avastin, Roche) or a VEGF receptor (e.g., a VEGFR2 antibody). In one example, the anti-angiogenic agent includes a VEGFR2 antibody, or DMXAA (also known as Vadimezan or ASA404; available commercially, e.g., from Sigma Corp., St. Louis, MO) or both. The anti-angiogenic agent can be bevacizumab, sunitinib, an anti-angiogenic tyrosine kinase inhibitor (TKI), such as sunitinib, xitinib and dasatinib. These can be used individually or in any combination.

Other therapeutic agents, for example anti-tumor agents, that may or may not fall under one or more of the classifications above, also are suitable for administration in combination with the disclosed methods. By way of example, such agents include adriamycin, apigenin, rapamycin, zebularine, cimetidine, and derivatives and analogs thereof.

In some examples, the subject receiving the therapeutic antibody-IR700 conjugate(s) is also administered interleukin-2 (IL-2), for example via intravenous administration. In particular examples, IL-2 (Chiron Corp., Emeryville, CA) is administered at a dose of at least 500,000 IU/kg as an intravenous bolus over a 15 minute period every eight hours beginning on the day after administration of the antibody-IR700 conjugate(s) and continuing for up to 5 days. Doses can be skipped depending on subject tolerance.

Exemplary additional therapeutic agents include anti-neoplastic agents, such as chemotherapeutic and anti-angiogenic agents or therapies, such as radiation therapy. In one example the agent is a chemotherapy immunosuppressant (such as Rituximab, steroids) or a cytokine (such as GM-CSF). Exemplary chemotherapeutic agents are provided in, for example, Slapak and Kufe, Principles of Cancer Therapy, Chapter 86 in Harrison's Principles of Internal Medicine. 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993). Combination chemotherapy is the administration of more than one agent to treat cancer.

Exemplary chemotherapeutic agents that can be used with the methods provided herein include but are not limited to, carboplatin, cisplatin, paclitaxel, docetaxel, doxorubicin, epirubicin, topotecan, irinotecan, gemcitabine, iazofurine, gemcitabine, etoposide, vinorelbine, tamoxifen, valspodar, cyclophosphamide, methotrexate, fluorouracil, mitoxantrone, Doxil (liposome encapsulated doxiorubicine) and vinorelbine. Additional examples of chemotherapeutic agents that can be used include alkylating agents, antimetabolites, natural products, or hormones and their antagonists. Examples of alkylating agents include nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine). Specific non-limiting examples of alkylating agents are temozolomide and dacarbazine. Examples of antimetabolites include folic acid analogs (such as methotrexate), pyrimidine analogs (such as 5-FU or cytarabine), and purine analogs, such as mercaptopurine or thioguanine. Examples of natural products include vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase). Examples of miscellaneous agents include platinum coordination complexes (such as cis-diamine-dichloroplatinum II also known as cisplatin), substituted ureas (such as hydroxyurea), methyl hydrazine derivatives (such as procarbazine), and adrenocrotical suppressants (such as mitotane and aminoglutethimide). Examples of hormones and antagonists include adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testosterone proprionate and fluoxymesterone).

Exemplary chemotherapy drugs include Adriamycin, Alkeran, Ara-C, BiCNU, Busulfan, CCNU, Carboplatinum, Cisplatinum, Cytoxan, Daunorubicin, DTIC, 5-fluoruracil (5-FU), Fludarabine, Hydrea, Idarubicin, Ifosfamide, Methotrexate, Mithramycin, Mitomycin, Mitoxantrone, Nitrogen Mustard. Taxol (or other taxanes, such as docetaxel), Velban, Vincristine, VP-16, Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin and calcitriol. Non-limiting examples of immunoactivators that can be used include AS-101 (Wyeth-Ayerst Labs.), bropirimine (Upjohn), gamma interferon (Genentech), GM-CSF (granulocyte macrophage colony stimulating factor; Genetics Institute), IL-2 (Cetus or Hoffman-LaRoche), human immune globulin (Cutter Biological), IMREG (from Imreg of New Orleans, La.), SK&F 106528, and TNF (tumor necrosis factor; Genentech).

In some examples, the additional therapeutic agent is conjugated to (or otherwise associated with) a nanoparticle, such as one at least 1 nm in diameter (for example at least 10 nm in diameter, at least 30 nm in diameter, at least 100 nm in diameter, at least 200 nm in diameter, at least 300 nm in diameter, at least 500 nm in diameter, or at least 750 nm in diameter, such as 1 nm to 500 nm, 1 nm to 300 nm, 1 nm to 100 nm, 10 nm to 500 nm, or 10 nm to 300 nm in diameter).

In one example, at least a portion of the tumor (such as a metastatic tumor) is surgically removed (for example via surgical resection and/or cryotherapy), irradiated (for example administration of radioactive material or energy (such as external beam therapy) to the tumor site to help eradicate the tumor or shrink it), chemically treated (for example via chemoembolization) or combinations thereof, prior to administration of the disclosed therapies (such as administration of one or more antibody-IR700 conjugates, for example in combination with one or more reducing agents and/or one or more immunoactivators, and irradiation). For example, a subject having a metastatic tumor can have all or part of the tumor surgically excised prior to administration of the disclosed therapies. In an example, one or more chemotherapeutic agents are administered following treatment with one or more antibody-IR700 conjugates, for example in combination with one or more reducing agents and/or one or more immunoactivators, and irradiation, and irradiation. In another particular example, the subject has a metastatic tumor and is administered radiation therapy, chemoembolization therapy, or both concurrently with the administration of the disclosed therapies.

In some examples, the additional therapeutic agent administered is a monoclonal antibody, for example, 3P8, Abagovomab, Adecatumumab, Afutuzumab, Alacizumab, Alemtuzumab, Altumomab pentetate, Anatumomab mafenatox, Apolizumab, Arcitumomab, Bavituximab, Bectumomab, Belimumab, Besilesomab, Bevacizumab, Bivatuzumab mertansine, Blinatumomab, Brentuximab vedotin, Cantuzumab mertansine, Capromab pendetide, Catumaxomab, CC49, Cetuximab, Citatuzumab bogatox, Cixutumumab, Clivatuzumab tetraxetan, Conatumumab, Dacetuzumab, Detumomab, Ecromeximab, Eculizumab, Edrecolomab, Epratuzumab, Ertumaxomab, Etaracizumab, Farletuzumab, Figitumumab, Galiximab, Gemtuzumab ozogamicin, Girentuximab, Glembatumumab vedotin, Ibritumomab tiuxetan, Igovomab, Imciromab, Intetumumab, Inotuzumab ozogamicin. Ipilimumab, Iratumumab, Labetuzumab, Lexatumumab, Lintuzumab, Lorvotuzumab mertansine, Lucatumumab, Lumiliximab, Mapatumumab, Matuzumab, Mepolizumab, Metelimumab, Milatuzumab, Mitumomab. Morolimumab, Nacolomab tafenatox, Naptumomab estafenatox, Necitumumab, Nimotuzumab, Nofetumomab merpentan, Ofatumumab, Olaratumab, Oportuzumab monatox. Oregovomab, Panitumumab, Pemtumomab, Pertuzumab, Pintumomab, Pritumumab, Ramucirumab. Rilotumnumab, Rituximab, Robatumunab, Satumrnomab pendetide, Sibrotuzumab, Sonepcizumab, Tacatuzumab tetraxetan, Taplitumomab paptox, Tenatumomab, TGN1412, Ticilimumab (tremelimumab), Tigatuzumab, TNX-650, Trastuzumab, Tremelimumab, Tucotuzumab celmoleukin, Veltuzumab, Volociximab, Votumumab, Zalutumumab, or combinations thereof.

### Exemplary Reducing Agents

Exemplary reducing agents that can be used in the methods provided herein include agents that loose (or “donate”) an electron to an electron recipient (oxidizing agent) in a redox chemical reaction. Exemplary reducing agents that can be used in the methods provided herein include L-cysteine, L-sodium ascorbate (L-NaAA), ascorbic acid (such as L- or R-ascorbic acid) and glutathione. In some examples, the reducing agent used in the disclosed methods is not sodium azide. In some examples, the reducing agent used in the disclosed methods is not L-cysteine. In one example, the reducing agent is L-NaAA (such as 5 to 50 g i.p.).

The following examples are provided to illustrate certain particular features and/or aspects. These examples should not be construed to limit the disclosure to the particular features or aspects described.

### EXAMPLES

### Example 1: Binding Affinity Measured by Surface Plasmon Resonance

This example describes affinity of antibodies 14564, 14569 and 14572 for CD25 as measured using BIACORE™ 8K.

### Samples

| Samples | MW(KDa) | Concentration (mg/ml) |
|---|---|---|
| Cynomolgus CD25 | 22.4 | 1 |
| VH2 + VL4 | 150 | 1.1 |
| 14564 | 150 | 2.728 |
| 14569 | 150 | 0.565 |
| 14572 | 150 | 0.27 |

### Instrument and Reagent

| Names | Cat. No. | Lot. No. | Vendor |
|---|---|---|---|
| BIACORE ™ 8K | N/A | N/A | GE Healthcare |
| Series S Sensor | 29-1275-55 | 10309880 | GE Healthcare |
| Chip Protein A | | | |
| HBS-EP+ buffer | BR-1006-69 | 31715 | GE Healthcare |
| Regeneration buffer: | 20211113 | 20211113 | GenScript |
| 10 mM Glycine-HCl pH 1.5 | | | |

### Affinity Measurement

Running buffer was prepared by diluting one volume of 10× buffer with 9 volumes of degassed, filtered MilliQ water. Regeneration buffer (10 mM glycine) was prepared by dissolving glycine in MilliQ water and adjusting the pH to approximately 1.5-1.7. The assay was performed at 25° C. using HBS-EP+ as the running buffer. Antibodies were injected as capture on the Series S Sensor Chip Protein A. Antigens were diluted to multiple concentrations and injected over the surface of flow cell 1 and 2 as association phase, followed by injection of running buffer as dissociation phase.

### Running Configuration

Capture

| Ligand | antibodies | |
|---|---|---|
| Capture Time | 30 | seconds |

Association & Dissociation

| | | |
|---|---|---|
| Association | 120 | seconds |
| contact time | | |
| Dissociation | 360 | seconds |
| contact time | | |
| Flow rate | 30 | μl/min |

| | |
|---|---|
| Sample | 800, 400, 200, 100, |
| concentrations | 50, 25, and 12.5 nM |

Surface Regeneration

| | | |
|---|---|---|
| Contact time | 30 | seconds |
| Flow rate | 30 | μl/min |

| | |
|---|---|
| Regeneration | 10 mM Glycine- |
| buffer | HCl pH 1.5 |

### Results

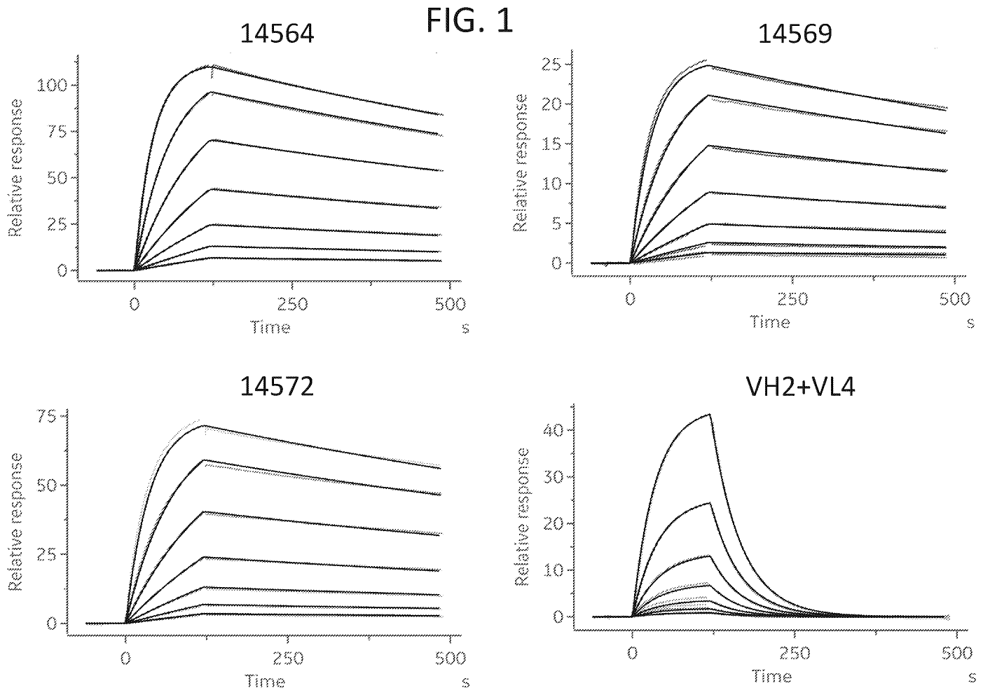

All data were processed using the BIACORE™ 8K Evaluation software version 3.0. Flow cell 1 and blank injection of buffer in each cycle were used as double reference for Response Units (RU) subtraction. Affinity of each antibody is listed in Table 2 and sensor-grams are shown in FIG. 1. Antibodies 14564, 14569 and 14572 bind CD25 with high affinity (KD=1.74×10⁻⁸ M, 1.85×10⁻⁸ M and 1.96×10⁻⁸ M, respectively). VH2+VL4 is a parental antibody (before affinity maturation).

TABLE 2
Affinity measurement of antibodies to CD25 (sIL-2 Receptor α)

| Ligand | Analyte | Chi² (RU²) | ka (1/Ms) | kd (1/s) | KD (M) | Rmax (RU) |
|---|---|---|---|---|---|---|
| 14564 | cynomolgus CD25 | 1.67 × 10⁻¹ | 4.26 × 10⁴ | 7.44 × 10⁻⁴ | 1.74 × 10⁻⁸ | 114.1 |
| 14569 | cynomolgus CD25 | 5.22 × 10⁻² | 3.85 × 10⁴ | 7.14 × 10⁻⁴ | 1.85 × 10⁻⁸ | 26.1 |
| 14572 | cynomolgus CD25 | 4.81 × 10⁻¹ | 3.47 × 10⁴ | 6.79 × 10⁻⁴ | 1.96 × 10⁻⁸ | 76.1 |
| VH2 + VL4 | cynomolgus CD25 | 1.26 × 10⁻¹ | 9.28 × 10³ | 2.17 × 10⁻² | 2.34 × 10⁻⁶ | 175.8 |

### Example 2: Binding of CD25-Specific Antibodies to CD25-Positive Cells

This example describes binding of antibodies 14564, 14569 and 14572 antibodies to CD25-expressing KIT-225 cells by fluorescence activated cell sorting (FACS). KIT-225 cells are an IL-2-dependent human T cell line.

FACS buffer: 1×PBS with 1% FBS (lx PBS, Accugene Cat. No. 51225, Lot No. 8MB103)

### Serial Dilution of Antibody

| | | | Volume needed (5 | |
|---|---|---|---|---|
| | Concentration | Starting | μg mAb) for 500 | |
| Vial label | (mg/mL) | concentration | μL | |
| Basiliximab | 1.000 | 10 μg/mL | 5 | μL |
| 14564 | 2.728 | 10 μg/mL | 1.9 | μL |
| 14569 | 0.38 | 10 μg/mL | 13.1 | μL |
| 14572 | 2.505 | 10 μg/mL | 2 | μL |

### FACS Procedure

KIT-225 cells were washed once with FACS buffer, counted, and resuspended at a concentration of 1×10⁶/mL in FACS buffer. To 5 mL (5×10⁶ total) of cells, 25 μL of viable dye (Thermo Fisher, L34974, equivalent to 1:200 dilution) was added and mixed well. After incubation at 4° C. for 30 minutes, cells were washed twice with FACS buffer (200×g, 5 minutes each). Cells were resuspended in 5 mL FACS buffer and 100 μL was added per well, followed by addition of 100 μL diluted antibody. The solution was mixed by pipetting up and down and then incubated at 4° C. for 60 minutes, after which cells were washed twice with FACS buffer (200×g, 5 minutes each). Cells were resuspended in 100 μL FACS buffer. Fifty μL of secondary antibody was diluted in 5 mL FACS buffer (equivalent to 1:100 dilution), and 100 μL was added to each well and mixed by pipetting up and down. Following incubation at 4° C. for 30 minutes, cells were washed twice with FACS buffer (200×g, 5 minutes each), and resuspended in approximately 250 μL FACS buffer. FACS analysis was performed with Attune Flow cytometer using tube reading module.

### Results

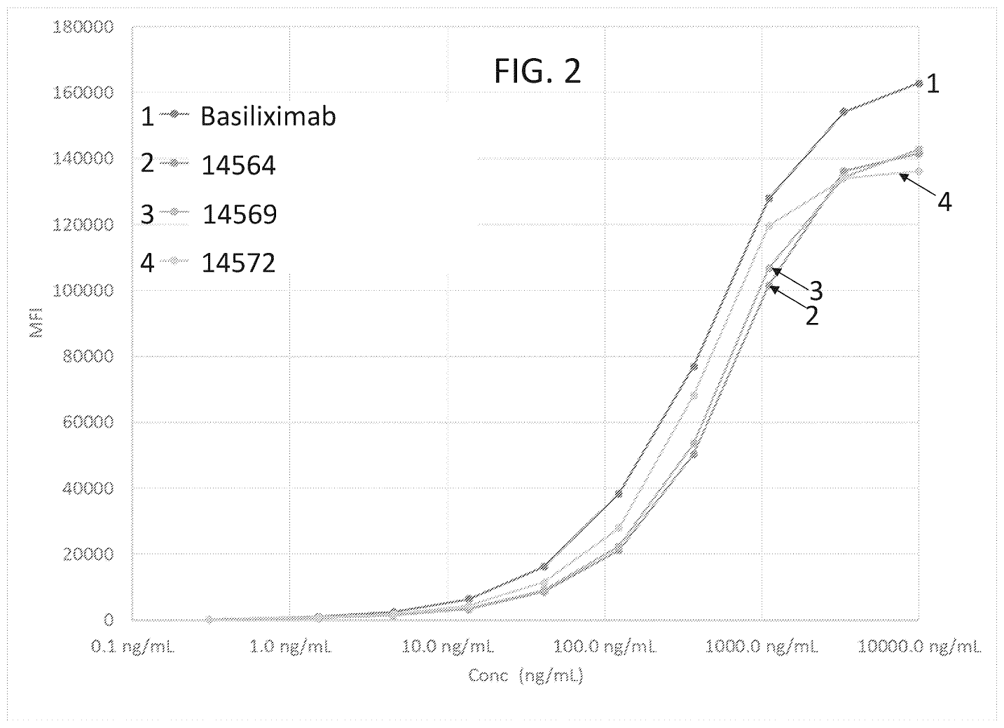

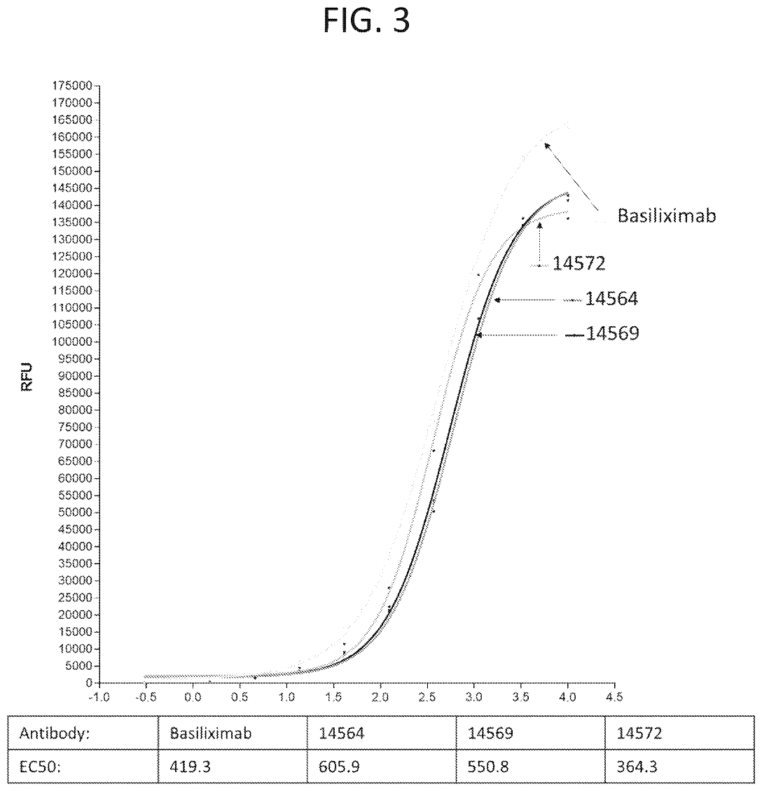

Results of the FACS analysis are shown in FIG. 2 and FIG. 3. Antibodies 14564, 14569 and 14572 exhibited strong binding to CD25-positive KIT-225 cells and had a binding affinity similar to Basiliximab (also known as SIMULECT©), a chimeric mouse-human monoclonal antibody that binds CD25 (Kahan et al., Transplantation 67(2):276-284, 1999).

### Example 3: ADCC

This example describes a study to determine whether antibodies 14564, 14569 and 14572 induce ADCC.

### Cell Lines

Jurkat/CD16.V175 cells (ADCC responder cells) were grown in RPMI-1640 with 10% FBS, 1 mg/mL Geneticin and 200 μg/mL Hygromycin. KIT-225 cells (target cells) were grown in RPMI-1640 with 10% FBS and 50 μM β-mercaptoethanol. ADCC assay medium was RPMI-1640 with 10% FBS.

### Antibody Working Solution Preparation

Basiliximab and the three anti-CD25 mAbs 14564, 14569 and 14572 were diluted to 50 μg/mL as shown below.

50 μg/mL, 360 μL = 18 μg

| | Concentration | mAb volume | |
|---|---|---|---|
| Antibody | (mg/mL) | needed (μL) | Medium |
| Basiliximab | 1.000 | 18.00 | 342.00 |
| 14564 | 2.728 | 6.60 | 353.40 |
| 14569 | 0.380 | 47.37 | 312.63 |
| 14572 | 2.505 | 7.19 | 352.81 |

Antibodies were serially diluted to 16.7 μg/mL, 5.5 μg/mL, 1.85 μg/mL, 0.6 μg/mL and 0.2 μg/mL (120 μL previous+240 μL medium). On the day of the assay, KIT-225 cells were harvested from culture. Cells were washed twice with medium to remove any IL-2. Cells were resuspended with assay medium to 4.2×10⁵ cells/mL (minimal 6 mL).

| Cell concentration | Cell volume | Viability | Volume to 4.2 × 10⁵/mL |
|---|---|---|---|
| 2.6 × 10⁵/mL | 20 mL | 55% | 12.3 mL |

KIT-225 cells (30 μL, equivalent to 12,500 cells) were added to a white clear-bottom 96-well microplate. Thirty μL of mAb working solution was added to each well of the plate and mixed. Cells were incubated with antibody at 37° C. for 1 hour.

The ADCC responder cells (Jurkat/CD16.V175) were harvested, washed once with assay medium, and resuspended to 1.9×10⁶/mL (minimal 8 mL).

| Cell concentration | Cell volume | Viability | Volume to 1.9 × 10⁶/mL |
|---|---|---|---|
| 2 × 10⁶/mL | 20 mL | 72% | 21 mL |

ADCC responder cells (40 μL, equivalent to 75,000 cells) were added to each well of the plate. Each treatment was tested in triplicate. After about 5-6 hours incubation, 100 μL of One-Step Luciferase reagent was added per well and the plate was rocked gently at room temperature for 30 minutes. Luminescence was measured using a luminometer.

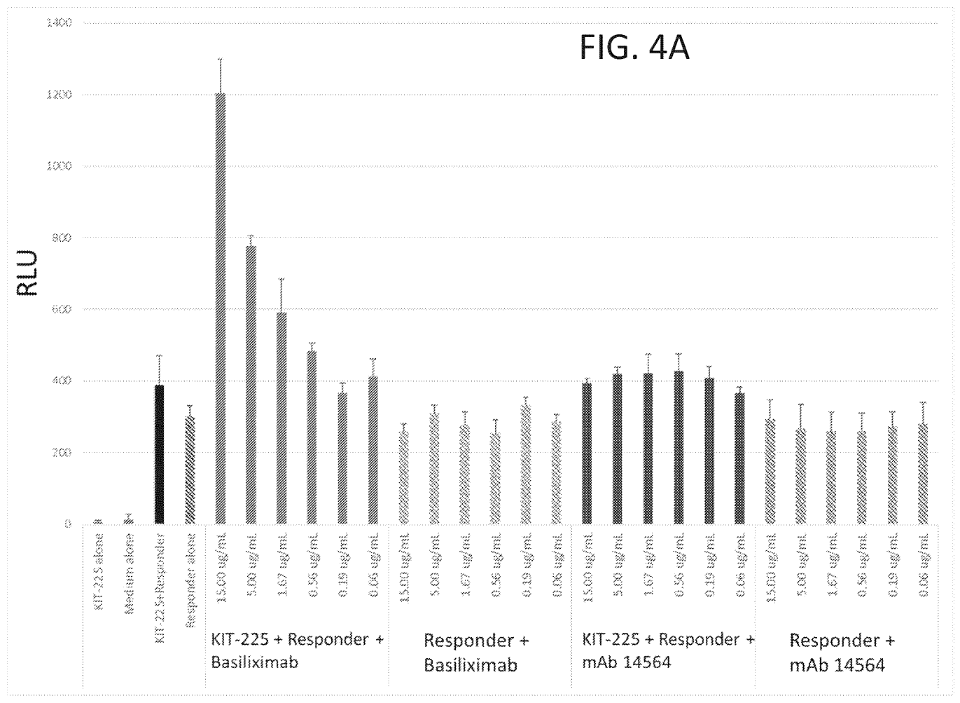

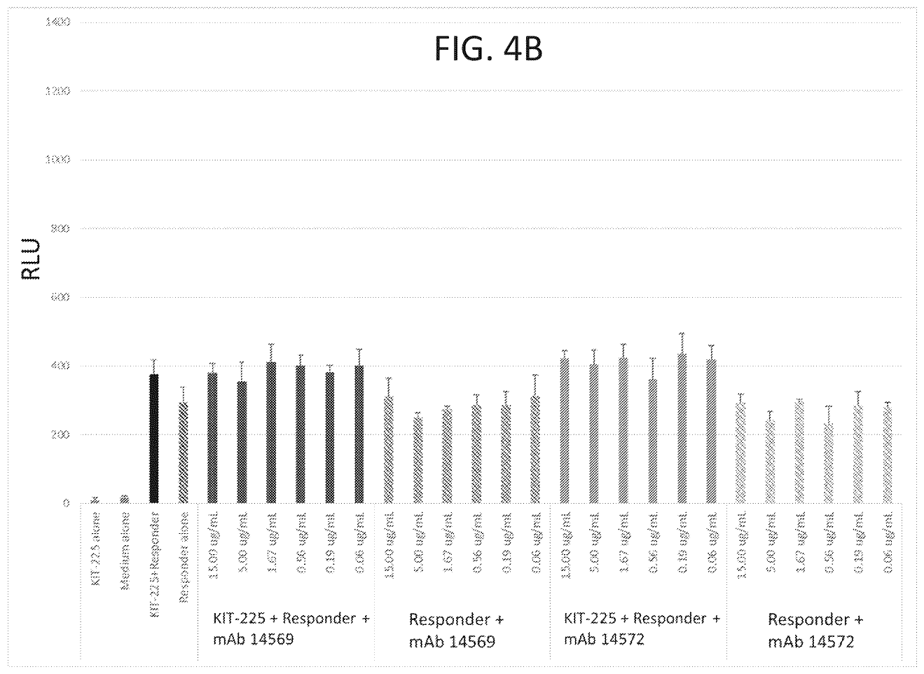

As shown in FIGS. 4A-4B, Basiliximab possesses ADCC activity, but none of antibodies 14564, 14569 and 14572 were capable of inducing ADCC activity.

### Example 4: Anti-CD25 Antibody-IR700 Conjugate is Capable of Binding to and Inducing Cell Killing of CD25-Expressing Cells

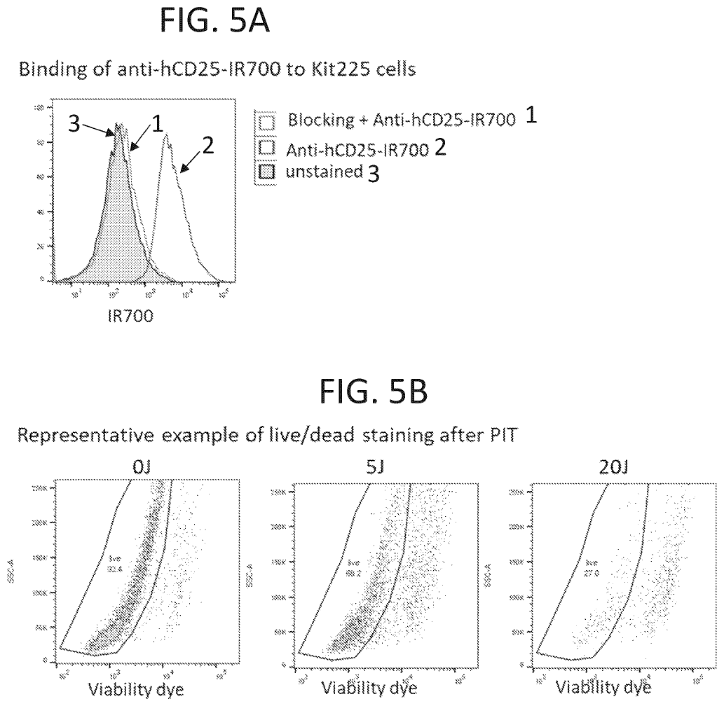

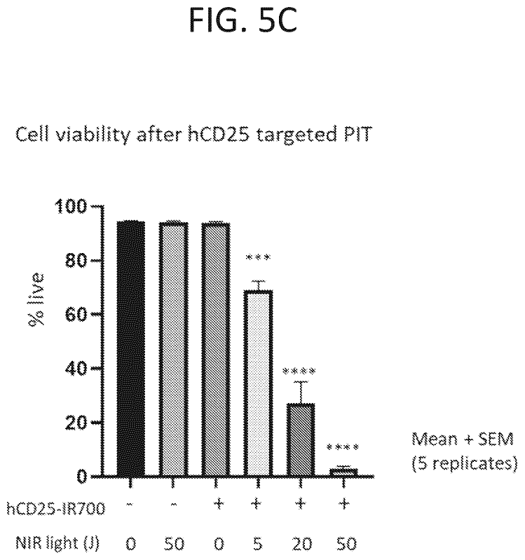

This example describes a study to evaluate targeting photoimmunotherapy (PIT) to CD25-expressing cells. First, binding of anti-hCD25-IR700 to CD25-positive KIT-225 cells (a human T cell line) was tested. KIT-225 cells (0.5×10⁶) were incubated with 1 μg of anti-hCD25 antibody (14564) conjugated to IR700 (anti-hCD25-IR700). As a blocking control, cells were incubated with 10 μg non-conjugated antibody prior to anti-hCD25-IR700 incubation. Cells were stained with viability dye and analyzed by fluorescence-activated cells sorting (FACS). Unstained cells were used as a negative control. The results demonstrate that conjugation of IR700 to anti-CD25 antibody did not diminish binding of the antibody to CD25-expressing cells (FIG. 5A).

Cell viability after hCD25-targeted PIT was also evaluated. KIT-225 cells (0.5×10⁶) were incubated with 1 μg of anti-hCD25-IR700 conjugate, followed by application of 0, 5, 20 or 50 J/cm² NIR light at 150 mW/cm². Cells were then stained with viability dye and analyzed by FACS. As shown in FIG. 5C, significant CD25-positive cell death occurred when cells were incubated with anti-hCD25-IR700 conjugate and exposed to 5, 20 or 50 J/cm² NIR light. FIG. 5B shows representative flow cytometry plots of live/dead staining after PIT using 0, 5 or 20 J/cm² NIR light.

### Example 5: Anti-CD25 Antibodies do not Block IL-2 Binding to CD25

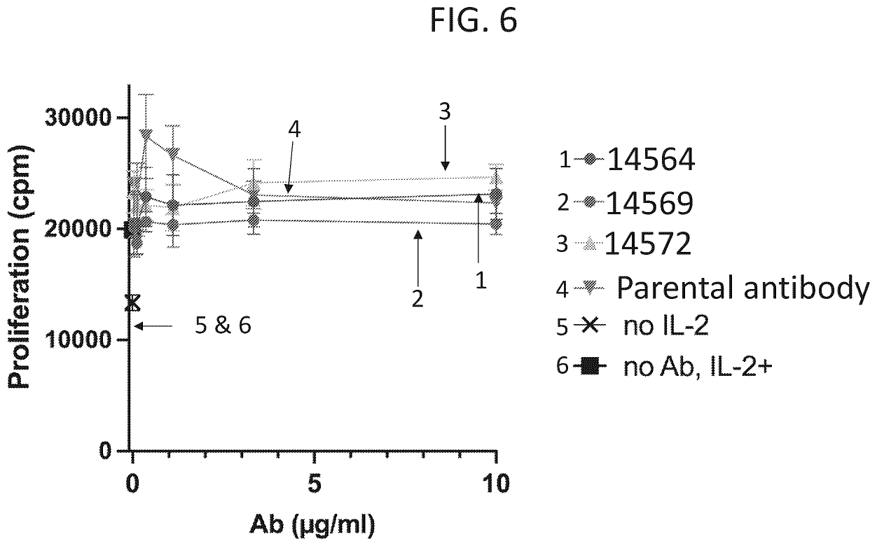

To test whether antibodies 14564, 14569, 14572 block binding of IL-2 to CD25, a proliferation assay was performed. KIT-225 cells underwent IL-2 fasting for five days prior to performing the proliferation assay. Fifty thousand fasted cells in culture medium were pre-incubated with 0, 5 or 10 μg/ml of 14564, 14569, 14572 or the parental anti-CD25 antibody for 30 minutes at 37° C., and then cultured with or without 50 pM human IL-2 for 1.5 days. Cells cultured without antibody and with or without IL-2 were included as controls. ³H-thymidine (37 kBq) was pulsed, and 4 hours later, the cells were harvested, and the incorporated activity was measured using a β-counter. As shown in FIG. 6, antibodies 14564, 14569, 14572 do not inhibit IL-2-induced proliferation of KIT-225, which demonstrates that these antibodies do not block binding of IL-2 to CD25.

### Example 6: Human CD25-Targeted PIT

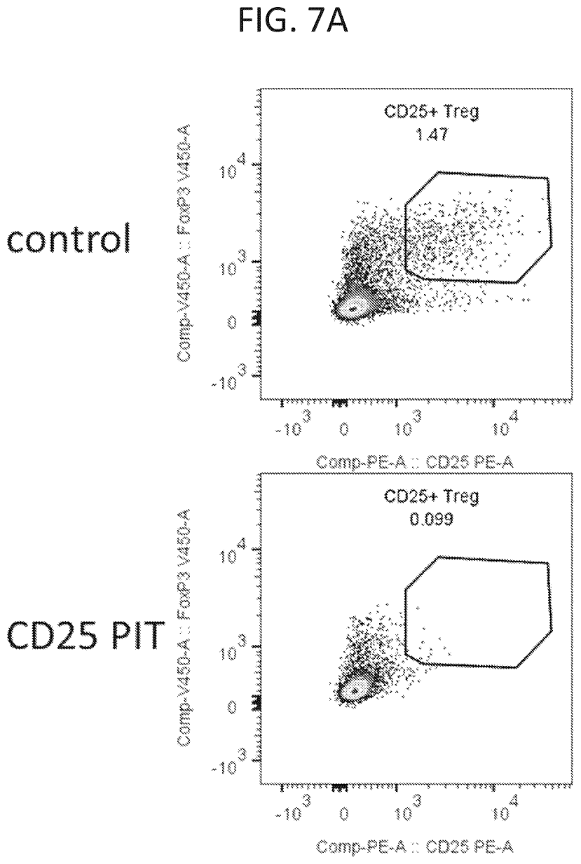

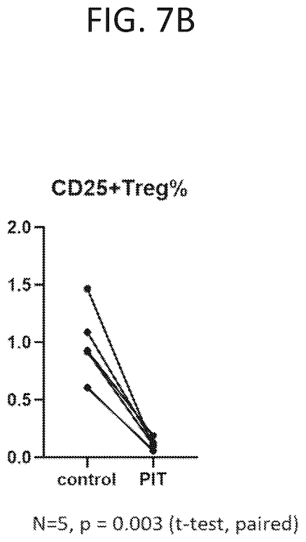

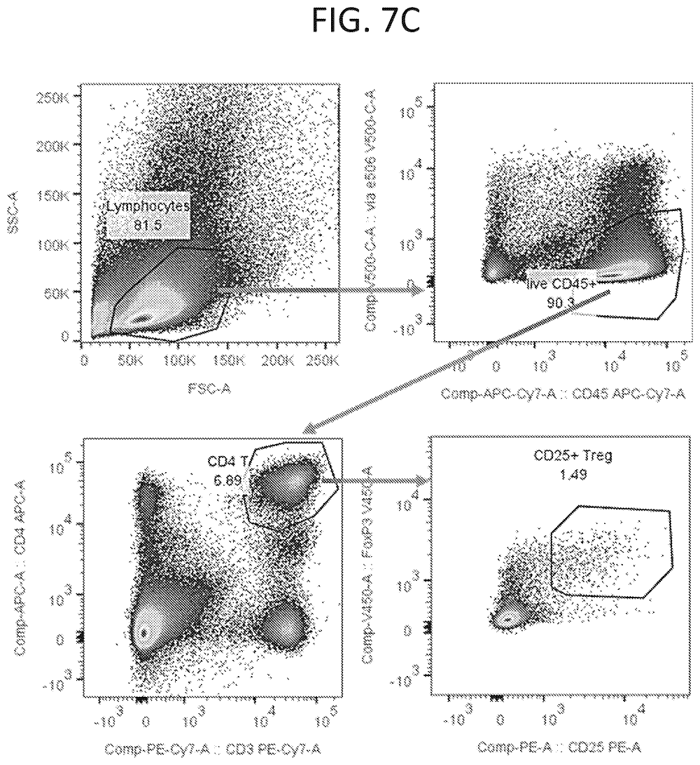

This example describes a study performed to determine whether hCD25-targeted PIT can kill human Treg cells. Splenocytes obtained from MDA-MD 231 tumor-bearing hCD34-NSG humanized mice were incubated with 5 g IR700-conjugated anti-CD25 antibody 14564 and then irradiated with 50 J/cm² NIR light at 150 mW/cm². Splenocytes cultured in the absence of antibody-IR700 conjugate and not irradiated were used as a control. The percentage of CD25+ Treg cells was evaluated by flow cytometry. The gating strategy for this study is shown in FIG. 7C and representative dot plots of CD25+ Treg cells in control and CD25 PIT splenocyte samples are shown in FIG. 7A. As shown in FIG. 7B, the percentage of CD25+ Treg cells present in the CD25 PIT splenocyte samples was significantly reduced compared to control splenocytes, indicating that hCD25-targeted PIT is effective for depleting human CD25-positive Treg cells.

### Example 7: Depletion of CD25-Expressing Treg Cells in a Humanized Mouse Model

This example describes an exemplary method to test the ability of anti-hCD25-IR700 NIR-PIT to deplete CD25-positive Treg cells in vivo using a mouse model with a human immune system (e.g., a humanized mouse model as described in WO 2019/199799; U.S. Publication No. 2019/0320633; Yin et al., Am J Cancer Res 10(12):4568-4584, 2020; or Serr et al., Front Immunol 12:643544, 2021).

Humanized mice are administered (such as by intravenous, intratumoral, intraperitoneal, or intrapleural administration) an anti-hCD25-IR700 conjugate (such as a conjugate in which the antibody includes the CDR sequences or VH/VL domain sequences of antibody 14564, 14569 or 14572) at a dose of g (or another suitable dose as described herein). The humanized mice are then exposed to NIR light (such as 50 J/cm² NIR light or another suitable dose as described herein) at a wavelength of about 660 to about 740 nm. Depletion of Treg cells in treated and untreated mice is evaluated by flow cytometry. It is expected that treatment with anti-hCD25-IR700 NIR-PIT will result in a significant reduction in the number of Treg cells compared to untreated control mice.

It will be apparent that the precise details of the methods or compositions described may be varied or modified without departing from the spirit of the described aspects of the disclosure. We claim all such modifications and variations that fall within the scope and spirit of the claims below.

1. A monoclonal antibody that specifically binds CD25, comprising a variable heavy (VH) domain and a variable light (VL) domain, wherein:
the VH domain comprises a complementarity determining region 1 (CDR1), CDR2 and CDR3, and the amino acid sequences of the VH domain CDR1, CDR2 and CDR3 respectively comprise SEQ ID NO: 49, SEQ ID NO: 51 and SEQ ID NO: 52; and the VL domain comprises a CDR1, CDR2 and CDR3, and the amino amino acid sequences of the CDR1, CDR2 and CDR3 respectively comprise SEQ ID NO: 54, SEQ ID NO: 55 and SEQ ID NO: 48;
the VH domain comprises a CDR1, CDR2 and CDR3 and the amino acid sequences of the VH domain CDR1, CDR2 and CDR3 respectively comprise SEQ ID NO: 49, SEQ ID NO: 54, SEQ ID NO: 56 and SEQ ID NO: 48; or
the VH domain comprises a CDR1, CDR2 and CDR3 and the amino acid sequences of the VH domain CDR1, CDR2 and CDR3 respectively comprise SEQ ID NO: 50, SEQ ID NO: 51, and SEQ ID NO: 53; and the VL domain comprises a CDR1, CDR2 and CDR3, and the amino acid sequences of the VL domain CDR1, CDR2 and CDR3 respectively comprise SEQ ID NO: 54, SEQ ID NO: 56, and SEQ ID NO: 48.

4. (canceled)

5. The monoclonal antibody of claim 1, wherein:
the amino acid sequence of the VH domain is at least 90% identical to SEQ ID NO: 31 and the VH domain CDR1, CDR2 and CDR3 respectively comprise SEQ ID NO: 49, SEQ ID NO: 51 and SEQ ID NO: 52; and/or
the amino acid sequence of the VL domain is at least 90% identical to SEQ ID NO: 32 and the VL domain CDR1, CDR2 and CDR3 respectively comprise SEQ ID NO: 54, SEQ ID NO: 56 and SEQ ID NO: 48.

6. (canceled)

7. The monoclonal antibody of claim 1, wherein:
the amino acid sequence of the VH domain is at least 90% identical to SEQ ID NO: 37 and the VH domain CDR1, CDR2 and CDR3 respectively comprise SEQ ID NO: 50, SEQ ID NO: 51 and SEQ ID NO: 53; and/or
the amino acid sequence of the VL domain is at least 90% identical to SEQ ID NO: 38 and the VL domain CDR1, CDR2 and CDR3 respectively comprise SEQ ID NO: 54, SEQ ID NO: 56 and SEQ ID NO: 48.

8. The monoclonal antibody of claim 1, wherein:
the amino acid sequence of the VH domain comprises SEQ ID NO: 21 and the amino acid sequence of the VL domain comprises SEQ ID NO: 22;
the amino acid sequence of the VH domain comprises SEQ ID NO: 31 and the amino acid sequence of the VL domain comprises SEQ ID NO: 32;
the amino acid sequence of the VH domain comprises SEQ ID NO: 37 and the amino acid sequence of the VL domain comprises SEQ ID NO: 38;
the amino acid sequence of the VH domain comprises SEQ ID NO: 1 and the amino acid sequence of the VL domain comprises SEQ ID NO: 2;
the amino acid sequence of the VH domain comprises SEQ ID NO: 3 and the amino acid sequence of the VL domain comprises SEQ ID NO: 4;
the amino acid sequence of the VH domain comprises SEQ ID NO: 5 and the amino acid sequence of the VL domain comprises SEQ ID NO: 6;
the amino acid sequence of the VH domain comprises SEQ ID NO: 7 and the amino acid sequence of the VL domain comprises SEQ ID NO: 8;
the amino acid sequence of the VH domain comprises SEQ ID NO: 9 and the amino acid sequence of the VL domain comprises SEQ ID NO: 10;
the amino acid sequence of the VH domain comprises SEQ ID NO: 11 and the amino acid sequence of the VL domain comprises SEQ ID NO: 12;
the amino acid sequence of the VH domain comprises SEQ ID NO: 13 and the amino acid sequence of the VL domain comprises SEQ ID NO: 14;
the amino acid sequence of the VH domain comprises SEQ ID NO: 15 and the amino acid sequence of the VL domain comprises SEQ ID NO: 16;
the amino acid sequence of the VH domain comprises SEQ ID NO: 17 and the amino acid sequence of the VL domain comprises SEQ ID NO: 18;
the amino acid sequence of the VH domain comprises SEQ ID NO: 19 and the amino acid sequence of the VL domain comprises SEQ ID NO: 20;
the amino acid sequence of the VH domain comprises SEQ ID NO: 23 and the amino acid sequence of the VL domain comprises SEQ ID NO: 24;
the amino acid sequence of the VH domain comprises SEQ ID NO: 25 and the amino acid sequence of the VL domain comprises SEQ ID NO: 26;
the amino acid sequence of the VH domain comprises SEQ ID NO: 27 and the amino acid sequence of the VL domain comprises SEQ ID NO: 28;
the amino acid sequence of the VH domain comprises SEQ ID NO: 29 and the amino acid sequence of the VL domain comprises SEQ ID NO: 30;
the amino acid sequence of the VH domain comprises SEQ ID NO: 33 and the amino acid sequence of the VL domain comprises SEQ ID NO: 34;
the amino acid sequence of the VH domain comprises SEQ ID NO: 35 and the amino acid sequence of the VL domain comprises SEQ ID NO: 36;
the amino acid sequence of the VH domain comprises SEQ ID NO: 39 and the amino acid sequence of the VL domain comprises SEQ ID NO: 40; or
the amino acid sequence of the VH domain comprises SEQ ID NO: 41 and the amino acid sequence of the VL domain comprises SEQ ID NO: 42.

9. The monoclonal antibody of claim 1, wherein the monoclonal antibody is an antigen-binding fragment selected from a Fab fragment, a Fab′ fragment, an F(ab)′2 fragment, a single chain variable fragment (scFv) and a disulfide stabilized variable fragment (dsFv).

10. The monoclonal antibody of claim 1, further comprising a constant region.

11. The monoclonal antibody of claim 10, wherein the constant region comprises at least one modification to increase half-life, stability and/or function of the monoclonal antibody.

12. The monoclonal antibody of claim 10, wherein the constant region comprises a heavy chain constant region and/or a light chain constant region.

13. The monoclonal antibody of claim 12, wherein the heavy chain constant region is a human IgG4 constant region comprising SEQ ID NO: 58.

14. The monoclonal antibody of claim 12, wherein the light chain constant region is a human Igκ light chain comprising SEQ ID NO: 59.

15. The monoclonal antibody of claim 1, comprising:
SEQ ID NO: 80 and SEQ ID NO: 81;
SEQ ID NO: 90 and SEQ ID NO: 91;
SEQ ID NO: 96 and SEQ ID NO: 97;
SEQ ID NO: 60 and SEQ ID NO: 61;
SEQ ID NO: 62 and SEQ ID NO: 63;
SEQ ID NO: 64 and SEQ ID NO: 65;
SEQ ID NO: 66 and SEQ ID NO: 67;
SEQ ID NO: 68 and SEQ ID NO: 69;
SEQ ID NO: 70 and SEQ ID NO: 71;
SEQ ID NO: 72 and SEQ ID NO: 73;
SEQ ID NO: 74 and SEQ ID NO: 75;
SEQ ID NO: 76 and SEQ ID NO: 77;
SEQ ID NO: 78 and SEQ ID NO: 79;
SEQ ID NO: 82 and SEQ ID NO: 83;
SEQ ID NO: 84 and SEQ ID NO: 85;
SEQ ID NO: 86 and SEQ ID NO: 87;
SEQ ID NO: 88 and SEQ ID NO: 89;
SEQ ID NO: 92 and SEQ ID NO: 93;
SEQ ID NO: 94 and SEQ ID NO: 95;
SEQ ID NO: 98 and SEQ ID NO: 99; or
SEQ ID NO: 100 and SEQ ID NO: 101.

16. The monoclonal antibody of claim 1, wherein:
(i) the antibody does not significantly inhibit binding of interleukin-2 (IL-2) to CD25;
(ii) the antibody induces little to no antibody-dependent cellular cytotoxicity (ATCC); or
(iii) both (i) and (ii).

17. A conjugate, comprising:
the monoclonal antibody of claim 1; and
a photon absorber, an effector molecule or a detectable label.

18. The conjugate of claim 17, wherein the photon absorber is IR700.

19. The conjugate of claim 17, wherein the effector molecule is a toxin.

20. The conjugate of claim 17, wherein the detectable label is a fluorescent, enzymatic, or radioactive label.

21. A composition, comprising:
the monoclonal antibody of claim 1; and
a pharmaceutically acceptable carrier.

22. A nucleic acid molecule encoding the monoclonal antibody of claim 1, or encoding a VH domain or a VL domain thereof.

23. The nucleic acid molecule of claim 22, comprising:
the nucleotide sequence of SEQ ID NO: 122, or a degenerate variant thereof;
the nucleotide sequence of SEQ ID NO: 123, or a degenerate variant thereof;
the nucleotide sequences of SEQ ID NO: 122 and SEQ ID NO: 123, or degenerate variants thereof;
the nucleotide sequence of SEQ ID NO: 132, or a degenerate variant thereof;
the nucleotide sequence of SEQ ID NO: 133, or a degenerate variant thereof;
the nucleotide sequences of SEQ ID NO: 132 and SEQ ID NO: 133, or degenerate variants thereof;
the nucleotide sequence of SEQ ID NO: 138, or a degenerate variant thereof;
the nucleotide sequence of SEQ ID NO: 139, or a degenerate variant thereof;
the nucleotide sequences of SEQ ID NO: 138 and SEQ ID NO: 139, or degenerate variants thereof;
the nucleotide sequence of SEQ ID NO: 168, or a degenerate variant thereof;
the nucleotide sequence of SEQ ID NO: 169, or a degenerate variant thereof;
the nucleotide sequences of SEQ ID NO: 168 and SEQ ID NO: 169, or degenerate variants thereof;
the nucleotide sequence of SEQ ID NO: 178, or a degenerate variant thereof;
the nucleotide sequence of SEQ ID NO: 179, or a degenerate variant thereof;
the nucleotide sequences of SEQ ID NO: 178 and SEQ ID NO: 179, or degenerate variants thereof;
the nucleotide sequence of SEQ ID NO: 184, or a degenerate variant thereof;
the nucleotide sequence of SEQ ID NO: 185, or a degenerate variant thereof; or
the nucleotide sequences of SEQ ID NO: 184 and SEQ ID NO: 185, or degenerate variants thereof.

24. A vector comprising the nucleic acid molecule of claim 22.

25. An isolated host cell comprising the vector of claim 24.

26. A kit comprising:
(i) the monoclonal antibody of claim 1; and
(ii) buffer, cell culture media, one or more check point inhibitors, one or more immunotherapies, one or more additional anti-cancer reagents, one or more reducing agents, one or more transfection reagents and/or instructional materials.

27. A method for treating cancer in a subject, comprising:
administering to the subject a therapeutically effective amount of the conjugate of claim 18; and
subsequently irradiating the subject and/or irradiating cancer cells in the subject at a wavelength of 660 to 740 nm and at a dose of at least 1 J/cm2,
thereby treating cancer in the subject.

28. The method of claim 27, wherein the conjugate is administered at or near the site of the cancer.

29. The method of claim 27, wherein administration of the conjugate is not systemic.

30. The method of claim 27, wherein administration of the conjugate is systemic.

31. The method of claim 27, further comprising administering a second monoclonal antibody conjugated to IR700, wherein the second monoclonal antibody specifically binds a tumor antigen expressed by cells of the cancer.

32. The method of claim 31, wherein the tumor antigen is selected from epidermal growth factor receptor (EGFR/HER1), mesothelin, prostate specific membrane antigen (PSMA), HER2/ERBB2, CD3, CD18, CD20, CD25 (IL-2Rα receptor), CD30, CD33, CD44, CD52, CD133, CD206, carcinoembryonic antigen (CEA), alpha-fetoprotein (AFP), Lewis Y, tumor-associated glycoprotein 72 (TAG72), vascular endothelial growth factor (VEGF), VEGF receptor (VEGFR), epithelial cell adhesion molecule (EpCAM), ephrin type-A receptor 2 (EphA2), glypican-1 (GPC1), glypican-2 (GPC2), glypican-3 (GPC3), gpA33, a mucin, CAIX, a folate-binding protein, a ganglioside, integrin αVβ3, integrin α5β3,1, Erb-B2 receptor tyrosine kinase 3 (ERBB3), MET proto-oncogene, receptor tyrosine kinase (MET), insulin like growth factor 1 receptor (IGF1R), ephrin type-A receptor 3 (EPHA3), tumor necrosis factor-related apoptosis-inducing ligand receptor 1 (TRAILR1), TRAILR2, receptor activator of nuclear factor kappa-B ligand (RANKL), fibroblast activation protein (FAP), tenascin, BCR complex, gp72, HLA-DR 10β, HLA-DR antigen, IgE, CA 242, polymorphic epithelial mucin (PEM) antigen, SK-1 antigen, programmed death 1 (PD-1), and programmed death ligand 2 (PD-L2).

33. The method of claim 31, wherein both the conjugate and the second monoclonal antibody conjugated to IR700 are administered prior to irradiation.

34. The method of claim 27, further comprising administering to the subject one or more additional cancer therapies.

35. The method of claim 34, wherein the one or more additional cancer therapies comprises an anti-CTLA4-IR700 conjugate, an anti-PD-L1-IR700 conjugate, an immunoactivator, a check point inhibitor, chemotherapy, radiotherapy and/or surgery.

36. The method of claim 31, wherein the conjugate and second monoclonal antibody conjugated to IR700 are administered intravenously.

37. The method of claim 27, wherein the cancer is a cancer of the breast, liver, lung, esophagus, stomach, colon, ovary, prostate, pancreas, brain, cervix, kidney, bone, skin, head and neck, oropharynx or blood.

38-40. (canceled)


SEQUENCE LISTING (189 sequences)

SEQ ID NO: 1 - protein, 123 aa, synthetic construct
EVQLVESGGGLVQPGGSLRLSCAASGFWFSSYGMSWVRQAPGKGLELVSTINGYGDTWYYPDSVKGRFTISRDNSKNTLYLQMNSLRAEDTAVYYCARDRDYTNSYYYAHDYWGQGTLVTVSS

SEQ ID NO: 2 - protein, 106 aa, synthetic construct
EIVLTQSPATLSLSPGERATLSCRASSYVSFMHWLQQKPGQAPRPWIYATANLASGVPARFSGSGSGTDYTLTISSLEPEDFAVYYCQQWSSNPPAFGGGTKVEIK

SEQ ID NO: 3 - protein, 123 aa, synthetic construct
EVQLVESGGGLVQPGGSLRLSCAASGFTFSSWGMSWVRQAPGKGLELVSTINGYGDTTYYPDSVKGRFTISRDNSKNTLYLQMNSLRAEDTAVYYCARDRDYTNSYYYAHDYWGQGTLVTVSS

SEQ ID NO: 4 - protein, 106 aa, synthetic construct; identical to SEQ ID NO: 2

SEQ ID NO: 5 - protein, 123 aa, synthetic construct
EVQLVESGGGLVQPGGSLRLSCAASGYTFSSYGMSWVRQAPGKGLELVSTINGYGDTTYYPDSVKGRFTISRDNSKNTLYLQMNSLRAEDTAVYYCARDRDYSNSYYYALDYWGQGTLVTVSS

SEQ ID NO: 6 - protein, 106 aa, synthetic construct; identical to SEQ ID NO: 2

SEQ ID NO: 7 - protein, 123 aa, synthetic construct
EVQLVESGGGLVQPGGSLRLSCAASGYTFSSYGMSWVRQAPGKGLELVSTINGYGDTWYYPDSVKGRFTISRDNSKNTLYLQMNSLRAEDTAVYYCARDRDYSNSYYYAHDYWGQGTLVTVSS

SEQ ID NO: 8 - protein, 106 aa, synthetic construct
EIVLTQSPATLSLSPGERATLSCRASSWVSFMHWLQQKPGQAPRPWIYATQNLASGVPARFIGSGSGTDYTLTISSLEPEDFAVYYCQQWSSNPPAFGGGTKVEIK

SEQ ID NO: 9 - protein, 123 aa, synthetic construct
EVQLVESGGGLVQPGGSLRLSCAASGYWFSSYGMSWVRQAPGKGLELVSTINGYGDTTYYPDSVKGRFTISRDNSKNTLYLQMNSLRAEDTAVYYCARDRDYTNSYYYAHDYWGQGTLVTVSS

SEQ ID NO: 10 - protein, 106 aa, synthetic construct
EIVLTQSPATLSLSPGERATLSCRASSSVSFMHWLQQKPGQAPRPWIYATMNLASGVPARFSGSGSGTDYTLTISSLEPEDFAVYYCQQWSSNPPAFGGGTKVEIK

SEQ ID NO: 11 - protein, 123 aa, synthetic construct
EVQLVESGGGLVQPGGSLRLSCAASGFTFSSYGMSWVRQAPGKGLELVSTINGYGDTTYYPDSVKGRFTISRDNSKNTLYLQMNSLRAEDTAVYYCARDRDYSNSYYYAHDYWGQGTLVTVSS

SEQ ID NO: 12 - protein, 106 aa, synthetic construct
EIVLTQSPATLSLSPGERATLSCRASSWVSFMHWLQQKPGQAPRPWIYATQNLASGVPARFSGSGSGTDYTLTISSLEPEDFAVYYCQQWSSNPPAFGGGTKVEIK

SEQ ID NO: 13 - protein, 123 aa, synthetic construct
EVQLVESGGGLVQPGGSLRLSCAASGFTFSSYGMSWVRQAPGKGLELVSTINGYGDTTYYPDSVKGRFTISRDNSKNTLYLQMNSLRAEDTAVYYCARDRDYTNSYYYAHDYWGQGTLVTVSS

SEQ ID NO: 14 - protein, 106 aa, synthetic construct
EIVLTQSPATLSLSPGERATLSCRASSSVSFMHWLQQKPGQAPRPWIYATQNLASGVPARFSGSGSGTDYTLTISSLEPEDFAVYYCQQWSSNPPAFGGGTKVEIK

SEQ ID NO: 15 - protein, 123 aa, synthetic construct
EVQLVESGGGLVQPGGSLRLSCAASGYTFSSWGMSWVRQAPGKGLELVSTINGYGDTWYYPDSVKGRFTISRDNSKNTLYLQMNSLRAEDTAVYYCARDRDYSNSYYYAHDYWGQGTLVTVSS

SEQ ID NO: 16 - protein, 106 aa, synthetic construct
EIVLTQSPATLSLSPGERATLSCRASSYVSFMHWLQQKPGQAPRPWIYATMNLASGVPARFSGSGSGTDYTLTISSLEPEDFAVYYCQQWSSNPPAFGGGTKVEIK

SEQ ID NO: 17 - protein, 123 aa, synthetic construct
EVQLVESGGGLVQPGGSLRLSCAASGFTFSSYGMSWVRQAPGKGLELVSTINGYGDTWYYPDSVKGRFTISRDNSKNTLYLQMNSLRAEDTAVYYCARDRDYSNSYYYAHDYWGQGTLVTVSS

SEQ ID NO: 18 - protein, 106 aa, synthetic construct; identical to SEQ ID NO: 10

SEQ ID NO: 19 - protein, 123 aa, synthetic construct
EVQLVESGGGLVQPGGSLRLSCAASGYTFSSYGMSWVRQAPGKGLELVSTINGYGDTTYYPDSVKGRFTISRDNSKNTLYLQMNSLRAEDTAVYYCARDRDYGNSYYYAHDYWGQGTLVTVSS

SEQ ID NO: 20 - protein, 106 aa, synthetic construct; identical to SEQ ID NO: 16

SEQ ID NO: 21 - protein, 123 aa, synthetic construct
EVQLVESGGGLVQPGGSLRLSCAASGFTFSSWGMSWVRQAPGKGLELVSTINGYGDTTYYPDSVKGRFTISRDNSKNTLYLQMNSLRAEDTAVYYCARDRDYSNSYYYAHDYWGQGTLVTVSS

SEQ ID NO: 22 - protein, 106 aa, synthetic construct; identical to SEQ ID NO: 2

SEQ ID NO: 23 - protein, 123 aa, synthetic construct
EVQLVESGGGLVQPGGSLRLSCAASGFTFSSYGMSWVRQAPGKGLELVSTINGYGDTWYYPDSVKGRFTISRDNSKNTLYLQMNSLRAEDTAVYYCARDRDYGNSYYYAHDYWGQGTLVTVSS

SEQ ID NO: 24 - protein, 106 aa, synthetic construct; identical to SEQ ID NO: 12

SEQ ID NO: 25 - protein, 123 aa, synthetic construct
EVQLVESGGGLVQPGGSLRLSCAASGFTFSSYGMSWVRQAPGKGLELVSTINGYGDTTYYPDSVKGRFTISRDNSKNTLYLQMNSLRAEDTAVYYCARDRDYGNSYYYAHDYWGQGTLVTVSS

SEQ ID NO: 26 - protein, 106 aa, synthetic construct
EIVLTQSPATLSLSPGERATLSCRASSWVSFMHWLQQKPGQAPRPWIYATMNLASGVPARFSGSGSGTDYTLTISSLEPEDFAVYYCQQWSSNPPAFGGGTKVEIK

SEQ ID NO: 27 - protein, 123 aa, synthetic construct
EVQLVESGGGLVQPGGSLRLSCAASGFWFSSWGMSWVRQAPGKGLELVSTINGYGDTTYYPDSVKGRFTISRDNSKNTLYLQMNSLRAEDTAVYYCARDRDYTNSYYYAHDYWGQGTLVTVSS

SEQ ID NO: 28 - protein, 106 aa, synthetic construct
EIVLTQSPATLSLSPGERATLSCRASSYVSFMHWLQQKPGQAPRPWIYATQNLASGVPARFSGSGSGTDYTLTISSLEPEDFAVYYCQQWSSNPPAFGGGTKVEIK

SEQ ID NO: 29 - protein, 123 aa, synthetic construct; identical to SEQ ID NO: 13

SEQ ID NO: 30 - protein, 106 aa, synthetic construct; identical to SEQ ID NO: 16

SEQ ID NO: 31 - protein, 123 aa, synthetic construct
EVQLVESGGGLVQPGGSLRLSCAASGYWFSSWGMSWVRQAPGKGLELVSTINGYGDTTYYPDSVKGRFTISRDNSKNTLYLQMNSLRAEDTAVYYCARDRDYSNSYYYAHDYWGQGTLVTVSS

SEQ ID NO: 32 - protein, 106 aa, synthetic construct; identical to SEQ ID NO: 16

SEQ ID NO: 33 - protein, 123 aa, synthetic construct
EVQLVESGGGLVQPGGSLRLSCAASGFWFSSYGMSWVRQAPGKGLELVSTINGYGDTTYYPDSVKGRFTISRDNSKNTLYLQMNSLRAEDTAVYYCARDRDYTNSYYYAHDYWGQGTLVTVSS

SEQ ID NO: 34 - protein, 106 aa, synthetic construct
EIVLTQSPATLSLSPGERATLSCRASSSVSFMHWLQQKPGQAPRPWIYATANLASGVPARFSGSGSGTDYTLTISSLEPEDFAVYYCQQWSSNPPAFGGGTKVEIK

SEQ ID NO: 35 - protein, 123 aa, synthetic construct; identical to SEQ ID NO: 19

SEQ ID NO: 36 - protein, 106 aa, synthetic construct; identical to SEQ ID NO: 26

SEQ ID NO: 37 - protein, 123 aa, synthetic construct; identical to SEQ ID NO: 9

SEQ ID NO: 38 - protein, 106 aa, synthetic construct; identical to SEQ ID NO: 16

SEQ ID NO: 39 - protein, 123 aa, synthetic construct; identical to SEQ ID NO: 3

SEQ ID NO: 40 - protein, 106 aa, synthetic construct
EIVLTQSPATLSLSPGERATLSCRASSYVSFMHWLQQKPGQAPRPWIYATSNLASGVPARFSGSGSGTDYTLTISSLEPEDFAVYYCQQWSSNPPAFGGGTKVEIK

SEQ ID NO: 41 - protein, 123 aa, synthetic construct
EVQLVESGGGLVQPGGSLRLSCAASGFTFSSYGMSWVRQAPGKGLELVSTINGYGDTTYYPDSVKGRFTISRDNSKNTLYLQMNSLRAEDTAVYYCARDRDYGNSYYYALDYWGQGTLVTVSS

SEQ ID NO: 42 - protein, 106 aa, synthetic construct
EIVLTQSPATLSLSPGERATLSCRASSSVSFMHWLQQKPGQAPRPWIYATSNLASGVPARFSGSGSGTDYTLTISSLEPEDFAVYYCQQWSSNPPAFGGGTKVEIK

SEQ ID NO: 43 - protein, 5 aa, synthetic construct
  VARIANT 2: note = X is W or Y
SXGMS

SEQ ID NO: 44 - protein, 17 aa, synthetic construct
  VARIANT 9: note = X is W or T
TINGYGDTXYYPDSVKG

SEQ ID NO: 45 - protein, 14 aa, synthetic construct
  VARIANT 5: note = X is S or G
  VARIANT 12: note = X is L or H
DRDYXNSYYYAXDY

SEQ ID NO: 46 - protein, 10 aa, synthetic construct
  VARIANT 5: note = X is S, W or Y
RASSXVSFMH

SEQ ID NO: 47 - protein, 7 aa, synthetic construct
  VARIANT 3: note = X is S, A, Q or M
ATXNLAS

SEQ ID NO: 48 - protein, 9 aa, synthetic construct
QQWSSNPPA

SEQ ID NO: 49 - protein, 5 aa, synthetic construct
SWGMS

SEQ ID NO: 50 - protein, 5 aa, synthetic construct
SYGMS

SEQ ID NO: 51 - protein, 17 aa, synthetic construct
TINGYGDTTYYPDSVKG

SEQ ID NO: 52 - protein, 14 aa, synthetic construct
DRDYSNSYYYAHDY

SEQ ID NO: 53 - protein, 14 aa, synthetic construct
DRDYTNSYYYAHDY

SEQ ID NO: 54 - protein, 10 aa, synthetic construct
RASSYVSFMH

SEQ ID NO: 55 - protein, 7 aa, synthetic construct
ATANLAS

SEQ ID NO: 56 - protein, 7 aa, synthetic construct
ATMNLAS

SEQ ID NO: 57 - protein, 19 aa, synthetic construct
MGWSCIILFLVATATGVHS

SEQ ID NO: 58 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 107 aa, Homo sapiens [sequence omitted]

SEQ ID NO: 60 - protein, 469 aa, synthetic construct
MGWSCIILFLVATATGVHSEVQLVESGGGLVQPGGSLRLSCAASGFWFSSYGMSWVRQAPGKGLELVSTINGYGDTWYYPDSVKGRFTISRDNSKNTLYLQMNSLRAEDTAVYYCARDRDYTNSYYYAHDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 61 - protein, 232 aa, synthetic construct
MGWSCIILFLVATATGVHSEIVLTQSPATLSLSPGERATLSCRASSYVSFMHWLQQKPGQAPRPWIYATANLASGVPARFSGSGSGTDYTLTISSLEPEDFAVYYCQQWSSNPPAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 469 aa, synthetic construct
MGWSCIILFLVATATGVHSEVQLVESGGGLVQPGGSLRLSCAASGFTFSSWGMSWVRQAPGKGLELVSTINGYGDTTYYPDSVKGRFTISRDNSKNTLYLQMNSLRAEDTAVYYCARDRDYTNSYYYAHDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 63 - protein, 232 aa, synthetic construct; identical to SEQ ID NO: 61

SEQ ID NO: 64 - protein, 469 aa, synthetic construct
MGWSCIILFLVATATGVHSEVQLVESGGGLVQPGGSLRLSCAASGYTFSSYGMSWVRQAPGKGLELVSTINGYGDTTYYPDSVKGRFTISRDNSKNTLYLQMNSLRAEDTAVYYCARDRDYSNSYYYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 65 - protein, 232 aa, synthetic construct; identical to SEQ ID NO: 61

SEQ ID NO: 66 - protein, 469 aa, synthetic construct
MGWSCIILFLVATATGVHSEVQLVESGGGLVQPGGSLRLSCAASGYTFSSYGMSWVRQAPGKGLELVSTINGYGDTWYYPDSVKGRFTISRDNSKNTLYLQMNSLRAEDTAVYYCARDRDYSNSYYYAHDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 67 - protein, 232 aa, synthetic construct
MGWSCIILFLVATATGVHSEIVLTQSPATLSLSPGERATLSCRASSWVSFMHWLQQKPGQAPRPWIYATQNLASGVPARFIGSGSGTDYTLTISSLEPEDFAVYYCQQWSSNPPAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 469 aa, synthetic construct
MGWSCIILFLVATATGVHSEVQLVESGGGLVQPGGSLRLSCAASGYWFSSYGMSWVRQAPGKGLELVSTINGYGDTTYYPDSVKGRFTISRDNSKNTLYLQMNSLRAEDTAVYYCARDRDYTNSYYYAHDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 69 - protein, 232 aa, synthetic construct
MGWSCIILFLVATATGVHSEIVLTQSPATLSLSPGERATLSCRASSSVSFMHWLQQKPGQAPRPWIYATMNLASGVPARFSGSGSGTDYTLTISSLEPEDFAVYYCQQWSSNPPAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 469 aa, synthetic construct
MGWSCIILFLVATATGVHSEVQLVESGGGLVQPGGSLRLSCAASGFTFSSYGMSWVRQAPGKGLELVSTINGYGDTTYYPDSVKGRFTISRDNSKNTLYLQMNSLRAEDTAVYYCARDRDYSNSYYYAHDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 71 - protein, 232 aa, synthetic construct
MGWSCIILFLVATATGVHSEIVLTQSPATLSLSPGERATLSCRASSWVSFMHWLQQKPGQAPRPWIYATQNLASGVPARFSGSGSGTDYTLTISSLEPEDFAVYYCQQWSSNPPAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 469 aa, synthetic construct
MGWSCIILFLVATATGVHSEVQLVESGGGLVQPGGSLRLSCAASGFTFSSYGMSWVRQAPGKGLELVSTINGYGDTTYYPDSVKGRFTISRDNSKNTLYLQMNSLRAEDTAVYYCARDRDYTNSYYYAHDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 73 - protein, 232 aa, synthetic construct
MGWSCIILFLVATATGVHSEIVLTQSPATLSLSPGERATLSCRASSSVSFMHWLQQKPGQAPRPWIYATQNLASGVPARFSGSGSGTDYTLTISSLEPEDFAVYYCQQWSSNPPAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 469 aa, synthetic construct
MGWSCIILFLVATATGVHSEVQLVESGGGLVQPGGSLRLSCAASGYTFSSWGMSWVRQAPGKGLELVSTINGYGDTWYYPDSVKGRFTISRDNSKNTLYLQMNSLRAEDTAVYYCARDRDYSNSYYYAHDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 75 - protein, 232 aa, synthetic construct
MGWSCIILFLVATATGVHSEIVLTQSPATLSLSPGERATLSCRASSYVSFMHWLQQKPGQAPRPWIYATMNLASGVPARFSGSGSGTDYTLTISSLEPEDFAVYYCQQWSSNPPAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 469 aa, synthetic construct
MGWSCIILFLVATATGVHSEVQLVESGGGLVQPGGSLRLSCAASGFTFSSYGMSWVRQAPGKGLELVSTINGYGDTWYYPDSVKGRFTISRDNSKNTLYLQMNSLRAEDTAVYYCARDRDYSNSYYYAHDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 77 - protein, 232 aa, synthetic construct; identical to SEQ ID NO: 69

SEQ ID NO: 78 - protein, 469 aa, synthetic construct
MGWSCIILFLVATATGVHSEVQLVESGGGLVQPGGSLRLSCAASGYTFSSYGMSWVRQAPGKGLELVSTINGYGDTTYYPDSVKGRFTISRDNSKNTLYLQMNSLRAEDTAVYYCARDRDYGNSYYYAHDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 79 - protein, 232 aa, synthetic construct; identical to SEQ ID NO: 75

SEQ ID NO: 80 - protein, 469 aa, synthetic construct
MGWSCIILFLVATATGVHSEVQLVESGGGLVQPGGSLRLSCAASGFTFSSWGMSWVRQAPGKGLELVSTINGYGDTTYYPDSVKGRFTISRDNSKNTLYLQMNSLRAEDTAVYYCARDRDYSNSYYYAHDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 81 - protein, 232 aa, synthetic construct; identical to SEQ ID NO: 61

SEQ ID NO: 82 - protein, 469 aa, synthetic construct
MGWSCIILFLVATATGVHSEVQLVESGGGLVQPGGSLRLSCAASGFTFSSYGMSWVRQAPGKGLELVSTINGYGDTWYYPDSVKGRFTISRDNSKNTLYLQMNSLRAEDTAVYYCARDRDYGNSYYYAHDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 83 - protein, 232 aa, synthetic construct; identical to SEQ ID NO: 71

SEQ ID NO: 84 - protein, 469 aa, synthetic construct
MGWSCIILFLVATATGVHSEVQLVESGGGLVQPGGSLRLSCAASGFTFSSYGMSWVRQAPGKGLELVSTINGYGDTTYYPDSVKGRFTISRDNSKNTLYLQMNSLRAEDTAVYYCARDRDYGNSYYYAHDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 85 - protein, 232 aa, synthetic construct
MGWSCIILFLVATATGVHSEIVLTQSPATLSLSPGERATLSCRASSWVSFMHWLQQKPGQAPRPWIYATMNLASGVPARFSGSGSGTDYTLTISSLEPEDFAVYYCQQWSSNPPAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 469 aa, synthetic construct
MGWSCIILFLVATATGVHSEVQLVESGGGLVQPGGSLRLSCAASGFWFSSWGMSWVRQAPGKGLELVSTINGYGDTTYYPDSVKGRFTISRDNSKNTLYLQMNSLRAEDTAVYYCARDRDYTNSYYYAHDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 87 - protein, 232 aa, synthetic construct
MGWSCIILFLVATATGVHSEIVLTQSPATLSLSPGERATLSCRASSYVSFMHWLQQKPGQAPRPWIYATQNLASGVPARFSGSGSGTDYTLTISSLEPEDFAVYYCQQWSSNPPAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 469 aa, synthetic construct; identical to SEQ ID NO: 72

SEQ ID NO: 89 - protein, 232 aa, synthetic construct; identical to SEQ ID NO: 75

SEQ ID NO: 90 - protein, 469 aa, synthetic construct
MGWSCIILFLVATATGVHSEVQLVESGGGLVQPGGSLRLSCAASGYWFSSWGMSWVRQAPGKGLELVSTINGYGDTTYYPDSVKGRFTISRDNSKNTLYLQMNSLRAEDTAVYYCARDRDYSNSYYYAHDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 91 - protein, 232 aa, synthetic construct; identical to SEQ ID NO: 75

SEQ ID NO: 92 - protein, 469 aa, synthetic construct
MGWSCIILFLVATATGVHSEVQLVESGGGLVQPGGSLRLSCAASGFWFSSYGMSWVRQAPGKGLELVSTINGYGDTTYYPDSVKGRFTISRDNSKNTLYLQMNSLRAEDTAVYYCARDRDYTNSYYYAHDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 93 - protein, 232 aa, synthetic construct
MGWSCIILFLVATATGVHSEIVLTQSPATLSLSPGERATLSCRASSSVSFMHWLQQKPGQAPRPWIYATANLASGVPARFSGSGSGTDYTLTISSLEPEDFAVYYCQQWSSNPPAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 469 aa, synthetic construct; identical to SEQ ID NO: 78

SEQ ID NO: 95 - protein, 232 aa, synthetic construct; identical to SEQ ID NO: 85

SEQ ID NO: 96 - protein, 469 aa, synthetic construct; identical to SEQ ID NO: 68

SEQ ID NO: 97 - protein, 232 aa, synthetic construct; identical to SEQ ID NO: 75

SEQ ID NO: 98 - protein, 469 aa, synthetic construct; identical to SEQ ID NO: 62

SEQ ID NO: 99 - protein, 232 aa, synthetic construct
MGWSCIILFLVATATGVHSEIVLTQSPATLSLSPGERATLSCRASSYVSFMHWLQQKPGQAPRPWIYATSNLASGVPARFSGSGSGTDYTLTISSLEPEDFAVYYCQQWSSNPPAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 469 aa, synthetic construct
MGWSCIILFLVATATGVHSEVQLVESGGGLVQPGGSLRLSCAASGFTFSSYGMSWVRQAPGKGLELVSTINGYGDTTYYPDSVKGRFTISRDNSKNTLYLQMNSLRAEDTAVYYCARDRDYGNSYYYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 101 - protein, 232 aa, synthetic construct
MGWSCIILFLVATATGVHSEIVLTQSPATLSLSPGERATLSCRASSSVSFMHWLQQKPGQAPRPWIYATSNLASGVPARFSGSGSGTDYTLTISSLEPEDFAVYYCQQWSSNPPAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - DNA, 369 bp, synthetic construct
  source 1..369: mol_type = other DNA
gaggttcaactagtagaatcaggtggagggttggtgcagccgggtggtagcctccgcctgagctgtgctgcgagcggtttctggttcagctcctacggcatgagctgggttcgtcaggcaccgggcaaaggcctggaactggtgtccaccatcaacggttatggcgatacgtggtattacccggatagcgttaaaggccgttttaccattagccgtgacaactctaagaacaccttatatctgcaaatgaattcactgcgcgcagaggacactgccgtttattactgcgcgcgtgacagagactacactaattcctattactacgcgcacgattactggggtcaaggtacgttggtcaccgtgtcttcg

SEQ ID NO: 103 - DNA, 318 bp, synthetic construct
  source 1..318: mol_type = other DNA
gaaatagtactaacacaatcacccgctactttgtcactctcgccgggtgagcgcgcgaccctgtcttgtcgtgccagctcctacgtgagctttatgcactggctgcagcaaaaaccgggtcaggcaccgcgtccgtggatttatgcgacggccaatctggctagcggtgttccggcgcgtttcagcggcagcggctctggcactgactacaccttgaccatcagctctctggaaccggaagatttcgctgtgtattactgccagcaatggagctccaacccaccggcgtttggtggtggcaccaaagttgagatcaag

SEQ ID NO: 104 - DNA, 369 bp, synthetic construct
  source 1..369: mol_type = other DNA
gaggttcaactagtagaatcaggtggagggttggtgcagccgggtggtagcctccgcctgagctgtgctgcgagcggtttcaccttcagctcctggggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactacactaattcctattactacgcgcacgattactggggtcaaggtacgttggtcaccgtgtcttcg

SEQ ID NO: 105 - DNA, 318 bp, synthetic construct; identical to SEQ ID NO: 103
  source 1..318: mol_type = other DNA

SEQ ID NO: 106 - DNA, 369 bp, synthetic construct
  source 1..369: mol_type = other DNA
gaggttcaactagtagaatcaggtggagggttggtgcagccgggtggtagcctccgcctgagctgtgctgcgagcggttacaccttcagctcctacggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactactctaattcctattactacgcgctggattactggggtcaaggtacgttggtcaccgtgtcttcg

SEQ ID NO: 107 - DNA, 318 bp, synthetic construct; identical to SEQ ID NO: 103
  source 1..318: mol_type = other DNA

SEQ ID NO: 108 - DNA, 369 bp, synthetic construct
  source 1..369: mol_type = other DNA
gaggttcaactagtagaatcaggtggagggttggtgcagccgggtggtagcctccgcctgagctgtgctgcgagcggttacaccttcagctcctacggcatgagctgggttcgtcaggcaccgggcaaaggcctggaactggtgtccaccatcaacggttatggcgatacgtggtattacccggatagcgttaaaggccgttttaccattagccgtgacaactctaagaacaccttatatctgcaaatgaattcactgcgcgcagaggacactgccgtttattactgcgcgcgtgacagagactactctaattcctattactacgcgcacgattactggggtcaaggtacgttggtcaccgtgtcttcg

SEQ ID NO: 109 - DNA, 318 bp, synthetic construct
  source 1..318: mol_type = other DNA
gaaatagtactaacacaatcacccgctactttgtcactctcgccgggtgagcgcgcgaccctgtcttgtcgtgccagctcctgggtgagctttatgcactggctgcagcaaaaaccgggtcaggcaccgcgtccgtggatttatgcgacgcagaatctggctagcggtgttccggcgcgtttcatcggcagcggctctggcactgactacaccttgaccatcagctctctggaaccggaagatttcgctgtgtattactgccagcaatggagctccaacccaccggcgtttggtggtggcaccaaagttgagatcaag

SEQ ID NO: 110 - DNA, 369 bp, synthetic construct
  source 1..369: mol_type = other DNA
gaggttcaactagtagaatcaggtggagggttggtgcagccgggtggtagcctccgcctgagctgtgctgcgagcggttactggttcagctcctacggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactacactaattcctattactacgcgcacgattactggggtcaaggtacgttggtcaccgtgtcttcg

SEQ ID NO: 111 - DNA, 318 bp, synthetic construct
  source 1..318: mol_type = other DNA
gaaatagtactaacacaatcacccgctactttgtcactctcgccgggtgagcgcgcgaccctgtcttgtcgtgccagctcctccgtgagctttatgcactggctgcagcaaaaaccgggtcaggcaccgcgtccgtggatttatgcgacgatgaatctggctagcggtgttccggcgcgtttcagcggcagcggctctggcactgactacaccttgaccatcagctctctggaaccggaagatttcgctgtgtattactgccagcaatggagctccaacccaccggcgtttggtggtggcaccaaagttgagatcaag

SEQ ID NO: 112 - DNA, 369 bp, synthetic construct
  source 1..369: mol_type = other DNA
gaggttcaactagtagaatcaggtggagggttggtgcagccgggtggtagcctccgcctgagctgtgctgcgagcggtttcaccttcagctcctacggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactactctaattcctattactacgcgcacgattactggggtcaaggtacgttggtcaccgtgtcttcg

SEQ ID NO: 113 - DNA, 318 bp, synthetic construct
  source 1..318: mol_type = other DNA
gaaatagtactaacacaatcacccgctactttgtcactctcgccgggtgagcgcgcgaccctgtcttgtcgtgccagctcctgggtgagctttatgcactggctgcagcaaaaaccgggtcaggcaccgcgtccgtggatttatgcgacgcagaatctggctagcggtgttccggcgcgtttcagcggcagcggctctggcactgactacaccttgaccatcagctctctggaaccggaagatttcgctgtgtattactgccagcaatggagctccaacccaccggcgtttggtggtggcaccaaagttgagatcaag

SEQ ID NO: 114 - DNA, 369 bp, synthetic construct
  source 1..369: mol_type = other DNA
gaggttcaactagtagaatcaggtggagggttggtgcagccgggtggtagcctccgcctgagctgtgctgcgagcggtttcaccttcagctcctacggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactacactaattcctattactacgcgcacgattactggggtcaaggtacgttggtcaccgtgtcttcg

SEQ ID NO: 115 - DNA, 318 bp, synthetic construct
  source 1..318: mol_type = other DNA
gaaatagtactaacacaatcacccgctactttgtcactctcgccgggtgagcgcgcgaccctgtcttgtcgtgccagctcctccgtgagctttatgcactggctgcagcaaaaaccgggtcaggcaccgcgtccgtggatttatgcgacgcagaatctggctagcggtgttccggcgcgtttcagcggcagcggctctggcactgactacaccttgaccatcagctctctggaaccggaagatttcgctgtgtattactgccagcaatggagctccaacccaccggcgtttggtggtggcaccaaagttgagatcaag

SEQ ID NO: 116 - DNA, 369 bp, synthetic construct
  source 1..369: mol_type = other DNA
gaggttcaactagtagaatcaggtggagggttggtgcagccgggtggtagcctccgcctgagctgtgctgcgagcggttacaccttcagctcctggggcatgagctgggttcgtcaggcaccgggcaaaggcctggaactggtgtccaccatcaacggttatggcgatacgtggtattacccggatagcgttaaaggccgttttaccattagccgtgacaactctaagaacaccttatatctgcaaatgaattcactgcgcgcagaggacactgccgtttattactgcgcgcgtgacagagactactctaattcctattactacgcgcacgattactggggtcaaggtacgttggtcaccgtgtcttcg

SEQ ID NO: 117 - DNA, 318 bp, synthetic construct
  source 1..318: mol_type = other DNA
gaaatagtactaacacaatcacccgctactttgtcactctcgccgggtgagcgcgcgaccctgtcttgtcgtgccagctcctacgtgagctttatgcactggctgcagcaaaaaccgggtcaggcaccgcgtccgtggatttatgcgacgatgaatctggctagcggtgttccggcgcgtttcagcggcagcggctctggcactgactacaccttgaccatcagctctctggaaccggaagatttcgctgtgtattactgccagcaatggagctccaacccaccggcgtttggtggtggcaccaaagttgagatcaag

SEQ ID NO: 118 - DNA, 369 bp, synthetic construct
  source 1..369: mol_type = other DNA
gaggttcaactagtagaatcaggtggagggttggtgcagccgggtggtagcctccgcctgagctgtgctgcgagcggtttcaccttcagctcctacggcatgagctgggttcgtcaggcaccgggcaaaggcctggaactggtgtccaccatcaacggttatggcgatacgtggtattacccggatagcgttaaaggccgttttaccattagccgtgacaactctaagaacaccttatatctgcaaatgaattcactgcgcgcagaggacactgccgtttattactgcgcgcgtgacagagactactctaattcctattactacgcgcacgattactggggtcaaggtacgttggtcaccgtgtcttcg

SEQ ID NO: 119 - DNA, 318 bp, synthetic construct; identical to SEQ ID NO: 111
  source 1..318: mol_type = other DNA

SEQ ID NO: 120 - DNA, 369 bp, synthetic construct
  source 1..369: mol_type = other DNA
gaggttcaactagtagaatcaggtggagggttggtgcagccgggtggtagcctccgcctgagctgtgctgcgagcggttacaccttcagctcctacggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactacggtaattcctattactacgcgcacgattactggggtcaaggtacgttggtcaccgtgtcttcg

SEQ ID NO: 121 - DNA, 318 bp, synthetic construct; identical to SEQ ID NO: 117
  source 1..318: mol_type = other DNA

SEQ ID NO: 122 - DNA, 369 bp, synthetic construct
  source 1..369: mol_type = other DNA
gaggttcaactagtagaatcaggtggagggttggtgcagccgggtggtagcctccgcctgagctgtgctgcgagcggtttcaccttcagctcctggggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactactctaattcctattactacgcgcacgattactggggtcaaggtacgttggtcaccgtgtcttcg

SEQ ID NO: 123 - DNA, 318 bp, synthetic construct; identical to SEQ ID NO: 103
  source 1..318: mol_type = other DNA

SEQ ID NO: 124 - DNA, 369 bp, synthetic construct
  source 1..369: mol_type = other DNA
gaggttcaactagtagaatcaggtggagggttggtgcagccgggtggtagcctccgcctgagctgtgctgcgagcggtttcaccttcagctcctacggcatgagctgggttcgtcaggcaccgggcaaaggcctggaactggtgtccaccatcaacggttatggcgatacgtggtattacccggatagcgttaaaggccgttttaccattagccgtgacaactctaagaacaccttatatctgcaaatgaattcactgcgcgcagaggacactgccgtttattactgcgcgcgtgacagagactacggtaattcctattactacgcgcacgattactggggtcaaggtacgttggtcaccgtgtcttcg

SEQ ID NO: 125 - DNA, 318 bp, synthetic construct; identical to SEQ ID NO: 113
  source 1..318: mol_type = other DNA

SEQ ID NO: 126 - DNA, 369 bp, synthetic construct
  source 1..369: mol_type = other DNA
gaggttcaactagtagaatcaggtggagggttggtgcagccgggtggtagcctccgcctgagctgtgctgcgagcggtttcaccttcagctcctacggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactacggtaattcctattactacgcgcacgattactggggtcaaggtacgttggtcaccgtgtcttcg

SEQ ID NO: 127 - DNA, 318 bp, synthetic construct
  source 1..318: mol_type = other DNA
gaaatagtactaacacaatcacccgctactttgtcactctcgccgggtgagcgcgcgaccctgtcttgtcgtgccagctcctgggtgagctttatgcactggctgcagcaaaaaccgggtcaggcaccgcgtccgtggatttatgcgacgatgaatctggctagcggtgttccggcgcgtttcagcggcagcggctctggcactgactacaccttgaccatcagctctctggaaccggaagatttcgctgtgtattactgccagcaatggagctccaacccaccggcgtttggtggtggcaccaaagttgagatcaag

SEQ ID NO: 128 - DNA, 369 bp, synthetic construct
  source 1..369: mol_type = other DNA
gaggttcaactagtagaatcaggtggagggttggtgcagccgggtggtagcctccgcctgagctgtgctgcgagcggtttctggttcagctcctggggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactacactaattcctattactacgcgcacgattactggggtcaaggtacgttggtcaccgtgtcttcg

SEQ ID NO: 129 - DNA, 318 bp, synthetic construct
  source 1..318: mol_type = other DNA
gaaatagtactaacacaatcacccgctactttgtcactctcgccgggtgagcgcgcgaccctgtcttgtcgtgccagctcctacgtgagctttatgcactggctgcagcaaaaaccgggtcaggcaccgcgtccgtggatttatgcgacgcagaatctggctagcggtgttccggcgcgtttcagcggcagcggctctggcactgactacaccttgaccatcagctctctggaaccggaagatttcgctgtgtattactgccagcaatggagctccaacccaccggcgtttggtggtggcaccaaagttgagatcaag

SEQ ID NO: 130 - DNA, 369 bp, synthetic construct; identical to SEQ ID NO: 114
  source 1..369: mol_type = other DNA

SEQ ID NO: 131 - DNA, 318 bp, synthetic construct; identical to SEQ ID NO: 117
  source 1..318: mol_type = other DNA

SEQ ID NO: 132 - DNA, 369 bp, synthetic construct
  source 1..369: mol_type = other DNA
gaggttcaactagtagaatcaggtggagggttggtgcagccgggtggtagcctccgcctgagctgtgctgcgagcggttactggttcagctcctggggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactactctaattcctattactacgcgcacgattactggggtcaaggtacgttggtcaccgtgtcttcg

SEQ ID NO: 133 - DNA, 318 bp, synthetic construct; identical to SEQ ID NO: 117
  source 1..318: mol_type = other DNA

SEQ ID NO: 134 - DNA, 369 bp, synthetic construct
  source 1..369: mol_type = other DNA
gaggttcaactagtagaatcaggtggagggttggtgcagccgggtggtagcctccgcctgagctgtgctgcgagcggtttctggttcagctcctacggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactacactaattcctattactacgcgcacgattactggggtcaaggtacgttggtcaccgtgtcttcg

SEQ ID NO: 135 - DNA, 318 bp, synthetic construct
  source 1..318: mol_type = other DNA
gaaatagtactaacacaatcacccgctactttgtcactctcgccgggtgagcgcgcgaccctgtcttgtcgtgccagctcctccgtgagctttatgcactggctgcagcaaaaaccgggtcaggcaccgcgtccgtggatttatgcgacggccaatctggctagcggtgttccggcgcgtttcagcggcagcggctctggcactgactacaccttgaccatcagctctctggaaccggaagatttcgctgtgtattactgccagcaatggagctccaacccaccggcgtttggtggtggcaccaaagttgagatcaag

SEQ ID NO: 136 - DNA, 369 bp, synthetic construct; identical to SEQ ID NO: 120
  source 1..369: mol_type = other DNA

SEQ ID NO: 137 - DNA, 318 bp, synthetic construct; identical to SEQ ID NO: 127
  source 1..318: mol_type = other DNA

SEQ ID NO: 138 - DNA, 369 bp, synthetic construct; identical to SEQ ID NO: 110
  source 1..369: mol_type = other DNA

SEQ ID NO: 139 - DNA, 318 bp, synthetic construct; identical to SEQ ID NO: 117
  source 1..318: mol_type = other DNA

SEQ ID NO: 140 - DNA, 369 bp, synthetic construct; identical to SEQ ID NO: 104
  source 1..369: mol_type = other DNA

SEQ ID NO: 141 - DNA, 318 bp, synthetic construct
  source 1..318: mol_type = other DNA
gaaatagtactaacacaatcacccgctactttgtcactctcgccgggtgagcgcgcgaccctgtcttgtcgtgccagctcctacgtgagctttatgcactggctgcagcaaaaaccgggtcaggcaccgcgtccgtggatttatgcgacgagcaatctggctagcggtgttccggcgcgtttcagcggcagcggctctggcactgactacaccttgaccatcagctctctggaaccggaagatttcgctgtgtattactgccagcaatggagctccaacccaccggcgtttggtggtggcaccaaagttgagatcaag

SEQ ID NO: 142 - DNA, 369 bp, synthetic construct
  source 1..369: mol_type = other DNA
gaggttcaactagtagaatcaggtggagggttggtgcagccgggtggtagcctccgcctgagctgtgctgcgagcggtttcaccttcagctcctacggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactacggtaattcctattactacgcgctggattactggggtcaaggtacgttggtcaccgtgtcttcg

SEQ ID NO: 143 - DNA, 318 bp, synthetic construct
  source 1..318: mol_type = other DNA
gaaatagtactaacacaatcacccgctactttgtcactctcgccgggtgagcgcgcgaccctgtcttgtcgtgccagctcctccgtgagctttatgcactggctgcagcaaaaaccgggtcaggcaccgcgtccgtggatttatgcgacgagcaatctggctagcggtgttccggcgcgtttcagcggcagcggctctggcactgactacaccttgaccatcagctctctggaaccggaagatttcgctgtgtattactgccagcaatggagctccaacccaccggcgtttggtggtggcaccaaagttgagatcaag

SEQ ID NO: 144 - DNA, 57 bp, synthetic construct
  source 1..57: mol_type = other DNA
atgggctggtcttgtattattctgtttctggtcgcaactgctacaggcgtgcattct

SEQ ID NO: 145 - DNA, 57 bp, synthetic construct
  source 1..57: mol_type = other DNA
atgggctggtcatgtattattctgtttctggtcgcaactgctacaggggtccatagt

SEQ ID NO: 146 - DNA, 981 bp, Homo sapiens
  source 1..981: mol_type = genomic DNA
gccagcaccaagggcccttccgtgtttcccctggccccttgctcccggtccacatctgagagcaccgccgccctgggctgtctggtgaaggactacttcccagagcccgtgaccgtgagctggaacagcggcgccctgacaagcggcgtgcacacatttcccgccgtgctgcagagctccggcctgtactccctgtctagcgtggtgacagtgccttcctctagcctgggcaccaagacatatacctgtaacgtggaccacaagccaagcaataccaaggtggataagcgggtggagtctaagtacggccctccttgccctccatgtcctgctccagagtttctgggcggcccttccgtgttcctgtttccacccaaaccaaaggacacactgatgatctctagaacaccagaggtgacctgcgtggtggtggacgtgagccaggaggatcccgaggtgcagttcaactggtacgtggatggcgtggaggtgcacaatgccaagaccaagccaagagaggagcagtttaactctacatacagggtggtgagcgtgctgaccgtgctgcaccaggattggctcaacggcaaggagtataagtgcaaggtgtccaataagggcctgccctcctctatcgagaagacaatctctaaggctaagggccagccaagagagcctcaggtgtacaccctgcctccaagccaggaggagatgacaaagaaccaggtgtccctgacatgtctggtgaagggcttctatccctccgacatcgccgtggagtgggagtctaatggccagcctgagaacaattacaagaccacaccccctgtgctggactctgatggcagcttctttctgtattccaggctgaccgtggataagtctcggtggcaggagggcaacgtgttcagctgctctgtgatgcacgaagccctgcataatcactatactcagaaaagtctgtcactgtcactgggaaag

SEQ ID NO: 147 - DNA, 321 bp, Homo sapiens
  source 1..321: mol_type = genomic DNA
aggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgc

SEQ ID NO: 148 - DNA, 1407 bp, synthetic construct
  source 1..1407: mol_type = other DNA
atgggctggtcttgtattattctgtttctggtcgcaactgctacaggcgtgcattctgaggttcaactagtagaatcaggtggagggttggtgcagccgggtggtagcctccgcctgagctgtgctgcgagcggtttctggttcagctcctacggcatgagctgggttcgtcaggcaccgggcaaaggcctggaactggtgtccaccatcaacggttatggcgatacgtggtattacccggatagcgttaaaggccgttttaccattagccgtgacaactctaagaacaccttatatctgcaaatgaattcactgcgcgcagaggacactgccgtttattactgcgcgcgtgacagagactacactaattcctattactacgcgcacgattactggggtcaaggtacgttggtcaccgtgtcttcggccagcaccaagggcccttccgtgtttcccctggccccttgctcccggtccacatctgagagcaccgccgccctgggctgtctggtgaaggactacttcccagagcccgtgaccgtgagctggaacagcggcgccctgacaagcggcgtgcacacatttcccgccgtgctgcagagctccggcctgtactccctgtctagcgtggtgacagtgccttcctctagcctgggcaccaagacatatacctgtaacgtggaccacaagccaagcaataccaaggtggataagcgggtggagtctaagtacggccctccttgccctccatgtcctgctccagagtttctgggcggcccttccgtgttcctgtttccacccaaaccaaaggacacactgatgatctctagaacaccagaggtgacctgcgtggtggtggacgtgagccaggaggatcccgaggtgcagttcaactggtacgtggatggcgtggaggtgcacaatgccaagaccaagccaagagaggagcagtttaactctacatacagggtggtgagcgtgctgaccgtgctgcaccaggattggctcaacggcaaggagtataagtgcaaggtgtccaataagggcctgccctcctctatcgagaagacaatctctaaggctaagggccagccaagagagcctcaggtgtacaccctgcctccaagccaggaggagatgacaaagaaccaggtgtccctgacatgtctggtgaagggcttctatccctccgacatcgccgtggagtgggagtctaatggccagcctgagaacaattacaagaccacaccccctgtgctggactctgatggcagcttctttctgtattccaggctgaccgtggataagtctcggtggcaggagggcaacgtgttcagctgctctgtgatgcacgaagccctgcataatcactatactcagaaaagtctgtcactgtcactgggaaag

SEQ ID NO: 149 - DNA, 696 bp, synthetic construct
  source 1..696: mol_type = other DNA
atgggctggtcatgtattattctgtttctggtcgcaactgctacaggggtccatagtgaaatagtactaacacaatcacccgctactttgtcactctcgccgggtgagcgcgcgaccctgtcttgtcgtgccagctcctacgtgagctttatgcactggctgcagcaaaaaccgggtcaggcaccgcgtccgtggatttatgcgacggccaatctggctagcggtgttccggcgcgtttcagcggcagcggctctggcactgactacaccttgaccatcagctctctggaaccggaagatttcgctgtgtattactgccagcaatggagctccaacccaccggcgtttggtggtggcaccaaagttgagatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgc

SEQ ID NO: 150 - DNA, 1407 bp, synthetic construct
  source 1..1407: mol_type = other DNA
atgggctggtcttgtattattctgtttctggtcgcaactgctacaggcgtgcattctgaggttcaactagtagaatcaggtggagggttggtgcagccgggtggtagcctccgcctgagctgtgctgcgagcggtttcaccttcagctcctggggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactacactaattcctattactacgcgcacgattactggggtcaaggtacgttggtcaccgtgtcttcggccagcaccaagggcccttccgtgtttcccctggccccttgctcccggtccacatctgagagcaccgccgccctgggctgtctggtgaaggactacttcccagagcccgtgaccgtgagctggaacagcggcgccctgacaagcggcgtgcacacatttcccgccgtgctgcagagctccggcctgtactccctgtctagcgtggtgacagtgccttcctctagcctgggcaccaagacatatacctgtaacgtggaccacaagccaagcaataccaaggtggataagcgggtggagtctaagtacggccctccttgccctccatgtcctgctccagagtttctgggcggcccttccgtgttcctgtttccacccaaaccaaaggacacactgatgatctctagaacaccagaggtgacctgcgtggtggtggacgtgagccaggaggatcccgaggtgcagttcaactggtacgtggatggcgtggaggtgcacaatgccaagaccaagccaagagaggagcagtttaactctacatacagggtggtgagcgtgctgaccgtgctgcaccaggattggctcaacggcaaggagtataagtgcaaggtgtccaataagggcctgccctcctctatcgagaagacaatctctaaggctaagggccagccaagagagcctcaggtgtacaccctgcctccaagccaggaggagatgacaaagaaccaggtgtccctgacatgtctggtgaagggcttctatccctccgacatcgccgtggagtgggagtctaatggccagcctgagaacaattacaagaccacaccccctgtgctggactctgatggcagcttctttctgtattccaggctgaccgtggataagtctcggtggcaggagggcaacgtgttcagctgctctgtgatgcacgaagccctgcataatcactatactcagaaaagtctgtcactgtcactgggaaag

SEQ ID NO: 151 - DNA, 696 bp, synthetic construct; identical to SEQ ID NO: 149
  source 1..696: mol_type = other DNA

SEQ ID NO: 152 - DNA, 1407 bp, synthetic construct
  source 1..1407: mol_type = other DNA
atgggctggtcttgtattattctgtttctggtcgcaactgctacaggcgtgcattctgaggttcaactagtagaatcaggtggagggttggtgcagccgggtggtagcctccgcctgagctgtgctgcgagcggttacaccttcagctcctacggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactactctaattcctattactacgcgctggattactggggtcaaggtacgttggtcaccgtgtcttcggccagcaccaagggcccttccgtgtttcccctggccccttgctcccggtccacatctgagagcaccgccgccctgggctgtctggtgaaggactacttcccagagcccgtgaccgtgagctggaacagcggcgccctgacaagcggcgtgcacacatttcccgccgtgctgcagagctccggcctgtactccctgtctagcgtggtgacagtgccttcctctagcctgggcaccaagacatatacctgtaacgtggaccacaagccaagcaataccaaggtggataagcgggtggagtctaagtacggccctccttgccctccatgtcctgctccagagtttctgggcggcccttccgtgttcctgtttccacccaaaccaaaggacacactgatgatctctagaacaccagaggtgacctgcgtggtggtggacgtgagccaggaggatcccgaggtgcagttcaactggtacgtggatggcgtggaggtgcacaatgccaagaccaagccaagagaggagcagtttaactctacatacagggtggtgagcgtgctgaccgtgctgcaccaggattggctcaacggcaaggagtataagtgcaaggtgtccaataagggcctgccctcctctatcgagaagacaatctctaaggctaagggccagccaagagagcctcaggtgtacaccctgcctccaagccaggaggagatgacaaagaaccaggtgtccctgacatgtctggtgaagggcttctatccctccgacatcgccgtggagtgggagtctaatggccagcctgagaacaattacaagaccacaccccctgtgctggactctgatggcagcttctttctgtattccaggctgaccgtggataagtctcggtggcaggagggcaacgtgttcagctgctctgtgatgcacgaagccctgcataatcactatactcagaaaagtctgtcactgtcactgggaaag

SEQ ID NO: 153 - DNA, 696 bp, synthetic construct; identical to SEQ ID NO: 149
  source 1..696: mol_type = other DNA

SEQ ID NO: 154 - DNA, 1407 bp, synthetic construct
  source 1..1407: mol_type = other DNA
atgggctggtcttgtattattctgtttctggtcgcaactgctacaggcgtgcattctgaggttcaactagtagaatcaggtggagggttggtgcagccgggtggtagcctccgcctgagctgtgctgcgagcggttacaccttcagctcctacggcatgagctgggttcgtcaggcaccgggcaaaggcctggaactggtgtccaccatcaacggttatggcgatacgtggtattacccggatagcgttaaaggccgttttaccattagccgtgacaactctaagaacaccttatatctgcaaatgaattcactgcgcgcagaggacactgccgtttattactgcgcgcgtgacagagactactctaattcctattactacgcgcacgattactggggtcaaggtacgttggtcaccgtgtcttcggccagcaccaagggcccttccgtgtttcccctggccccttgctcccggtccacatctgagagcaccgccgccctgggctgtctggtgaaggactacttcccagagcccgtgaccgtgagctggaacagcggcgccctgacaagcggcgtgcacacatttcccgccgtgctgcagagctccggcctgtactccctgtctagcgtggtgacagtgccttcctctagcctgggcaccaagacatatacctgtaacgtggaccacaagccaagcaataccaaggtggataagcgggtggagtctaagtacggccctccttgccctccatgtcctgctccagagtttctgggcggcccttccgtgttcctgtttccacccaaaccaaaggacacactgatgatctctagaacaccagaggtgacctgcgtggtggtggacgtgagccaggaggatcccgaggtgcagttcaactggtacgtggatggcgtggaggtgcacaatgccaagaccaagccaagagaggagcagtttaactctacatacagggtggtgagcgtgctgaccgtgctgcaccaggattggctcaacggcaaggagtataagtgcaaggtgtccaataagggcctgccctcctctatcgagaagacaatctctaaggctaagggccagccaagagagcctcaggtgtacaccctgcctccaagccaggaggagatgacaaagaaccaggtgtccctgacatgtctggtgaagggcttctatccctccgacatcgccgtggagtgggagtctaatggccagcctgagaacaattacaagaccacaccccctgtgctggactctgatggcagcttctttctgtattccaggctgaccgtggataagtctcggtggcaggagggcaacgtgttcagctgctctgtgatgcacgaagccctgcataatcactatactcagaaaagtctgtcactgtcactgggaaag

SEQ ID NO: 155 - DNA, 696 bp, synthetic construct
  source 1..696: mol_type = other DNA
atgggctggtcatgtattattctgtttctggtcgcaactgctacaggggtccatagtgaaatagtactaacacaatcacccgctactttgtcactctcgccgggtgagcgcgcgaccctgtcttgtcgtgccagctcctgggtgagctttatgcactggctgcagcaaaaaccgggtcaggcaccgcgtccgtggatttatgcgacgcagaatctggctagcggtgttccggcgcgtttcatcggcagcggctctggcactgactacaccttgaccatcagctctctggaaccggaagatttcgctgtgtattactgccagcaatggagctccaacccaccggcgtttggtggtggcaccaaagttgagatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgc

SEQ ID NO: 156 - DNA, 1407 bp, synthetic construct
  source 1..1407: mol_type = other DNA
atgggctggtcttgtattattctgtttctggtcgcaactgctacaggcgtgcattctgaggttcaactagtagaatcaggtggagggttggtgcagccgggtggtagcctccgcctgagctgtgctgcgagcggttactggttcagctcctacggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactacactaattcctattactacgcgcacgattactggggtcaaggtacgttggtcaccgtgtcttcggccagcaccaagggcccttccgtgtttcccctggccccttgctcccggtccacatctgagagcaccgccgccctgggctgtctggtgaaggactacttcccagagcccgtgaccgtgagctggaacagcggcgccctgacaagcggcgtgcacacatttcccgccgtgctgcagagctccggcctgtactccctgtctagcgtggtgacagtgccttcctctagcctgggcaccaagacatatacctgtaacgtggaccacaagccaagcaataccaaggtggataagcgggtggagtctaagtacggccctccttgccctccatgtcctgctccagagtttctgggcggcccttccgtgttcctgtttccacccaaaccaaaggacacactgatgatctctagaacaccagaggtgacctgcgtggtggtggacgtgagccaggaggatcccgaggtgcagttcaactggtacgtggatggcgtggaggtgcacaatgccaagaccaagccaagagaggagcagtttaactctacatacagggtggtgagcgtgctgaccgtgctgcaccaggattggctcaacggcaaggagtataagtgcaaggtgtccaataagggcctgccctcctctatcgagaagacaatctctaaggctaagggccagccaagagagcctcaggtgtacaccctgcctccaagccaggaggagatgacaaagaaccaggtgtccctgacatgtctggtgaagggcttctatccctccgacatcgccgtggagtgggagtctaatggccagcctgagaacaattacaagaccacaccccctgtgctggactctgatggcagcttctttctgtattccaggctgaccgtggataagtctcggtggcaggagggcaacgtgttcagctgctctgtgatgcacgaagccctgcataatcactatactcagaaaagtctgtcactgtcactgggaaag

SEQ ID NO: 157 - DNA, 696 bp, synthetic construct
  source 1..696: mol_type = other DNA
atgggctggtcatgtattattctgtttctggtcgcaactgctacaggggtccatagtgaaatagtactaacacaatcacccgctactttgtcactctcgccgggtgagcgcgcgaccctgtcttgtcgtgccagctcctccgtgagctttatgcactggctgcagcaaaaaccgggtcaggcaccgcgtccgtggatttatgcgacgatgaatctggctagcggtgttccggcgcgtttcagcggcagcggctctggcactgactacaccttgaccatcagctctctggaaccggaagatttcgctgtgtattactgccagcaatggagctccaacccaccggcgtttggtggtggcaccaaagttgagatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgc

SEQ ID NO: 158 - DNA, 1407 bp, synthetic construct
  source 1..1407: mol_type = other DNA
atgggctggtcttgtattattctgtttctggtcgcaactgctacaggcgtgcattctgaggttcaactagtagaatcaggtggagggttggtgcagccgggtggtagcctccgcctgagctgtgctgcgagcggtttcaccttcagctcctacggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactactctaattcctattactacgcgcacgattactggggtcaaggtacgttggtcaccgtgtcttcggccagcaccaagggcccttccgtgtttcccctggccccttgctcccggtccacatctgagagcaccgccgccctgggctgtctggtgaaggactacttcccagagcccgtgaccgtgagctggaacagcggcgccctgacaagcggcgtgcacacatttcccgccgtgctgcagagctccggcctgtactccctgtctagcgtggtgacagtgccttcctctagcctgggcaccaagacatatacctgtaacgtggaccacaagccaagcaataccaaggtggataagcgggtggagtctaagtacggccctccttgccctccatgtcctgctccagagtttctgggcggcccttccgtgttcctgtttccacccaaaccaaaggacacactgatgatctctagaacaccagaggtgacctgcgtggtggtggacgtgagccaggaggatcccgaggtgcagttcaactggtacgtggatggcgtggaggtgcacaatgccaagaccaagccaagagaggagcagtttaactctacatacagggtggtgagcgtgctgaccgtgctgcaccaggattggctcaacggcaaggagtataagtgcaaggtgtccaataagggcctgccctcctctatcgagaagacaatctctaaggctaagggccagccaagagagcctcaggtgtacaccctgcctccaagccaggaggagatgacaaagaaccaggtgtccctgacatgtctggtgaagggcttctatccctccgacatcgccgtggagtgggagtctaatggccagcctgagaacaattacaagaccacaccccctgtgctggactctgatggcagcttctttctgtattccaggctgaccgtggataagtctcggtggcaggagggcaacgtgttcagctgctctgtgatgcacgaagccctgcataatcactatactcagaaaagtctgtcactgtcactgggaaag

SEQ ID NO: 159 - DNA, 696 bp, synthetic construct
  source 1..696: mol_type = other DNA
atgggctggtcatgtattattctgtttctggtcgcaactgctacaggggtccatagtgaaatagtactaacacaatcacccgctactttgtcactctcgccgggtgagcgcgcgaccctgtcttgtcgtgccagctcctgggtgagctttatgcactggctgcagcaaaaaccgggtcaggcaccgcgtccgtggatttatgcgacgcagaatctggctagcggtgttccggcgcgtttcagcggcagcggctctggcactgactacaccttgaccatcagctctctggaaccggaagatttcgctgtgtattactgccagcaatggagctccaacccaccggcgtttggtggtggcaccaaagttgagatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgc

SEQ ID NO: 160 - DNA, 1407 bp, synthetic construct
  source 1..1407: mol_type = other DNA
atgggctggtcttgtattattctgtttctggtcgcaactgctacaggcgtgcattctgaggttcaactagtagaatcaggtggagggttggtgcagccgggtggtagcctccgcctgagctgtgctgcgagcggtttcaccttcagctcctacggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactacactaattcctattactacgcgcacgattactggggtcaaggtacgttggtcaccgtgtcttcggccagcaccaagggcccttccgtgtttcccctggccccttgctcccggtccacatctgagagcaccgccgccctgggctgtctggtgaaggactacttcccagagcccgtgaccgtgagctggaacagcggcgccctgacaagcggcgtgcacacatttcccgccgtgctgcagagctccggcctgtactccctgtctagcgtggtgacagtgccttcctctagcctgggcaccaagacatatacctgtaacgtggaccacaagccaagcaataccaaggtggataagcgggtggagtctaagtacggccctccttgccctccatgtcctgctccagagtttctgggcggcccttccgtgttcctgtttccacccaaaccaaaggacacactgatgatctctagaacaccagaggtgacctgcgtggtggtggacgtgagccaggaggatcccgaggtgcagttcaactggtacgtggatggcgtggaggtgcacaatgccaagaccaagccaagagaggagcagtttaactctacatacagggtggtgagcgtgctgaccgtgctgcaccaggattggctcaacggcaaggagtataagtgcaaggtgtccaataagggcctgccctcctctatcgagaagacaatctctaaggctaagggccagccaagagagcctcaggtgtacaccctgcctccaagccaggaggagatgacaaagaaccaggtgtccctgacatgtctggtgaagggcttctatccctccgacatcgccgtggagtgggagtctaatggccagcctgagaacaattacaagaccacaccccctgtgctggactctgatggcagcttctttctgtattccaggctgaccgtggataagtctcggtggcaggagggcaacgtgttcagctgctctgtgatgcacgaagccctgcataatcactatactcagaaaagtctgtcactgtcactgggaaag

SEQ ID NO: 161 - DNA, 696 bp, synthetic construct
  source 1..696: mol_type = other DNA
atgggctggtcatgtattattctgtttctggtcgcaactgctacaggggtccatagtgaaatagtactaacacaatcacccgctactttgtcactctcgccgggtgagcgcgcgaccctgtcttgtcgtgccagctcctccgtgagctttatgcactggctgcagcaaaaaccgggtcaggcaccgcgtccgtggatttatgcgacgcagaatctggctagcggtgttccggcgcgtttcagcggcagcggctctggcactgactacaccttgaccatcagctctctggaaccggaagatttcgctgtgtattactgccagcaatggagctccaacccaccggcgtttggtggtggcaccaaagttgagatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgc

SEQ ID NO: 162 - DNA, 1407 bp, synthetic construct
  source 1..1407: mol_type = other DNA
atgggctggtcttgtattattctgtttctggtcgcaactgctacaggcgtgcattctgaggttcaactagtagaatcaggtggagggttggtgcagccgggtggtagcctccgcctgagctgtgctgcgagcggttacaccttcagctcctggggcatgagctgggttcgtcaggcaccgggcaaaggcctggaactggtgtccaccatcaacggttatggcgatacgtggtattacccggatagcgttaaaggccgttttaccattagccgtgacaactctaagaacaccttatatctgcaaatgaattcactgcgcgcagaggacactgccgtttattactgcgcgcgtgacagagactactctaattcctattactacgcgcacgattactggggtcaaggtacgttggtcaccgtgtcttcggccagcaccaagggcccttccgtgtttcccctggccccttgctcccggtccacatctgagagcaccgccgccctgggctgtctggtgaaggactacttcccagagcccgtgaccgtgagctggaacagcggcgccctgacaagcggcgtgcacacatttcccgccgtgctgcagagctccggcctgtactccctgtctagcgtggtgacagtgccttcctctagcctgggcaccaagacatatacctgtaacgtggaccacaagccaagcaataccaaggtggataagcgggtggagtctaagtacggccctccttgccctccatgtcctgctccagagtttctgggcggcccttccgtgttcctgtttccacccaaaccaaaggacacactgatgatctctagaacaccagaggtgacctgcgtggtggtggacgtgagccaggaggatcccgaggtgcagttcaactggtacgtggatggcgtggaggtgcacaatgccaagaccaagccaagagaggagcagtttaactctacatacagggtggtgagcgtgctgaccgtgctgcaccaggattggctcaacggcaaggagtataagtgcaaggtgtccaataagggcctgccctcctctatcgagaagacaatctctaaggctaagggccagccaagagagcctcaggtgtacaccctgcctccaagccaggaggagatgacaaagaaccaggtgtccctgacatgtctggtgaagggcttctatccctccgacatcgccgtggagtgggagtctaatggccagcctgagaacaattacaagaccacaccccctgtgctggactctgatggcagcttctttctgtattccaggctgaccgtggataagtctcggtggcaggagggcaacgtgttcagctgctctgtgatgcacgaagccctgcataatcactatactcagaaaagtctgtcactgtcactgggaaag

SEQ ID NO: 163 - DNA, 696 bp, synthetic construct
  source 1..696: mol_type = other DNA
atgggctggtcatgtattattctgtttctggtcgcaactgctacaggggtccatagtgaaatagtactaacacaatcacccgctactttgtcactctcgccgggtgagcgcgcgaccctgtcttgtcgtgccagctcctacgtgagctttatgcactggctgcagcaaaaaccgggtcaggcaccgcgtccgtggatttatgcgacgatgaatctggctagcggtgttccggcgcgtttcagcggcagcggctctggcactgactacaccttgaccatcagctctctggaaccggaagatttcgctgtgtattactgccagcaatggagctccaacccaccggcgtttggtggtggcaccaaagttgagatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgc

SEQ ID NO: 164 - DNA, 1407 bp, synthetic construct
  source 1..1407: mol_type = other DNA
atgggctggtcttgtattattctgtttctggtcgcaactgctacaggcgtgcattctgaggttcaactagtagaatcaggtggagggttggtgcagccgggtggtagcctccgcctgagctgtgctgcgagcggtttcaccttcagctcctacggcatgagctgggttcgtcaggcaccgggcaaaggcctggaactggtgtccaccatcaacggttatggcgatacgtggtattacccggatagcgttaaaggccgttttaccattagccgtgacaactctaagaacaccttatatctgcaaatgaattcactgcgcgcagaggacactgccgtttattactgcgcgcgtgacagagactactctaattcctattactacgcgcacgattactggggtcaaggtacgttggtcaccgtgtcttcggccagcaccaagggcccttccgtgtttcccctggccccttgctcccggtccacatctgagagcaccgccgccctgggctgtctggtgaaggactacttcccagagcccgtgaccgtgagctggaacagcggcgccctgacaagcggcgtgcacacatttcccgccgtgctgcagagctccggcctgtactccctgtctagcgtggtgacagtgccttcctctagcctgggcaccaagacatatacctgtaacgtggaccacaagccaagcaataccaaggtggataagcgggtggagtctaagtacggccctccttgccctccatgtcctgctccagagtttctgggcggcccttccgtgttcctgtttccacccaaaccaaaggacacactgatgatctctagaacaccagaggtgacctgcgtggtggtggacgtgagccaggaggatcccgaggtgcagttcaactggtacgtggatggcgtggaggtgcacaatgccaagaccaagccaagagaggagcagtttaactctacatacagggtggtgagcgtgctgaccgtgctgcaccaggattggctcaacggcaaggagtataagtgcaaggtgtccaataagggcctgccctcctctatcgagaagacaatctctaaggctaagggccagccaagagagcctcaggtgtacaccctgcctccaagccaggaggagatgacaaagaaccaggtgtccctgacatgtctggtgaagggcttctatccctccgacatcgccgtggagtgggagtctaatggccagcctgagaacaattacaagaccacaccccctgtgctggactctgatggcagcttctttctgtattccaggctgaccgtggataagtctcggtggcaggagggcaacgtgttcagctgctctgtgatgcacgaagccctgcataatcactatactcagaaaagtctgtcactgtcactgggaaag

SEQ ID NO: 165 - DNA, 696 bp, synthetic construct; identical to SEQ ID NO: 157
  source 1..696: mol_type = other DNA

SEQ ID NO: 166 - DNA, 1407 bp, synthetic construct
  source 1..1407: mol_type = other DNA
atgggctggtcttgtattattctgtttctggtcgcaactgctacaggcgtgcattctgaggttcaactagtagaatcaggtggagggttggtgcagccgggtggtagcctccgcctgagctgtgctgcgagcggttacaccttcagctcctacggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactacggtaattcctattactacgcgcacgattactggggtcaaggtacgttggtcaccgtgtcttcggccagcaccaagggcccttccgtgtttcccctggccccttgctcccggtccacatctgagagcaccgccgccctgggctgtctggtgaaggactacttcccagagcccgtgaccgtgagctggaacagcggcgccctgacaagcggcgtgcacacatttcccgccgtgctgcagagctccggcctgtactccctgtctagcgtggtgacagtgccttcctctagcctgggcaccaagacatatacctgtaacgtggaccacaagccaagcaataccaaggtggataagcgggtggagtctaagtacggccctccttgccctccatgtcctgctccagagtttctgggcggcccttccgtgttcctgtttccacccaaaccaaaggacacactgatgatctctagaacaccagaggtgacctgcgtggtggtggacgtgagccaggaggatcccgaggtgcagttcaactggtacgtggatggcgtggaggtgcacaatgccaagaccaagccaagagaggagcagtttaactctacatacagggtggtgagcgtgctgaccgtgctgcaccaggattggctcaacggcaaggagtataagtgcaaggtgtccaataagggcctgccctcctctatcgagaagacaatctctaaggctaagggccagccaagagagcctcaggtgtacaccctgcctccaagccaggaggagatgacaaagaaccaggtgtccctgacatgtctggtgaagggcttctatccctccgacatcgccgtggagtgggagtctaatggccagcctgagaacaattacaagaccacaccccctgtgctggactctgatggcagcttctttctgtattccaggctgaccgtggataagtctcggtggcaggagggcaacgtgttcagctgctctgtgatgcacgaagccctgcataatcactatactcagaaaagtctgtcactgtcactgggaaag

SEQ ID NO: 167 - DNA, 696 bp, synthetic construct; identical to SEQ ID NO: 163
  source 1..696: mol_type = other DNA

SEQ ID NO: 168 - DNA, 1407 bp, synthetic construct
  source 1..1407: mol_type = other DNA
atgggctggtcttgtattattctgtttctggtcgcaactgctacaggcgtgcattctgaggttcaactagtagaatcaggtggagggttggtgcagccgggtggtagcctccgcctgagctgtgctgcgagcggtttcaccttcagctcctggggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactactctaattcctattactacgcgcacgattactggggtcaaggtacgttggtcaccgtgtcttcggccagcaccaagggcccttccgtgtttcccctggccccttgctcccggtccacatctgagagcaccgccgccctgggctgtctggtgaaggactacttcccagagcccgtgaccgtgagctggaacagcggcgccctgacaagcggcgtgcacacatttcccgccgtgctgcagagctccggcctgtactccctgtctagcgtggtgacagtgccttcctctagcctgggcaccaagacatatacctgtaacgtggaccacaagccaagcaataccaaggtggataagcgggtggagtctaagtacggccctccttgccctccatgtcctgctccagagtttctgggcggcccttccgtgttcctgtttccacccaaaccaaaggacacactgatgatctctagaacaccagaggtgacctgcgtggtggtggacgtgagccaggaggatcccgaggtgcagttcaactggtacgtggatggcgtggaggtgcacaatgccaagaccaagccaagagaggagcagtttaactctacatacagggtggtgagcgtgctgaccgtgctgcaccaggattggctcaacggcaaggagtataagtgcaaggtgtccaataagggcctgccctcctctatcgagaagacaatctctaaggctaagggccagccaagagagcctcaggtgtacaccctgcctccaagccaggaggagatgacaaagaaccaggtgtccctgacatgtctggtgaagggcttctatccctccgacatcgccgtggagtgggagtctaatggccagcctgagaacaattacaagaccacaccccctgtgctggactctgatggcagcttctttctgtattccaggctgaccgtggataagtctcggtggcaggagggcaacgtgttcagctgctctgtgatgcacgaagccctgcataatcactatactcagaaaagtctgtcactgtcactgggaaag

SEQ ID NO: 169 - DNA, 696 bp, synthetic construct; identical to SEQ ID NO: 149
  source 1..696: mol_type = other DNA

SEQ ID NO: 170 - DNA, 1407 bp, synthetic construct
  source 1..1407: mol_type = other DNA
atgggctggtcttgtattattctgtttctggtcgcaactgctacaggcgtgcattctgaggttcaactagtagaatcaggtggagggttggtgcagccgggtggtagcctccgcctgagctgtgctgcgagcggtttcaccttcagctcctacggcatgagctgggttcgtcaggcaccgggcaaaggcctggaactggtgtccaccatcaacggttatggcgatacgtggtattacccggatagcgttaaaggccgttttaccattagccgtgacaactctaagaacaccttatatctgcaaatgaattcactgcgcgcagaggacactgccgtttattactgcgcgcgtgacagagactacggtaattcctattactacgcgcacgattactggggtcaaggtacgttggtcaccgtgtcttcggccagcaccaagggcccttccgtgtttcccctggccccttgctcccggtccacatctgagagcaccgccgccctgggctgtctggtgaaggactacttcccagagcccgtgaccgtgagctggaacagcggcgccctgacaagcggcgtgcacacatttcccgccgtgctgcagagctccggcctgtactccctgtctagcgtggtgacagtgccttcctctagcctgggcaccaagacatatacctgtaacgtggaccacaagccaagcaataccaaggtggataagcgggtggagtctaagtacggccctccttgccctccatgtcctgctccagagtttctgggcggcccttccgtgttcctgtttccacccaaaccaaaggacacactgatgatctctagaacaccagaggtgacctgcgtggtggtggacgtgagccaggaggatcccgaggtgcagttcaactggtacgtggatggcgtggaggtgcacaatgccaagaccaagccaagagaggagcagtttaactctacatacagggtggtgagcgtgctgaccgtgctgcaccaggattggctcaacggcaaggagtataagtgcaaggtgtccaataagggcctgccctcctctatcgagaagacaatctctaaggctaagggccagccaagagagcctcaggtgtacaccctgcctccaagccaggaggagatgacaaagaaccaggtgtccctgacatgtctggtgaagggcttctatccctccgacatcgccgtggagtgggagtctaatggccagcctgagaacaattacaagaccacaccccctgtgctggactctgatggcagcttctttctgtattccaggctgaccgtggataagtctcggtggcaggagggcaacgtgttcagctgctctgtgatgcacgaagccctgcataatcactatactcagaaaagtctgtcactgtcactgggaaag

SEQ ID NO: 171 - DNA, 696 bp, synthetic construct; identical to SEQ ID NO: 159
  source 1..696: mol_type = other DNA

SEQ ID NO: 172 - DNA, 1407 bp, synthetic construct
  source 1..1407: mol_type = other DNA
atgggctggtcttgtattattctgtttctggtcgcaactgctacaggcgtgcattctgaggttcaactagtagaatcaggtggagggttggtgcagccgggtggtagcctccgcctgagctgtgctgcgagcggtttcaccttcagctcctacggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactacggtaattcctattactacgcgcacgattactggggtcaaggtacgttggtcaccgtgtcttcggccagcaccaagggcccttccgtgtttcccctggccccttgctcccggtccacatctgagagcaccgccgccctgggctgtctggtgaaggactacttcccagagcccgtgaccgtgagctggaacagcggcgccctgacaagcggcgtgcacacatttcccgccgtgctgcagagctccggcctgtactccctgtctagcgtggtgacagtgccttcctctagcctgggcaccaagacatatacctgtaacgtggaccacaagccaagcaataccaaggtggataagcgggtggagtctaagtacggccctccttgccctccatgtcctgctccagagtttctgggcggcccttccgtgttcctgtttccacccaaaccaaaggacacactgatgatctctagaacaccagaggtgacctgcgtggtggtggacgtgagccaggaggatcccgaggtgcagttcaactggtacgtggatggcgtggaggtgcacaatgccaagaccaagccaagagaggagcagtttaactctacatacagggtggtgagcgtgctgaccgtgctgcaccaggattggctcaacggcaaggagtataagtgcaaggtgtccaataagggcctgccctcctctatcgagaagacaatctctaaggctaagggccagccaagagagcctcaggtgtacaccctgcctccaagccaggaggagatgacaaagaaccaggtgtccctgacatgtctggtgaagggcttctatccctccgacatcgccgtggagtgggagtctaatggccagcctgagaacaattacaagaccacaccccctgtgctggactctgatggcagcttctttctgtattccaggctgaccgtggataagtctcggtggcaggagggcaacgtgttcagctgctctgtgatgcacgaagccctgcataatcactatactcagaaaagtctgtcactgtcactgggaaag

SEQ ID NO: 173 - DNA, 696 bp, synthetic construct
  source 1..696: mol_type = other DNA
atgggctggtcatgtattattctgtttctggtcgcaactgctacaggggtccatagtgaaatagtactaacacaatcacccgctactttgtcactctcgccgggtgagcgcgcgaccctgtcttgtcgtgccagctcctgggtgagctttatgcactggctgcagcaaaaaccgggtcaggcaccgcgtccgtggatttatgcgacgatgaatctggctagcggtgttccggcgcgtttcagcggcagcggctctggcactgactacaccttgaccatcagctctctggaaccggaagatttcgctgtgtattactgccagcaatggagctccaacccaccggcgtttggtggtggcaccaaagttgagatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgc

SEQ ID NO: 174 - DNA, 1407 bp, synthetic construct
  source 1..1407: mol_type = other DNA
atgggctggtcttgtattattctgtttctggtcgcaactgctacaggcgtgcattctgaggttcaactagtagaatcaggtggagggttggtgcagccgggtggtagcctccgcctgagctgtgctgcgagcggtttctggttcagctcctggggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactacactaattcctattactacgcgcacgattactggggtcaaggtacgttggtcaccgtgtcttcggccagcaccaagggcccttccgtgtttcccctggccccttgctcccggtccacatctgagagcaccgccgccctgggctgtctggtgaaggactacttcccagagcccgtgaccgtgagctggaacagcggcgccctgacaagcggcgtgcacacatttcccgccgtgctgcagagctccggcctgtactccctgtctagcgtggtgacagtgccttcctctagcctgggcaccaagacatatacctgtaacgtggaccacaagccaagcaataccaaggtggataagcgggtggagtctaagtacggccctccttgccctccatgtcctgctccagagtttctgggcggcccttccgtgttcctgtttccacccaaaccaaaggacacactgatgatctctagaacaccagaggtgacctgcgtggtggtggacgtgagccaggaggatcccgaggtgcagttcaactggtacgtggatggcgtggaggtgcacaatgccaagaccaagccaagagaggagcagtttaactctacatacagggtggtgagcgtgctgaccgtgctgcaccaggattggctcaacggcaaggagtataagtgcaaggtgtccaataagggcctgccctcctctatcgagaagacaatctctaaggctaagggccagccaagagagcctcaggtgtacaccctgcctccaagccaggaggagatgacaaagaaccaggtgtccctgacatgtctggtgaagggcttctatccctccgacatcgccgtggagtgggagtctaatggccagcctgagaacaattacaagaccacaccccctgtgctggactctgatggcagcttctttctgtattccaggctgaccgtggataagtctcggtggcaggagggcaacgtgttcagctgctctgtgatgcacgaagccctgcataatcactatactcagaaaagtctgtcactgtcactgggaaag

SEQ ID NO: 175 - DNA, 696 bp, synthetic construct
  source 1..696: mol_type = other DNA
atgggctggtcatgtattattctgtttctggtcgcaactgctacaggggtccatagtgaaatagtactaacacaatcacccgctactttgtcactctcgccgggtgagcgcgcgaccctgtcttgtcgtgccagctcctacgtgagctttatgcactggctgcagcaaaaaccgggtcaggcaccgcgtccgtggatttatgcgacgcagaatctggctagcggtgttccggcgcgtttcagcggcagcggctctggcactgactacaccttgaccatcagctctctggaaccggaagatttcgctgtgtattactgccagcaatggagctccaacccaccggcgtttggtggtggcaccaaagttgagatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgc

SEQ ID NO: 176 - DNA, 1407 bp, synthetic construct; identical to SEQ ID NO: 160
  source 1..1407: mol_type = other DNA

SEQ ID NO: 177 - DNA, 696 bp, synthetic construct; identical to SEQ ID NO: 163
  source 1..696: mol_type = other DNA

SEQ ID NO: 178 - DNA, 1407 bp, synthetic construct
  source 1..1407: mol_type = other DNA
atgggctggtcttgtattattctgtttctggtcgcaactgctacaggcgtgcattctgaggttcaactagtagaatcaggtggagggttggtgcagccgggtggtagcctccgcctgagctgtgctgcgagcggttactggttcagctcctggggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactactctaattcctattactacgcgcacgattactggggtcaaggtacgttggtcaccgtgtcttcggccagcaccaagggcccttccgtgtttcccctggccccttgctcccggtccacatctgagagcaccgccgccctgggctgtctggtgaaggactacttcccagagcccgtgaccgtgagctggaacagcggcgccctgacaagcggcgtgcacacatttcccgccgtgctgcagagctccggcctgtactccctgtctagcgtggtgacagtgccttcctctagcctgggcaccaagacatatacctgtaacgtggaccacaagccaagcaataccaaggtggataagcgggtggagtctaagtacggccctccttgccctccatgtcctgctccagagtttctgggcggcccttccgtgttcctgtttccacccaaaccaaaggacacactgatgatctctagaacaccagaggtgacctgcgtggtggtggacgtgagccaggaggatcccgaggtgcagttcaactggtacgtggatggcgtggaggtgcacaatgccaagaccaagccaagagaggagcagtttaactctacatacagggtggtgagcgtgctgaccgtgctgcaccaggattggctcaacggcaaggagtataagtgcaaggtgtccaataagggcctgccctcctctatcgagaagacaatctctaaggctaagggccagccaagagagcctcaggtgtacaccctgcctccaagccaggaggagatgacaaagaaccaggtgtccctgacatgtctggtgaagggcttctatccctccgacatcgccgtggagtgggagtctaatggccagcctgagaacaattacaagaccacaccccctgtgctggactctgatggcagcttctttctgtattccaggctgaccgtggataagtctcggtggcaggagggcaacgtgttcagctgctctgtgatgcacgaagccctgcataatcactatactcagaaaagtctgtcactgtcactgggaaag

SEQ ID NO: 179 - DNA, 696 bp, synthetic construct; identical to SEQ ID NO: 163
  source 1..696: mol_type = other DNA

SEQ ID NO: 180 - DNA, 1407 bp, synthetic construct
  source 1..1407: mol_type = other DNA
atgggctggtcttgtattattctgtttctggtcgcaactgctacaggcgtgcattctgaggttcaactagtagaatcaggtggagggttggtgcagccgggtggtagcctccgcctgagctgtgctgcgagcggtttctggttcagctcctacggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactacactaattcctattactacgcgcacgattactggggtcaaggtacgttggtcaccgtgtcttcggccagcaccaagggcccttccgtgtttcccctggccccttgctcccggtccacatctgagagcaccgccgccctgggctgtctggtgaaggactacttcccagagcccgtgaccgtgagctggaacagcggcgccctgacaagcggcgtgcacacatttcccgccgtgctgcagagctccggcctgtactccctgtctagcgtggtgacagtgccttcctctagcctgggcaccaagacatatacctgtaacgtggaccacaagccaagcaataccaaggtggataagcgggtggagtctaagtacggccctccttgccctccatgtcctgctccagagtttctgggcggcccttccgtgttcctgtttccacccaaaccaaaggacacactgatgatctctagaacaccagaggtgacctgcgtggtggtggacgtgagccaggaggatcccgaggtgcagttcaactggtacgtggatggcgtggaggtgcacaatgccaagaccaagccaagagaggagcagtttaactctacatacagggtggtgagcgtgctgaccgtgctgcaccaggattggctcaacggcaaggagtataagtgcaaggtgtccaataagggcctgccctcctctatcgagaagacaatctctaaggctaagggccagccaagagagcctcaggtgtacaccctgcctccaagccaggaggagatgacaaagaaccaggtgtccctgacatgtctggtgaagggcttctatccctccgacatcgccgtggagtgggagtctaatggccagcctgagaacaattacaagaccacaccccctgtgctggactctgatggcagcttctttctgtattccaggctgaccgtggataagtctcggtggcaggagggcaacgtgttcagctgctctgtgatgcacgaagccctgcataatcactatactcagaaaagtctgtcactgtcactgggaaag

SEQ ID NO: 181 - DNA, 696 bp, synthetic construct
  source 1..696: mol_type = other DNA
atgggctggtcatgtattattctgtttctggtcgcaactgctacaggggtccatagtgaaatagtactaacacaatcacccgctactttgtcactctcgccgggtgagcgcgcgaccctgtcttgtcgtgccagctcctccgtgagctttatgcactggctgcagcaaaaaccgggtcaggcaccgcgtccgtggatttatgcgacggccaatctggctagcggtgttccggcgcgtttcagcggcagcggctctggcactgactacaccttgaccatcagctctctggaaccggaagatttcgctgtgtattactgccagcaatggagctccaacccaccggcgtttggtggtggcaccaaagttgagatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgc

SEQ ID NO: 182 - DNA, 1407 bp, synthetic construct; identical to SEQ ID NO: 166
  source 1..1407: mol_type = other DNA

SEQ ID NO: 183 - DNA, 696 bp, synthetic construct; identical to SEQ ID NO: 173
  source 1..696: mol_type = other DNA

SEQ ID NO: 184 - DNA, 1407 bp, synthetic construct; identical to SEQ ID NO: 156
  source 1..1407: mol_type = other DNA

SEQ ID NO: 185 - DNA, 696 bp, synthetic construct; identical to SEQ ID NO: 163
  source 1..696: mol_type = other DNA

SEQ ID NO: 186 - DNA, 1407 bp, synthetic construct; identical to SEQ ID NO: 150
  source 1..1407: mol_type = other DNA

SEQ ID NO: 187 - DNA, 696 bp, synthetic construct
  source 1..696: mol_type = other DNA
atgggctggtcatgtattattctgtttctggtcgcaactgctacaggggtccatagtgaaatagtactaacacaatcacccgctactttgtcactctcgccgggtgagcgcgcgaccctgtcttgtcgtgccagctcctacgtgagctttatgcactggctgcagcaaaaaccgggtcaggcaccgcgtccgtggatttatgcgacgagcaatctggctagcggtgttccggcgcgtttcagcggcagcggctctggcactgactacaccttgaccatcagctctctggaaccggaagatttcgctgtgtattactgccagcaatggagctccaacccaccggcgtttggtggtggcaccaaagttgagatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgc

SEQ ID NO: 188 - DNA, 1407 bp, synthetic construct
  source 1..1407: mol_type = other DNA
atgggctggtcttgtattattctgtttctggtcgcaactgctacaggcgtgcattctgaggttcaactagtagaatcaggtggagggttggtgcagccgggtggtagcctccgcctgagctgtgctgcgagcggtttcaccttcagctcctacggcatgagctgggttcgtcaggcaccgggcaaaggcctggaactggtgtccaccatcaacggttatggcgatacgacctattacccggatagcgttaaaggccgttttaccattagccgtgacaactctaagaacaccttatatctgcaaatgaattcactgcgcgcagaggacactgccgtttattactgcgcgcgtgacagagactacggtaattcctattactacgcgctggattactggggtcaaggtacgttggtcaccgtgtcttcggccagcaccaagggcccttccgtgtttcccctggccccttgctcccggtccacatctgagagcaccgccgccctgggctgtctggtgaaggactacttcccagagcccgtgaccgtgagctggaacagcggcgccctgacaagcggcgtgcacacatttcccgccgtgctgcagagctccggcctgtactccctgtctagcgtggtgacagtgccttcctctagcctgggcaccaagacatatacctgtaacgtggaccacaagccaagcaataccaaggtggataagcgggtggagtctaagtacggccctccttgccctccatgtcctgctccagagtttctgggcggcccttccgtgttcctgtttccacccaaaccaaaggacacactgatgatctctagaacaccagaggtgacctgcgtggtggtggacgtgagccaggaggatcccgaggtgcagttcaactggtacgtggatggcgtggaggtgcacaatgccaagaccaagccaagagaggagcagtttaactctacatacagggtggtgagcgtgctgaccgtgctgcaccaggattggctcaacggcaaggagtataagtgcaaggtgtccaataagggcctgccctcctctatcgagaagacaatctctaaggctaagggccagccaagagagcctcaggtgtacaccctgcctccaagccaggaggagatgacaaagaaccaggtgtccctgacatgtctggtgaagggcttctatccctccgacatcgccgtggagtgggagtctaatggccagcctgagaacaattacaagaccacaccccctgtgctggactctgatggcagcttctttctgtattccaggctgaccgtggataagtctcggtggcaggagggcaacgtgttcagctgctctgtgatgcacgaagccctgcataatcactatactcagaaaagtctgtcactgtcactgggaaag

SEQ ID NO: 189 - DNA, 696 bp, synthetic construct
  source 1..696: mol_type = other DNA
atgggctggtcatgtattattctgtttctggtcgcaactgctacaggggtccatagtgaaatagtactaacacaatcacccgctactttgtcactctcgccgggtgagcgcgcgaccctgtcttgtcgtgccagctcctccgtgagctttatgcactggctgcagcaaaaaccgggtcaggcaccgcgtccgtggatttatgcgacgagcaatctggctagcggtgttccggcgcgtttcagcggcagcggctctggcactgactacaccttgaccatcagctctctggaaccggaagatttcgctgtgtattactgccagcaatggagctccaacccaccggcgtttggtggtggcaccaaagttgagatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgc